(19) United States
(12) Patent Application Publication
(10) Pub. No.: US 2024/0254089 A1
(43) Pub. Date: Aug. 1, 2024

(54) MODULATORS OF MAS-RELATED G-PROTEIN RECEPTOR X4 AND RELATED PRODUCTS AND METHODS

(71) Applicant: ESCIENT PHARMACEUTICALS, INC., San Diego, CA (US)

(72) Inventors: Brandon SELFRIDGE, San Diego, CA (US); Liming HUANG, San Diego, CA (US); Adam YEAGER, San Diego, CA (US); Marcos SAINZ, San Diego, CA (US); Marion LANIER, San Diego, CA (US); Esther MARTINBOROUGH, San Diego, CA (US); Marcus BOEHM, San Diego, CA (US)

(21) Appl. No.: 18/245,700

(22) PCT Filed: Sep. 16, 2021
(86) PCT No.: PCT/US2021/050706

§ 371 Date: Mar. 16, 2023

Related U.S. Application Data
(60) Provisional application No. 63/079,870, filed on Sep. 17, 2020.

(51) Int. Cl. C07D 231/56; A61K 31/416; A61K 31/4192; A61K 31/4196; A61K 31/422; A61K 31/423; A61K 31/4245; A61K 31/427; A61K 31/428; A61K 31/429; A61K 31/4355; A61K 31/437; A61K 31/4439; A61K 31/4709; A61K 31/497; A61K 31/506; A61K 31/519; A61K 31/5377; A61K 31/55; C07D 401/12; C07D 403/12; C07D 405/14; C07D 407/12; C07D 409/12; C07D 413/12; C07D 417/12; C07D 471/04; C07D 487/04; C07D 513/04

(52) U.S. Cl. CPC C07D 231/56; A61K 31/416; A61K 31/4192; A61K 31/4196; A61K 31/422; A61K 31/423; A61K 31/4245; A61K 31/427; A61K 31/428; A61K 31/429; A61K 31/4355; A61K 31/437; A61K 31/4439; A61K 31/4709; A61K 31/497; A61K 31/506; A61K 31/519; A61K 31/5377; A61K 31/55; C07D 401/12; C07D 403/12; C07D 405/14; C07D 407/12; C07D 409/12; C07D 413/12; C07D 417/12; C07D 471/04; C07D 487/04; C07D 513/04

(57) ABSTRACT

Methods are provided for modulating MRGPR X4 generally, or for treating a MRGPR X4-dependent condition more specifically, by contacting MRGPR X4 or administering to a subject in need thereof, respectively, an effective amount of a compound having structure (I): or a pharmaceutically acceptable salt, isomer, hydrate, solvate or isotope thereof, wherein E, Q, W, Z, R¹, R², R³, and R⁴ are as defined herein. Pharmaceutical compositions containing such compounds, as well as compounds themselves, are also provided.

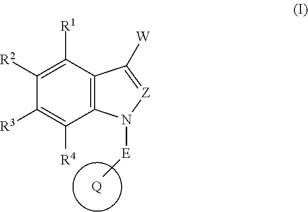

### BACKGROUND

### Technical Field

The invention relates to modulators of the Mas-related G-protein coupled receptor X4, to products containing the same, as well as to methods of their use and preparation.

### Description of the Related Art

Mas-related G protein receptors (MRGPRs) are a group of orphan receptors with limited expression in very specialized tissues. Very little is known about the function of most of these receptors. There are eight related receptors in this class expressed in humans, only four of which have readily identifiable orthologs in other species (i.e., MRGPR D, E, F and G). The other four receptors (MRGPR X1, X2, X3 and X4) have no counterpart, based on homology, in species other than human. Considerable differences exist in the MRGPR family of receptors between humans and non-clinical species (18 genes and pseudogenes exist in humans and ˜50 in mice), resulting in considerable challenges to both pharmacodynamic and on-target safety characterizations. Mouse MRGPRa1 and Monkey MRGPRX3-like or MRGPRX8 receptors are proposed to be putative orthologs of the human MRGPRX4 given that they are activated by bilirubin. However, these primate receptors only are likely partial functional orthologs of the human MRGPRX4 receptor as they do not respond to all the human MRGPRX4 agonists.

### BRIEF SUMMARY

This invention is based, in part, on the identification that functionally in mice MRGPR A1 corresponds, at least in part, to the human MRGPR X4. These receptors mediate disorders including chronic itch (e.g., pruritus), inflammation disorders, autoimmunity, skin disorders, cardiovascular disease, lung inflammation/COPD, and adverse skin reactions to drugs. More specifically, both MRGPR A1 and MRGPR X4 are expressed in sensory neurons, skin melanocytes, dendritic cells, polymorphonuclear cells, macrophages, bronchial epithelial cells, lung smooth muscle and dorsal root ganglia. It has now been identified that both MRGPR A1 and MRGPR X4 are receptors for (or sensitive to activation by) circulating bilirubin and its metabolites, and thus are important for itch sensation in conditions of elevated bilirubin such as cholestatic pruritus. In addition, MRGPR X4 is activated by multiple additional components of bile including bile acids and metabolites thereof and heme metabolites including bilirubin and urobilin. Bile acids and bilirubin are highly elevated in cholestatic pruritus while urobilin, which is a potent mediator of itch induction in a mouse model, and thus may be important for itch sensation in conditions of elevated urobilin such as uremic pruritus. Furthermore, MRGPR X4 is a receptor for urobilin, which is a potent mediator of itch induction in a mouse model, and thus may be important for itch sensation in conditions of elevated urobilin such as uremic pruritus. Thus, modulating MRGPR X4 allows for treatment of autoimmune diseases such as psoriasis, multiple sclerosis, Steven Johnson's Syndrome, and other chronic itch conditions as explained in more detail below.

Accordingly, in an embodiment, methods are provided for modulating a MRGPR X4 by contacting the MRGPR X4 with an effective amount of a compound having structure (I):

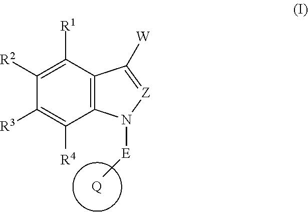

or a pharmaceutically acceptable salt, isomer, hydrate, solvate or isotope thereof, wherein E, Q, W, Z, R¹, R², R³, and R⁴ are as defined herein.

In another embodiment, methods are provided for treating an MRGPR X4-dependent condition by administering to a subject in need thereof an effective amount of a compound having structure (I), or a pharmaceutically acceptable salt, isomer, hydrate, solvate or isotope thereof.

In more specific embodiments, the MRGPR X4-dependent condition is one or more of an itch associated condition, a pain associated condition, an inflammation-associated condition, or an autoimmune disorder.

In one embodiment, the methods of treating the MRGPR X4-dependent condition are provided which comprise administering an effective amount of a compound of structure (I) with formula (IA), (IB), (IC), (ID), (IE), (IF), (IG) or (IH) as defined herein or a pharmaceutically acceptable salt, isomer, hydrate, solvate or isotope thereof.

In yet another embodiment, pharmaceutical compositions are provided comprising a carrier or excipient and a compound having structure (I), or a pharmaceutically acceptable salt, isomer, hydrate, solvate or isotope thereof.

In specific embodiments, pharmaceutical compositions are provided comprising substructures of structure (I) with formula (IA), (IB), (IC), (ID), (IE), (IF), (IG) or (IH) as defined herein or a pharmaceutically acceptable salt, isomer, hydrate, solvate or isotope thereof.

In yet other embodiments, compounds are provided having formula (IA), (IB), (IC), (ID), (IE), (IF), (IG) or (IH) as defined herein, or a pharmaceutically acceptable salt, isomer, hydrate, solvate or isotope thereof.

In another embodiment, compounds are provided having one or more of the structures disclosed herein, or a pharmaceutically acceptable salt, isomer, hydrate, solvate or isotope thereof.

### DETAILED DESCRIPTION

As mentioned above, the invention relates to modulators of the MRGPR X4, to products containing the same, as well as to methods of their use and preparation. This invention is based, in part, on the identification that in mice MRGPR A1 functionally corresponds to the human MRGPR X4. These receptors mediate disorders including chronic and intermittent itch (e.g., pruritus), inflammation disorders, autoimmunity, skin disorders, and adverse skin reactions to drugs and infectious diseases. More specifically, both MRGPR A1 and MRGPR X4 are expressed in sensory neurons and dorsal root ganglia. It has now been identified that both MRGPR A1 and MRGPR X4 are receptors for (or sensitive to activation by) circulating bilirubin and its metabolites, and thus are important for itch sensation in conditions of elevated bilirubin such as cholestatic pruritus and end-stage renal failure. In addition, MRGPR X4 is also activated by bile acids, which are also elevated in cholestatic pruritus. Furthermore, urobilin, an oxidative product of the heme metabolite urobilinogen solely excreted by the kidney, is a potent agonist of MRGPR X4 and pruritogen, and thus may be important for itch sensation in conditions of elevated urobilin such as uremic pruritus, kidney disease and end-stage renal failure. Thus, modulating MRGPR X4 allows for treatment of autoimmune diseases such as psoriasis, multiple sclerosis, Steven Johnson's Syndrome, atopic disorders such as atopic dermatitis and other chronic itch conditions as explained in more detail below.

MRGPRs appear to be sensory receptors that recognize their external environment to exogenous or endogenous signals/chemicals. These receptors likely respond to multiple chemical ligands/agonists. For example, MRGPR X4 recognizes bilirubin, bile acids, and urobilin as agonist signals. In certain embodiments, molecules of this invention modulate MRGPR X4 by functioning as inverse agonists that are capable of blocking multiple chemical entities, and/or as competitive antagonists that can specifically block individual ligands. In one embodiment, such modulations are selective against other MRGPRs, such as MRGPR X1, X2 and/or X3.

Accordingly, in one embodiment a method is provided for modulating a Mas-Related G-Protein Receptor (MRGPR) X4 by contacting the MRGPR X4 with an effective amount of a compound having structure (I):

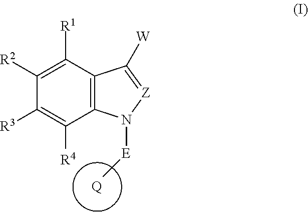

or a pharmaceutically acceptable salt, isomer, hydrate, solvate or isotope thereof, wherein:
W is

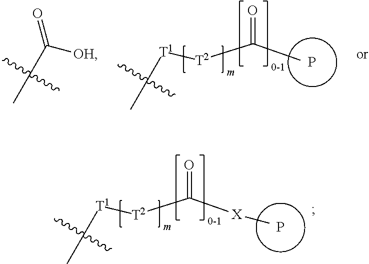

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or (C(H)R^{e})ₙ—X²—(C(R^{t})(R^{t′})ₚ;
Z is C or N;
X is —X¹—(C(R^{t})(R^{t′})—;
X¹ is —O—, —NH—, —N(H)C(O)—, —C(O)NH— or —S(═O)₂—;
X² is —O—, —C(═O)—, —N(H)C(O)— or —C(O)NH—;
R^{p} and R^{q} are at each occurrence independently H, —OH, —NH₂, —N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH, or C₁-C₄ alkyl;
R^{t} is at each occurrence independently H, C₁-C₄ alkyl, or cycloalkyl;
R^{t′} is at each occurrence independently H, OH, C₁-C₄ alkyl, cycloalkyl or R^{t} and R^{t′} together with the atom to which they are bonded form a ring;
R¹, R², R³, and R⁴ are at each occurrence independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

As used herein, the following terms have the meaning defined below, unless the context indicates otherwise.

“Modulating” MRGPR X4 means that the compound interacts with the MRGPR X4 in a manner such that it functions as an inverse agonist to the receptor, and/or as a competitive antagonist to the receptor. In one embodiment, such modulation is partially or fully selective against other MRGPRs, such as MRGPR X1, X2 and/or X3.

“MRGPR” refers to one or more of the Mas-related G protein coupled receptors, which are a group of orphan receptors with limited expression in very specialized tissues (e.g., in sensory neurons and dorsal root ganglia) and barrier tissues. There are eight related receptors in this class expressed in humans, only 4 of which have readily identifiable orthologs in other species (i.e., MRGPR D, E, F and G). The other four receptors (MRGPR X1, X2, X3 and X4) have no counterpart, based on homology, in non-human species.

“Effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

“Alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, see-butyl, t-butyl, neopentyl, isopentyl, and 2,2-dimethylpropyl groups. An unsaturated alkyl includes alkenyl and alkynyl as defined below.

“Alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated hydrocarbons that contain at least one carbon-carbon double bond. Examples of lower alkenyl groups include, but are not limited to, vinyl, propenyl, butenyl, pentenyl, and hexenyl.

“Alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated hydrocarbons that contain at least one carbon-carbon triple bond. Examples of alkynyl groups include, but are not limited to, ethynyl, propynyl, butynyl, pentynyl, and hexynyl.

“Halo” or “halogen” refers to fluorine, chlorine, bromine, and iodine.

“Hydroxy” refers to —OH.

“Oxo” refers to ═O.

“Cyano” refers to —CN.

Amino refers to —NH₂, -NHalkyl or N(alkyl)₂, wherein alkyl is as defined above. Examples of amino include, but are not limited to —NH₂, —NHCH₃, —N(CH₃)₂, and the like.

“Haloalkyl” refers to alkyl as defined above with one or more hydrogen atoms replaced with halogen. Examples of lower haloalkyl groups include, but are not limited to, —CF₃, —CHF₂, and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, see-butoxy, tert-butoxy, and the like.

“Haloalkoxy” refers to haloalkyl as defined above joined by way of an oxygen atom (i.e., —O-haloalkyl). Examples of lower haloalkoxy groups include, but are not limited to, —OCF₃, and the like.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Carbocycle” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined above. In another embodiment, carbocycle includes aryl as defined above.

“Heterocycle” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

“Isomer” is used herein to encompass all chiral, diastereomeric or racemic forms of a structure (also referred to as a stereoisomer, as opposed to a structural or positional isomer), unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of nonsuperimposable-isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure by weight. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound of structure (I) includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine-19 is longest-lived. Thus, an isotope of a compound having the structure of structure (I) includes, but not limited to, compounds of structure (I) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, Phydroxybutyric, salicylic, -galactaric, and galacturonic acid.

Although pharmaceutically unacceptable salts are not generally useful as medicaments, such salts may be useful, for example as intermediates in the synthesis of compounds having the structure of Formula I, for example in their purification by recrystallization.

One embodiment provides a method for treating an MRGPR X4-dependent condition by administering to a subject in need thereof an effective amount of a compound having structure (I):

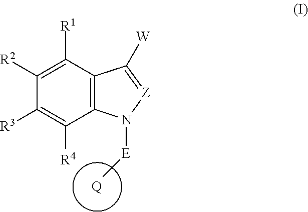

or a pharmaceutically acceptable salt, isomer, hydrate, solvate or isotope thereof, wherein:
W is

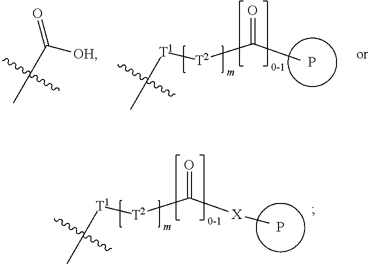

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ;
Z is C or N;
X is —X¹—(C(R^{t})(R^{t′})—;
X¹ is —O—, —NH—, —N(H)C(O)—, —C(O)NH— or —S(═O)₂—;
X² is —O—, —C(═O)—, —N(H)C(O)— or —C(O)NH—;
R^{p} and R^{q} are at each occurrence independently H, —OH, —NH₂, —N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl; R^{t′} is at each occurrence independently H, OH, C₁-C₄ alkyl, cycloalkyl or R^{t} and R^{t′} together with the atom to which they are bonded form a ring;
R¹, R², R³, and R⁴ are at each occurrence independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

As used herein, the phrase “MRGPR X4-dependent condition” means a condition where the activation, over sensitization, or desensitization of MRGPR X4 by a natural or synthetic ligand initiates, mediates, sustains, or augments a pathological condition. For example, it is known that some itch or pain sensations are caused by elevated bilirubin and its metabolites or bile acids in patients suffering from pruritus, atopic or other autoimmune or inflammatory diseases. It has been found that MRGPR X4 is sensitive to (or activated by) bilirubin and its metabolites, including urobilin, or bile acids. Without limited by theory, it is to be understood that by modulating MRGPR X4, the itch or pain sensations can be eased.

In some embodiments, the MRGPR X4-dependent condition is a condition that is caused by the activation of MRGPR X4 by a bile acid. As used herein, the term “bile acid” includes primary bile acids (e.g., cholic acid, chenodeoxycholic acid), conjugated bile acids, also referred to as bile salts (e.g., taurocholic acid, glycocholic acid, taurochenodeoxycholic acid, glycochenodeoxycholic acid), secondary bile acids (e.g., deoxycholic acid, lithocholic acid), and bile acid analogs. In some embodiments, a bile acid analog is a farnesoid X-receptor (FXR) agonist. Thus, the compounds of the present disclosure may be used for treating an MRGPR X4 dependent condition caused by activation of MRGPR X4 by a bile acid and that would benefit from modulating MRGPR X4.

In some embodiments, the MRGPR X4-dependent condition is an itch associated condition, a pain associated condition, an autoimmune condition, or an autoimmune or inflammatory disorder.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; cholestatic pruritus; contact dermatitis; Allergic blepharitis; Anemia; Atopic dermatitis; Bullous pemphigoid; Candidiasis; Chicken pox; Cholestasis; end-stage renal failure; hemodialysis; Contact dermatitis, Atopic Dermatitis; Dermatitis herpetiformis; Diabetes; Drug allergy, Dry skin; Dyshidrotic dermatitis; Ectopic eczema; Erythrasma; Folliculitis; Fungal skin infection; Hemorrhoids; Herpes; HIV infection; Hodgkin's disease; Hyperthyroidism; Iron deficiency anemia; Kidney disease; Leukemia, porphyrias; Liver disease, including primary biliary cholangitis, primary sclerosing cholangitis, Alagille syndrome, Progressive familial intrahepatic cholestasis, Intrahepatic cholestasis of pregnancy, nonalcoholic steatohepatitis (NASH), nonalcoholic fatty liver disease (NAFLD), biliary atresia, chronic B hepatitis, drug-chronic viral hepatitis, induced liver injury (DILI), liver fibrosis, cholestatic liver disease, and alcoholic liver disease; Lymphoma; Malignancy; Multiple myeloma; Neurodermatitis; Onchocerciasis; Paget's disease; Pediculosis; Polycythemia rubra vera; Lichen Planus; Lichen Sclerosis; Pruritus ani; Pseudorabies; Psoriasis; Rectal prolapse; Scabies; Schistosomiasis; Scleroderma, Severe stress, Stasia dermatitis; Swimmer's itch; Thyroid disease; Tinea cruris; Uremic Pruritus; Rosacea; Cutaneous amyloidosis; Scleroderma; Acne; wound healing; ocular itch; and Urticaria.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Collapsed Lung (Pneumothorax), Complex Regional Pain Syndrome (RSD), Corneal Neuropathic Pain, Crohn's Disease, Degenerative Disc Disease, Dercum's Disease, Dermatomyositis, Diabetic Peripheral Neuropathy (DPN), Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Osteoarthritis, Paget's Disease, Parsonage Turner Syndrome, Pelvic Pain, Peripheral Neuropathy, Phantom Limb Pain, Pinched Nerve, Polycystic Kidney Disease, Polymyalgia Rhuematica, Polymyositis, Porphyria, Post Herniorraphy Pain Syndrome, Post Mastectomy, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain and Vulvodynia.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues. Thus, in one embodiment, the method of present invention is provided to treat an autoimmune disorder, such as chronic inflammation, Multiple Sclerosis, Steven Johnson's Syndrome, appendicitis, bursitis, colitis, cystitis, dermatitis, phlebitis, reflex sympathetic dystrophy/complex regional pain syndrome (rsd/crps), rhinitis, tendonitis, tonsillitis, acne vulgaris, reactive airway disorder, asthma, airway infection, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, colitis, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, cardiovascular disease, and vasculitis.

As used herein, the term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal), such as a human, as well as to animals typically treated in the veterinary context, such as companion animals, livestock, zoo animals or equines. A subject to be treated according to the methods described herein may be one who has been diagnosed with a MRGPR X4 dependent condition, such as an itch associated condition, a pain associated condition, or an autoimmune disorder. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.”

A subject may refer to an adult or pediatric subject. The Federal Food, Drug, and Cosmetic Act defines “pediatric” as a subject aged 21 or younger at the time of their diagnosis or treatment. Pediatric subpopulations are further characterized as: (i) neonates from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

In some embodiments, the subject is a pediatric subject that has Progressive familial intrahepatic cholestasis, Alagille Syndrome, or Biliary Atresia.

In another embodiment, the method of treating a subject having a MRGPR X4-dependent condition (e.g., an itch associated condition, a pain associated condition, an autoimmune condition, or an autoimmune disorder) described herein further comprises administering to the subject a pharmaceutically effective amount of a second therapeutic agent. In one embodiment, the itch associated condition is a liver disease. In one embodiment, the second therapeutic agent is a liver disease therapeutic agent. In one embodiment, the liver disease therapeutic agent is ursodeoxycholic acid (UDCA), norUrsodeoxycholic acid, cholestyramine, stanozolol, naltrexone, rifampicin, Alisol B 23-acetate (AB23A), curcumin, dihydroartemisinin, fenofibrate, bezafibrate, metronidazole, methotrexate, colchicine, metformin, betaine, glucagon, naltrexone, a farnesoid X-receptor (FXR) agonist, a peroxisome proliferator-activated receptor (PPAR) agonist, a thyroid hormone receptor beta (TRO) agonist, or any combination thereof.

Examples of FXR agonists that may be used in the methods described herein include obeticholic acid, Turofexorate isopropyl (WAY-362450), 3-(2,6-dichlorophenyl)-4-(3′-carboxy-2-chlorostilben-4-yl)oxymethyl-5-isopropylisoxazole (GW4064), PX20606 (PX-102), PX-101, INT-767, INT-787, TERN-101, altenusin, tropifexor (LJN452), nidufexor, turofexorate isopropyl, fexaramine, silymarin, silybin, hedragonic acid, cafestol, Cilofexor (GS-9674 or Px-104), EDP-305, BAR704, BAR502, EYP-001, RDX-023, AGN-242266, HPG-1860, MET-409, AGN-242256, EP-024297, IOT-022, M-480, INV-33, RDX023-02, or any combination thereof. In one embodiment, a FXR agonist is a bile acid or analog thereof (e.g., obeticholic acid, INT-767, INT-787, turofexorate isopropyl (WAY-362450), BAR502, hedragonic acid or BAR704) or a non-bile acid agonist (e.g., EDP-305, tropifexor, nidufexor, cilofexor, GW4064, Turofexorate isopropyl, fexaramine, PX20606 (PX-102), TERN-101, altenusin, silymarin, silybin, hedragonic acid, BAR502, EYP-001, RDX023-2, AGN-242266, HPG-1860, MET-409, EP-024297, M-480, or cafestol). In one embodiment, a PPAR agonist is a PPAR-alpha agonist, a PPAR-gamma agonist, a PPAR-delta agonist, a PPAR-alpha/gamma dual agonist, a PPAR alpha/delta dual agonist, a PPAR gamma/delta dual agonist, or PPAR alpha/gamma/delta pan agonist.

Examples of PPAR alpha agonists that may be used in the methods described herein include fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, and SRI 0171.

Examples of PPAR gamma agonists that may be used in the methods described herein include rosiglitazone, pioglitazone, deuterium-stabilized R-pioglitazone, efatutazone, ATx08-001, OMS-405, CHS-131, THR-0921, SER-150-DN, KDT-501, GED-0507-34-Levo, CLC-3001, and ALL-4.

Examples of PPAR delta agonists that may be used in the methods described herein include GW501516 (endurabol or ({4-[({4-methyl-2-[4-(trifluoromethyl)phenyl]-1,3-thiazol-5-yl}methyl)sulfanyl]-2-methylphenoxy} acetic acid)), MBX8025 (seladelpar or {2-methyl-4-[5-methyl-2-(4-trifluoromethyl-phenyl)-2H-[1,2,3]triazol-4-ylmethylsylfanyl]-phenoxy}-acetic acid), GW0742 ([4-[[[2-[3-fluoro-4-(trifluoromethyl)phenyl]-4-methyl-5-thiazolyl]methyl]thio]-2-methyl phenoxy] acetic acid), L165041, HPP-593, and NCP-1046.

Examples of PPAR alpha/gamma agonists that may be used in the methods described herein include saroglitazar, aleglitazar, muraglitazar, tesaglitazar, and DSP-8658.

Examples of PPAR alpha/delta agonists that may be used in the methods described herein include elafibranor and T913659.

Examples of PPAR gamma/delta agonists that may be used in the methods described herein include a conjugated linoleic acid (CLA) and T3D-959.

Examples of PPAR alpha/gamma/delta agonists that may be used in the methods described herein include IVA337 (lanifibranor), TTA (tetradecylthioacetic acid), bavachinin, GW4148, GW9135, bezafibrate, lobeglitazone, 2-(4-(5,6-methylenedioxybenzo[d]thiazol-2-yl)-2-methylphenoxy)-2-methylpropanoic acid (MHY2013), and CS038.

Examples of thyroid hormone receptor beta agonists that may be used in the methods described herein include sobetirome, eprotirome, GC-24, MGL-3196, MGL-3745, VK-2809, KB141 [3,5-dichloro-4-(4-hydroxy-3-isopropylphenoxy) phenylacetic acid], and MB07811 (2R,4S)-4-(3-chlorophenyl)-2-[(3,5-dimethyl-4-(4′-hydroxy-3′-isopropylbenzyl)phenoxy)methyl]-2-oxido-[1,3,2]-dioxaphosphonane).

The second therapeutic agent may be administered simultaneously, separately, or sequentially with the compounds of the present disclosure. If administered simultaneously, the second therapeutic agent and compound of the present disclosure may be administered in separate dosage forms or in the same dosage form.

In another embodiment, a method of treating a subject having an itch associated condition is provided, the method comprising administering to the subject a pharmaceutically effective amount of a compound having structure (I) or pharmaceutically acceptable salt or stereoisomer thereof, or a pharmaceutical composition thereof. In one embodiment, the itch associated condition is cholestatic pruritus, uremic pruritus, atopic dermatitis, dry skin, psoriasis, contact dermatitis, or eczema.

Another embodiment provides a method for treating an MRGPR X4-dependent condition by administering to a subject in need thereof an effective amount of a compound having formula (IA):

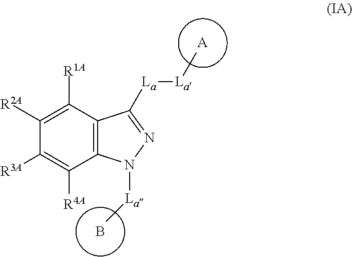

or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{a};
B is C₆-C₁₀ aryl, C₃-C₁₀ cycloalkyl, 3-10 membered monocyclic or bicyclic heterocyclyl, or 5-10 membered monocyclic or bicyclic heteroaryl, each of which is optionally substituted with one or more R^{a′};
Lₐ is —C(O)NH—, or —N(H)C(O)—;
L_{a′} is a bond, —CH₂C(O)— or —(C(R^{2a})(R^{2a′})₂—.
L_{a″} is —(C(R^{3a})(R^{3a′}))ₙ—;
n is 1 or 2;
R^{a} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{3a} is at each occurrence independently H, OH or C₁-C₄ alkyl;
R^{3a′} is at each occurrence independently H, OH or C₁-C₄ alkyl;
R^{2a} is at each occurrence independently H or C₁-C₄ alkyl;
R^{2a′} is at each occurrence H;
R^{a″} is at each occurrence independently H, —OH, —NH₂, halo, —CO₂H, —C(O)Me-, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl; and
R^{1A}, R^{2A}, R^{3A}, and R^{4A} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl.

Yet another embodiment provides a method for treating an MRGPR X4-dependent condition by administering to a subject in need thereof an effective amount of a compound having formula (IB):

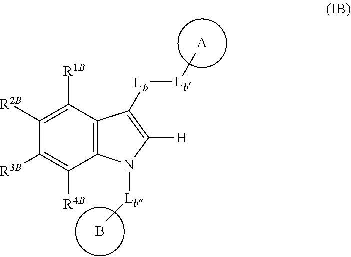

or a pharmaceutically acceptable salt, isomer, hydrate, solvate or isotope thereof, wherein:
A is C₆-C₁₀ aryl, C₃-C₁ cycloalkyl, 7-10 membered monocyclic or bicyclic heterocyclyl, 5-10 membered monocyclic or bicyclic heteroaryl, each of which is optionally substituted with one or more R^{b};
B is phenyl or heterocyclyl, optionally substituted with one or more R^{b′};
L_{b} is —C(O)NH—;
L_{b′} is —(C(R^{2b})(R^{2b′}))—, —(C(R^{2b})(R^{2b′}))₂— or —(C(R^{2b})(R^{2b′}))₃—
L_{b″} is —(CH₂)ₙ—;
n is 1 or 2;
R^{b} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2b} is at each occurrence independently H, C₁-C₄ alkyl or cycloalkyl;
R^{2b′} is at each occurrence independently H, OH, C₁-C₄ alkyl, cycloalkyl or R^{2b} and R^{2b′} together with the atom to which they are bonded form a ring;
R^{b′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
R^{1B}, R^{2B}, R^{3B}, and R^{4B} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

In some embodiments a method is provided method for treating an MRGPR X4-dependent condition by administering to a subject in need thereof an effective amount of a compound having formula (IC):

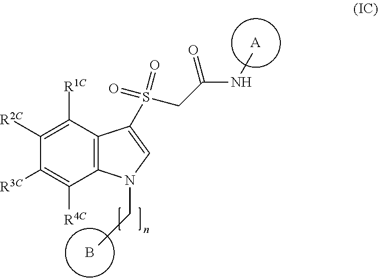

or a pharmaceutically acceptable salt, isomer, hydrate, solvate or isotope thereof, wherein:
A is phenyl, pyridyl or C₁-C₄ alkyl, each of which is optionally substituted with one or more R^{c};
B is phenyl or pyrrolidine optionally substituted with one or more R^{c′};
R^{c} is at each occurrence independently H, —OH, —NH₂, halo, —CO₂H, alkoxy, cyano or C₁-C₆ alkyl;
R^{c′} is at each occurrence independently H or chloro; R^{1c}, R^{2c}, R^{3C}, and R^{4C} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₂-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 1 or 2.

Another embodiment provides a method for treating an MRGPR X4-dependent condition by administering to a subject in need thereof an effective amount of a compound having formula (ID):

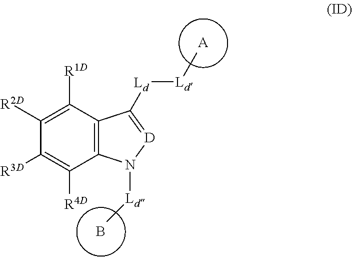

or a pharmaceutically acceptable salt, isomer, hydrate, solvate or isotope thereof, wherein:
A is phenyl, or 5-6 membered heteroaryl, each of which is optionally substituted with one or more R^{d};
B is phenyl optionally substituted with one or more R^{d′};
D is N or C;
L_{d} is —C(O)NHS(O)₂—;
L_{d′} is —(C(R^{2d})(R^{2d′}))ₙ—;
L_{d″} is —(CH₂)—;
R^{d} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2d} is at each occurrence H;
R^{2d′} is at each occurrence independently H or C₁-C₄ alkyl;
R^{d′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
R^{1D}, R^{2D}, R^{3D}, and R^{4D} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and n is 0 or 1.

Yet another embodiment provides a method for treating an MRGPR X4-dependent condition by administering to a subject in need thereof an effective amount of a compound having formula (IE):

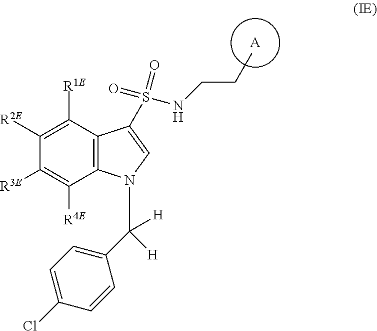

or a pharmaceutically acceptable salt, isomer, hydrate, solvate or isotope thereof, wherein:
A is phenyl optionally substituted with one or more R^{e};
R^{e} is at each occurrence independently H, —OH, —NH₂, halo, —CO₂H, alkoxy, cyano or C₁-C₆ alkyl; and
R^{1E}, R^{2E}, R^{3E}, and R^{4E} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

In some embodiment a method is provided for treating an MRGPR X4-dependent condition by administering to a subject in need thereof an effective amount of a compound having formula (IF):

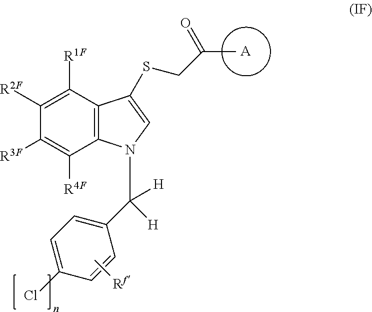

or a pharmaceutically acceptable salt, isomer, hydrate, solvate or isotope thereof, wherein:
A is an isolated pyrrolidine bonded through its nitrogen atom to the carbon chain, and which is optionally substituted with one or more R^{f};
R^{f} is H, —OH, halo, alkoxy or C₁-C₆ alkyl;
R^{f} is chloro or bromo;
R^{1F}, R^{2F}, R^{3F}, and R^{4F} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 0 or 1.

Yet another embodiment a method as disclosed for treating an MRGPR X4-dependent condition by administering to a subject in need thereof an effective amount of a compound having formula (IG):

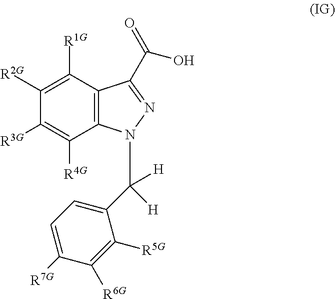

or a pharmaceutically acceptable salt, isomer, hydrate, solvate or isotope thereof, wherein:
R^{1G} is H or fluoro;
R^{2G} is H, chloro or fluoro;
R^{3G} is H or fluoro;
R^{4G} is H;
R^{5G} is H or chloro;
R^{6G} is H, methoxy or trifluoromethoxy; and
R^{7G} is H, chloro, methyl, isopropyl, trifluoromethoxy, trifluoromethyl or difluoromethoxy.

Still another embodiment provides a method for treating an MRGPR X4-dependent condition by administering to a subject in need thereof an effective amount of a compound having formula (IH):

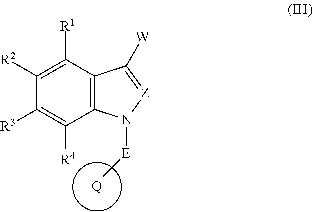

or a pharmaceutically acceptable salt, isomer, hydrate, solvate or isotope thereof, wherein:
W is

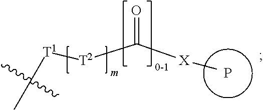

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or (C(H)R^{e})ₙ—X²—(C(R^{t})(R^{t′});
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence, independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl; R^{t′} is at each occurrence independently H, OH, C₁-C₄ alkyl, cycloalkyl or R^{t} and R^{t′} together with the atom to which they are bonded form a ring;
R¹, R², R³, and R⁴ are at each occurrence, independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

One embodiment provides a pharmaceutical composition comprising a pharmaceutically acceptable carrier and a compound having structure (I):

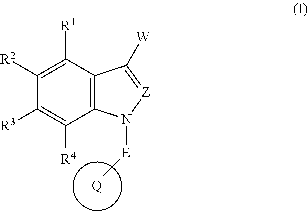

or a pharmaceutically acceptable salt, isomer, hydrate, solvate or isotope thereof, wherein:
W is

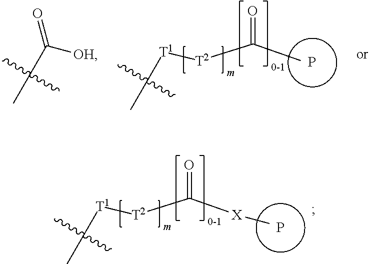

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl;
R^{t′} is at each occurrence independently H, OH, C₁-C₄ alkyl, cycloalkyl or R^{t} and R^{t′} together with the atom to which they are bonded form a ring;
R¹, R², R³, and R⁴ are at each occurrence independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

Another embodiment provides a pharmaceutical composition comprising a pharmaceutically acceptable carrier and where the compound of structure (I) has formula (IA):

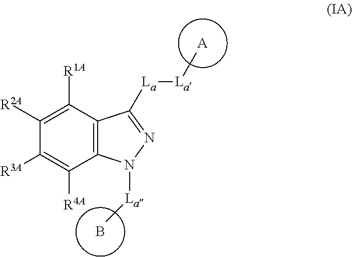

or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{a};
B is C₆-C₁₀ aryl, C₃-C₁₀ cycloalkyl, 3-10 membered monocyclic or bicyclic heterocyclyl, or 5-10 membered monocyclic or bicyclic heteroaryl, each of which is optionally substituted with one or more R^{a′};
Lₐ is —C(O)NH—, or —N(H)C(O)—;
L_{a′} is a bond, —CH₂C(O)—, or —(C(R^{2a})(R^{2a′})₂—.
L_{a″} is —(C(R^{3a})(R^{3a′})ₙ—;
R^{a} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{3a} is at each occurrence independently H, OH or C₁-C₄ alkyl;
R^{3a′} is at each occurrence independently H, OH or C₁-C₄ alkyl;
R^{2a} is at each occurrence independently H or C₁-C₄ alkyl;
R^{2a′} is at each occurrence H;
R^{a′} is at each occurrence, independently H, —OH, —NH₂, halo, —CO₂H, —C(O)Me-, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl;
R^{1A}, R^{2A}, R^{3A}, and R^{4A} are at each occurrence, independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl;
n is 1 or 2;
when n is 1, then B is C₆-C₁₀ aryl, C₃-C₁₀ cycloalkyl, 3-10 membered heterocyclyl, or 5-10 membered monocyclic or bicyclic heteroaryl; or when n is 2, then B is C₆-C₁₀ aryl, C₃-C₁₀ cycloalkyl, or 5-10 membered monocyclic or bicyclic heteroaryl;
with the proviso that when L_{a′} is a bond, then L_{a″} is CH₂, A is pyridine or pyrimidine; and R^{a} at each occurrence cannot both be chloro; and
with the proviso that when L_{a′} is —CH₂C(O)—, then L_{a″} is CH₂ and A is a pyridine or pyrimidine.

Some embodiments provide a pharmaceutical composition comprising a pharmaceutically acceptable carrier and where the compound of structure (I) has formula (IB):

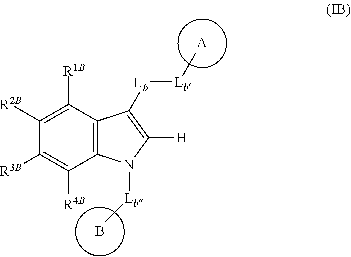

or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{b};
B is phenyl or heterocyclyl, optionally substituted with one or more R^{b′};
L_{b} is —C(O)NH—;
L_{b′} is —(C(R^{2b})(R^{2b′}))—, —(C(R^{2b})(R^{2b′}))₂— or —(C(R^{2b})(R^{2b′}))₃—
L_{b″} is —(CH₂)ₙ—;
R^{b} is at each occurrence, independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2b} is at each occurrence independently H, C₁-C₄ alkyl or cycloalkyl;
R^{2b′} is at each occurrence independently H, OH, C₁-C₄ alkyl, cycloalkyl or R^{2b} and R^{2b′} together with the atom to which they are bonded form a ring;
R^{b′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
R^{1B}, R^{2B}, R^{3B}, and R^{4B} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 1, when B is phenyl or n is 2, when B is heterocyclyl;
when n is 1 and R^{2b′} is OH, then L_{b′} is —(C(R^{2b})(R^{2b′}))₂—;
when n is 1, A is oxazole, L_{b′} is —(C(R^{2b})(R^{2b′}))₂—, R^{2b} and R^{2b′} are H, then R^{b} is not aryl;
when n is 1, L_{b}, is —(C(R^{2b})(R^{2b′}))₃—, then A is phenyl;
when n is 1, L^{b′} is —(C(R^{2b})(R^{2b′}))₂, R^{2b} and R^{2b} are H, then both A and B cannot both be unsubstituted phenyl; and
when n is 2, B cannot be an oxygen containing monocyclic heterocyclyl or a nitrogen containing bicyclic heterocyclyl.

In yet another embodiment is provided a pharmaceutical composition comprising a pharmaceutically acceptable carrier and where the compound of structure (I) has formula (IC):

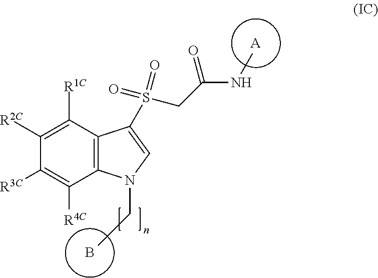

or a pharmaceutically acceptable salt, isomer, hydrate, solvate or isotope thereof, wherein:
A is phenyl, pyridyl or C₁-C₄ alkyl, each of which is optionally substituted with one or more R^{c};
B is phenyl or pyrrolidine optionally substituted with one or more R^{c′};
R^{c} is at each occurrence independently H, —OH, —NH₂, halo, —CO₂H, alkoxy, cyano, or C₁-C₆ alkyl; R^{c′} is at each occurrence independently H, or chloro; R^{1C}, R^{2C}, R^{3C}, and R^{4C} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₂-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 1 or 2;
when A and B are both phenyl, then n is 1, R^{c′} is chloro, and R^{c} is selected from the group consisting of a fluoro at the ortho or meta positions, a chloro at the ortho, meta or para positions, a methoxy at the ortho or meta positions, a methyl at the para position, a dichloro at the meta and para positions, or a dichloro at the ortho and para positions;
when A is pyridyl, then n is 1 and B is phenyl with a para substituted chloro group;
when A is C₁-C₄ alkyl, then n is 1 and B is phenyl with a para substituted chloro group; and
when B is pyrrolidine, then n is 2 and A is phenyl.

One embodiment provides a pharmaceutical composition comprising a pharmaceutically acceptable carrier and where the compound of structure (I) has formula (ID):

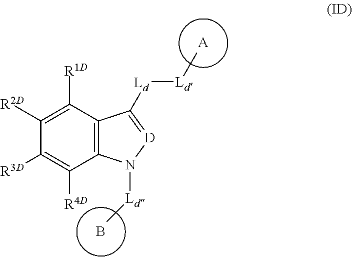

or a pharmaceutically acceptable salt, isomer, hydrate, solvate or isotope thereof, wherein:
A is phenyl, or 5-6 membered heteroaryl, each of which is optionally substituted with one or more R^{d};
B is phenyl optionally substituted with one or more R^{d′};
D is N or C;
L_{d} is —C(O)NHS(O)₂—;
L_{d′} is —(C(R^{2d})(R^{2d′}))ₙ—;
L_{d″} is —(CH₂)—;
R^{d} is at each occurrence, independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2d} is at each occurrence H;
R^{2d′} is at each occurrence independently H or C₁-C₄ alkyl;
R^{d′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; R^{1D}, R^{2D}, R^{3D}, and R^{4D} are at each occurrence, independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 0 or 1; and
when A and B are both phenyl rings; then both phenyl must be substituted.

Another embodiment provides a pharmaceutical composition comprising a pharmaceutically acceptable carrier and where the compound of structure (I) has formula (IE):

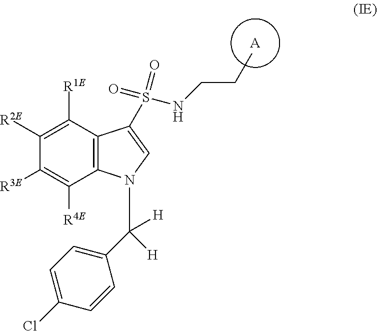

or a pharmaceutically acceptable salt, isomer, hydrate, solvate or isotope thereof, wherein:
A is phenyl optionally substituted with one or more R^{e}.
R^{e} is at each occurrence independently H, —OH, —NH₂, halo, —CO₂H, alkoxy, cyano or C₁-C₆ alkyl; and
R^{1E}, R^{2E}, R^{3E}, and R^{4E} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

Some embodiments provide a pharmaceutical composition comprising a pharmaceutically acceptable carrier and where the compound of structure (I) has formula (IF):

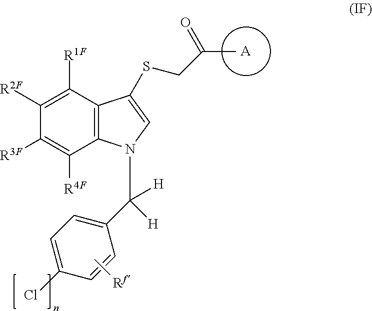

or a pharmaceutically acceptable salt, isomer, hydrate, solvate or isotope thereof, wherein:
A is an isolated pyrrolidine bonded through its nitrogen atom to the carbon chain, and which is optionally substituted with one or more R^{f};
R^{f} is H, —OH, halo, alkoxy or C₁-C₆ alkyl;
R^{f′} is chloro or bromo;
R^{1F}, R^{2F}, R^{3F}, and R^{4F} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 0 or 1; and
when n is 0, then Rr cannot be chloro.

One embodiment provides a pharmaceutical composition comprising a pharmaceutically acceptable carrier and where the compound of structure (I) has formula (IG):

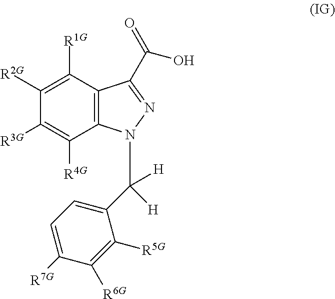

or a pharmaceutically acceptable salt, isomer, hydrate, solvate or isotope thereof, wherein:
R^{1G} is H or fluoro;
R^{2G} is H, chloro or fluoro;
R^{3G} is H or fluoro;
R^{4G} is H;
R^{5G} is H or chloro;
R^{6G} is H, methoxy or trifluoromethoxy;
R^{7G} is H, chloro, methyl, isopropyl, trifluoromethoxy, trifluoromethyl or difluoromethoxy;
with the proviso that when R^{5G} is H and R^{6G} is H, then R^{7G} is isopropyl, trifluoromethoxy or difluoromethoxy;
when R^{5G} is chloro, then R^{6G} is H and R^{7G} is trifluoromethyl;
when R^{6G} is methoxy, then R^{7G} is chloro; or when R^{6G} is trifluoromethoxy then R^{5G} and R^{7G} are both H;
when R^{7G} is trifluoromethyl, then R^{5G} is chloro;
when R^{7G} is chloro, then R^{5G} is not chloro, and one of R^{1G} or R^{2G} or R^{3G} is fluoro;
when R^{7G} is methyl, then R^{2G} is chloro; and
when R^{7G} is difluoromethoxy, then R^{2G} is not fluoro.

One embodiment provides a pharmaceutical composition comprising a pharmaceutically acceptable carrier and where the compound of structure (I) has formula (IH):

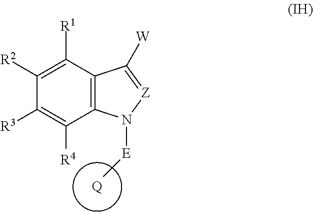

or a pharmaceutically acceptable salt, isomer, hydrate, solvate or isotope thereof, wherein:
W is

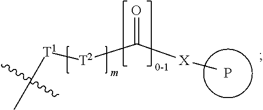

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or (C(H)R^{e})ₙ—X²—(C(R^{t})(R^{t′})ₚ;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence, independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl; R^{t′} is at each occurrence independently H, OH, C₁-C₄ alkyl, cycloalkyl or R^{t} and R^{t′} together with the atom to which they are bonded form a ring; R¹, R², R³, and R⁴ are at each occurrence, independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

One embodiment provides a compound having formula (IA):

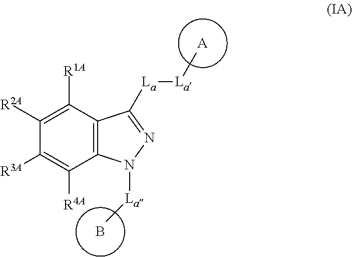

or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{a};
B is C₆-C₁₀ aryl, C₃-C₁₀ cycloalkyl, 3-10 membered monocyclic or bicyclic heterocyclyl, or 5-10 membered monocyclic or bicyclic heteroaryl, each of which is optionally substituted with one or more R^{a′};
Lₐ is —C(O)NH—, or —N(H)C(O)—;
L_{a′} is a bond, —CH₂C(O)—, or —(C(R^{2a})(R^{2a′})₂—.
L_{a″} is —(C(R^{3a})(R^{3a′}))ₙ—;
R^{a} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{3a} is at each occurrence independently H, OH or C₁-C₄ alkyl;
R^{3a′} is at each occurrence independently H, OH or C₁-C₄ alkyl;
R^{2a} is at each occurrence independently H or C₁-C₄ alkyl;
R^{2a′} is at each occurrence H;
R^{a′} is at each occurrence independently H, —OH, —NH₂, halo, —CO₂H, —C(O)Me-, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl;
R^{1A}, R^{2A}, R^{3A}, and R^{4A} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl; and
n is 1 or 2;
when n is 1, then B is C₆-C₁₀ aryl, C₃-C₁₀ cycloalkyl, 3-10 membered heterocyclyl, or 5-10 membered monocyclic or bicyclic heteroaryl; or when n is 2, then B is C₆-C₁₀ aryl, C₃-C₁₀ cycloalkyl, or 5-10 membered monocyclic or bicyclic heteroaryl;
with the proviso that when L_{a′} is a bond, then L_{a″} is CH₂, A is pyridine or pyrimidine; and R^{a} at each occurrence cannot both be chloro; and
with the proviso that when L_{a′} is —CH₂C(O)—, then L_{a″} is CH₂ and A is a pyridine or pyrimidine.

Another embodiment provides a compound having formula (IB).

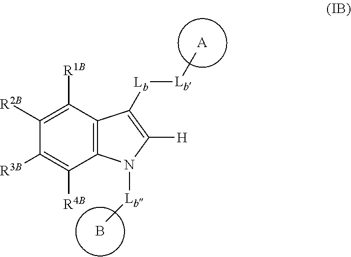

or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{b};
B is phenyl or heterocyclyl, optionally substituted with one or more R^{b′};
L_{b} is —C(O)NH—;
L_{b′} is —(C(R^{2b})(R^{2b′}))—, —(C(R^{2b})(R^{2b′}))₂— or —(C(R^{2b})(R^{2b′}))₃—
L_{b″} is —(CH₂)ₙ—;
R^{b} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2b} is at each occurrence independently H, C₁-C₄ alkyl or cycloalkyl;
R^{2b′} is at each occurrence independently H, OH, C₁-C₄ alkyl, cycloalkyl or R^{2b} and R^{2b′} together with the atom to which they are bonded form a ring;
R^{b′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
R^{1B}, R^{2B}, R^{3B}, and R^{4B} are at each occurrence, independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 1, when B is phenyl or n is 2, when B is heterocyclyl;
when n is 1 and R^{2b′} is OH, then L_{b′} is —(C(R^{2b})(R^{2b′}))₂—;
when n is 1, A is oxazole, L_{b′} is —(C(R^{2b})(R^{2b′}))₂—, R^{2b} and R^{2b′} are H, then R^{b} is not aryl;
when n is 1, L_{b}, is —(C(R^{2b})(R^{2b′}))₃—, then A is phenyl;
when n is 1, L_{b′} is —(C(R^{2b})(R^{2b′}))₂, R^{2b} and R^{2b′} are H, then both A and B cannot both be unsubstituted phenyl;
when n is 1, L_{b}, is CH₂, then A is an unsubstituted pyridyl wherein R^{b′} is methyl; and
when n is 2, B cannot be an oxygen containing monocyclic heterocyclyl or a nitrogen containing bicyclic heterocyclyl.

Some embodiments provide a compound having formula (IC):

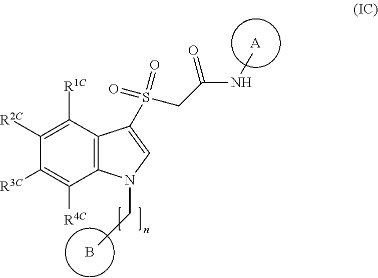

or a pharmaceutically acceptable salt, isomer, hydrate, solvate or isotope thereof, wherein:
A is phenyl, pyridyl or C₁-C₄ alkyl, each of which is optionally substituted with one or more R^{c};
B is phenyl or pyrrolidine optionally substituted with one or more R^{c′};
R^{c} is at each occurrence independently H, —OH, —NH₂, halo, —CO₂H, alkoxy, cyano or C₁-C₆ alkyl;
R^{c′} is at each occurrence, independently H or chloro;
R^{1c}, R^{2C}, R^{3C}, and R^{4C} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₂-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 1 or 2;
when A and B are both phenyl, then n is 1, R^{c′} is chloro, and R^{c} is selected from the group consisting of a fluoro at the ortho or meta positions, a chloro at the ortho, meta or para positions, a methoxy at the ortho or meta positions, a methyl at the para position, a dichloro at the meta and para positions, or a dichloro at the ortho and para positions,
when A is pyridyl, then n is 1 and B is phenyl with a para substituted chloro group;
when A is C₁-C₄ alkyl, then n is 1 and B is phenyl with a para substituted chloro group; and
when B is pyrrolidine, then n is 2 and A is phenyl.

Yet other embodiments provide a compound having formula (ID).

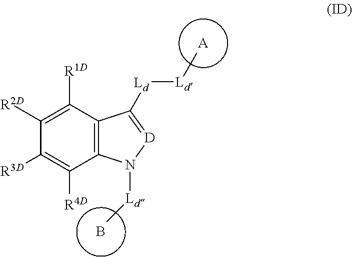

or a pharmaceutically acceptable salt, isomer, hydrate, solvate or isotope thereof, wherein:
A is phenyl, or 5-6 membered heteroaryl, each of which is optionally substituted with one or more R^{d};
B is phenyl optionally substituted with one or more R^{d′};
D is N or C;
L_{d} is —C(O)NHS(O)₂—;
L_{d′} is —(C(R^{2d})(R^{2d′}))ₙ—;
L_{d″} is —(CH₂)—;
R^{d} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2d} is at each occurrence H;
R^{2d′} is at each occurrence independently H or C₁-C₄ alkyl;
R^{d′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; R^{1D}, R^{2D}, R^{3D}, and R^{4D} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
n is 0 or 1;
when A and B are both phenyl rings; then both phenyl must be substituted; and
when A is a 5-membered heteroaryl, then the heteroaryl must contain 2 heteroatoms.

One embodiment provides a compound having formula (IE):

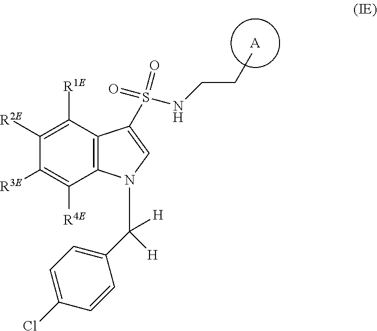

or a pharmaceutically acceptable salt, isomer, hydrate, solvate or isotope thereof, wherein:
A is phenyl optionally substituted with one or more R^{e}.
R^{e} is at each occurrence independently H, —OH, —NH₂, halo, —CO₂H, alkoxy, cyano or C₁-C₆ alkyl; and
R^{1E}, R^{2E}, R^{3E}, and R^{4E} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

Another embodiment provides a compound having formula (IF):

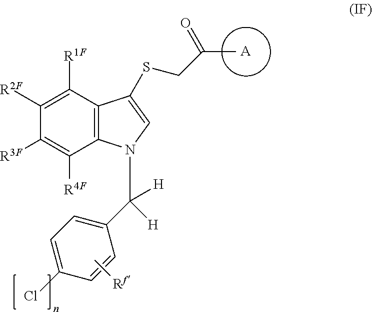

or a pharmaceutically acceptable salt, isomer, hydrate, solvate or isotope thereof, wherein:
A is an isolated pyrrolidine bonded through its nitrogen atom to the carbon chain, and which is optionally substituted with one or more R^{f};
R^{f} is H, —OH, halo, alkoxy or C₁-C₆ alkyl;
R^{f′} is chloro or bromo;
R^{1F}, R^{2F}, R^{3F}, and R^{4F} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 1.

Some embodiments provide a compound having formula (IG):

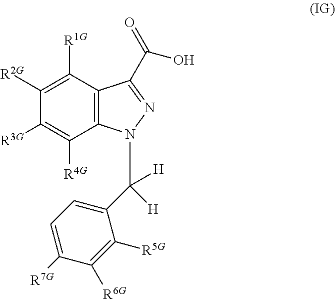

or a pharmaceutically acceptable salt, isomer, hydrate, solvate or isotope thereof, wherein:
R^{1G} is H or fluoro;
R^{2G} is H, chloro or fluoro;
R^{3G} is H or fluoro;
R^{4G} is H;
R^{5G} is H or chloro;
R^{6G} is H, methoxy or trifluoromethoxy; and
R^{7G} is H, chloro, methyl, isopropyl, trifluoromethoxy, trifluoromethyl or difluoromethoxy;
with the proviso that when R^{5G} is H and R^{6G} is H, then R^{7G} is isopropyl, trifluoromethoxy or difluoromethoxy;
when R^{5G} is chloro, then R^{6G} is H and R^{7G} is trifluoromethyl;
when R^{6G} is methoxy, then R^{7G} is chloro; or when R^{6G} is trifluoromethoxy then R^{5G} and R^{7G} are both H;
when R^{7G} is trifluoromethyl, then R^{5G} is chloro;
when R^{7G} is chloro, then R^{5G} is not chloro, and one of R^{1G} or R^{2G} or R^{3G} is fluoro;
when R^{7G} is methyl, then R^{2G} is chloro; and
when R^{7G} is difluoromethoxy, then R^{2G} is not fluoro.

Some embodiments provide a compound having formula (IH):

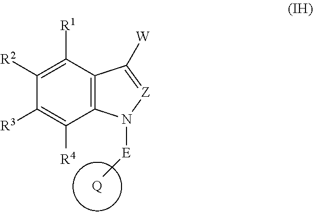

or a pharmaceutically acceptable salt, isomer, hydrate, solvate or isotope thereof, wherein.
W is

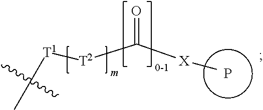

P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or (C(H)R^{e})ₙ—X²—(C(R^{t})(R^{t′});
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence, independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl;
R^{t′} is at each occurrence independently H, OH, C₁-C₄ alkyl, cycloalkyl or R^{t} and R^{t′} together with the atom to which they are bonded form a ring;
R¹, R², R³, and R⁴ are at each occurrence, independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and p is 0 or 1.

Representative compounds of structure (I), as well as Formulas (IA) through (IH) as applicable, include any one of the compounds listed in Table A below, as well as a pharmaceutically acceptable salt, isomer, hydrate, solvate or isotope thereof. To this end, representative compounds are identified herein by their respective “Compound Number”, which is sometimes abbreviated as “Compound No.” or “Cpd. No.”

TABLE A
Representative Compounds

| Cpd. | |
|---|---|
| No. | Structure |
| 1 | 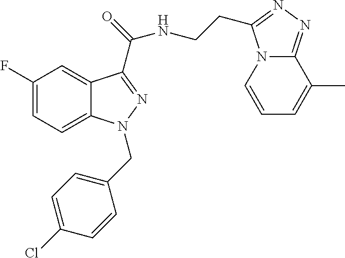 [SMILES: Cc1cccn2c(CCNC(=O)c3nn(Cc4ccc(Cl)cc4)c4ccc(F)cc34)nnc12] |
| 2 | 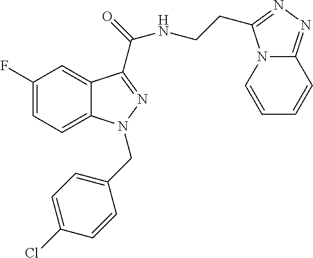 [SMILES: O=C(NCCc1nnc2ccccn12)c1nn(Cc2ccc(Cl)cc2)c2ccc(F)cc12] |
| 3 | 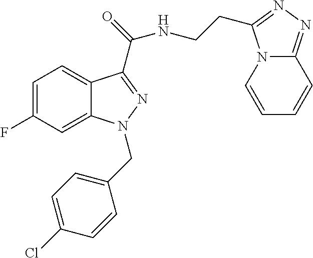 [SMILES: O=C(NCCc1nnc2ccccn12)c1nn(Cc2ccc(Cl)cc2)c2cc(F)ccc12] |
| 4 | 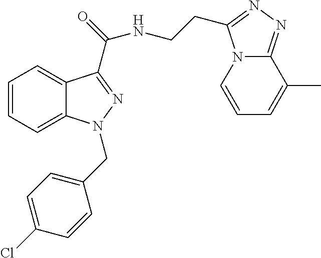 [SMILES: Cc1cccn2c(CCNC(=O)c3nn(Cc4ccc(Cl)cc4)c4ccccc34)nnc12] |
| 5 | 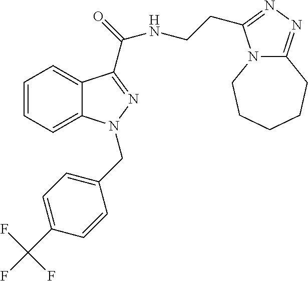 [SMILES: O=C(NCCc1nnc2n1CCCCC2)c1nn(Cc2ccc(C(F)(F)F)cc2)c2ccccc12] |
| 6 | 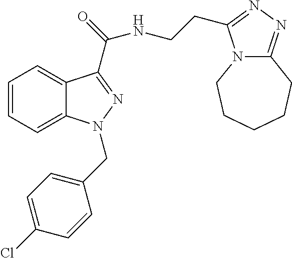 [SMILES: O=C(NCCc1nnc2n1CCCCC2)c1nn(Cc2ccc(Cl)cc2)c2ccccc12] |
| 7 | 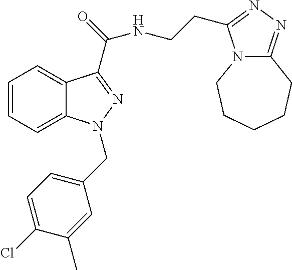 [SMILES: Cc1cc(Cn2nc(C(=O)NCCc3nnc4n3CCCCC4)c3ccccc32)ccc1Cl] |
| 8 | 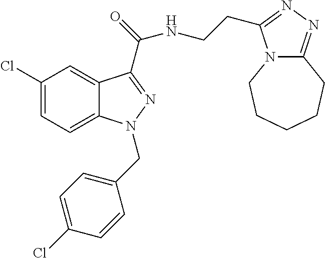 [SMILES: O=C(NCCc1nnc2n1CCCCC2)c1nn(Cc2ccc(Cl)cc2)c2ccc(Cl)cc12] |
| 9 | 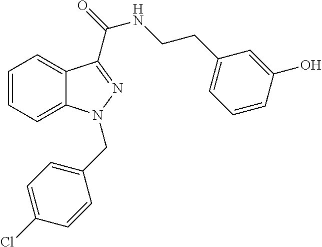 [SMILES: O=C(NCCc1cccc(O)c1)c1nn(Cc2ccc(Cl)cc2)c2ccccc12] |
| 10 | 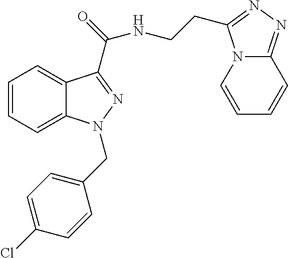 [SMILES: O=C(NCCc1nnc2ccccn12)c1nn(Cc2ccc(Cl)cc2)c2ccccc12] |
| 11 | 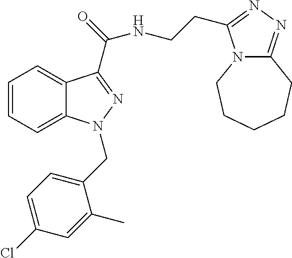 [SMILES: Cc1cc(Cl)ccc1Cn1nc(C(=O)NCCc2nnc3n2CCCCC3)c2ccccc21] |
| 12 | 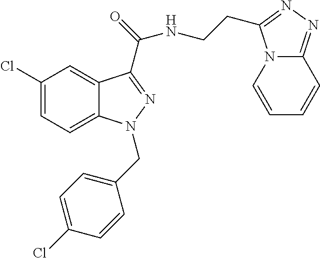 [SMILES: O=C(NCCc1nnc2ccccn12)c1nn(Cc2ccc(Cl)cc2)c2ccc(Cl)cc12] |
| 13 | 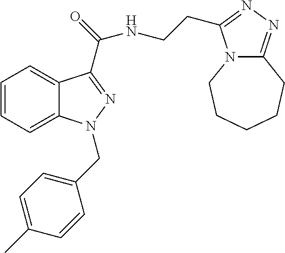 [SMILES: Cc1ccc(Cn2nc(C(=O)NCCc3nnc4n3CCCCC4)c3ccccc32)cc1] |
| 14 | 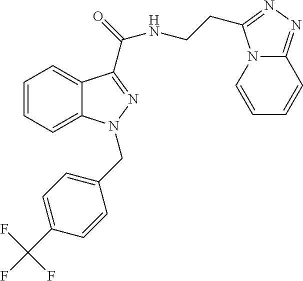 [SMILES: O=C(NCCc1nnc2ccccn12)c1nn(Cc2ccc(C(F)(F)F)cc2)c2ccccc12] |
| 15 | 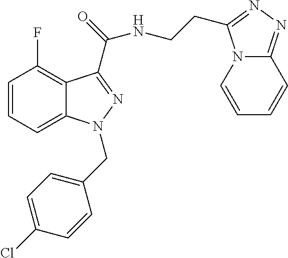 [SMILES: O=C(NCCc1nnc2ccccn12)c1nn(Cc2ccc(Cl)cc2)c2cccc(F)c12] |
| 16 | 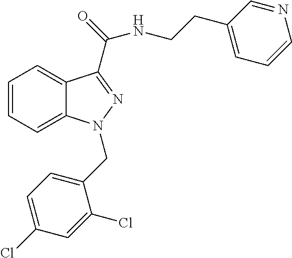 [SMILES: O=C(NCCc1cccnc1)c1nn(Cc2ccc(Cl)cc2Cl)c2ccccc12] |
| 17 | 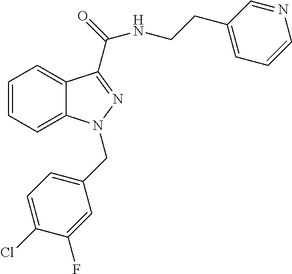 [SMILES: O=C(NCCc1cccnc1)c1nn(Cc2ccc(Cl)c(F)c2)c2ccccc12] |
| 18 | 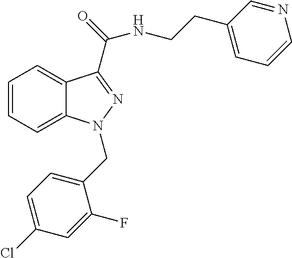 [SMILES: O=C(NCCc1cccnc1)c1nn(Cc2ccc(Cl)cc2F)c2ccccc12] |
| 19 | 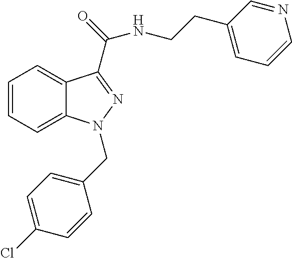 [SMILES: O=C(NCCc1cccnc1)c1nn(Cc2ccc(Cl)cc2)c2ccccc12] |
| 20 | 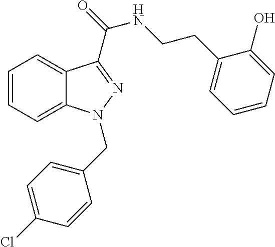 [SMILES: O=C(NCCc1ccccc1O)c1nn(Cc2ccc(Cl)cc2)c2ccccc12] |
| 21 | 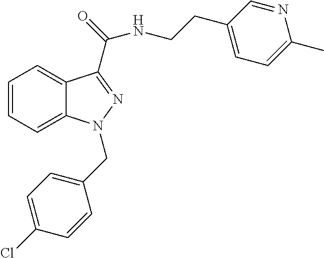 [SMILES: Cc1ccc(CCNC(=O)c2nn(Cc3ccc(Cl)cc3)c3ccccc23)cn1] |
| 22 | 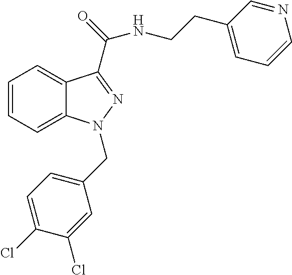 [SMILES: O=C(NCCc1cccnc1)c1nn(Cc2ccc(Cl)c(Cl)c2)c2ccccc12] |
| 23 | 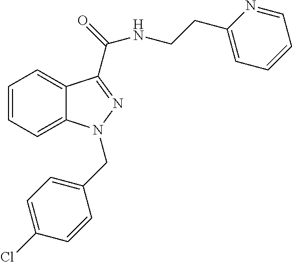 [SMILES: O=C(NCCc1ccccn1)c1nn(Cc2ccc(Cl)cc2)c2ccccc12] |
| 24 | 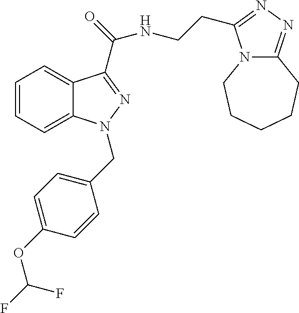 [SMILES: O=C(NCCc1nnc2n1CCCCC2)c1nn(Cc2ccc(OC(F)F)cc2)c2ccccc12] |
| 25 | 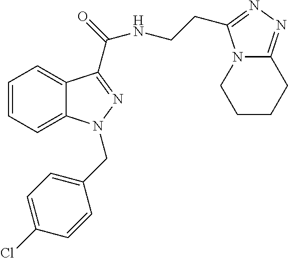 [SMILES: O=C(NCCc1nnc2n1CCCC2)c1nn(Cc2ccc(Cl)cc2)c2ccccc12] |
| 26 | 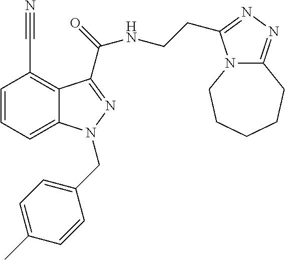 [SMILES: Cc1ccc(Cn2nc(C(=O)NCCc3nnc4n3CCCCC4)c3c(C#N)cccc32)cc1] |
| 27 | 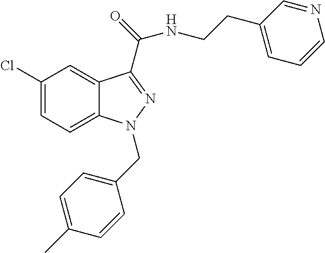 [SMILES: Cc1ccc(Cn2nc(C(=O)NCCc3cccnc3)c3cc(Cl)ccc32)cc1] |
| 28 | 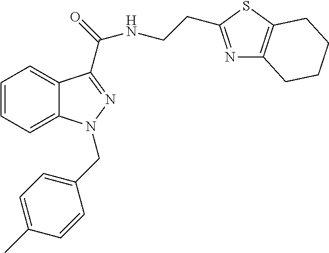 [SMILES: Cc1ccc(Cn2nc(C(=O)NCCc3nc4c(s3)CCCC4)c3ccccc32)cc1] |
| 29 | 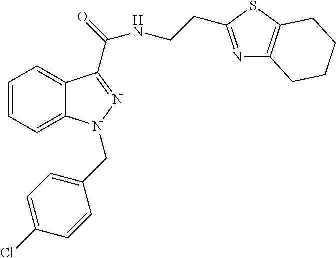 [SMILES: O=C(NCCc1nc2c(s1)CCCC2)c1nn(Cc2ccc(Cl)cc2)c2ccccc12] |
| 30 | 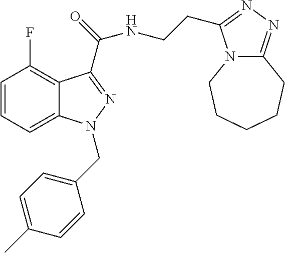 [SMILES: Cc1ccc(Cn2nc(C(=O)NCCc3nnc4n3CCCCC4)c3c(F)cccc32)cc1] |
| 31 | 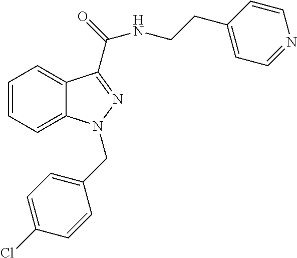 [SMILES: O=C(NCCc1ccncc1)c1nn(Cc2ccc(Cl)cc2)c2ccccc12] |
| 32 | 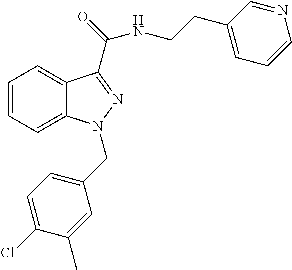 [SMILES: Cc1cc(Cn2nc(C(=O)NCCc3cccnc3)c3ccccc32)ccc1Cl] |
| 33 | 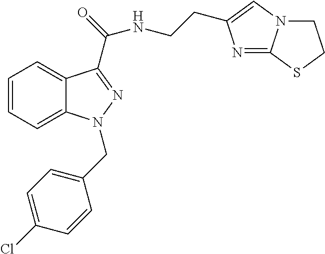 [SMILES: O=C(NCCc1cn2c(n1)SCC2)c1nn(Cc2ccc(Cl)cc2)c2ccccc12] |
| 34 | 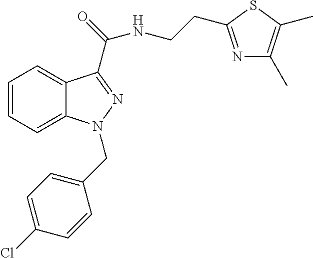 [SMILES: Cc1nc(CCNC(=O)c2nn(Cc3ccc(Cl)cc3)c3ccccc23)sc1C] |
| 35 | 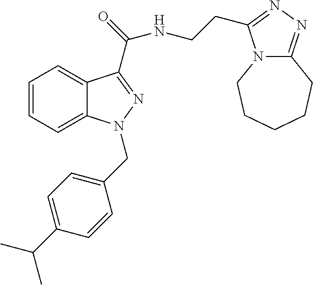 [SMILES: CC(C)c1ccc(Cn2nc(C(=O)NCCc3nnc4n3CCCCC4)c3ccccc32)cc1] |
| 36 | 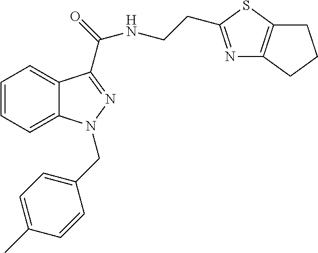 [SMILES: Cc1ccc(Cn2nc(C(=O)NCCc3nc4c(s3)CCC4)c3ccccc32)cc1] |
| 37 | 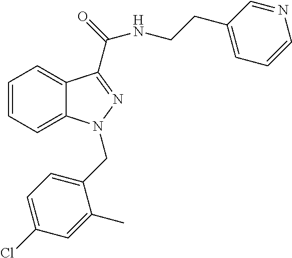 [SMILES: Cc1cc(Cl)ccc1Cn1nc(C(=O)NCCc2cccnc2)c2ccccc21] |
| 38 | 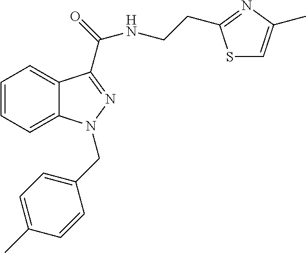 [SMILES: Cc1ccc(Cn2nc(C(=O)NCCc3nc(C)cs3)c3ccccc32)cc1] |
| 39 | 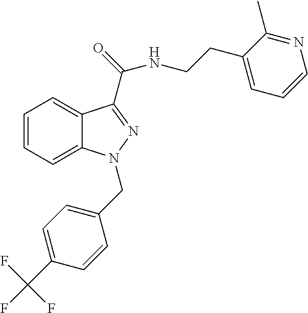 [SMILES: Cc1ncccc1CCNC(=O)c1nn(Cc2ccc(C(F)(F)F)cc2)c2ccccc12] |
| 40 | 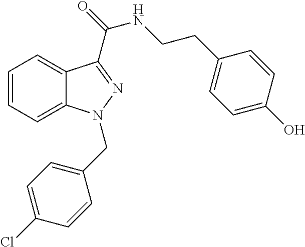 [SMILES: O=C(NCCc1ccc(O)cc1)c1nn(Cc2ccc(Cl)cc2)c2ccccc12] |
| 41 | 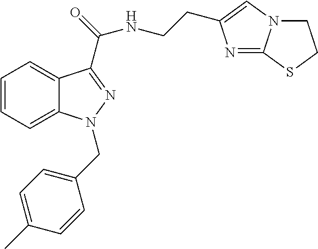 [SMILES: Cc1ccc(Cn2nc(C(=O)NCCc3cn4c(n3)SCC4)c3ccccc32)cc1] |
| 42 | 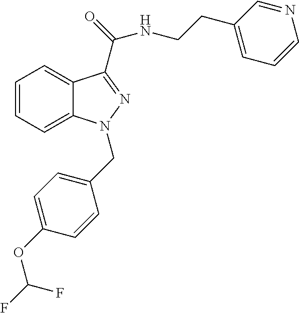 [SMILES: O=C(NCCc1cccnc1)c1nn(Cc2ccc(OC(F)F)cc2)c2ccccc12] |
| 43 | 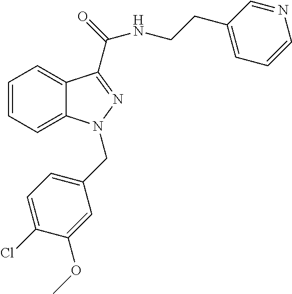 [SMILES: COc1cc(Cn2nc(C(=O)NCCc3cccnc3)c3ccccc32)ccc1Cl] |
| 44 | 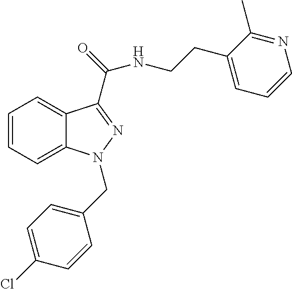 [SMILES: Cc1ncccc1CCNC(=O)c1nn(Cc2ccc(Cl)cc2)c2ccccc12] |
| 45 | 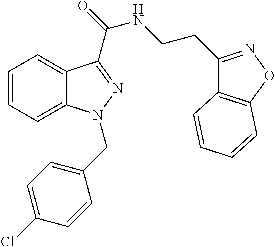 [SMILES: O=C(NCCc1noc2ccccc12)c1nn(Cc2ccc(Cl)cc2)c2ccccc12] |
| 46 | 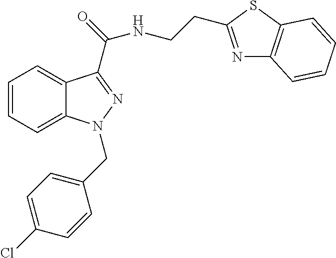 [SMILES: O=C(NCCc1nc2ccccc2s1)c1nn(Cc2ccc(Cl)cc2)c2ccccc12] |
| 47 | 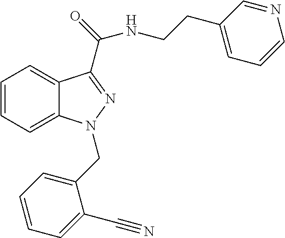 [SMILES: N#Cc1ccccc1Cn1nc(C(=O)NCCc2cccnc2)c2ccccc21] |
| 48 | 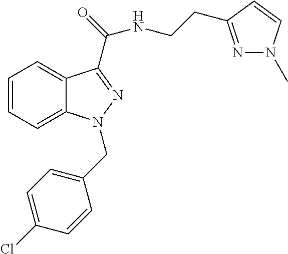 [SMILES: Cn1ccc(CCNC(=O)c2nn(Cc3ccc(Cl)cc3)c3ccccc23)n1] |
| 49 | 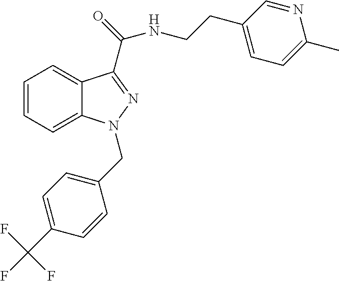 [SMILES: Cc1ccc(CCNC(=O)c2nn(Cc3ccc(C(F)(F)F)cc3)c3ccccc23)cn1] |
| 50 | 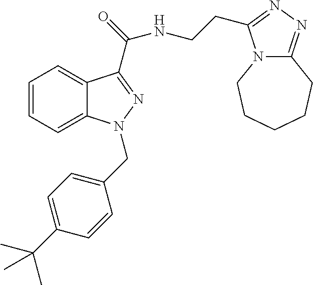 [SMILES: CC(C)(C)c1ccc(Cn2nc(C(=O)NCCc3nnc4n3CCCCC4)c3ccccc32)cc1] |
| 51 | 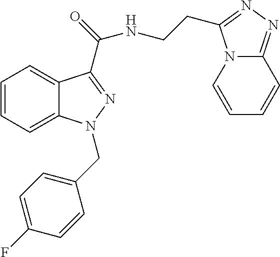 [SMILES: O=C(NCCc1nnc2ccccn12)c1nn(Cc2ccc(F)cc2)c2ccccc12] |
| 52 | 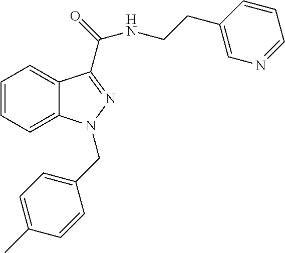 [SMILES: Cc1ccc(Cn2nc(C(=O)NCCc3cccnc3)c3ccccc32)cc1] |
| 53 | 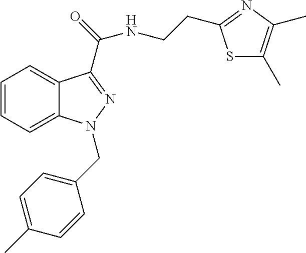 [SMILES: Cc1ccc(Cn2nc(C(=O)NCCc3nc(C)c(C)s3)c3ccccc32)cc1] |
| 54 | 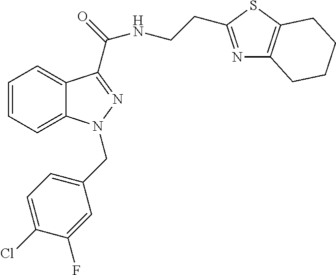 [SMILES: O=C(NCCc1nc2c(s1)CCCC2)c1nn(Cc2ccc(Cl)c(F)c2)c2ccccc12] |
| 55 | 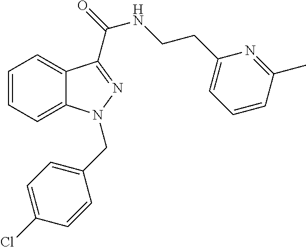 [SMILES: Cc1cccc(CCNC(=O)c2nn(Cc3ccc(Cl)cc3)c3ccccc23)n1] |
| 56 | 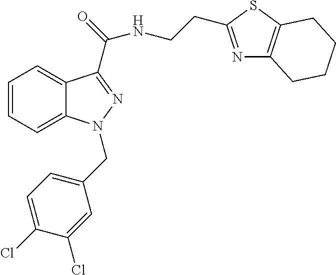 [SMILES: O=C(NCCc1nc2c(s1)CCCC2)c1nn(Cc2ccc(Cl)c(Cl)c2)c2ccccc12] |
| 57 | 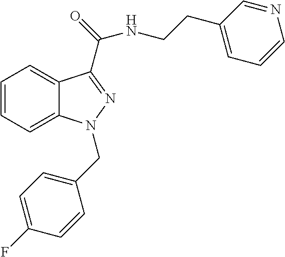 [SMILES: O=C(NCCc1cccnc1)c1nn(Cc2ccc(F)cc2)c2ccccc12] |
| 58 | 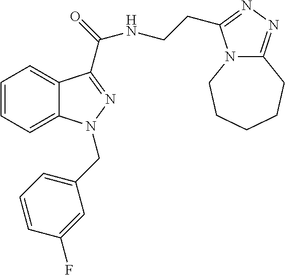 [SMILES: O=C(NCCc1nnc2n1CCCCC2)c1nn(Cc2cccc(F)c2)c2ccccc12] |
| 59 | 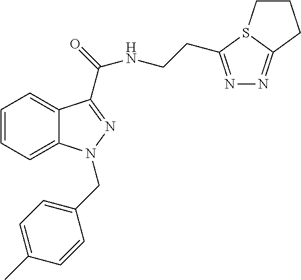 |
| 60 | 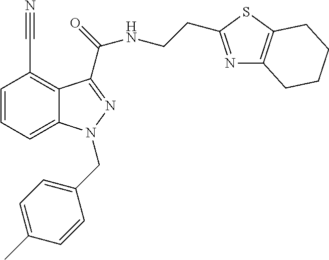 [SMILES: Cc1ccc(Cn2nc(C(=O)NCCc3nc4c(s3)CCCC4)c3c(C#N)cccc32)cc1] |
| 61 | 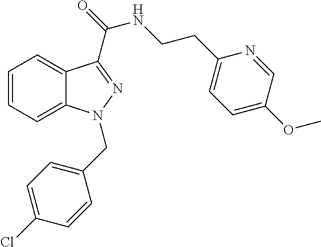 [SMILES: COc1ccc(CCNC(=O)c2nn(Cc3ccc(Cl)cc3)c3ccccc23)nc1] |
| 62 | 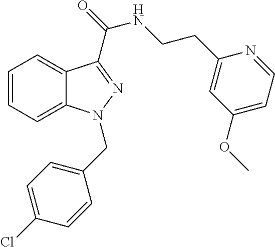 [SMILES: COc1ccnc(CCNC(=O)c2nn(Cc3ccc(Cl)cc3)c3ccccc23)c1] |
| 63 | 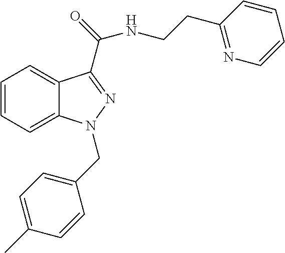 [SMILES: Cc1ccc(Cn2nc(C(=O)NCCc3ccccn3)c3ccccc32)cc1] |
| 64 | 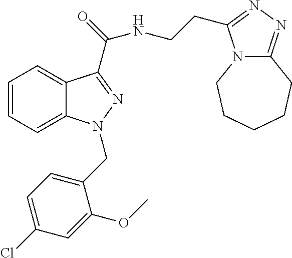 [SMILES: COc1cc(Cl)ccc1Cn1nc(C(=O)NCCc2nnc3n2CCCCC3)c2ccccc21] |
| 65 | 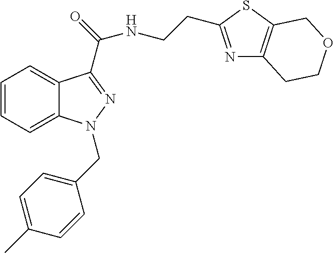 [SMILES: Cc1ccc(Cn2nc(C(=O)NCCc3nc4c(s3)COCC4)c3ccccc32)cc1] |
| 6 | 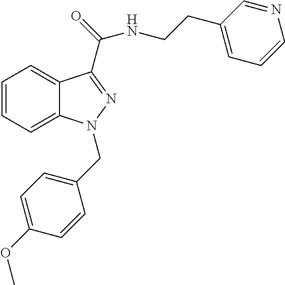 [SMILES: COc1ccc(Cn2nc(C(=O)NCCc3cccnc3)c3ccccc32)cc1] |
| 67 | 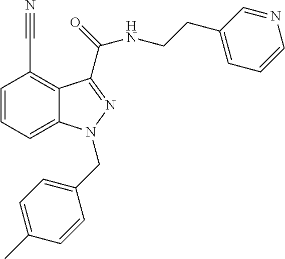 [SMILES: Cc1ccc(Cn2nc(C(=O)NCCc3cccnc3)c3c(C#N)cccc32)cc1] |
| 68 | 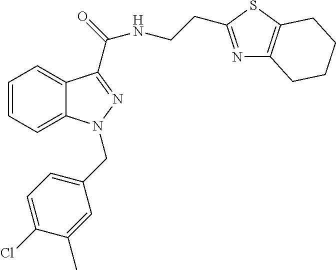 [SMILES: Cc1cc(Cn2nc(C(=O)NCCc3nc4c(s3)CCCC4)c3ccccc32)ccc1Cl] |
| 69 | 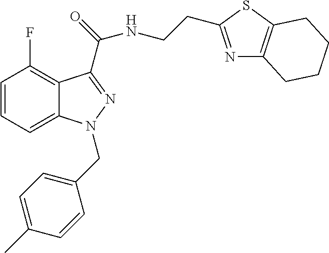 [SMILES: Cc1ccc(Cn2nc(C(=O)NCCc3nc4c(s3)CCCC4)c3c(F)cccc32)cc1] |
| 70 | 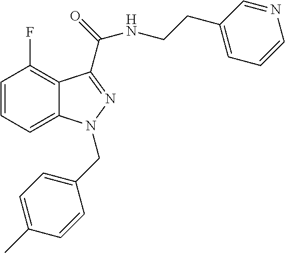 [SMILES: Cc1ccc(Cn2nc(C(=O)NCCc3cccnc3)c3c(F)cccc32)cc1] |
| 71 | 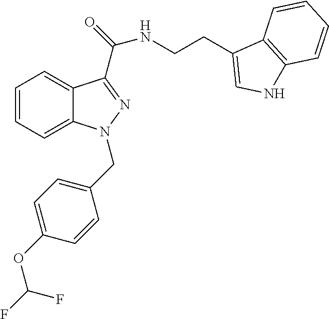 [SMILES: O=C(NCCc1c[nH]c2ccccc12)c1nn(Cc2ccc(OC(F)F)cc2)c2ccccc12] |
| 72 | 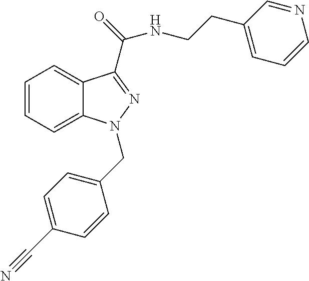 [SMILES: N#Cc1ccc(Cn2nc(C(=O)NCCc3cccnc3)c3ccccc32)cc1] |
| 73 | 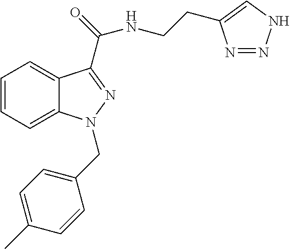 [SMILES: Cc1ccc(Cn2nc(C(=O)NCCc3c[nH]nn3)c3ccccc32)cc1] |
| 74 | 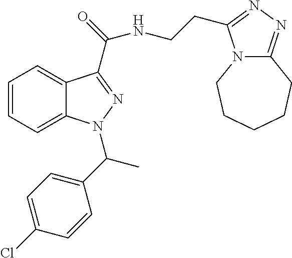 [SMILES: CC(c1ccc(Cl)cc1)n1nc(C(=O)NCCc2nnc3n2CCCCC3)c2ccccc21] |
| 75 | 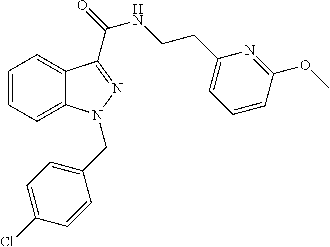 [SMILES: COc1cccc(CCNC(=O)c2nn(Cc3ccc(Cl)cc3)c3ccccc23)n1] |
| 76 | 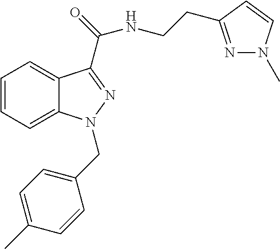 [SMILES: Cc1ccc(Cn2nc(C(=O)NCCc3ccn(C)n3)c3ccccc32)cc1] |
| 77 | 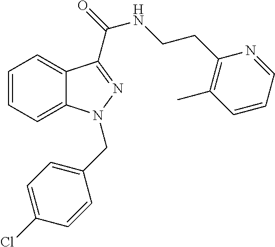 [SMILES: Cc1cccnc1CCNC(=O)c1nn(Cc2ccc(Cl)cc2)c2ccccc12] |
| 78 | 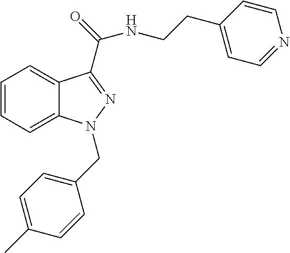 [SMILES: Cc1ccc(Cn2nc(C(=O)NCCc3ccncc3)c3ccccc32)cc1] |
| 79 | 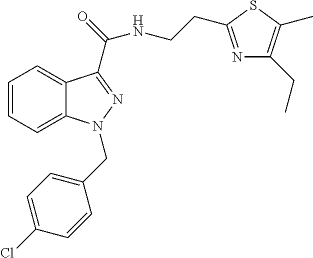 [SMILES: CCc1nc(CCNC(=O)c2nn(Cc3ccc(Cl)cc3)c3ccccc23)sc1C] |
| 80 | 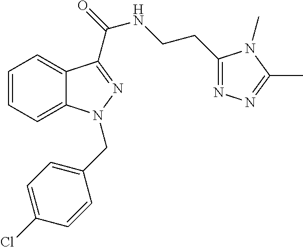 [SMILES: Cc1nnc(CCNC(=O)c2nn(Cc3ccc(Cl)cc3)c3ccccc23)n1C] |
| 81 | 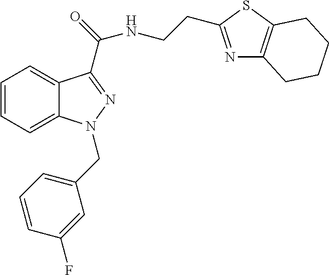 [SMILES: O=C(NCCc1nc2c(s1)CCCC2)c1nn(Cc2cccc(F)c2)c2ccccc12] |
| 82 | 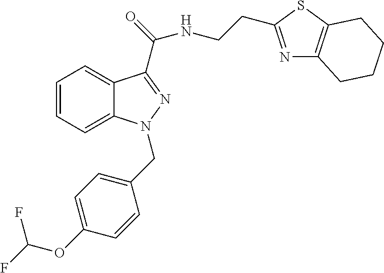 [SMILES: O=C(NCCc1nc2c(s1)CCCC2)c1nn(Cc2ccc(OC(F)F)cc2)c2ccccc12] |
| 83 | 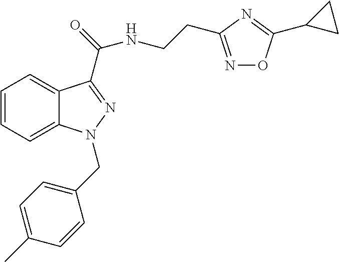 [SMILES: Cc1ccc(Cn2nc(C(=O)NCCc3noc(C4CC4)n3)c3ccccc32)cc1] |
| 84 | 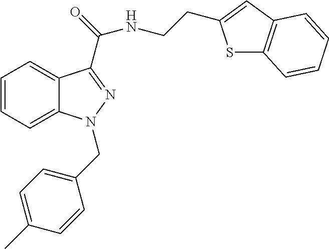 [SMILES: Cc1ccc(Cn2nc(C(=O)NCCc3cc4ccccc4s3)c3ccccc32)cc1] |
| 85 | 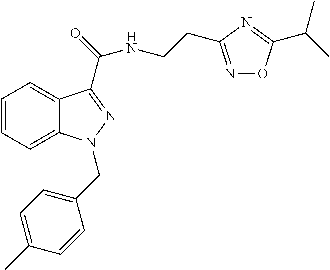 [SMILES: Cc1ccc(Cn2nc(C(=O)NCCc3noc(C(C)C)n3)c3ccccc32)cc1] |
| 86 | 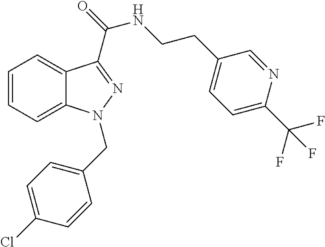 [SMILES: O=C(NCCc1ccc(C(F)(F)F)nc1)c1nn(Cc2ccc(Cl)cc2)c2ccccc12] |
| 87 | 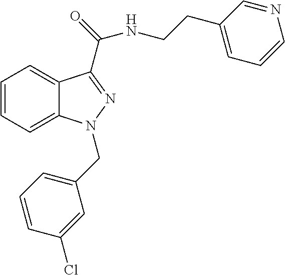 [SMILES: O=C(NCCc1cccnc1)c1nn(Cc2cccc(Cl)c2)c2ccccc12] |
| 88 | 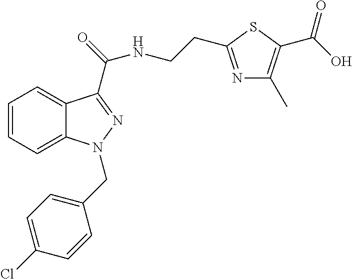 [SMILES: Cc1nc(CCNC(=O)c2nn(Cc3ccc(Cl)cc3)c3ccccc23)sc1C(=O)O] |
| 89 | 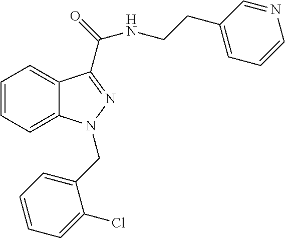 [SMILES: O=C(NCCc1cccnc1)c1nn(Cc2ccccc2Cl)c2ccccc12] |
| 90 | 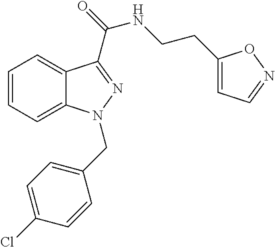 [SMILES: O=C(NCCc1ccno1)c1nn(Cc2ccc(Cl)cc2)c2ccccc12] |
| 91 | 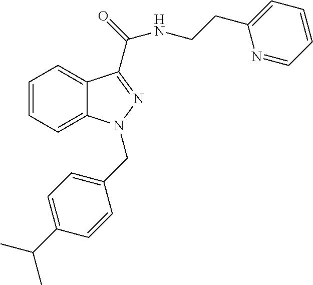 [SMILES: CC(C)c1ccc(Cn2nc(C(=O)NCCc3ccccn3)c3ccccc32)cc1] |
| 92 | 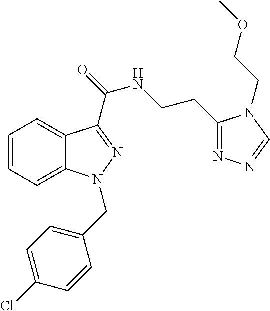 [SMILES: COCCn1cnnc1CCNC(=O)c1nn(Cc2ccc(Cl)cc2)c2ccccc12] |
| 93 | 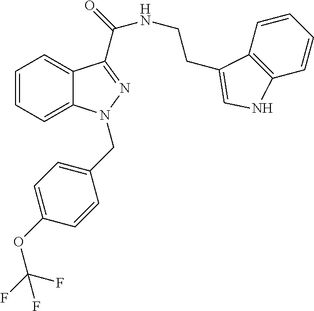 [SMILES: O=C(NCCc1c[nH]c2ccccc12)c1nn(Cc2ccc(OC(F)(F)F)cc2)c2ccccc12] |
| 94 | 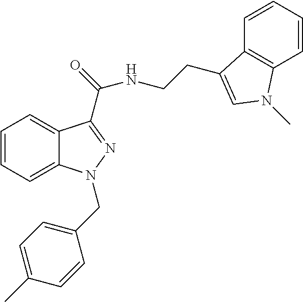 [SMILES: Cc1ccc(Cn2nc(C(=O)NCCc3cn(C)c4ccccc34)c3ccccc32)cc1] |
| 95 | 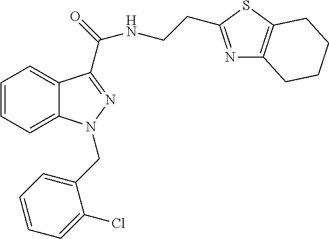 [SMILES: O=C(NCCc1nc2c(s1)CCCC2)c1nn(Cc2ccccc2Cl)c2ccccc12] |
| 96 | 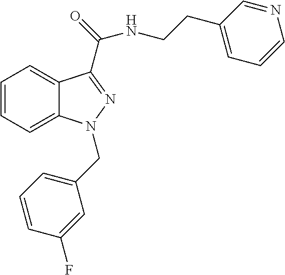 [SMILES: O=C(NCCc1cccnc1)c1nn(Cc2cccc(F)c2)c2ccccc12] |
| 97 | 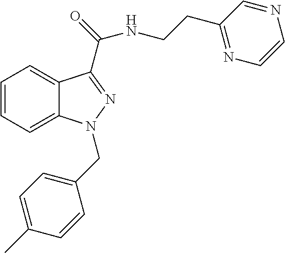 [SMILES: Cc1ccc(Cn2nc(C(=O)NCCc3cnccn3)c3ccccc32)cc1] |
| 98 | 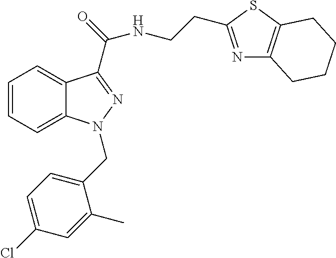 [SMILES: Cc1cc(Cl)ccc1Cn1nc(C(=O)NCCc2nc3c(s2)CCCC3)c2ccccc21] |
| 99 | 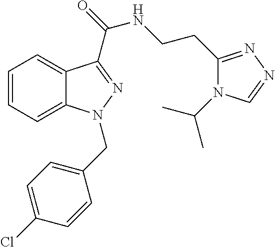 [SMILES: CC(C)n1cnnc1CCNC(=O)c1nn(Cc2ccc(Cl)cc2)c2ccccc12] |
| 100 | 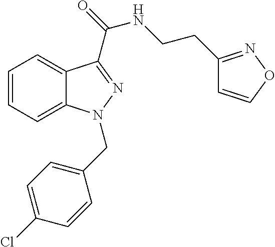 [SMILES: O=C(NCCc1ccon1)c1nn(Cc2ccc(Cl)cc2)c2ccccc12] |
| 101 | 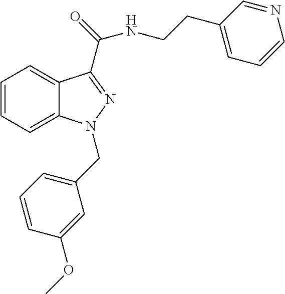 [SMILES: COc1cccc(Cn2nc(C(=O)NCCc3cccnc3)c3ccccc32)c1] |
| 102 | 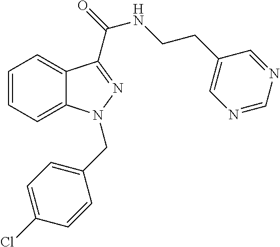 [SMILES: O=C(NCCc1cncnc1)c1nn(Cc2ccc(Cl)cc2)c2ccccc12] |
| 103 | 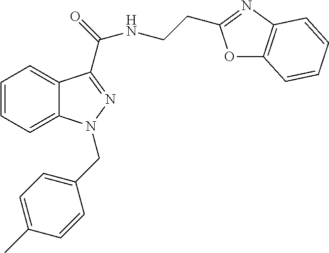 [SMILES: Cc1ccc(Cn2nc(C(=O)NCCc3nc4ccccc4o3)c3ccccc32)cc1] |
| 104 | 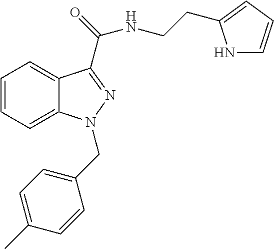 [SMILES: Cc1ccc(Cn2nc(C(=O)NCCc3ccc[nH]3)c3ccccc32)cc1] |
| 105 | 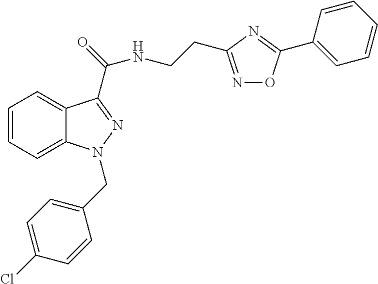 [SMILES: O=C(NCCc1noc(-c2ccccc2)n1)c1nn(Cc2ccc(Cl)cc2)c2ccccc12] |
| 106 | 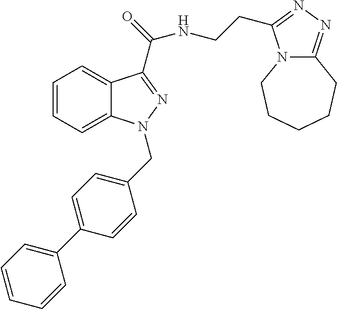 [SMILES: O=C(NCCc1nnc2n1CCCCC2)c1nn(Cc2ccc(-c3ccccc3)cc2)c2ccccc12] |
| 107 | 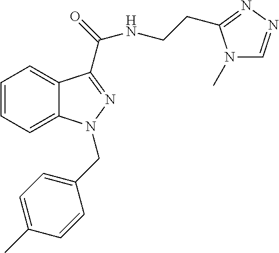 [SMILES: Cc1ccc(Cn2nc(C(=O)NCCc3nncn3C)c3ccccc32)cc1] |
| 108 | 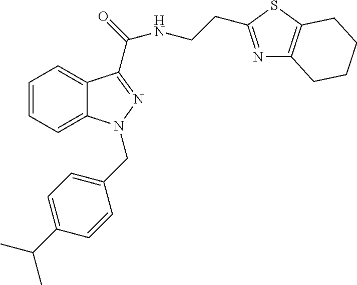 [SMILES: CC(C)c1ccc(Cn2nc(C(=O)NCCc3nc4c(s3)CCCC4)c3ccccc32)cc1] |
| 109 | 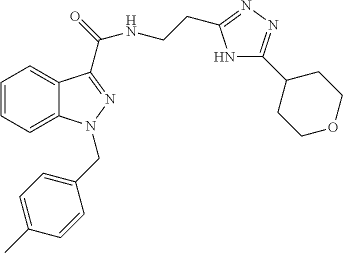 [SMILES: Cc1ccc(Cn2nc(C(=O)NCCc3nnc(C4CCOCC4)[nH]3)c3ccccc32)cc1] |
| 110 | 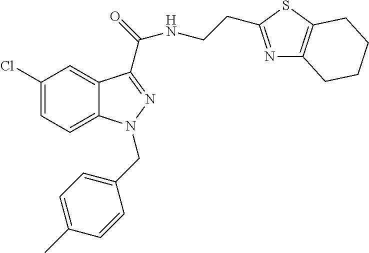 [SMILES: Cc1ccc(Cn2nc(C(=O)NCCc3nc4c(s3)CCCC4)c3cc(Cl)ccc32)cc1] |
| 111 | 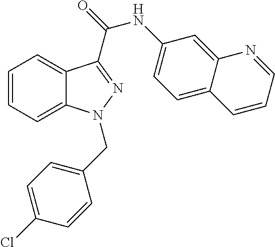 [SMILES: O=C(Nc1ccc2cccnc2c1)c1nn(Cc2ccc(Cl)cc2)c2ccccc12] |
| 112 | 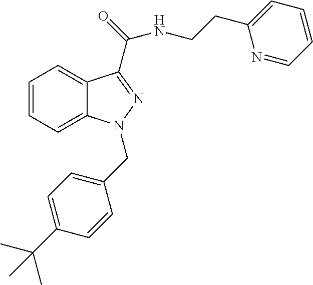 [SMILES: CC(C)(C)c1ccc(Cn2nc(C(=O)NCCc3ccccn3)c3ccccc32)cc1] |
| 113 | 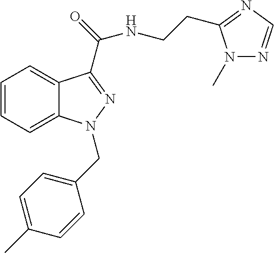 [SMILES: Cc1ccc(Cn2nc(C(=O)NCCc3ncnn3C)c3ccccc32)cc1] |
| 114 | 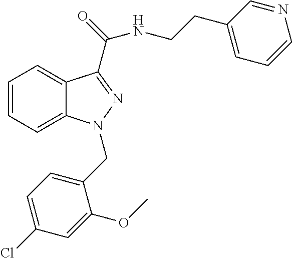 [SMILES: COc1cc(Cl)ccc1Cn1nc(C(=O)NCCc2cccnc2)c2ccccc21] |
| 115 | 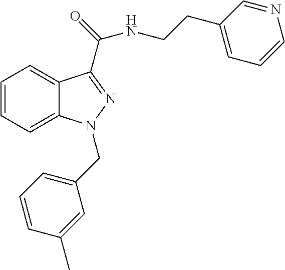 [SMILES: Cc1cccc(Cn2nc(C(=O)NCCc3cccnc3)c3ccccc32)c1] |
| 116 | 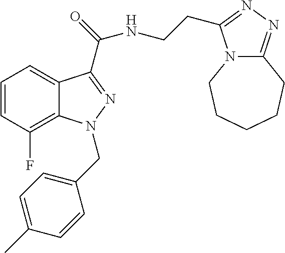 [SMILES: Cc1ccc(Cn2nc(C(=O)NCCc3nnc4n3CCCCC4)c3cccc(F)c32)cc1] |
| 117 | 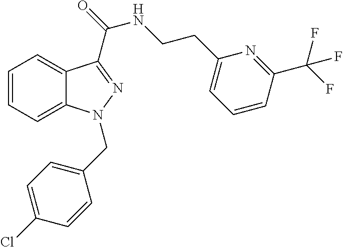 [SMILES: O=C(NCCc1cccc(C(F)(F)F)n1)c1nn(Cc2ccc(Cl)cc2)c2ccccc12] |
| 118 | 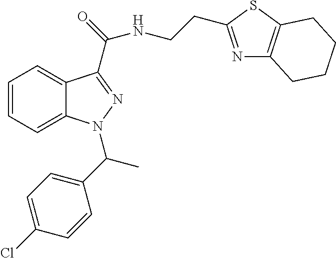 [SMILES: CC(c1ccc(Cl)cc1)n1nc(C(=O)NCCc2nc3c(s2)CCCC3)c2ccccc21] |
| 119 | 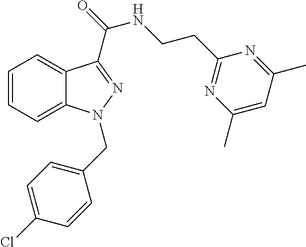 [SMILES: Cc1cc(C)nc(CCNC(=O)c2nn(Cc3ccc(Cl)cc3)c3ccccc23)n1] |
| 120 | 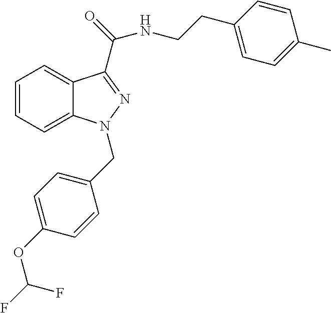 [SMILES: Cc1ccc(CCNC(=O)c2nn(Cc3ccc(OC(F)F)cc3)c3ccccc23)cc1] |
| 121 | 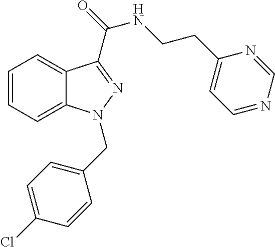 [SMILES: O=C(NCCc1ccncn1)c1nn(Cc2ccc(Cl)cc2)c2ccccc12] |
| 122 | 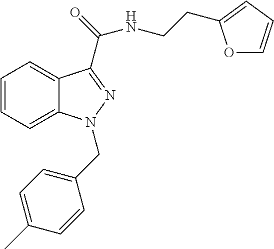 [SMILES: Cc1ccc(Cn2nc(C(=O)NCCc3ccco3)c3ccccc32)cc1] |
| 123 | 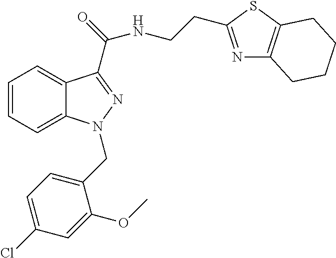 [SMILES: COc1cc(Cl)ccc1Cn1nc(C(=O)NCCc2nc3c(s2)CCCC3)c2ccccc21] |
| 124 | 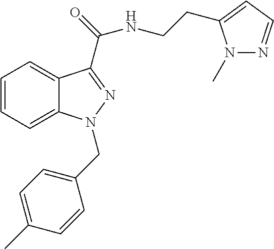 [SMILES: Cc1ccc(Cn2nc(C(=O)NCCc3ccnn3C)c3ccccc32)cc1] |
| 125 | 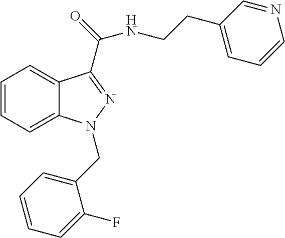 [SMILES: O=C(NCCc1cccnc1)c1nn(Cc2ccccc2F)c2ccccc12] |
| 126 | 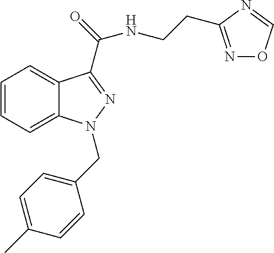 [SMILES: Cc1ccc(Cn2nc(C(=O)NCCc3ncon3)c3ccccc32)cc1] |
| 127 | 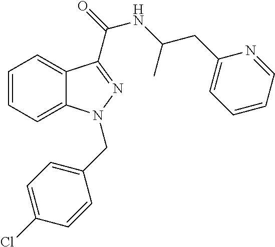 [SMILES: CC(Cc1ccccn1)NC(=O)c1nn(Cc2ccc(Cl)cc2)c2ccccc12] |
| 128 | 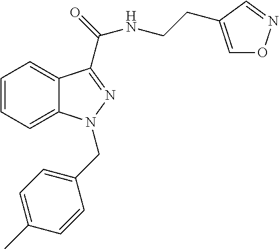 [SMILES: Cc1ccc(Cn2nc(C(=O)NCCc3cnoc3)c3ccccc32)cc1] |
| 129 | 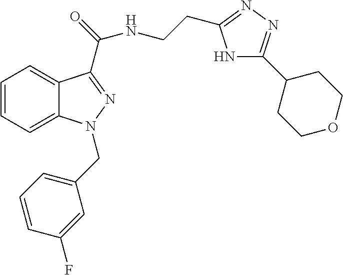 [SMILES: O=C(NCCc1nnc(C2CCOCC2)[nH]1)c1nn(Cc2cccc(F)c2)c2ccccc12] |
| 130 | 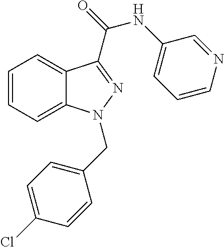 [SMILES: O=C(Nc1cccnc1)c1nn(Cc2ccc(Cl)cc2)c2ccccc12] |
| 131 | 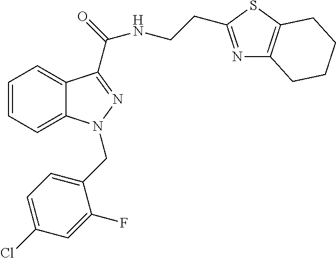 [SMILES: O=C(NCCc1nc2c(s1)CCCC2)c1nn(Cc2ccc(Cl)cc2F)c2ccccc12] |
| 132 | 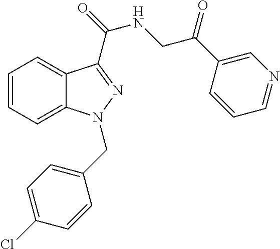 [SMILES: O=C(CNC(=O)c1nn(Cc2ccc(Cl)cc2)c2ccccc12)c1cccnc1] |
| 133 | 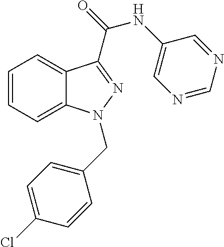 [SMILES: O=C(Nc1cncnc1)c1nn(Cc2ccc(Cl)cc2)c2ccccc12] |
| 134 | 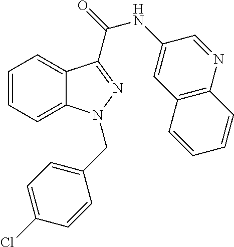 [SMILES: O=C(Nc1cnc2ccccc2c1)c1nn(Cc2ccc(Cl)cc2)c2ccccc12] |
| 135 | 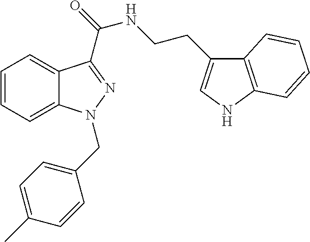 [SMILES: Cc1ccc(Cn2nc(C(=O)NCCc3c[nH]c4ccccc34)c3ccccc32)cc1] |
| 136 | 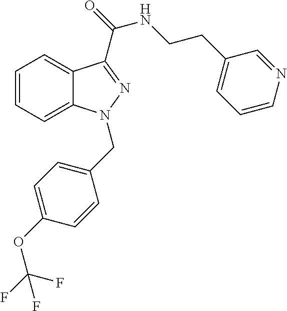 [SMILES: O=C(NCCc1cccnc1)c1nn(Cc2ccc(OC(F)(F)F)cc2)c2ccccc12] |
| 137 | 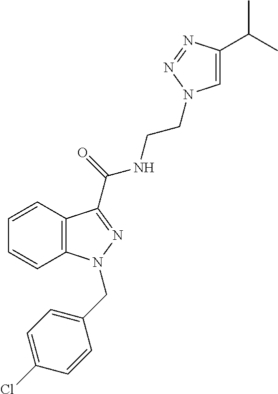 [SMILES: CC(C)c1cn(CCNC(=O)c2nn(Cc3ccc(Cl)cc3)c3ccccc23)nn1] |
| 138 | 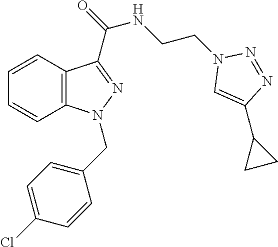 [SMILES: O=C(NCCn1cc(C2CC2)nn1)c1nn(Cc2ccc(Cl)cc2)c2ccccc12] |
| 139 | 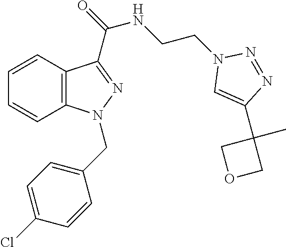 [SMILES: CC1(c2cn(CCNC(=O)c3nn(Cc4ccc(Cl)cc4)c4ccccc34)nn2)COC1] |
| 140 | 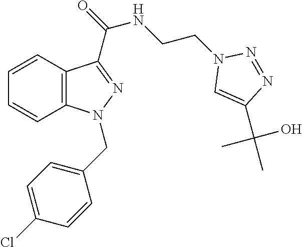 [SMILES: CC(C)(O)c1cn(CCNC(=O)c2nn(Cc3ccc(Cl)cc3)c3ccccc23)nn1] |
| 141 | 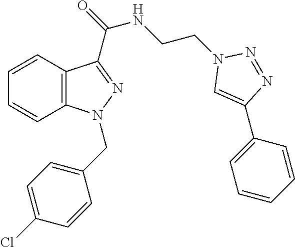 [SMILES: O=C(NCCn1cc(-c2ccccc2)nn1)c1nn(Cc2ccc(Cl)cc2)c2ccccc12] |
| 142 | 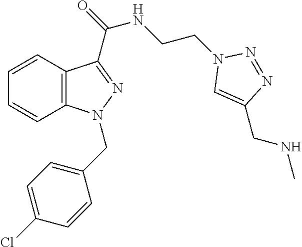 [SMILES: CNCc1cn(CCNC(=O)c2nn(Cc3ccc(Cl)cc3)c3ccccc23)nn1] |
| 143 | 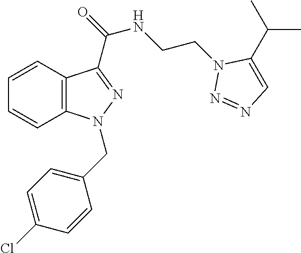 [SMILES: CC(C)c1cnnn1CCNC(=O)c1nn(Cc2ccc(Cl)cc2)c2ccccc12] |
| 144 | 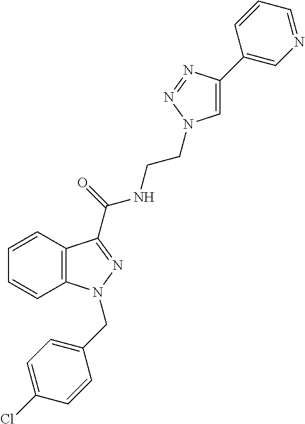 [SMILES: O=C(NCCn1cc(-c2cccnc2)nn1)c1nn(Cc2ccc(Cl)cc2)c2ccccc12] |
| 145 | 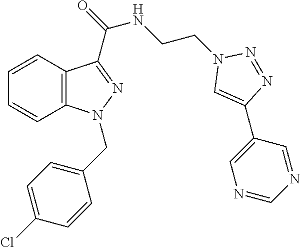 [SMILES: O=C(NCCn1cc(-c2cncnc2)nn1)c1nn(Cc2ccc(Cl)cc2)c2ccccc12] |
| 146 | 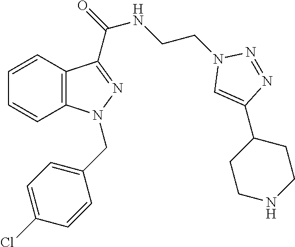 [SMILES: O=C(NCCn1cc(C2CCNCC2)nn1)c1nn(Cc2ccc(Cl)cc2)c2ccccc12] |
| 147 | 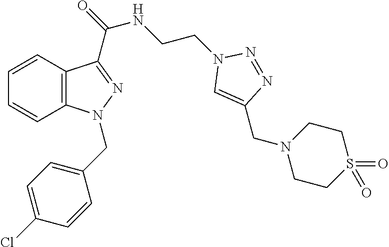 [SMILES: O=C(NCCn1cc(CN2CCS(=O)(=O)CC2)nn1)c1nn(Cc2ccc(Cl)cc2)c2ccccc12] |
| 148 | 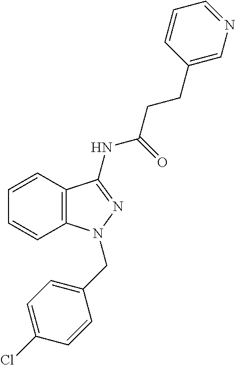 [SMILES: O=C(CCc1cccnc1)Nc1nn(Cc2ccc(Cl)cc2)c2ccccc12] |
| 149 | 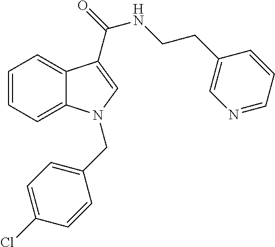 [SMILES: O=C(NCCc1cccnc1)c1cn(Cc2ccc(Cl)cc2)c2ccccc12] |
| 150 | 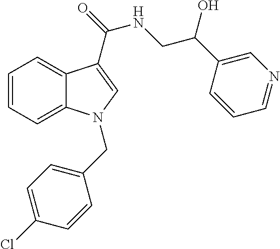 [SMILES: O=C(NCC(O)c1cccnc1)c1cn(Cc2ccc(Cl)cc2)c2ccccc12] |
| 151 | 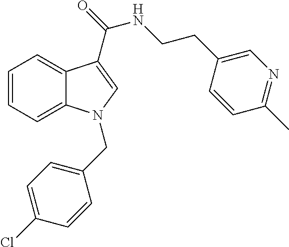 [SMILES: Cc1ccc(CCNC(=O)c2cn(Cc3ccc(Cl)cc3)c3ccccc23)cn1] |
| 152 | 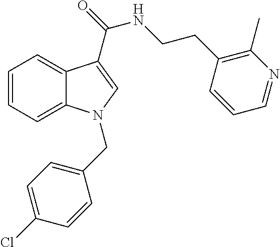 [SMILES: Cc1ncccc1CCNC(=O)c1cn(Cc2ccc(Cl)cc2)c2ccccc12] |
| 153 | 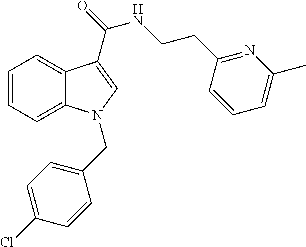 [SMILES: Cc1cccc(CCNC(=O)c2cn(Cc3ccc(Cl)cc3)c3ccccc23)n1] |
| 154 | 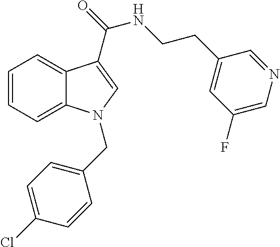 [SMILES: O=C(NCCc1cncc(F)c1)c1cn(Cc2ccc(Cl)cc2)c2ccccc12] |
| 155 | 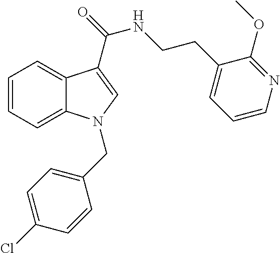 [SMILES: COc1ncccc1CCNC(=O)c1cn(Cc2ccc(Cl)cc2)c2ccccc12] |
| 156 | 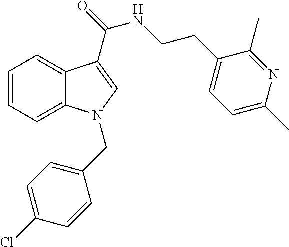 [SMILES: Cc1ccc(CCNC(=O)c2cn(Cc3ccc(Cl)cc3)c3ccccc23)c(C)n1] |
| 157 | 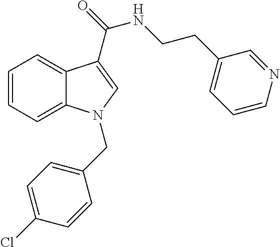 [SMILES: O=C(NCCc1cccnc1)c1cn(Cc2ccc(Cl)cc2)c2ccccc12] |
| 158 | 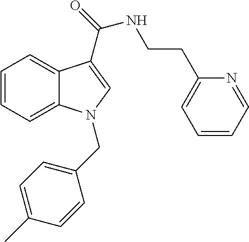 [SMILES: Cc1ccc(Cn2cc(C(=O)NCCc3ccccn3)c3ccccc32)cc1] |
| 159 | 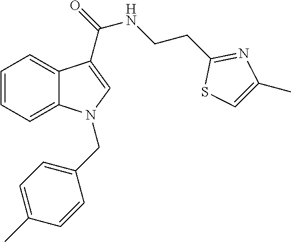 [SMILES: Cc1ccc(Cn2cc(C(=O)NCCc3nc(C)cs3)c3ccccc32)cc1] |
| 160 | 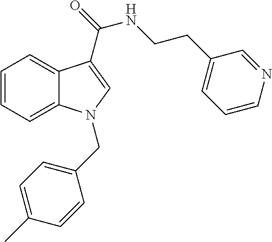 [SMILES: Cc1ccc(Cn2cc(C(=O)NCCc3cccnc3)c3ccccc32)cc1] |
| 161 | 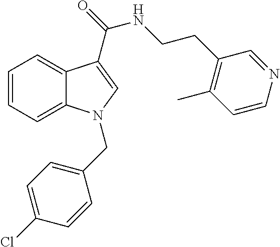 [SMILES: Cc1ccncc1CCNC(=O)c1cn(Cc2ccc(Cl)cc2)c2ccccc12] |
| 162 | 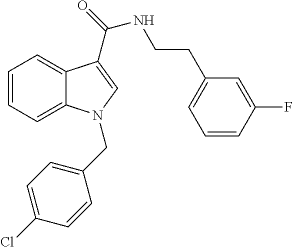 [SMILES: O=C(NCCc1cccc(F)c1)c1cn(Cc2ccc(Cl)cc2)c2ccccc12] |
| 163 | 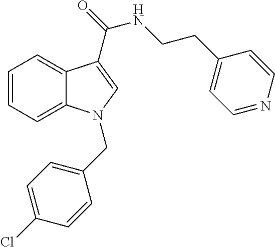 [SMILES: O=C(NCCc1ccncc1)c1cn(Cc2ccc(Cl)cc2)c2ccccc12] |
| 164 | 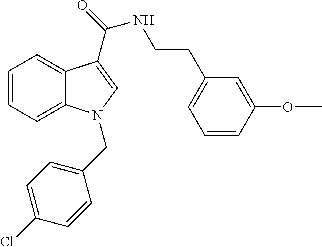 [SMILES: COc1cccc(CCNC(=O)c2cn(Cc3ccc(Cl)cc3)c3ccccc23)c1] |
| 165 | 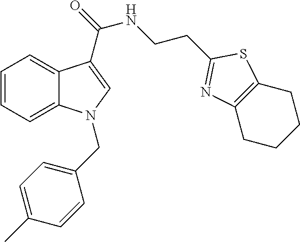 [SMILES: Cc1ccc(Cn2cc(C(=O)NCCc3nc4c(s3)CCCC4)c3ccccc32)cc1] |
| 166 | 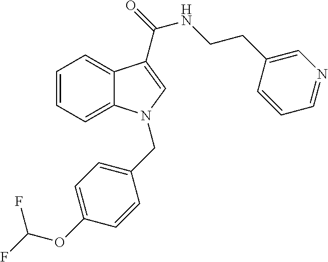 [SMILES: O=C(NCCc1cccnc1)c1cn(Cc2ccc(OC(F)F)cc2)c2ccccc12] |
| 167 | 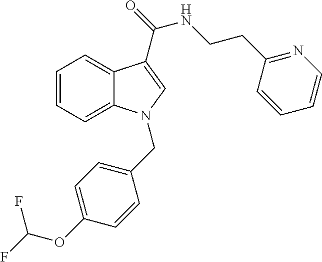 [SMILES: O=C(NCCc1ccccn1)c1cn(Cc2ccc(OC(F)F)cc2)c2ccccc12] |
| 168 | 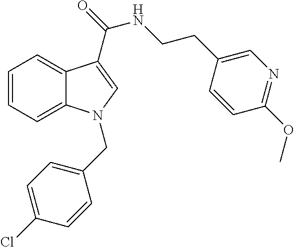 [SMILES: COc1ccc(CCNC(=O)c2cn(Cc3ccc(Cl)cc3)c3ccccc23)cn1] |
| 169 | 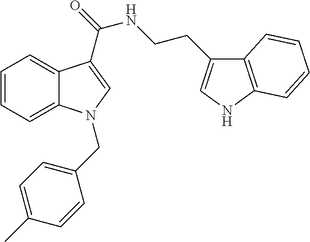 [SMILES: Cc1ccc(Cn2cc(C(=O)NCCc3c[nH]c4ccccc34)c3ccccc32)cc1] |
| 170 | 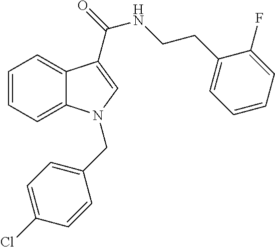 [SMILES: O=C(NCCc1ccccc1F)c1cn(Cc2ccc(Cl)cc2)c2ccccc12] |
| 171 | 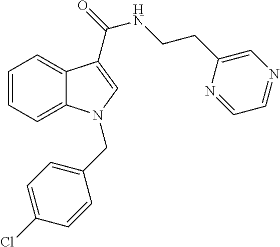 [SMILES: O=C(NCCc1cnccn1)c1cn(Cc2ccc(Cl)cc2)c2ccccc12] |
| 172 | 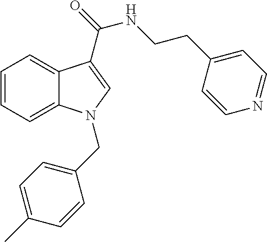 [SMILES: Cc1ccc(Cn2cc(C(=O)NCCc3ccncc3)c3ccccc32)cc1] |
| 173 | 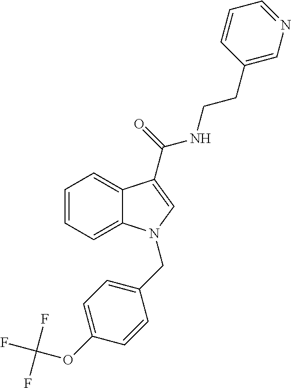 [SMILES: O=C(NCCc1cccnc1)c1cn(Cc2ccc(OC(F)(F)F)cc2)c2ccccc12] |
| 174 | 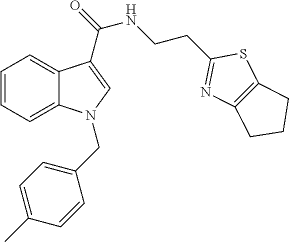 [SMILES: Cc1ccc(Cn2cc(C(=O)NCCc3nc4c(s3)CCC4)c3ccccc32)cc1] |
| 175 | 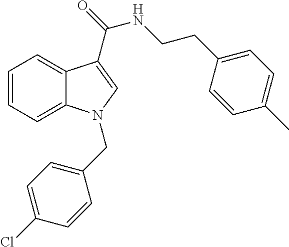 [SMILES: Cc1ccc(CCNC(=O)c2cn(Cc3ccc(Cl)cc3)c3ccccc23)cc1] |
| 176 | 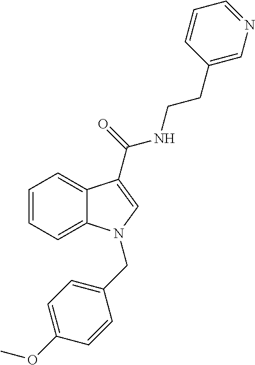 [SMILES: COc1ccc(Cn2cc(C(=O)NCCc3cccnc3)c3ccccc32)cc1] |
| 177 | 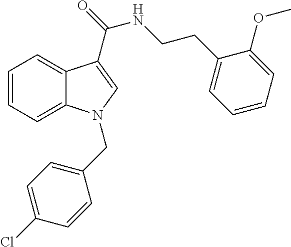 [SMILES: COc1ccccc1CCNC(=O)c1cn(Cc2ccc(Cl)cc2)c2ccccc12] |
| 178 | 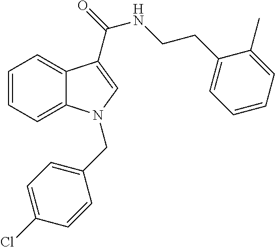 [SMILES: Cc1ccccc1CCNC(=O)c1cn(Cc2ccc(Cl)cc2)c2ccccc12] |
| 179 | 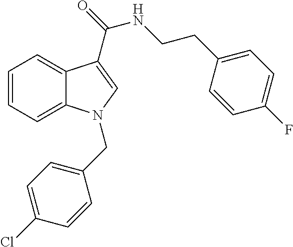 [SMILES: O=C(NCCc1ccc(F)cc1)c1cn(Cc2ccc(Cl)cc2)c2ccccc12] |
| 180 | 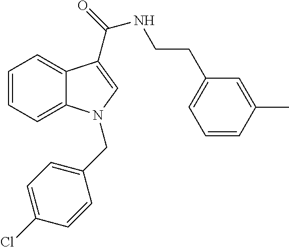 [SMILES: Cc1cccc(CCNC(=O)c2cn(Cc3ccc(Cl)cc3)c3ccccc23)c1] |
| 181 | 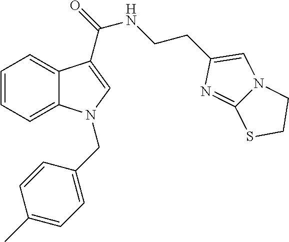 [SMILES: Cc1ccc(Cn2cc(C(=O)NCCc3cn4c(n3)SCC4)c3ccccc32)cc1] |
| 182 | 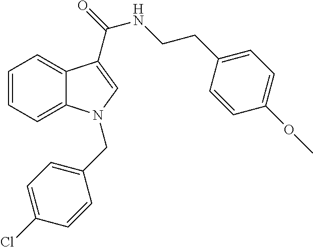 [SMILES: COc1ccc(CCNC(=O)c2cn(Cc3ccc(Cl)cc3)c3ccccc23)cc1] |
| 183 | 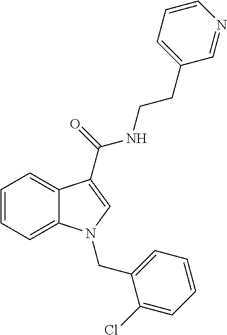 [SMILES: O=C(NCCc1cccnc1)c1cn(Cc2ccccc2Cl)c2ccccc12] |
| 184 | 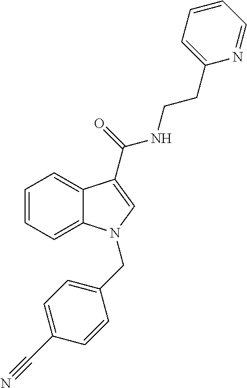 [SMILES: N#Cc1ccc(Cn2cc(C(=O)NCCc3ccccn3)c3ccccc32)cc1] |
| 185 | 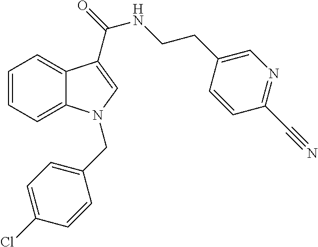 [SMILES: N#Cc1ccc(CCNC(=O)c2cn(Cc3ccc(Cl)cc3)c3ccccc23)cn1] |
| 186 | 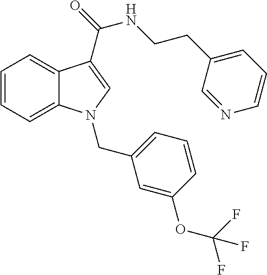 [SMILES: O=C(NCCc1cccnc1)c1cn(Cc2cccc(OC(F)(F)F)c2)c2ccccc12] |
| 187 | 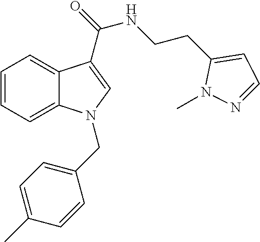 [SMILES: Cc1ccc(Cn2cc(C(=O)NCCc3ccnn3C)c3ccccc32)cc1] |
| 188 | 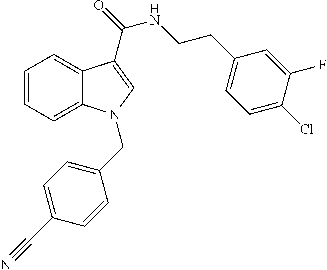 [SMILES: N#Cc1ccc(Cn2cc(C(=O)NCCc3ccc(Cl)c(F)c3)c3ccccc32)cc1] |
| 189 | 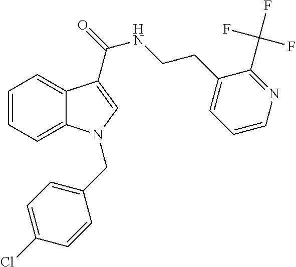 [SMILES: O=C(NCCc1cccnc1C(F)(F)F)c1cn(Cc2ccc(Cl)cc2)c2ccccc12] |
| 190 | 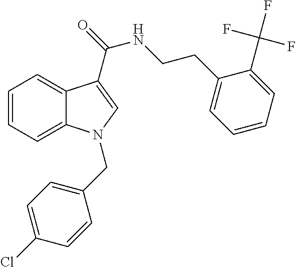 [SMILES: O=C(NCCc1ccccc1C(F)(F)F)c1cn(Cc2ccc(Cl)cc2)c2ccccc12] |
| 191 | 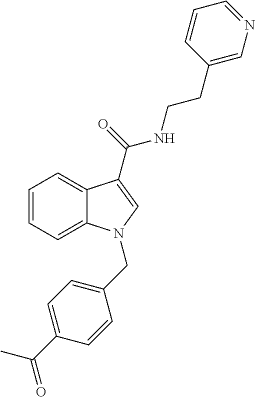 [SMILES: CC(=O)c1ccc(Cn2cc(C(=O)NCCc3cccnc3)c3ccccc32)cc1] |
| 192 | 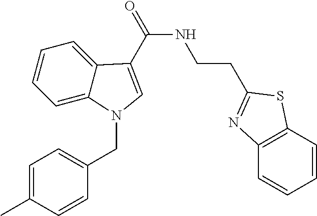 [SMILES: Cc1ccc(Cn2cc(C(=O)NCCc3nc4ccccc4s3)c3ccccc32)cc1] |
| 193 | 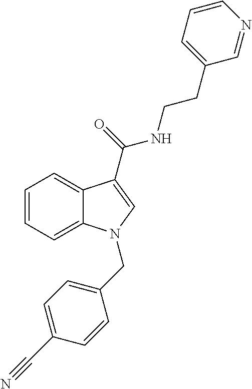 [SMILES: N#Cc1ccc(Cn2cc(C(=O)NCCc3cccnc3)c3ccccc32)cc1] |
| 194 | 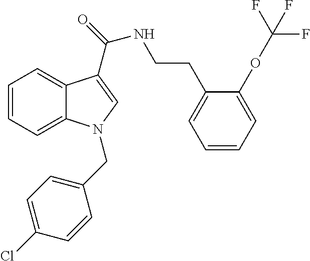 [SMILES: O=C(NCCc1ccccc1OC(F)(F)F)c1cn(Cc2ccc(Cl)cc2)c2ccccc12] |
| 195 | 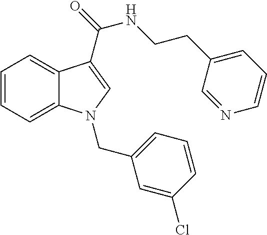 [SMILES: O=C(NCCc1cccnc1)c1cn(Cc2cccc(Cl)c2)c2ccccc12] |
| 196 | 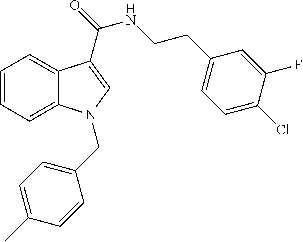 [SMILES: Cc1ccc(Cn2cc(C(=O)NCCc3ccc(Cl)c(F)c3)c3ccccc32)cc1] |
| 197 | 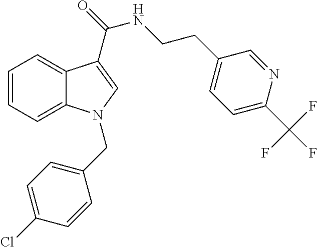 [SMILES: O=C(NCCc1ccc(C(F)(F)F)nc1)c1cn(Cc2ccc(Cl)cc2)c2ccccc12] |
| 198 | 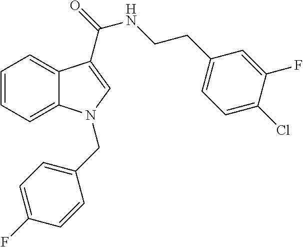 [SMILES: O=C(NCCc1ccc(Cl)c(F)c1)c1cn(Cc2ccc(F)cc2)c2ccccc12] |
| 199 | 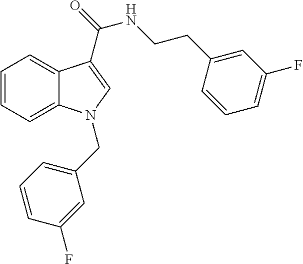 [SMILES: O=C(NCCc1cccc(F)c1)c1cn(Cc2cccc(F)c2)c2ccccc12] |
| 200 | 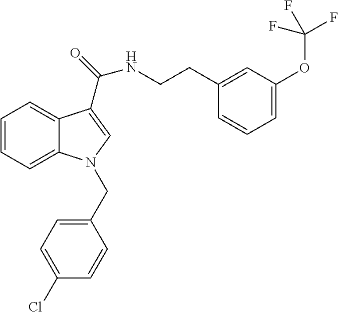 [SMILES: O=C(NCCc1cccc(OC(F)(F)F)c1)c1cn(Cc2ccc(Cl)cc2)c2ccccc12] |
| 201 | 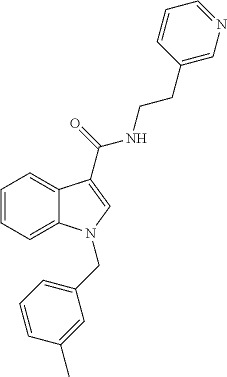 [SMILES: Cc1cccc(Cn2cc(C(=O)NCCc3cccnc3)c3ccccc32)c1] |
| 202 | 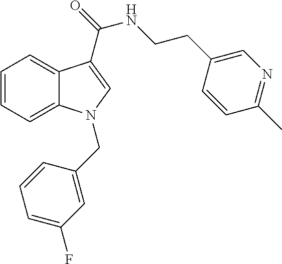 [SMILES: Cc1ccc(CCNC(=O)c2cn(Cc3cccc(F)c3)c3ccccc23)cn1] |
| 203 | 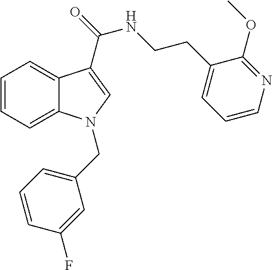 [SMILES: COc1ncccc1CCNC(=O)c1cn(Cc2cccc(F)c2)c2ccccc12] |
| 204 | 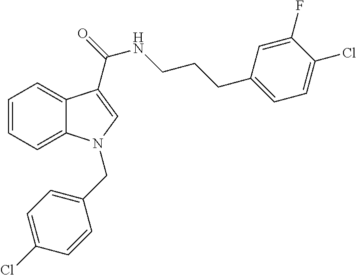 [SMILES: O=C(NCCCc1ccc(Cl)c(F)c1)c1cn(Cc2ccc(Cl)cc2)c2ccccc12] |
| 205 | 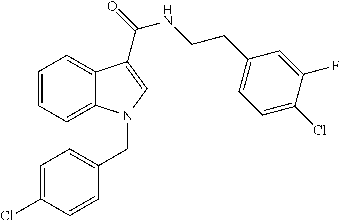 [SMILES: O=C(NCCc1ccc(Cl)c(F)c1)c1cn(Cc2ccc(Cl)cc2)c2ccccc12] |
| 206 | 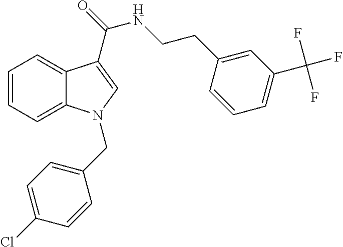 [SMILES: O=C(NCCc1cccc(C(F)(F)F)c1)c1cn(Cc2ccc(Cl)cc2)c2ccccc12] |
| 207 | 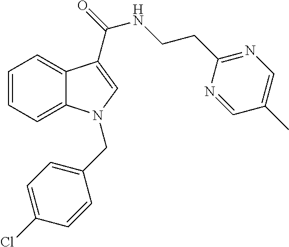 [SMILES: Cc1cnc(CCNC(=O)c2cn(Cc3ccc(Cl)cc3)c3ccccc23)nc1] |
| 208 | 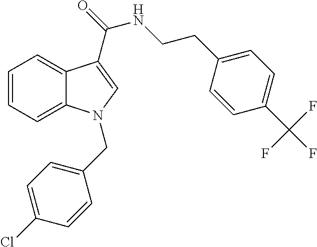 [SMILES: O=C(NCCc1ccc(C(F)(F)F)cc1)c1cn(Cc2ccc(Cl)cc2)c2ccccc12] |
| 209 | 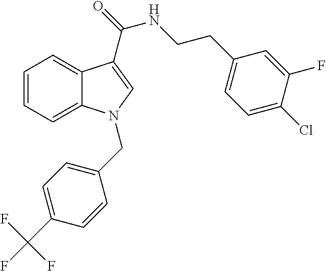 [SMILES: O=C(NCCc1ccc(Cl)c(F)c1)c1cn(Cc2ccc(C(F)(F)F)cc2)c2ccccc12] |
| 210 | 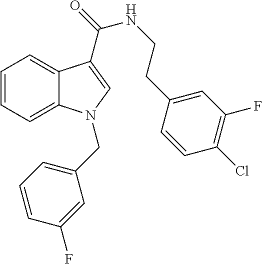 [SMILES: O=C(NCCc1ccc(Cl)c(F)c1)c1cn(Cc2cccc(F)c2)c2ccccc12] |
| 211 | 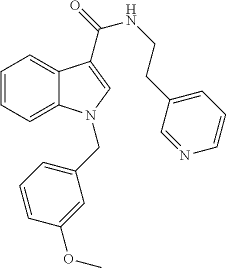 [SMILES: COc1cccc(Cn2cc(C(=O)NCCc3cccnc3)c3ccccc32)c1] |
| 212 | 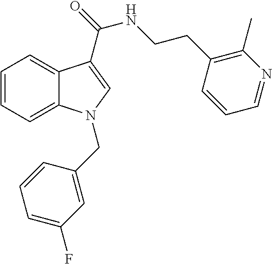 [SMILES: Cc1ncccc1CCNC(=O)c1cn(Cc2cccc(F)c2)c2ccccc12] |
| 213 | 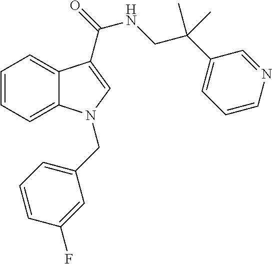 [SMILES: CC(C)(CNC(=O)c1cn(Cc2cccc(F)c2)c2ccccc12)c1cccnc1] |
| 214 | 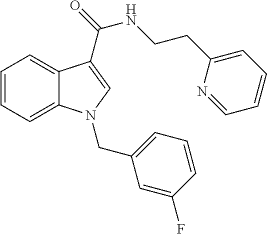 [SMILES: O=C(NCCc1ccccn1)c1cn(Cc2cccc(F)c2)c2ccccc12] |
| 215 | 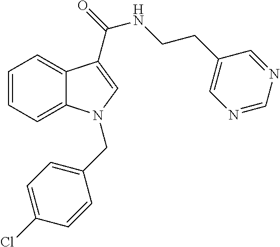 [SMILES: O=C(NCCc1cncnc1)c1cn(Cc2ccc(Cl)cc2)c2ccccc12] |
| 216 | 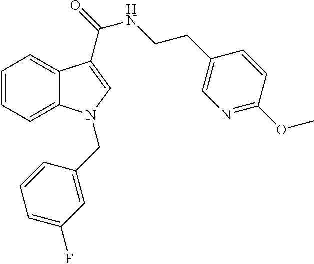 [SMILES: COc1ccc(CCNC(=O)c2cn(Cc3cccc(F)c3)c3ccccc23)cn1] |
| 217 | 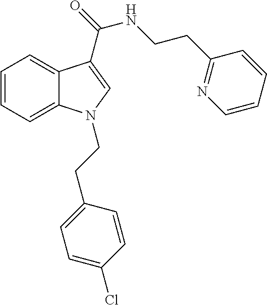 [SMILES: O=C(NCCc1ccccn1)c1cn(CCc2ccc(Cl)cc2)c2ccccc12] |
| 218 | 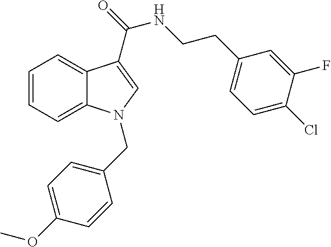 [SMILES: COc1ccc(Cn2cc(C(=O)NCCc3ccc(Cl)c(F)c3)c3ccccc32)cc1] |
| 219 | 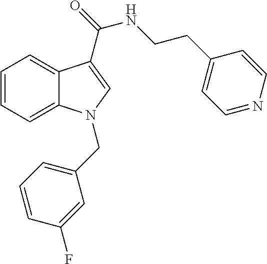 [SMILES: O=C(NCCc1ccncc1)c1cn(Cc2cccc(F)c2)c2ccccc12] |
| 220 | 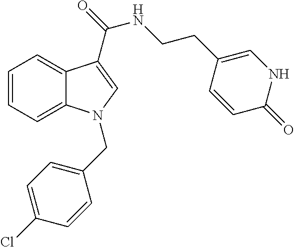 [SMILES: O=C(NCCc1ccc(=O)[nH]c1)c1cn(Cc2ccc(Cl)cc2)c2ccccc12] |
| 221 | 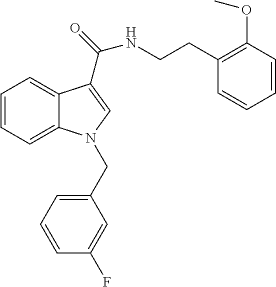 [SMILES: COc1ccccc1CCNC(=O)c1cn(Cc2cccc(F)c2)c2ccccc12] |
| 222 | 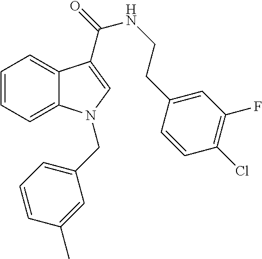 [SMILES: Cc1cccc(Cn2cc(C(=O)NCCc3ccc(Cl)c(F)c3)c3ccccc32)c1] |
| 223 | 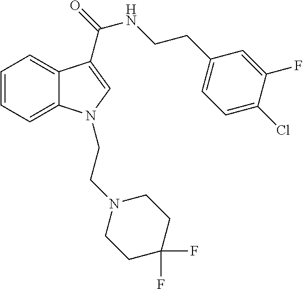 [SMILES: O=C(NCCc1ccc(Cl)c(F)c1)c1cn(CCN2CCC(F)(F)CC2)c2ccccc12] |
| 224 | 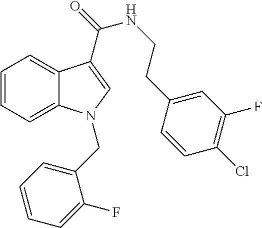 [SMILES: O=C(NCCc1ccc(Cl)c(F)c1)c1cn(Cc2ccccc2F)c2ccccc12] |
| 225 | 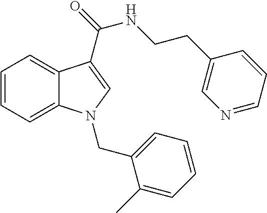 [SMILES: Cc1ccccc1Cn1cc(C(=O)NCCc2cccnc2)c2ccccc21] |
| 226 | 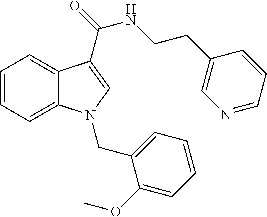 [SMILES: COc1ccccc1Cn1cc(C(=O)NCCc2cccnc2)c2ccccc21] |
| 227 | 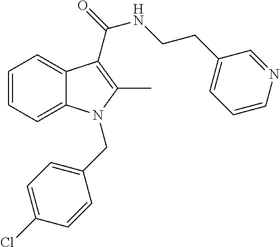 [SMILES: Cc1c(C(=O)NCCc2cccnc2)c2ccccc2n1Cc1ccc(Cl)cc1] |
| 228 | 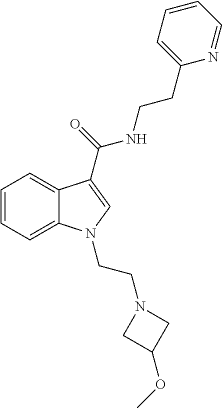 [SMILES: COC1CN(CCn2cc(C(=O)NCCc3ccccn3)c3ccccc32)C1] |
| 229 | 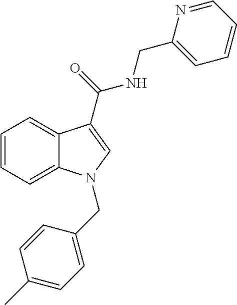 [SMILES: Cc1ccc(Cn2cc(C(=O)NCc3ccccn3)c3ccccc32)cc1] |
| 230 | 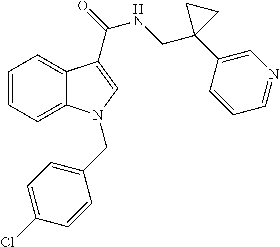 [SMILES: O=C(NCC1(c2cccnc2)CC1)c1cn(Cc2ccc(Cl)cc2)c2ccccc12] |
| 231 | 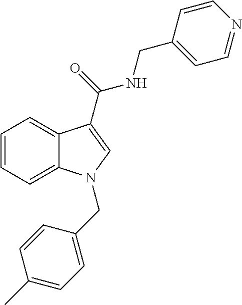 [SMILES: Cc1ccc(Cn2cc(C(=O)NCc3ccncc3)c3ccccc32)cc1] |
| 232 | 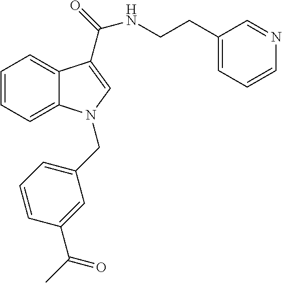 [SMILES: CC(=O)c1cccc(Cn2cc(C(=O)NCCc3cccnc3)c3ccccc32)c1] |
| 233 | 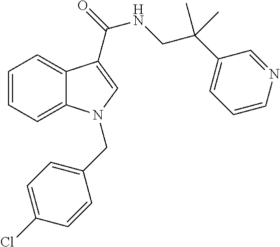 [SMILES: CC(C)(CNC(=O)c1cn(Cc2ccc(Cl)cc2)c2ccccc12)c1cccnc1] |
| 234 | 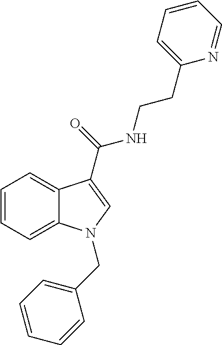 [SMILES: O=C(NCCc1ccccn1)c1cn(Cc2ccccc2)c2ccccc12] |
| 235 | 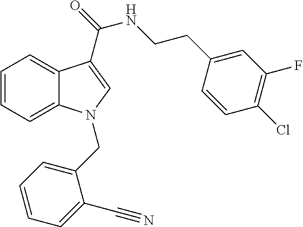 [SMILES: N#Cc1ccccc1Cn1cc(C(=O)NCCc2ccc(Cl)c(F)c2)c2ccccc21] |
| 236 | 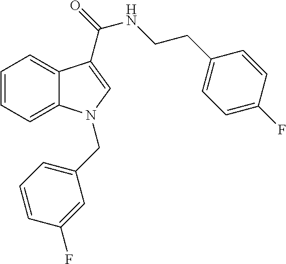 [SMILES: O=C(NCCc1ccc(F)cc1)c1cn(Cc2cccc(F)c2)c2ccccc12] |
| 237 | 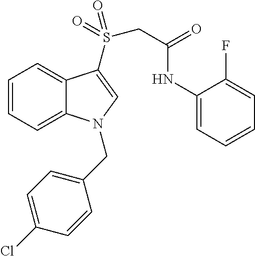 [SMILES: O=C(CS(=O)(=O)c1cn(Cc2ccc(Cl)cc2)c2ccccc12)Nc1ccccc1F] |
| 238 | 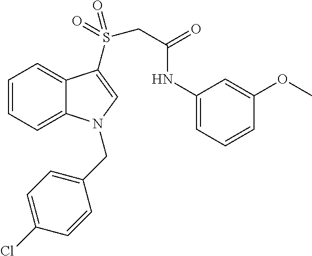 [SMILES: COc1cccc(NC(=O)CS(=O)(=O)c2cn(Cc3ccc(Cl)cc3)c3ccccc23)c1] |
| 239 | 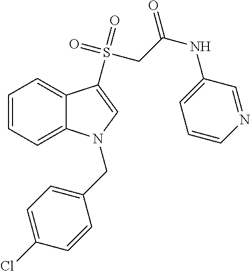 [SMILES: O=C(CS(=O)(=O)c1cn(Cc2ccc(Cl)cc2)c2ccccc12)Nc1cccnc1] |
| 240 | 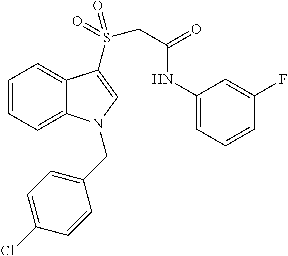 [SMILES: O=C(CS(=O)(=O)c1cn(Cc2ccc(Cl)cc2)c2ccccc12)Nc1cccc(F)c1] |
| 241 | 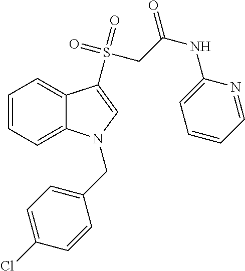 [SMILES: O=C(CS(=O)(=O)c1cn(Cc2ccc(Cl)cc2)c2ccccc12)Nc1ccccn1] |
| 242 | 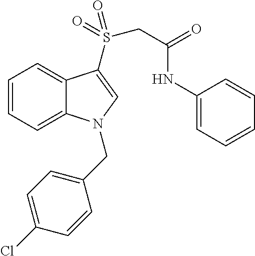 [SMILES: O=C(CS(=O)(=O)c1cn(Cc2ccc(Cl)cc2)c2ccccc12)Nc1ccccc1] |
| 243 | 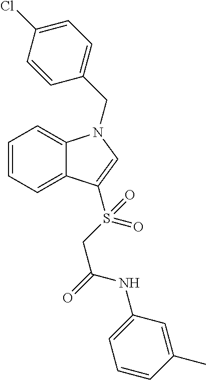 [SMILES: Cc1cccc(NC(=O)CS(=O)(=O)c2cn(Cc3ccc(Cl)cc3)c3ccccc23)c1] |
| 244 | 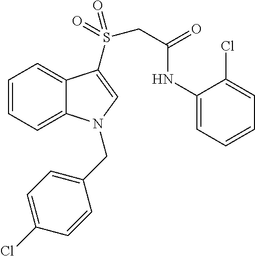 [SMILES: O=C(CS(=O)(=O)c1cn(Cc2ccc(Cl)cc2)c2ccccc12)Nc1ccccc1Cl] |
| 245 | 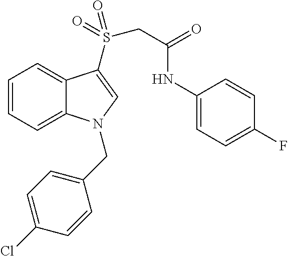 [SMILES: O=C(CS(=O)(=O)c1cn(Cc2ccc(Cl)cc2)c2ccccc12)Nc1ccc(F)cc1] |
| 246 | 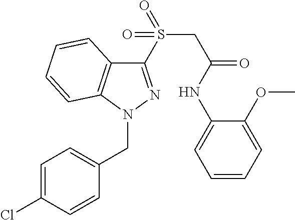 [SMILES: COc1ccccc1NC(=O)CS(=O)(=O)c1nn(Cc2ccc(Cl)cc2)c2ccccc12] |
| 247 | 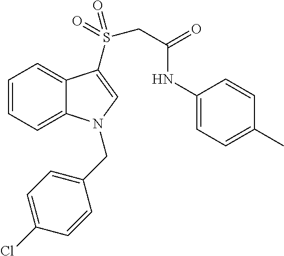 [SMILES: Cc1ccc(NC(=O)CS(=O)(=O)c2cn(Cc3ccc(Cl)cc3)c3ccccc23)cc1] |
| 248 | 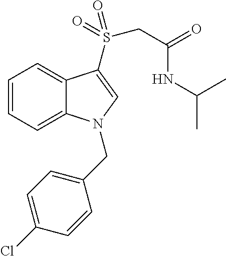 [SMILES: CC(C)NC(=O)CS(=O)(=O)c1cn(Cc2ccc(Cl)cc2)c2ccccc12] |
| 249 | 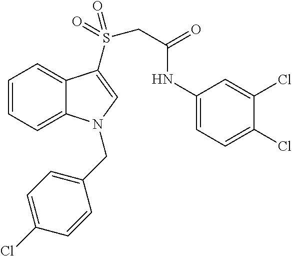 [SMILES: O=C(CS(=O)(=O)c1cn(Cc2ccc(Cl)cc2)c2ccccc12)Nc1ccc(Cl)c(Cl)c1] |
| 250 | 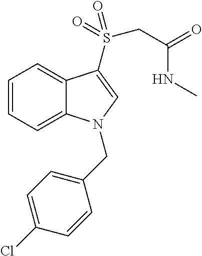 [SMILES: CNC(=O)CS(=O)(=O)c1cn(Cc2ccc(Cl)cc2)c2ccccc12] |
| 251 | 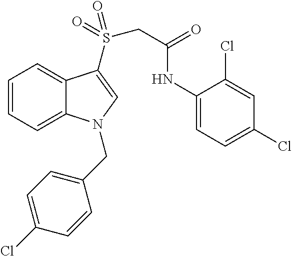 [SMILES: O=C(CS(=O)(=O)c1cn(Cc2ccc(Cl)cc2)c2ccccc12)Nc1ccc(Cl)cc1Cl] |
| 252 | 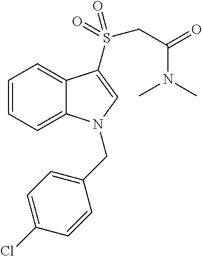 [SMILES: CN(C)C(=O)CS(=O)(=O)c1cn(Cc2ccc(Cl)cc2)c2ccccc12] |
| 253 | 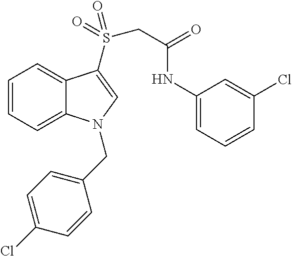 [SMILES: O=C(CS(=O)(=O)c1cn(Cc2ccc(Cl)cc2)c2ccccc12)Nc1cccc(Cl)c1] |
| 254 | 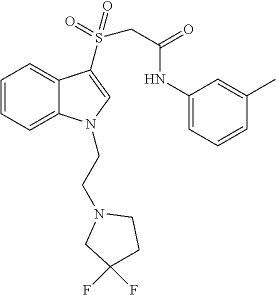 [SMILES: Cc1cccc(NC(=O)CS(=O)(=O)c2cn(CCN3CCC(F)(F)C3)c3ccccc23)c1] |
| 255 | 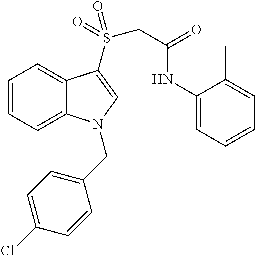 [SMILES: Cc1ccccc1NC(=O)CS(=O)(=O)c1cn(Cc2ccc(Cl)cc2)c2ccccc12] |
| 256 | 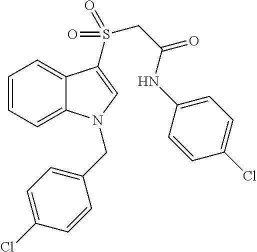 [SMILES: O=C(CS(=O)(=O)c1cn(Cc2ccc(Cl)cc2)c2ccccc12)Nc1ccc(Cl)cc1] |
| 257 | 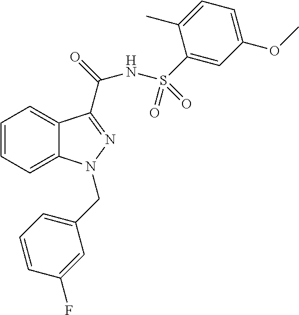 [SMILES: COc1ccc(C)c(S(=O)(=O)NC(=O)c2nn(Cc3cccc(F)c3)c3ccccc23)c1] |
| 258 | 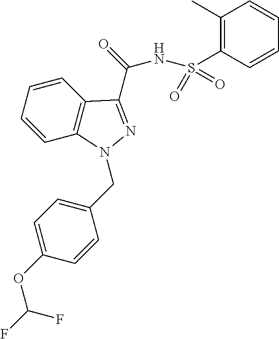 [SMILES: Cc1ccccc1S(=O)(=O)NC(=O)c1nn(Cc2ccc(OC(F)F)cc2)c2ccccc12] |
| 259 | 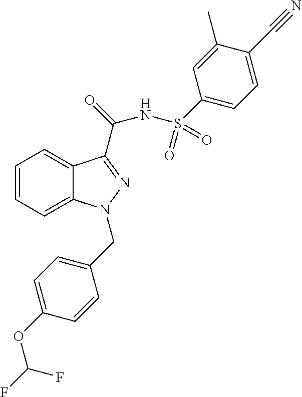 [SMILES: Cc1cc(S(=O)(=O)NC(=O)c2nn(Cc3ccc(OC(F)F)cc3)c3ccccc23)ccc1C#N] |
| 260 | 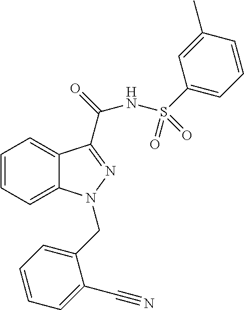 [SMILES: Cc1cccc(S(=O)(=O)NC(=O)c2nn(Cc3ccccc3C#N)c3ccccc23)c1] |
| 261 | 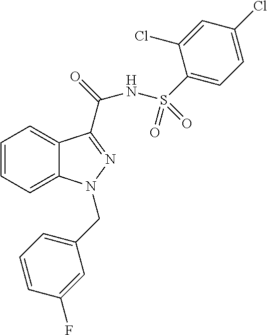 [SMILES: O=C(NS(=O)(=O)c1ccc(Cl)cc1Cl)c1nn(Cc2cccc(F)c2)c2ccccc12] |
| 262 | 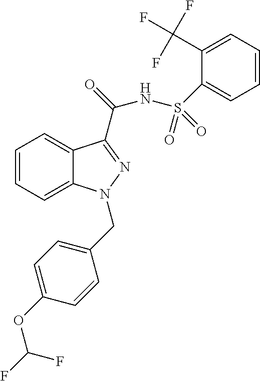 [SMILES: O=C(NS(=O)(=O)c1ccccc1C(F)(F)F)c1nn(Cc2ccc(OC(F)F)cc2)c2ccccc12] |
| 263 | 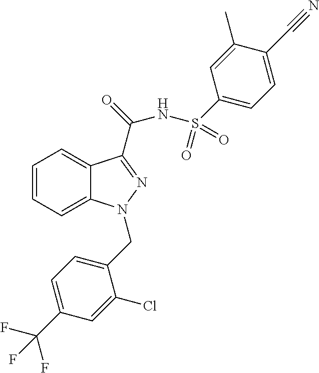 [SMILES: Cc1cc(S(=O)(=O)NC(=O)c2nn(Cc3ccc(C(F)(F)F)cc3Cl)c3ccccc23)ccc1C#N] |
| 264 | 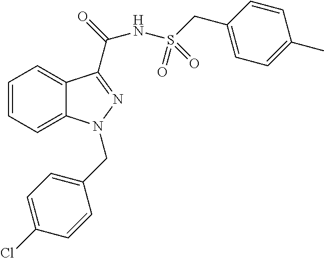 [SMILES: Cc1ccc(CS(=O)(=O)NC(=O)c2nn(Cc3ccc(Cl)cc3)c3ccccc23)cc1] |
| 265 | 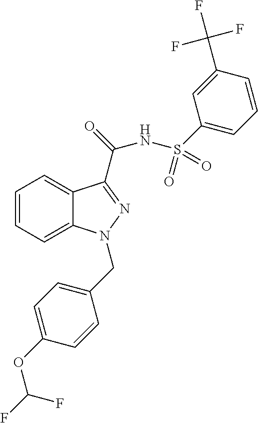 [SMILES: O=C(NS(=O)(=O)c1cccc(C(F)(F)F)c1)c1nn(Cc2ccc(OC(F)F)cc2)c2ccccc12] |
| 266 | 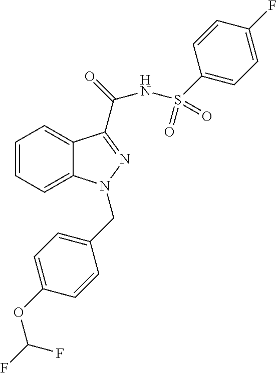 [SMILES: O=C(NS(=O)(=O)c1ccc(F)cc1)c1nn(Cc2ccc(OC(F)F)cc2)c2ccccc12] |
| 267 | 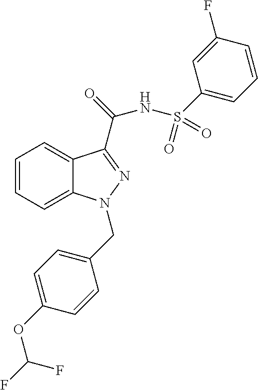 [SMILES: O=C(NS(=O)(=O)c1cccc(F)c1)c1nn(Cc2ccc(OC(F)F)cc2)c2ccccc12] |
| 268 | 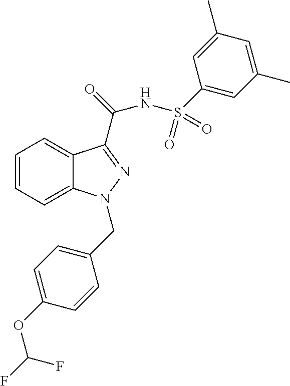 [SMILES: Cc1cc(C)cc(S(=O)(=O)NC(=O)c2nn(Cc3ccc(OC(F)F)cc3)c3ccccc23)c1] |
| 269 | 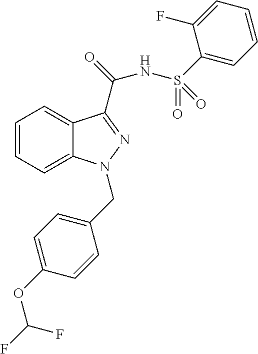 [SMILES: O=C(NS(=O)(=O)c1ccccc1F)c1nn(Cc2ccc(OC(F)F)cc2)c2ccccc12] |
| 270 | 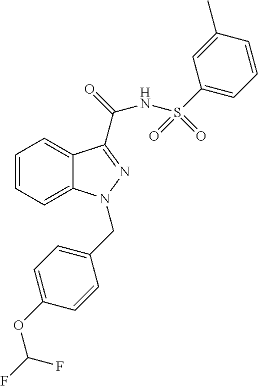 [SMILES: Cc1cccc(S(=O)(=O)NC(=O)c2nn(Cc3ccc(OC(F)F)cc3)c3ccccc23)c1] |
| 271 | 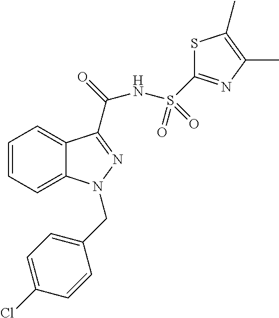 [SMILES: Cc1nc(S(=O)(=O)NC(=O)c2nn(Cc3ccc(Cl)cc3)c3ccccc23)sc1C] |
| 272 | 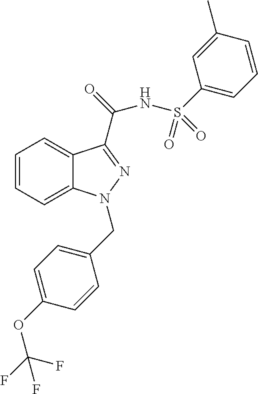 [SMILES: Cc1cccc(S(=O)(=O)NC(=O)c2nn(Cc3ccc(OC(F)(F)F)cc3)c3ccccc23)c1] |
| 273 | 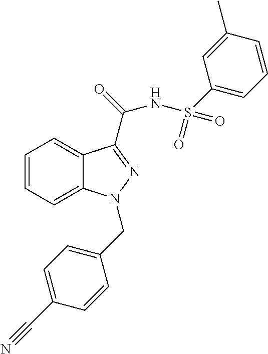 [SMILES: Cc1cccc(S(=O)(=O)NC(=O)c2nn(Cc3ccc(C#N)cc3)c3ccccc23)c1] |
| 274 | 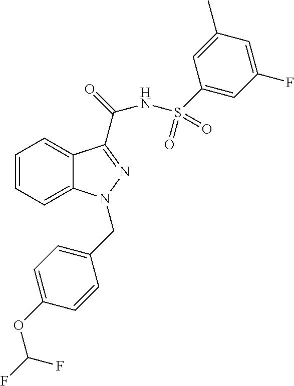 [SMILES: Cc1cc(F)cc(S(=O)(=O)NC(=O)c2nn(Cc3ccc(OC(F)F)cc3)c3ccccc23)c1] |
| 275 | 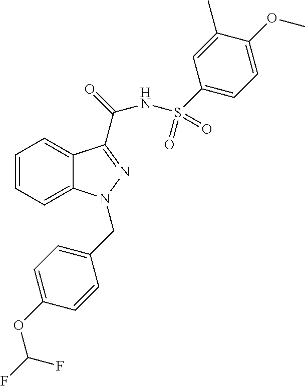 [SMILES: COc1ccc(S(=O)(=O)NC(=O)c2nn(Cc3ccc(OC(F)F)cc3)c3ccccc23)cc1C] |
| 276 | 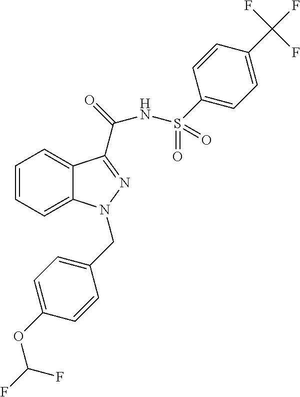 [SMILES: O=C(NS(=O)(=O)c1ccc(C(F)(F)F)cc1)c1nn(Cc2ccc(OC(F)F)cc2)c2ccccc12] |
| 277 | 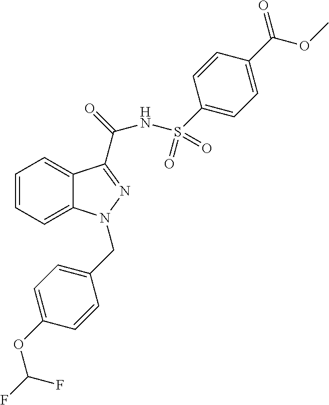 [SMILES: COC(=O)c1ccc(S(=O)(=O)NC(=O)c2nn(Cc3ccc(OC(F)F)cc3)c3ccccc23)cc1] |
| 278 | 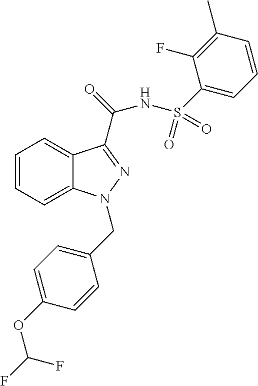 [SMILES: Cc1cccc(S(=O)(=O)NC(=O)c2nn(Cc3ccc(OC(F)F)cc3)c3ccccc23)c1F] |
| 279 | 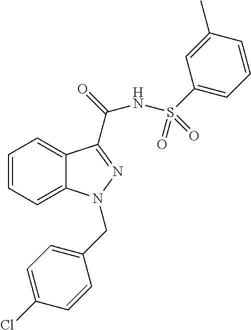 [SMILES: Cc1cccc(S(=O)(=O)NC(=O)c2nn(Cc3ccc(Cl)cc3)c3ccccc23)c1] |
| 280 | 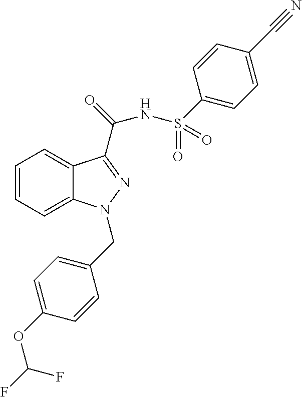 [SMILES: N#Cc1ccc(S(=O)(=O)NC(=O)c2nn(Cc3ccc(OC(F)F)cc3)c3ccccc23)cc1] |
| 281 | 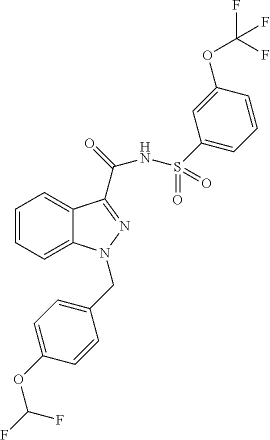 [SMILES: O=C(NS(=O)(=O)c1cccc(OC(F)(F)F)c1)c1nn(Cc2ccc(OC(F)F)cc2)c2ccccc12] |
| 282 | 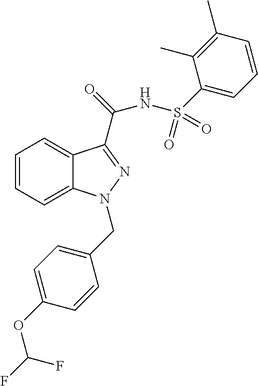 [SMILES: Cc1cccc(S(=O)(=O)NC(=O)c2nn(Cc3ccc(OC(F)F)cc3)c3ccccc23)c1C] |
| 283 | 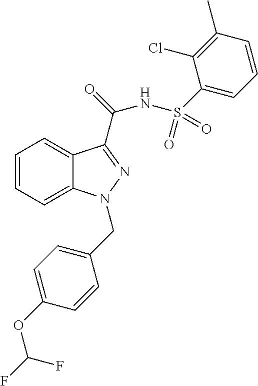 [SMILES: Cc1cccc(S(=O)(=O)NC(=O)c2nn(Cc3ccc(OC(F)F)cc3)c3ccccc23)c1Cl] |
| 284 | 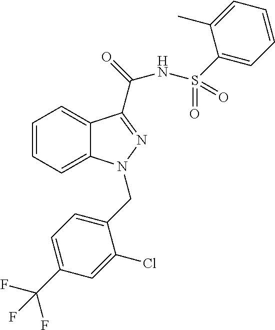 [SMILES: Cc1ccccc1S(=O)(=O)NC(=O)c1nn(Cc2ccc(C(F)(F)F)cc2Cl)c2ccccc12] |
| 285 | 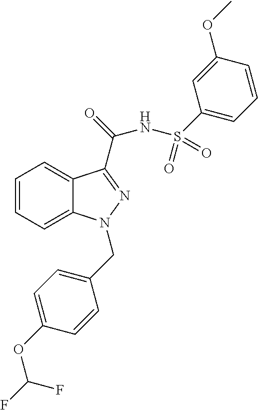 [SMILES: COc1cccc(S(=O)(=O)NC(=O)c2nn(Cc3ccc(OC(F)F)cc3)c3ccccc23)c1] |
| 286 | 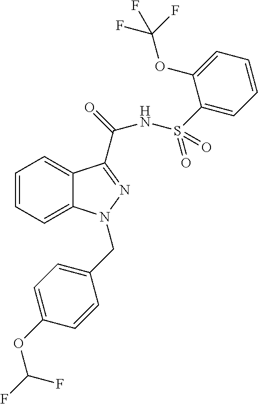 [SMILES: O=C(NS(=O)(=O)c1ccccc1OC(F)(F)F)c1nn(Cc2ccc(OC(F)F)cc2)c2ccccc12] |
| 287 | 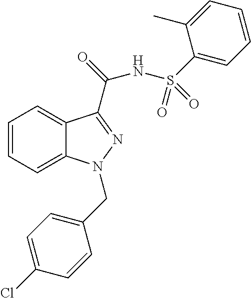 [SMILES: Cc1ccccc1S(=O)(=O)NC(=O)c1nn(Cc2ccc(Cl)cc2)c2ccccc12] |
| 288 | 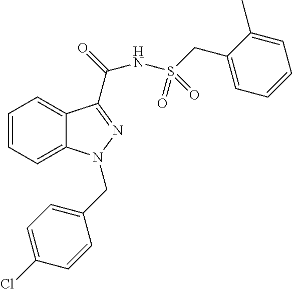 [SMILES: Cc1ccccc1CS(=O)(=O)NC(=O)c1nn(Cc2ccc(Cl)cc2)c2ccccc12] |
| 289 | 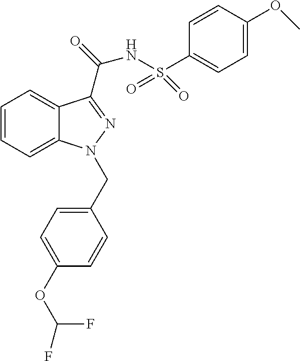 [SMILES: COc1ccc(S(=O)(=O)NC(=O)c2nn(Cc3ccc(OC(F)F)cc3)c3ccccc23)cc1] |
| 290 | 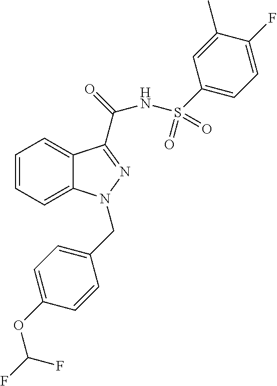 [SMILES: Cc1cc(S(=O)(=O)NC(=O)c2nn(Cc3ccc(OC(F)F)cc3)c3ccccc23)ccc1F] |
| 291 | 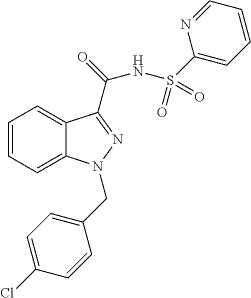 [SMILES: O=C(NS(=O)(=O)c1ccccn1)c1nn(Cc2ccc(Cl)cc2)c2ccccc12] |
| 292 | 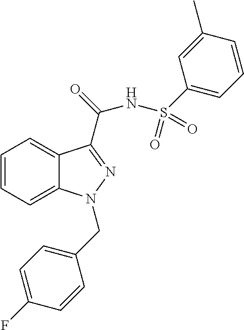 [SMILES: Cc1cccc(S(=O)(=O)NC(=O)c2nn(Cc3ccc(F)cc3)c3ccccc23)c1] |
| 293 | 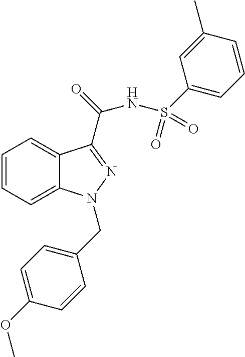 [SMILES: COc1ccc(Cn2nc(C(=O)NS(=O)(=O)c3cccc(C)c3)c3ccccc32)cc1] |
| 294 | 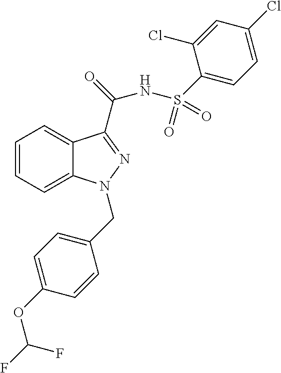 [SMILES: O=C(NS(=O)(=O)c1ccc(Cl)cc1Cl)c1nn(Cc2ccc(OC(F)F)cc2)c2ccccc12] |
| 295 | 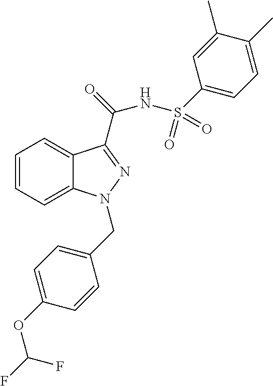 [SMILES: Cc1ccc(S(=O)(=O)NC(=O)c2nn(Cc3ccc(OC(F)F)cc3)c3ccccc23)cc1C] |
| 296 | 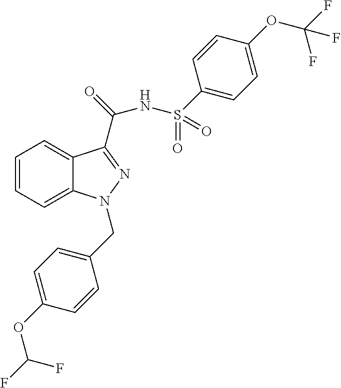 [SMILES: O=C(NS(=O)(=O)c1ccc(OC(F)(F)F)cc1)c1nn(Cc2ccc(OC(F)F)cc2)c2ccccc12] |
| 297 | 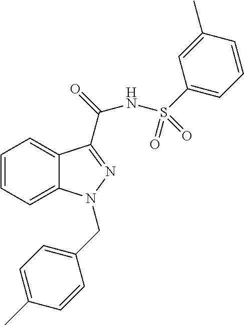 [SMILES: Cc1ccc(Cn2nc(C(=O)NS(=O)(=O)c3cccc(C)c3)c3ccccc32)cc1] |
| 298 | 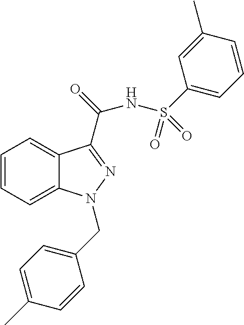 [SMILES: Cc1ccc(Cn2nc(C(=O)NS(=O)(=O)c3cccc(C)c3)c3ccccc32)cc1] |
| 299 | 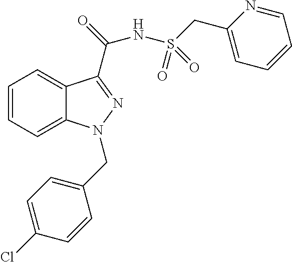 [SMILES: O=C(NS(=O)(=O)Cc1ccccn1)c1nn(Cc2ccc(Cl)cc2)c2ccccc12] |
| 300 | 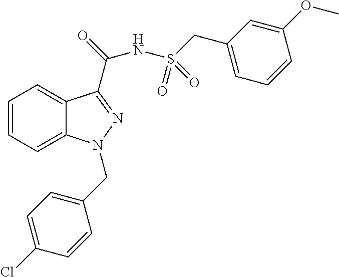 [SMILES: COc1cccc(CS(=O)(=O)NC(=O)c2nn(Cc3ccc(Cl)cc3)c3ccccc23)c1] |
| 301 | 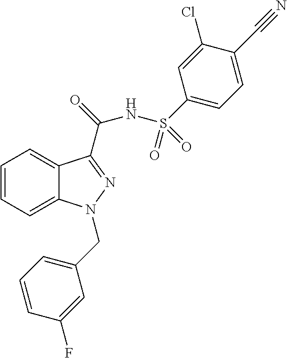 [SMILES: N#Cc1ccc(S(=O)(=O)NC(=O)c2nn(Cc3cccc(F)c3)c3ccccc23)cc1Cl] |
| 302 | 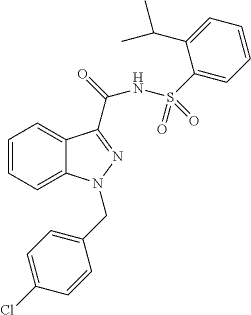 [SMILES: CC(C)c1ccccc1S(=O)(=O)NC(=O)c1nn(Cc2ccc(Cl)cc2)c2ccccc12] |
| 303 | 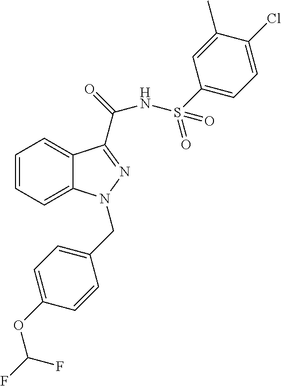 [SMILES: Cc1cc(S(=O)(=O)NC(=O)c2nn(Cc3ccc(OC(F)F)cc3)c3ccccc23)ccc1Cl] |
| 304 | 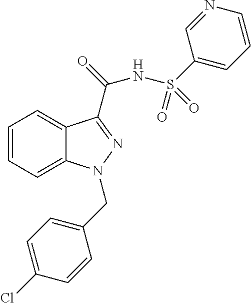 [SMILES: O=C(NS(=O)(=O)c1cccnc1)c1nn(Cc2ccc(Cl)cc2)c2ccccc12] |
| 305 | 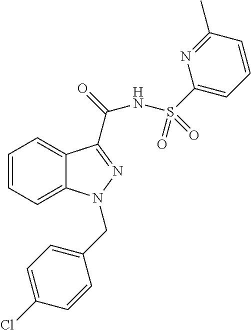 [SMILES: Cc1cccc(S(=O)(=O)NC(=O)c2nn(Cc3ccc(Cl)cc3)c3ccccc23)n1] |
| 306 | 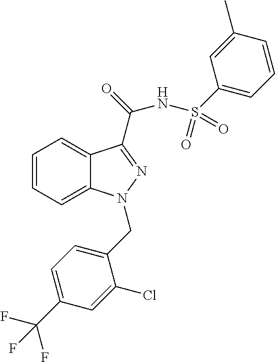 [SMILES: Cc1cccc(S(=O)(=O)NC(=O)c2nn(Cc3ccc(C(F)(F)F)cc3Cl)c3ccccc23)c1] |
| 307 | 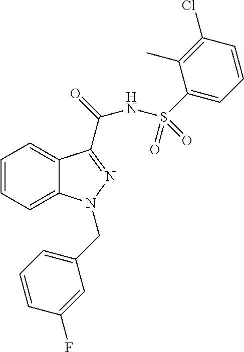 [SMILES: Cc1c(Cl)cccc1S(=O)(=O)NC(=O)c1nn(Cc2cccc(F)c2)c2ccccc12] |
| 308 | 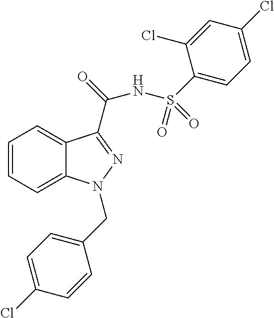 [SMILES: O=C(NS(=O)(=O)c1ccc(Cl)cc1Cl)c1nn(Cc2ccc(Cl)cc2)c2ccccc12] |
| 309 | 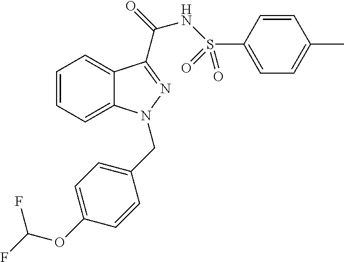 [SMILES: Cc1ccc(S(=O)(=O)NC(=O)c2nn(Cc3ccc(OC(F)F)cc3)c3ccccc23)cc1] |
| 310 | 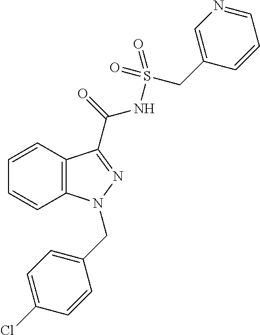 [SMILES: O=C(NS(=O)(=O)Cc1cccnc1)c1nn(Cc2ccc(Cl)cc2)c2ccccc12] |
| 311 | 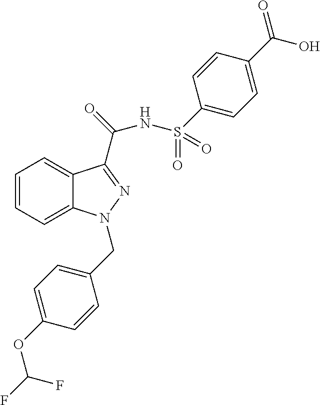 [SMILES: O=C(O)c1ccc(S(=O)(=O)NC(=O)c2nn(Cc3ccc(OC(F)F)cc3)c3ccccc23)cc1] |
| 312 | 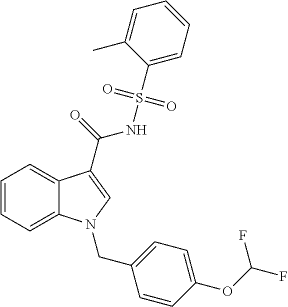 [SMILES: Cc1ccccc1S(=O)(=O)NC(=O)c1cn(Cc2ccc(OC(F)F)cc2)c2ccccc12] |
| 313 | 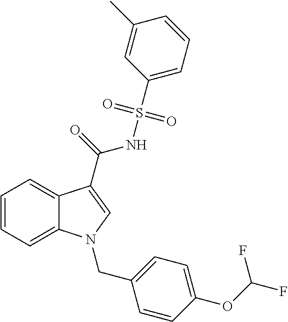 [SMILES: Cc1cccc(S(=O)(=O)NC(=O)c2cn(Cc3ccc(OC(F)F)cc3)c3ccccc23)c1] |
| 314 | 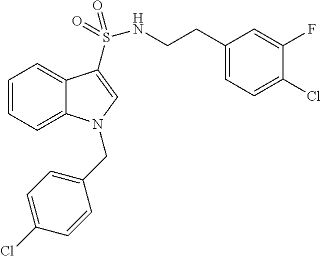 [SMILES: O=S(=O)(NCCc1ccc(Cl)c(F)c1)c1cn(Cc2ccc(Cl)cc2)c2ccccc12] |
| 315 | 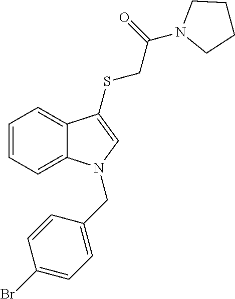 [SMILES: O=C(CSc1cn(Cc2ccc(Br)cc2)c2ccccc12)N1CCCC1] |
| 316 | 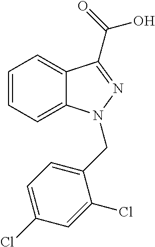 [SMILES: O=C(O)c1nn(Cc2ccc(Cl)cc2Cl)c2ccccc12] |
| 317 | 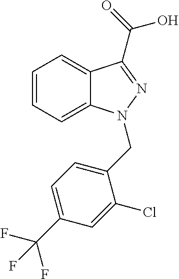 [SMILES: O=C(O)c1nn(Cc2ccc(C(F)(F)F)cc2Cl)c2ccccc12] |
| 318 | 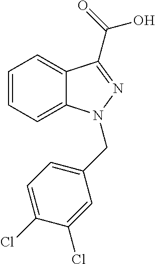 [SMILES: O=C(O)c1nn(Cc2ccc(Cl)c(Cl)c2)c2ccccc12] |
| 319 | 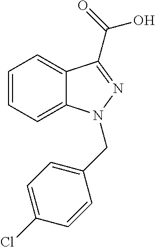 [SMILES: O=C(O)c1nn(Cc2ccc(Cl)cc2)c2ccccc12] |
| 320 | 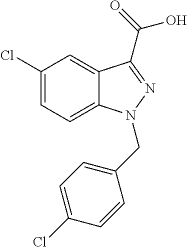 [SMILES: O=C(O)c1nn(Cc2ccc(Cl)cc2)c2ccc(Cl)cc12] |
| 321 | 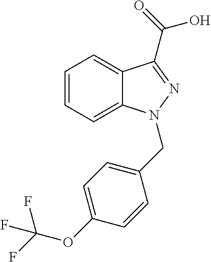 [SMILES: O=C(O)c1nn(Cc2ccc(OC(F)(F)F)cc2)c2ccccc12] |
| 322 | 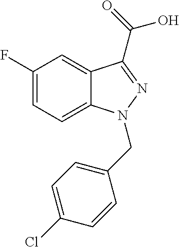 [SMILES: O=C(O)c1nn(Cc2ccc(Cl)cc2)c2ccc(F)cc12] |
| 323 | 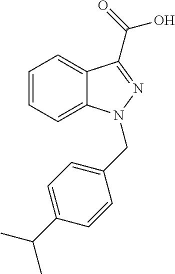 [SMILES: CC(C)c1ccc(Cn2nc(C(=O)O)c3ccccc32)cc1] |
| 324 | 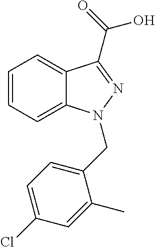 [SMILES: Cc1cc(Cl)ccc1Cn1nc(C(=O)O)c2ccccc21] |
| 325 | 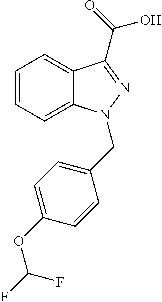 [SMILES: O=C(O)c1nn(Cc2ccc(OC(F)F)cc2)c2ccccc12] |
| 326 | 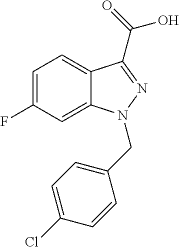 [SMILES: O=C(O)c1nn(Cc2ccc(Cl)cc2)c2cc(F)ccc12] |
| 327 | 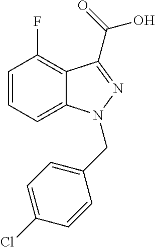 [SMILES: O=C(O)c1nn(Cc2ccc(Cl)cc2)c2cccc(F)c12] |
| 328 | 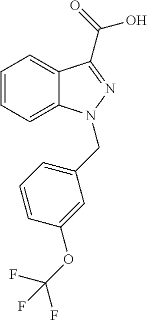 [SMILES: O=C(O)c1nn(Cc2cccc(OC(F)(F)F)c2)c2ccccc12] |
| 329 | 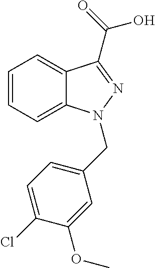 [SMILES: COc1cc(Cn2nc(C(=O)O)c3ccccc32)ccc1Cl] |
| 330 | 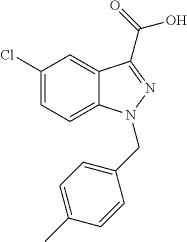 [SMILES: Cc1ccc(Cn2nc(C(=O)O)c3cc(Cl)ccc32)cc1] |
| 331 | 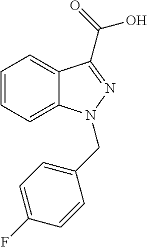 [SMILES: O=C(O)c1nn(Cc2ccc(F)cc2)c2ccccc12] |
| 332 | 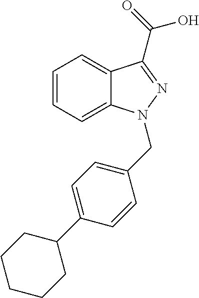 [SMILES: O=C(O)c1nn(Cc2ccc(C3CCCCC3)cc2)c2ccccc12] |
| 333 | 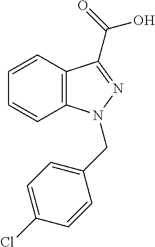 [SMILES: O=C(O)c1nn(Cc2ccc(Cl)cc2)c2ccccc12] |
| 334 | 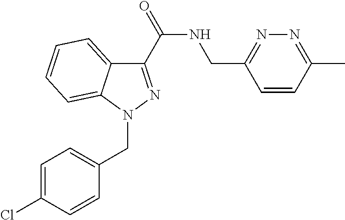 [SMILES: Cc1ccc(CNC(=O)c2nn(Cc3ccc(Cl)cc3)c3ccccc23)nn1] |
| 335 | 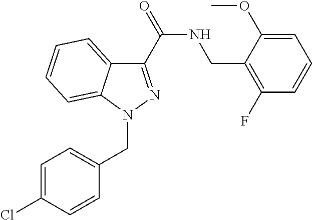 [SMILES: COc1cccc(F)c1CNC(=O)c1nn(Cc2ccc(Cl)cc2)c2ccccc12] |
| 336 | 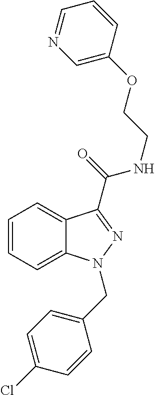 [SMILES: O=C(NCCOc1cccnc1)c1nn(Cc2ccc(Cl)cc2)c2ccccc12] |
| 337 | 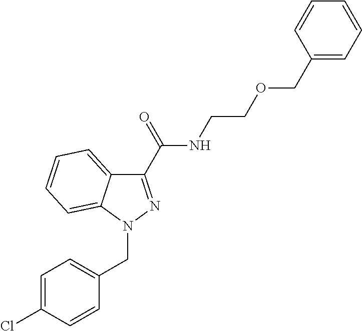 [SMILES: O=C(NCCOCc1ccccc1)c1nn(Cc2ccc(Cl)cc2)c2ccccc12] |
| 338 | 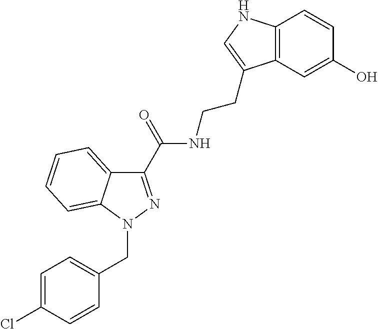 [SMILES: O=C(NCCc1c[nH]c2ccc(O)cc12)c1nn(Cc2ccc(Cl)cc2)c2ccccc12] |
| 339 | 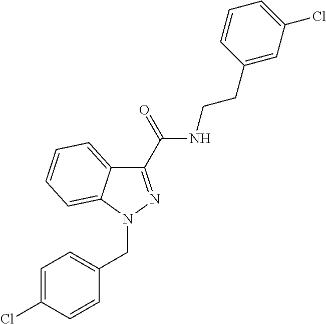 [SMILES: O=C(NCCc1cccc(Cl)c1)c1nn(Cc2ccc(Cl)cc2)c2ccccc12] |
| 340 | 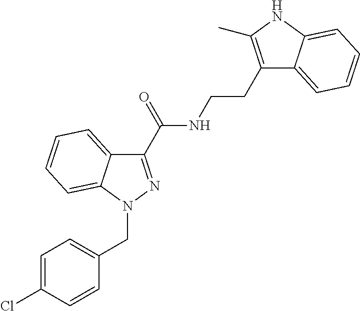 [SMILES: Cc1[nH]c2ccccc2c1CCNC(=O)c1nn(Cc2ccc(Cl)cc2)c2ccccc12] |
| 341 | 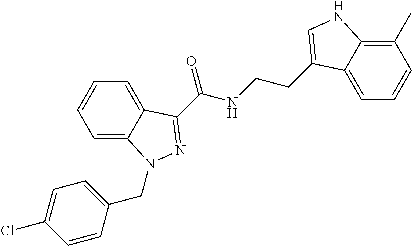 [SMILES: Cc1cccc2c(CCNC(=O)c3nn(Cc4ccc(Cl)cc4)c4ccccc34)c[nH]c12] |
| 342 | 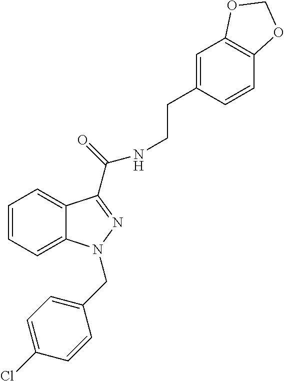 [SMILES: O=C(NCCc1ccc2c(c1)OCO2)c1nn(Cc2ccc(Cl)cc2)c2ccccc12] |
| 343 | 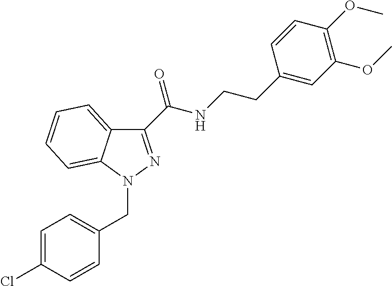 [SMILES: COc1ccc(CCNC(=O)c2nn(Cc3ccc(Cl)cc3)c3ccccc23)cc1OC] |
| 344 | 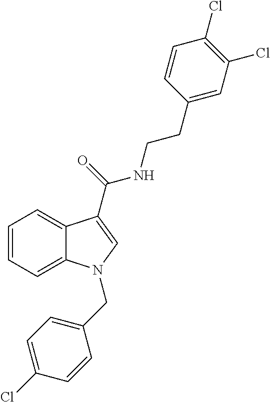 [SMILES: O=C(NCCc1ccc(Cl)c(Cl)c1)c1cn(Cc2ccc(Cl)cc2)c2ccccc12] |
| 345 | 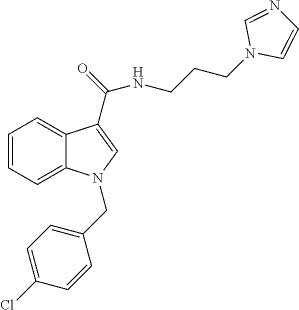 [SMILES: O=C(NCCCn1ccnc1)c1cn(Cc2ccc(Cl)cc2)c2ccccc12] |
| 346 | 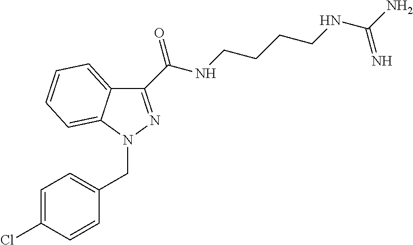 [SMILES: N=C(N)NCCCCNC(=O)c1nn(Cc2ccc(Cl)cc2)c2ccccc12] |
| 347 | 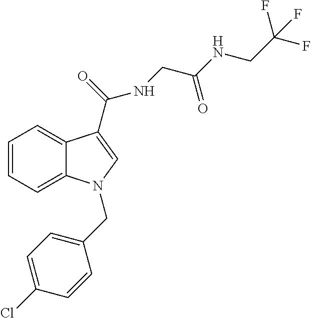 [SMILES: O=C(CNC(=O)c1cn(Cc2ccc(Cl)cc2)c2ccccc12)NCC(F)(F)F] |
| 348 | 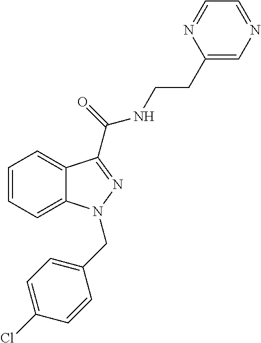 [SMILES: O=C(NCCc1cnccn1)c1nn(Cc2ccc(Cl)cc2)c2ccccc12] |
| 349 | 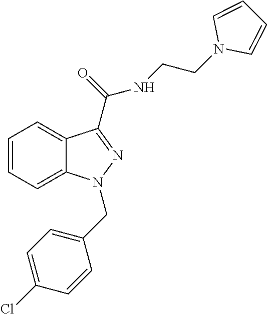 [SMILES: O=C(NCCn1cccc1)c1nn(Cc2ccc(Cl)cc2)c2ccccc12] |
| 350 | 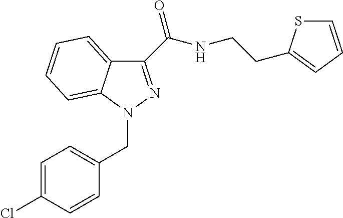 [SMILES: O=C(NCCc1cccs1)c1nn(Cc2ccc(Cl)cc2)c2ccccc12] |
| 351 | 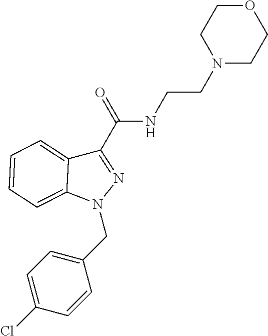 [SMILES: O=C(NCCN1CCOCC1)c1nn(Cc2ccc(Cl)cc2)c2ccccc12] |
| 352 | 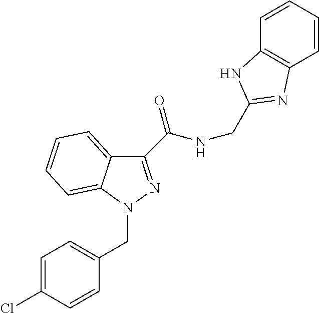 [SMILES: O=C(NCc1nc2ccccc2[nH]1)c1nn(Cc2ccc(Cl)cc2)c2ccccc12] |
| 353 | 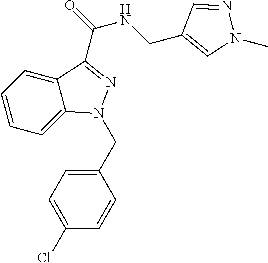 [SMILES: Cn1cc(CNC(=O)c2nn(Cc3ccc(Cl)cc3)c3ccccc23)cn1] |
| 354 | 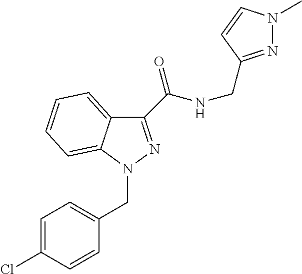 [SMILES: Cn1ccc(CNC(=O)c2nn(Cc3ccc(Cl)cc3)c3ccccc23)n1] |
| 355 | 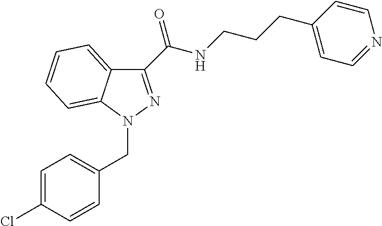 [SMILES: O=C(NCCCc1ccncc1)c1nn(Cc2ccc(Cl)cc2)c2ccccc12] |
| 356 | 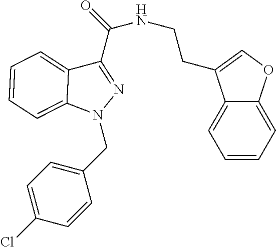 [SMILES: O=C(NCCc1coc2ccccc12)c1nn(Cc2ccc(Cl)cc2)c2ccccc12] |
| 357 | 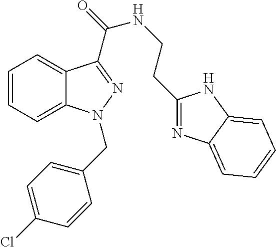 [SMILES: O=C(NCCc1nc2ccccc2[nH]1)c1nn(Cc2ccc(Cl)cc2)c2ccccc12] |
| 358 | 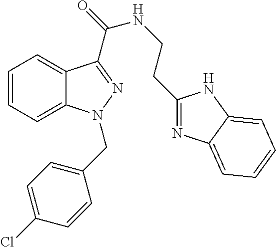 [SMILES: O=C(NCCc1nc2ccccc2[nH]1)c1nn(Cc2ccc(Cl)cc2)c2ccccc12] |
| 359 | 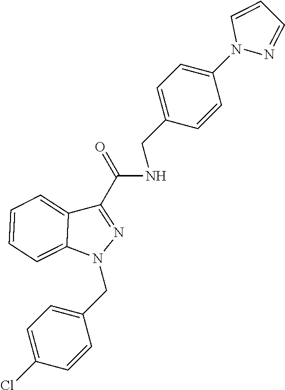 [SMILES: O=C(NCc1ccc(-n2cccn2)cc1)c1nn(Cc2ccc(Cl)cc2)c2ccccc12] |
| 360 | 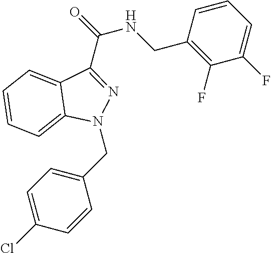 [SMILES: O=C(NCc1cccc(F)c1F)c1nn(Cc2ccc(Cl)cc2)c2ccccc12] |
| 361 | 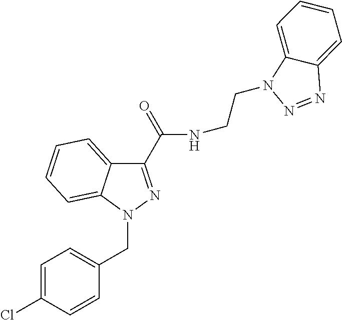 [SMILES: O=C(NCCn1nnc2ccccc21)c1nn(Cc2ccc(Cl)cc2)c2ccccc12] |
| 362 | 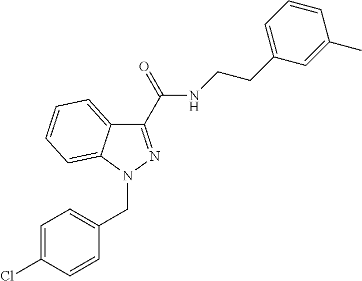 [SMILES: Cc1cccc(CCNC(=O)c2nn(Cc3ccc(Cl)cc3)c3ccccc23)c1] |
| 363 | 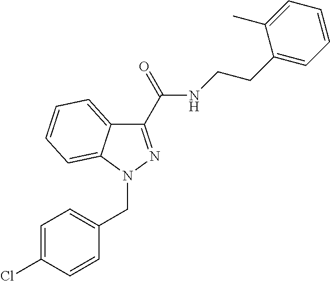 [SMILES: Cc1ccccc1CCNC(=O)c1nn(Cc2ccc(Cl)cc2)c2ccccc12] |
| 364 | 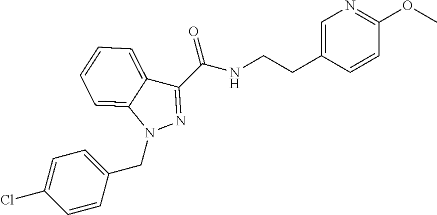 [SMILES: COc1ccc(CCNC(=O)c2nn(Cc3ccc(Cl)cc3)c3ccccc23)cn1] |
| 365 | 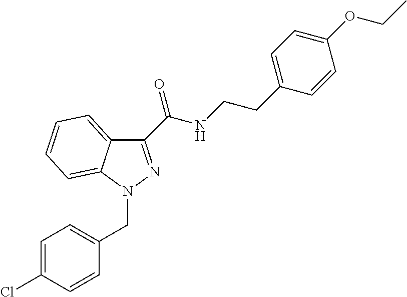 [SMILES: CCOc1ccc(CCNC(=O)c2nn(Cc3ccc(Cl)cc3)c3ccccc23)cc1] |
| 366 | 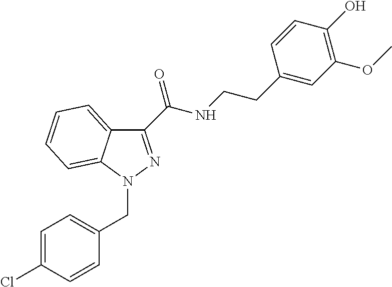 [SMILES: COc1cc(CCNC(=O)c2nn(Cc3ccc(Cl)cc3)c3ccccc23)ccc1O] |
| 367 | 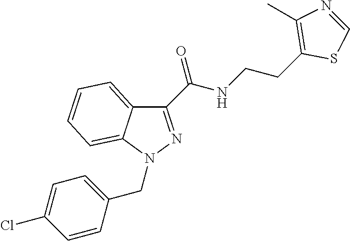 [SMILES: Cc1ncsc1CCNC(=O)c1nn(Cc2ccc(Cl)cc2)c2ccccc12] |
| 368 | 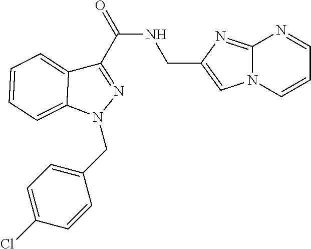 [SMILES: O=C(NCc1cn2cccnc2n1)c1nn(Cc2ccc(Cl)cc2)c2ccccc12] |
| 369 | 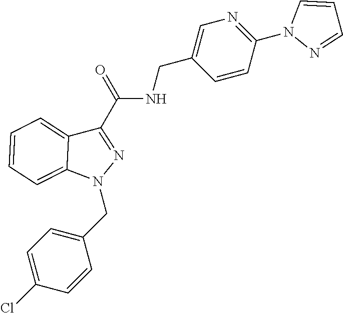 [SMILES: O=C(NCc1ccc(-n2cccn2)nc1)c1nn(Cc2ccc(Cl)cc2)c2ccccc12] |
| 370 | 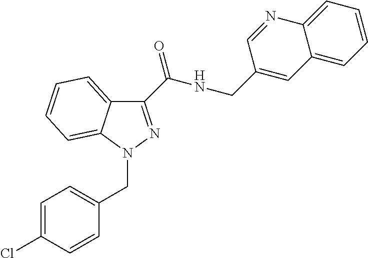 [SMILES: O=C(NCc1cnc2ccccc2c1)c1nn(Cc2ccc(Cl)cc2)c2ccccc12] |
| 371 | 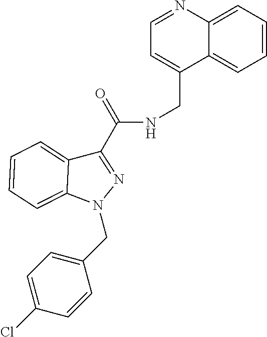 [SMILES: O=C(NCc1ccnc2ccccc12)c1nn(Cc2ccc(Cl)cc2)c2ccccc12] |
| 372 | 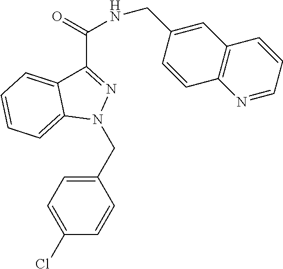 [SMILES: O=C(NCc1ccc2ncccc2c1)c1nn(Cc2ccc(Cl)cc2)c2ccccc12] |
| 373 | 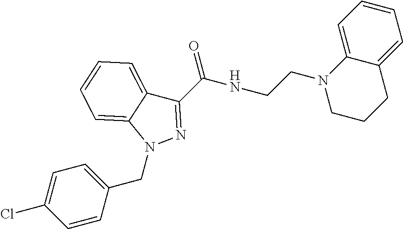 [SMILES: O=C(NCCN1CCCc2ccccc21)c1nn(Cc2ccc(Cl)cc2)c2ccccc12] |
| 374 | 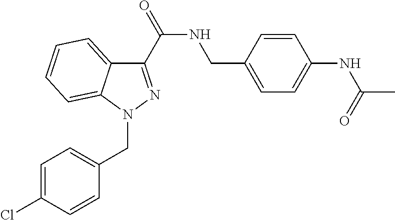 [SMILES: CC(=O)Nc1ccc(CNC(=O)c2nn(Cc3ccc(Cl)cc3)c3ccccc23)cc1] |
| 375 | 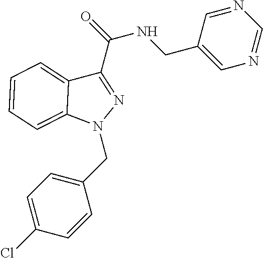 [SMILES: O=C(NCc1cncnc1)c1nn(Cc2ccc(Cl)cc2)c2ccccc12] |
| 376 | 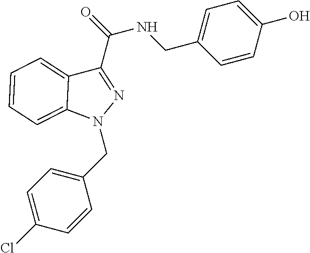 [SMILES: O=C(NCc1ccc(O)cc1)c1nn(Cc2ccc(Cl)cc2)c2ccccc12] |
| 377 | 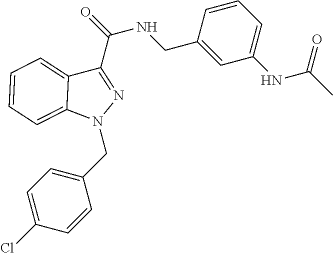 [SMILES: CC(=O)Nc1cccc(CNC(=O)c2nn(Cc3ccc(Cl)cc3)c3ccccc23)c1] |
| 378 | 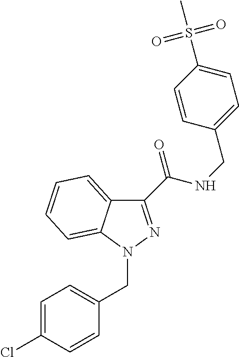 [SMILES: CS(=O)(=O)c1ccc(CNC(=O)c2nn(Cc3ccc(Cl)cc3)c3ccccc23)cc1] |
| 379 | 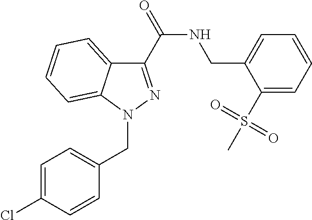 [SMILES: CS(=O)(=O)c1ccccc1CNC(=O)c1nn(Cc2ccc(Cl)cc2)c2ccccc12] |
| 380 | 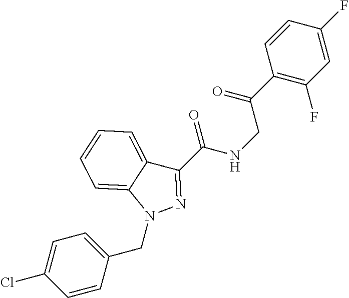 [SMILES: O=C(CNC(=O)c1nn(Cc2ccc(Cl)cc2)c2ccccc12)c1ccc(F)cc1F] |
| 381 | 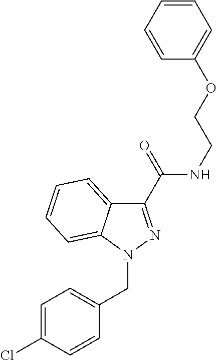 [SMILES: O=C(NCCOc1ccccc1)c1nn(Cc2ccc(Cl)cc2)c2ccccc12] |
| 382 | 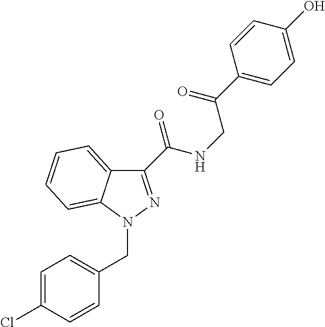 [SMILES: O=C(CNC(=O)c1nn(Cc2ccc(Cl)cc2)c2ccccc12)c1ccc(O)cc1] |
| 383 | 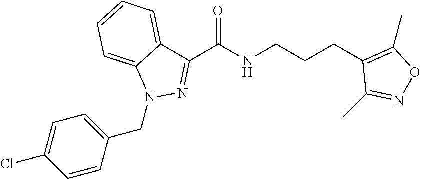 [SMILES: Cc1noc(C)c1CCCNC(=O)c1nn(Cc2ccc(Cl)cc2)c2ccccc12] |
| 384 | 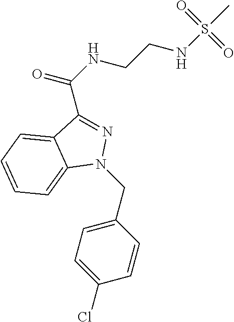 [SMILES: CS(=O)(=O)NCCNC(=O)c1nn(Cc2ccc(Cl)cc2)c2ccccc12] |
| 385 | 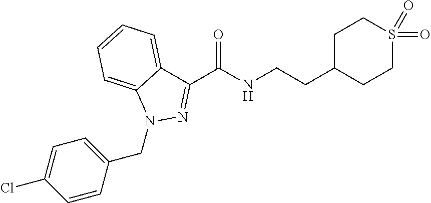 [SMILES: O=C(NCCC1CCS(=O)(=O)CC1)c1nn(Cc2ccc(Cl)cc2)c2ccccc12] |
| 386 | 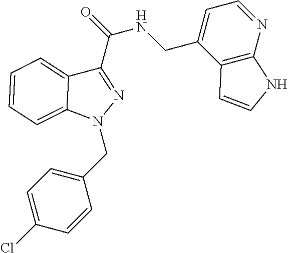 [SMILES: O=C(NCc1ccnc2[nH]ccc12)c1nn(Cc2ccc(Cl)cc2)c2ccccc12] |
| 387 | 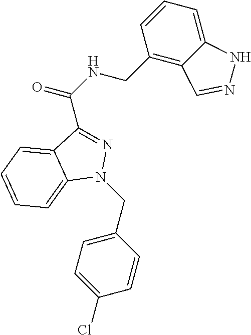 [SMILES: O=C(NCc1cccc2[nH]ncc12)c1nn(Cc2ccc(Cl)cc2)c2ccccc12] |
| 388 | 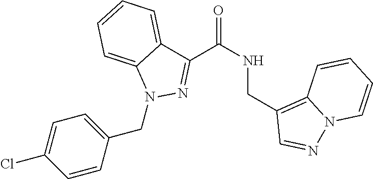 [SMILES: O=C(NCc1cnn2ccccc12)c1nn(Cc2ccc(Cl)cc2)c2ccccc12] |
| 389 | 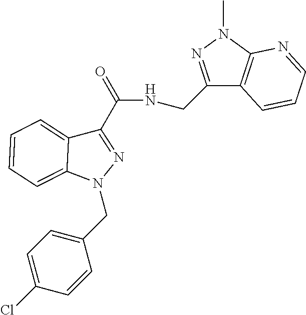 [SMILES: Cn1nc(CNC(=O)c2nn(Cc3ccc(Cl)cc3)c3ccccc23)c2cccnc21] |
| 390 | 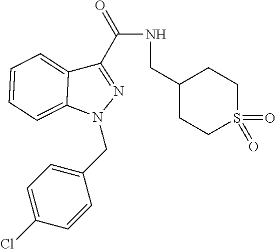 [SMILES: O=C(NCC1CCS(=O)(=O)CC1)c1nn(Cc2ccc(Cl)cc2)c2ccccc12] |
| 391 | 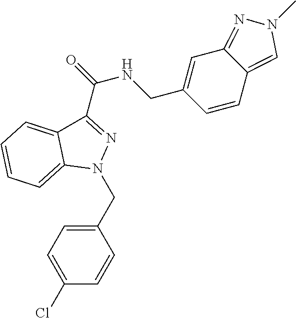 [SMILES: Cn1cc2ccc(CNC(=O)c3nn(Cc4ccc(Cl)cc4)c4ccccc34)cc2n1] |
| 392 | 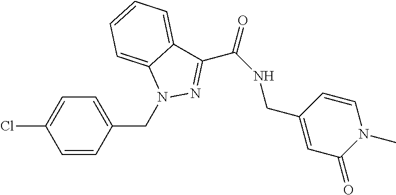 [SMILES: Cn1ccc(CNC(=O)c2nn(Cc3ccc(Cl)cc3)c3ccccc23)cc1=O] |
| 393 | 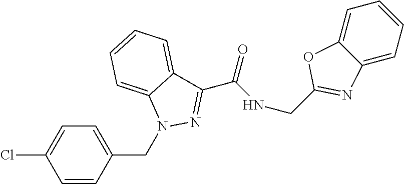 [SMILES: O=C(NCc1nc2ccccc2o1)c1nn(Cc2ccc(Cl)cc2)c2ccccc12] |
| 395 | 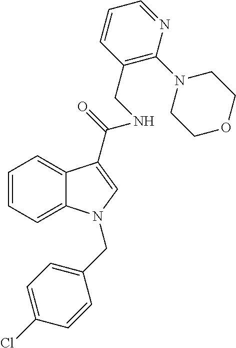 [SMILES: O=C(NCc1cccnc1N1CCOCC1)c1cn(Cc2ccc(Cl)cc2)c2ccccc12] |
| 396 | 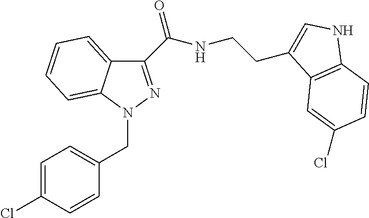 [SMILES: O=C(NCCc1c[nH]c2ccc(Cl)cc12)c1nn(Cc2ccc(Cl)cc2)c2ccccc12] |
| 397 | 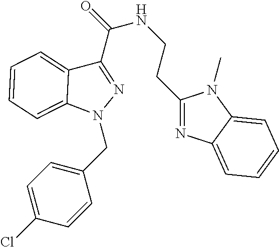 [SMILES: Cn1c(CCNC(=O)c2nn(Cc3ccc(Cl)cc3)c3ccccc23)nc2ccccc21] |
| 398 | 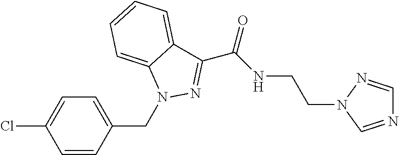 [SMILES: O=C(NCCn1cncn1)c1nn(Cc2ccc(Cl)cc2)c2ccccc12] |
| 399 | 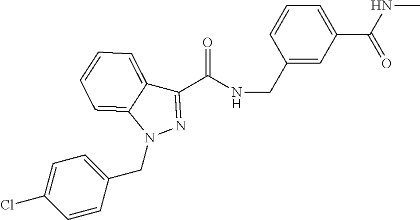 [SMILES: CNC(=O)c1cccc(CNC(=O)c2nn(Cc3ccc(Cl)cc3)c3ccccc23)c1] |
| 400 | 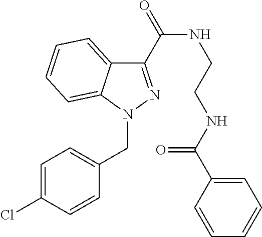 [SMILES: O=C(NCCNC(=O)c1nn(Cc2ccc(Cl)cc2)c2ccccc12)c1ccccc1] |
| 401 | 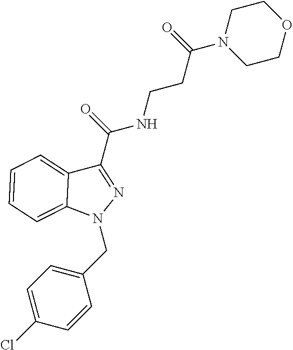 [SMILES: O=C(NCCC(=O)N1CCOCC1)c1nn(Cc2ccc(Cl)cc2)c2ccccc12] |
| 402 | 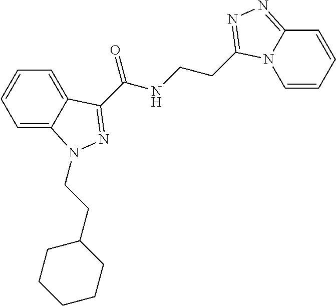 [SMILES: O=C(NCCc1nnc2ccccn12)c1nn(CCC2CCCCC2)c2ccccc12] |
| 403 | 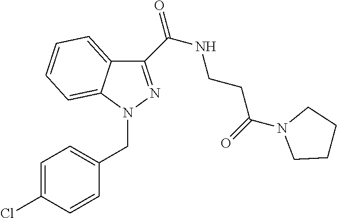 [SMILES: O=C(NCCC(=O)N1CCCC1)c1nn(Cc2ccc(Cl)cc2)c2ccccc12] |
| 404 | 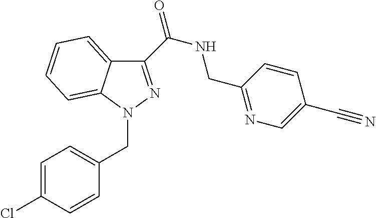 [SMILES: N#Cc1ccc(CNC(=O)c2nn(Cc3ccc(Cl)cc3)c3ccccc23)nc1] |
| 405 | 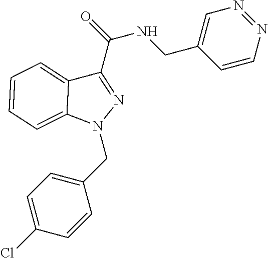 [SMILES: O=C(NCc1ccnnc1)c1nn(Cc2ccc(Cl)cc2)c2ccccc12] |
| 406 | 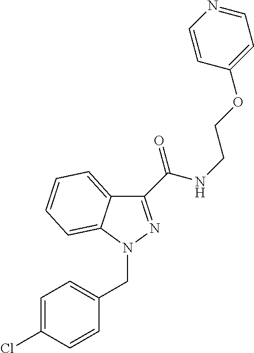 [SMILES: O=C(NCCOc1ccncc1)c1nn(Cc2ccc(Cl)cc2)c2ccccc12] |
| 407 | 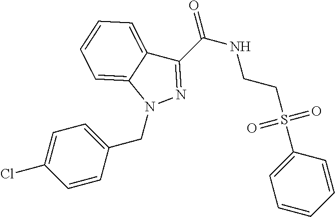 [SMILES: O=C(NCCS(=O)(=O)c1ccccc1)c1nn(Cc2ccc(Cl)cc2)c2ccccc12] |
| 408 | 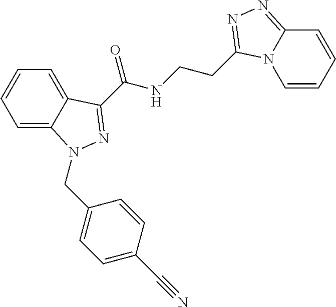 [SMILES: N#Cc1ccc(Cn2nc(C(=O)NCCc3nnc4ccccn34)c3ccccc32)cc1] |
| 409 | 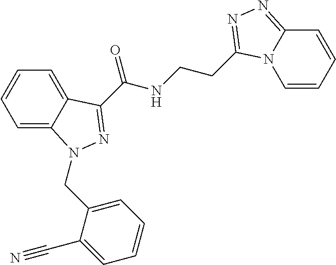 [SMILES: N#Cc1ccccc1Cn1nc(C(=O)NCCc2nnc3ccccn23)c2ccccc21] |
| 410 | 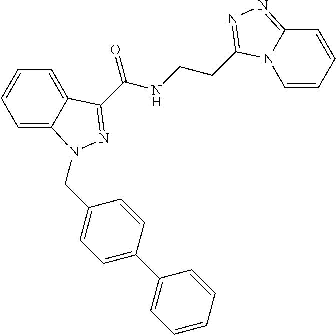 [SMILES: O=C(NCCc1nnc2ccccn12)c1nn(Cc2ccc(-c3ccccc3)cc2)c2ccccc12] |
| 411 | 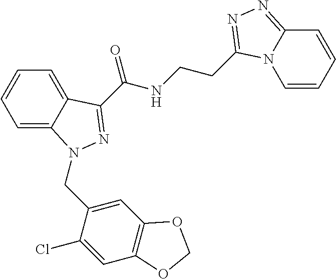 [SMILES: O=C(NCCc1nnc2ccccn12)c1nn(Cc2cc3c(cc2Cl)OCO3)c2ccccc12] |
| 412 | 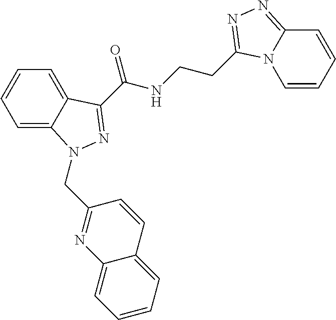 [SMILES: O=C(NCCc1nnc2ccccn12)c1nn(Cc2ccc3ccccc3n2)c2ccccc12] |
| 413 | 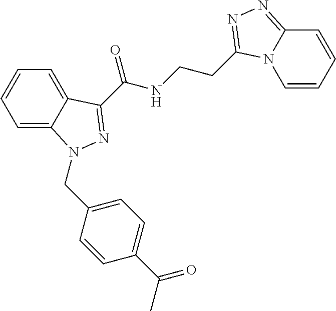 [SMILES: CC(=O)c1ccc(Cn2nc(C(=O)NCCc3nnc4ccccn34)c3ccccc32)cc1] |
| 414 | 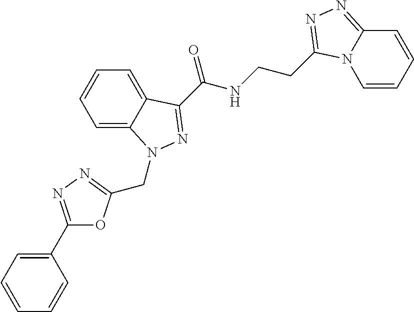 [SMILES: O=C(NCCc1nnc2ccccn12)c1nn(Cc2nnc(-c3ccccc3)o2)c2ccccc12] |
| 415 | 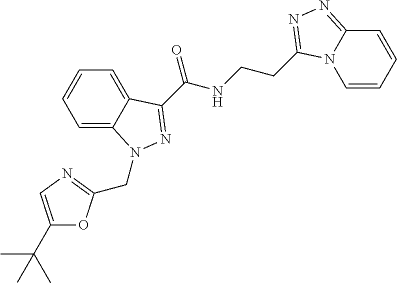 [SMILES: CC(C)(C)c1cnc(Cn2nc(C(=O)NCCc3nnc4ccccn34)c3ccccc32)o1] |
| 416 | 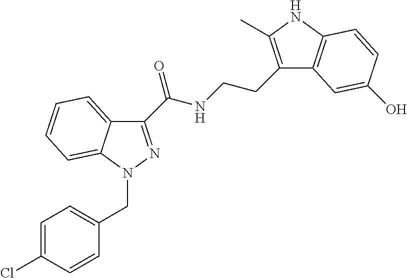 [SMILES: Cc1[nH]c2ccc(O)cc2c1CCNC(=O)c1nn(Cc2ccc(Cl)cc2)c2ccccc12] |
| 418 | 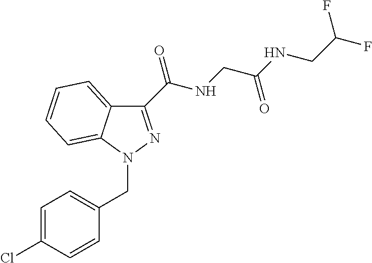 [SMILES: O=C(CNC(=O)c1nn(Cc2ccc(Cl)cc2)c2ccccc12)NCC(F)F] |
| 419 | 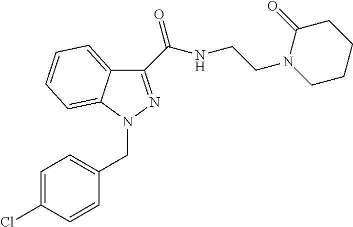 [SMILES: O=C(NCCN1CCCCC1=O)c1nn(Cc2ccc(Cl)cc2)c2ccccc12] |
| 420 | 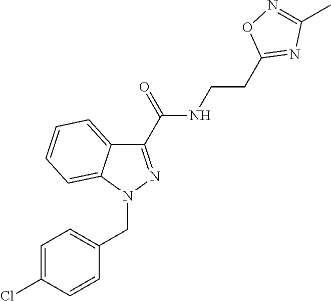 [SMILES: Cc1noc(CCNC(=O)c2nn(Cc3ccc(Cl)cc3)c3ccccc23)n1] |
| 421 | 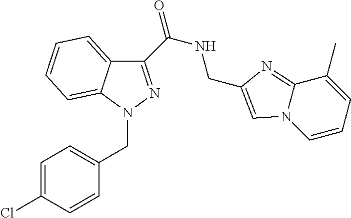 [SMILES: Cc1cccn2cc(CNC(=O)c3nn(Cc4ccc(Cl)cc4)c4ccccc34)nc12] |
| 422 | 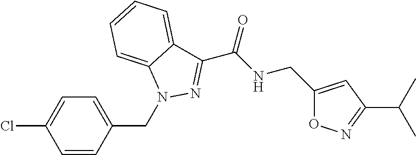 [SMILES: CC(C)c1cc(CNC(=O)c2nn(Cc3ccc(Cl)cc3)c3ccccc23)on1] |
| 423 | 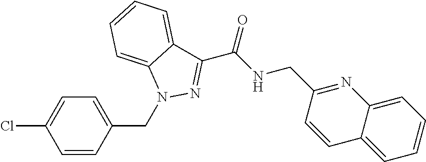 [SMILES: O=C(NCc1ccc2ccccc2n1)c1nn(Cc2ccc(Cl)cc2)c2ccccc12] |
| 424 | 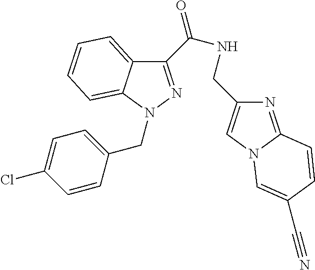 [SMILES: N#Cc1ccc2nc(CNC(=O)c3nn(Cc4ccc(Cl)cc4)c4ccccc34)cn2c1] |
| 425 | 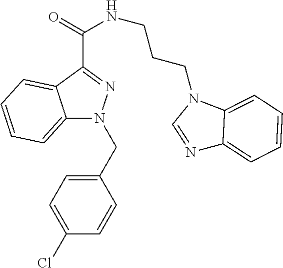 [SMILES: O=C(NCCCn1cnc2ccccc21)c1nn(Cc2ccc(Cl)cc2)c2ccccc12] |
| 426 | 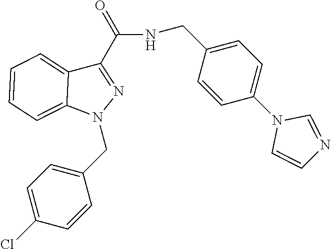 [SMILES: O=C(NCc1ccc(-n2ccnc2)cc1)c1nn(Cc2ccc(Cl)cc2)c2ccccc12] |
| 427 | 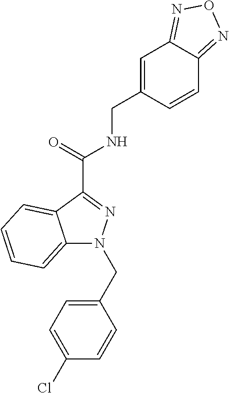 [SMILES: O=C(NCc1ccc2nonc2c1)c1nn(Cc2ccc(Cl)cc2)c2ccccc12] |
| 428 | 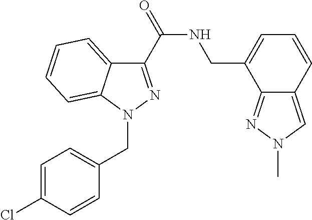 [SMILES: Cn1cc2cccc(CNC(=O)c3nn(Cc4ccc(Cl)cc4)c4ccccc34)c2n1] |
| 429 | 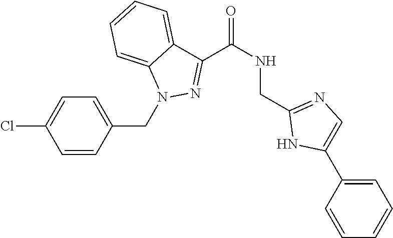 [SMILES: O=C(NCc1ncc(-c2ccccc2)[nH]1)c1nn(Cc2ccc(Cl)cc2)c2ccccc12] |
| 430 | 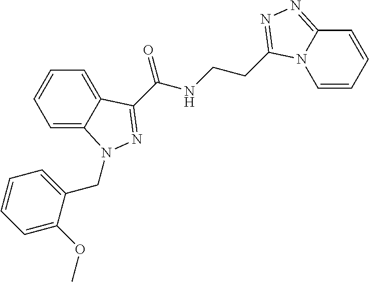 [SMILES: COc1ccccc1Cn1nc(C(=O)NCCc2nnc3ccccn23)c2ccccc21] |
| 431 | 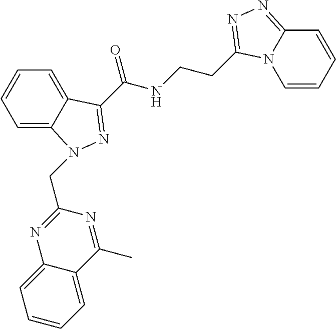 [SMILES: Cc1nc(Cn2nc(C(=O)NCCc3nnc4ccccn34)c3ccccc32)nc2ccccc12] |
| 432 | 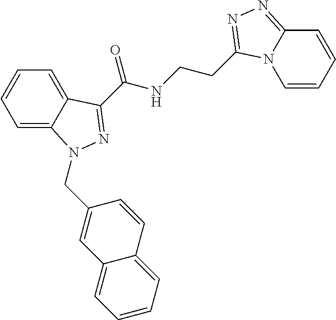 [SMILES: O=C(NCCc1nnc2ccccn12)c1nn(Cc2ccc3ccccc3c2)c2ccccc12] |
| 433 | 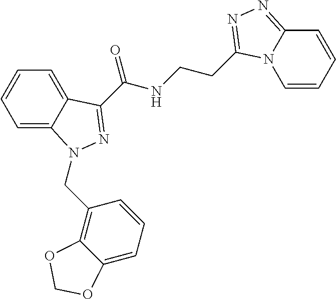 [SMILES: O=C(NCCc1nnc2ccccn12)c1nn(Cc2cccc3c2OCO3)c2ccccc12] |
| 434 | 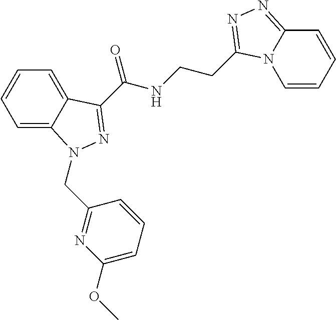 [SMILES: COc1cccc(Cn2nc(C(=O)NCCc3nnc4ccccn34)c3ccccc32)n1] |
| 435 | 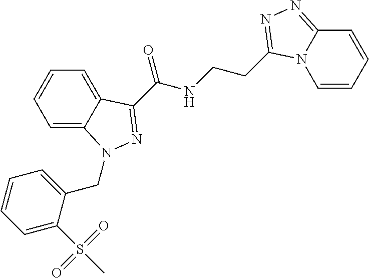 [SMILES: CS(=O)(=O)c1ccccc1Cn1nc(C(=O)NCCc2nnc3ccccn23)c2ccccc21] |
| 436 | 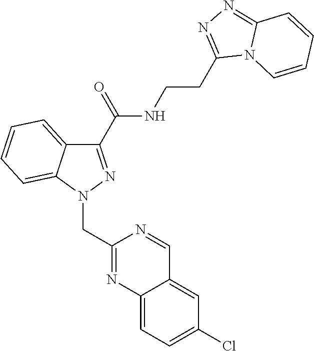 [SMILES: O=C(NCCc1nnc2ccccn12)c1nn(Cc2ncc3cc(Cl)ccc3n2)c2ccccc12] |
| 437 | 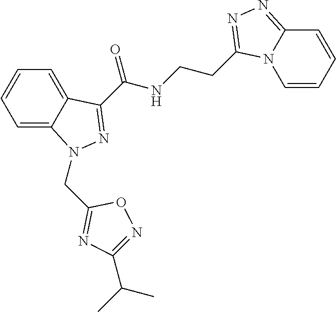 [SMILES: CC(C)c1noc(Cn2nc(C(=O)NCCc3nnc4ccccn34)c3ccccc32)n1] |
| 438 | 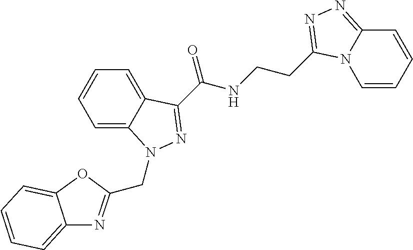 [SMILES: O=C(NCCc1nnc2ccccn12)c1nn(Cc2nc3ccccc3o2)c2ccccc12] |
| 439 | 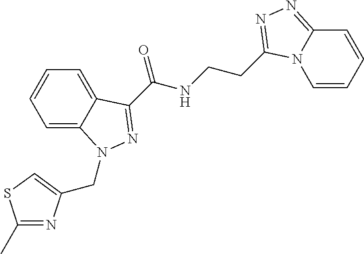 [SMILES: Cc1nc(Cn2nc(C(=O)NCCc3nnc4ccccn34)c3ccccc32)cs1] |
| 440 | 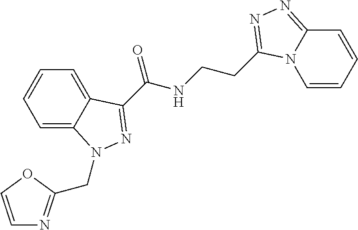 [SMILES: O=C(NCCc1nnc2ccccn12)c1nn(Cc2ncco2)c2ccccc12] |
| 441 | 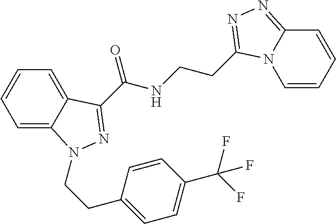 [SMILES: O=C(NCCc1nnc2ccccn12)c1nn(CCc2ccc(C(F)(F)F)cc2)c2ccccc12] |
| 442 | 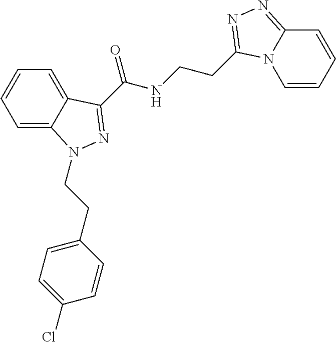 [SMILES: O=C(NCCc1nnc2ccccn12)c1nn(CCc2ccc(Cl)cc2)c2ccccc12] |
| 443 | 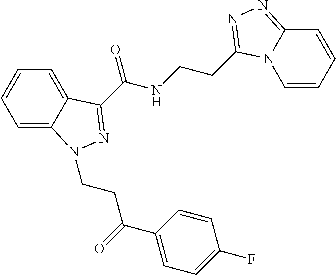 [SMILES: O=C(CCn1nc(C(=O)NCCc2nnc3ccccn23)c2ccccc21)c1ccc(F)cc1] |
| 444 | 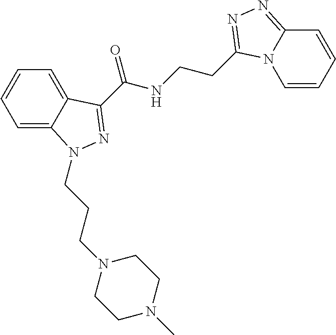 [SMILES: CN1CCN(CCCn2nc(C(=O)NCCc3nnc4ccccn34)c3ccccc32)CC1] |
| 445 | 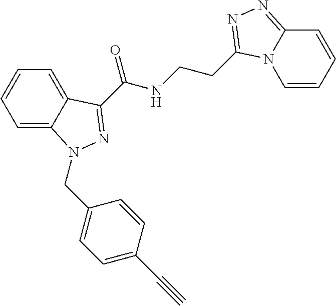 [SMILES: C#Cc1ccc(Cn2nc(C(=O)NCCc3nnc4ccccn34)c3ccccc32)cc1] |
| 446 | 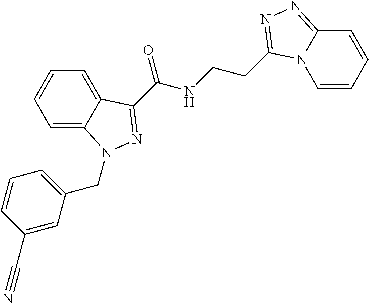 [SMILES: N#Cc1cccc(Cn2nc(C(=O)NCCc3nnc4ccccn34)c3ccccc32)c1] |
| 447 | 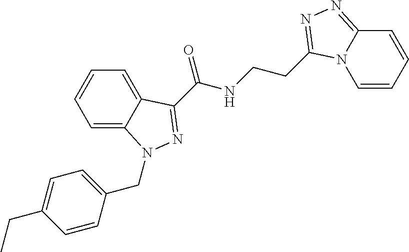 [SMILES: CCc1ccc(Cn2nc(C(=O)NCCc3nnc4ccccn34)c3ccccc32)cc1] |
| 448 | 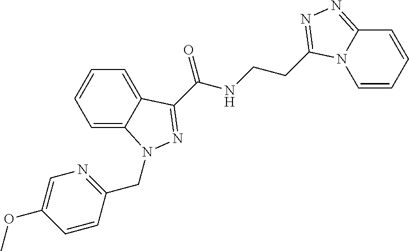 [SMILES: COc1ccc(Cn2nc(C(=O)NCCc3nnc4ccccn34)c3ccccc32)nc1] |
| 449 | 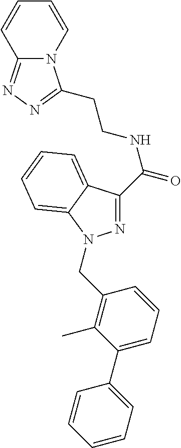 [SMILES: Cc1c(Cn2nc(C(=O)NCCc3nnc4ccccn34)c3ccccc32)cccc1-c1ccccc1] |
| 450 | 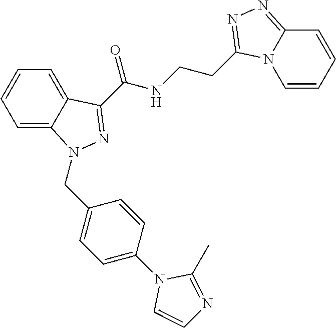 [SMILES: Cc1nccn1-c1ccc(Cn2nc(C(=O)NCCc3nnc4ccccn34)c3ccccc32)cc1] |
| 451 | 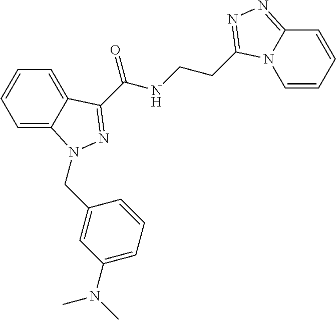 [SMILES: CN(C)c1cccc(Cn2nc(C(=O)NCCc3nnc4ccccn34)c3ccccc32)c1] |
| 452 | 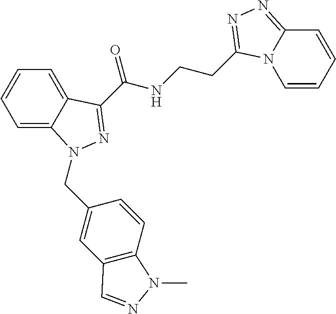 [SMILES: Cn1ncc2cc(Cn3nc(C(=O)NCCc4nnc5ccccn45)c4ccccc43)ccc21] |
| 453 | 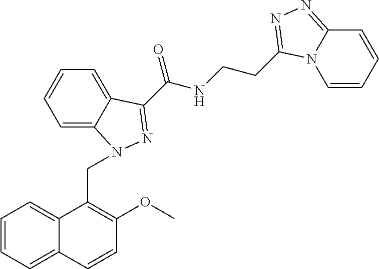 [SMILES: COc1ccc2ccccc2c1Cn1nc(C(=O)NCCc2nnc3ccccn23)c2ccccc21] |
| 454 | 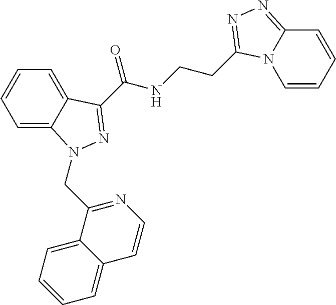 [SMILES: O=C(NCCc1nnc2ccccn12)c1nn(Cc2nccc3ccccc23)c2ccccc12] |
| 455 | 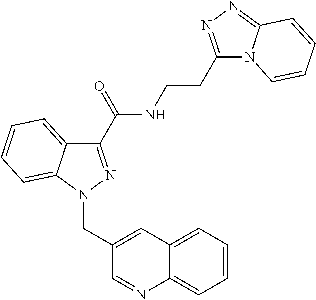 [SMILES: O=C(NCCc1nnc2ccccn12)c1nn(Cc2cnc3ccccc3c2)c2ccccc12] |
| 456 | 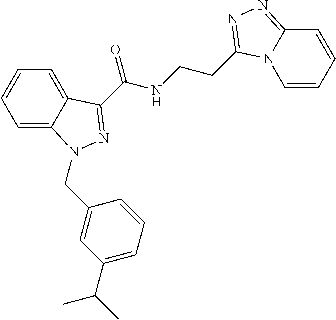 [SMILES: CC(C)c1cccc(Cn2nc(C(=O)NCCc3nnc4ccccn34)c3ccccc32)c1] |
| 457 | 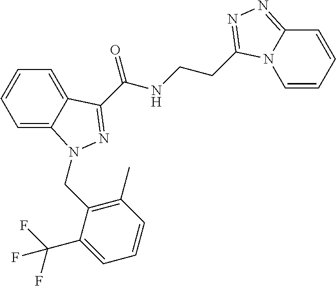 [SMILES: Cc1cccc(C(F)(F)F)c1Cn1nc(C(=O)NCCc2nnc3ccccn23)c2ccccc21] |
| 458 | 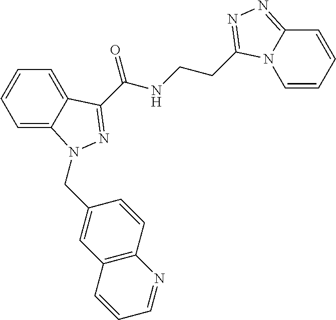 [SMILES: O=C(NCCc1nnc2ccccn12)c1nn(Cc2ccc3ncccc3c2)c2ccccc12] |
| 459 | 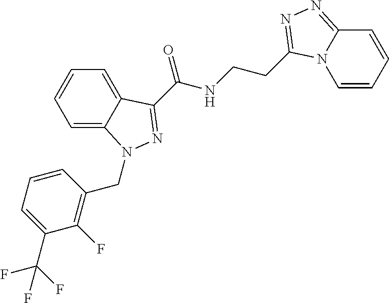 [SMILES: O=C(NCCc1nnc2ccccn12)c1nn(Cc2cccc(C(F)(F)F)c2F)c2ccccc12] |
| 460 | 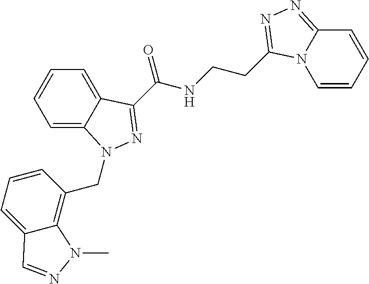 [SMILES: Cn1ncc2cccc(Cn3nc(C(=O)NCCc4nnc5ccccn45)c4ccccc43)c21] |
| 461 | 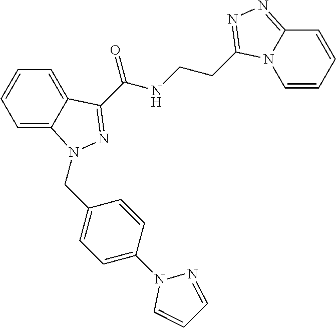 [SMILES: O=C(NCCc1nnc2ccccn12)c1nn(Cc2ccc(-n3cccn3)cc2)c2ccccc12] |
| 462 | 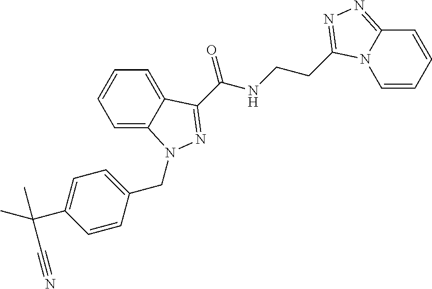 [SMILES: CC(C)(C#N)c1ccc(Cn2nc(C(=O)NCCc3nnc4ccccn34)c3ccccc32)cc1] |
| 463 | 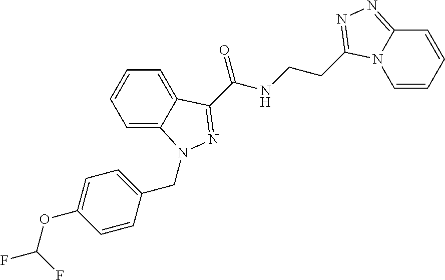 [SMILES: O=C(NCCc1nnc2ccccn12)c1nn(Cc2ccc(OC(F)F)cc2)c2ccccc12] |
| 464 | 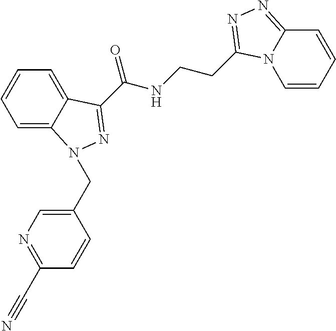 [SMILES: N#Cc1ccc(Cn2nc(C(=O)NCCc3nnc4ccccn34)c3ccccc32)cn1] |
| 465 | 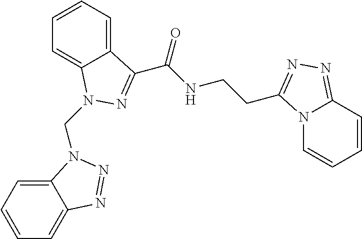 [SMILES: O=C(NCCc1nnc2ccccn12)c1nn(Cn2nnc3ccccc32)c2ccccc12] |
| 466 | 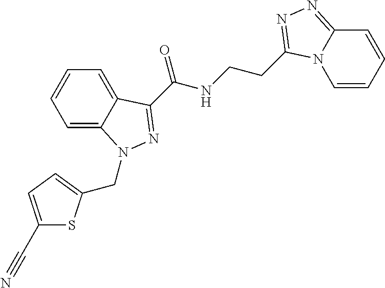 [SMILES: N#Cc1ccc(Cn2nc(C(=O)NCCc3nnc4ccccn34)c3ccccc32)s1] |
| 467 | 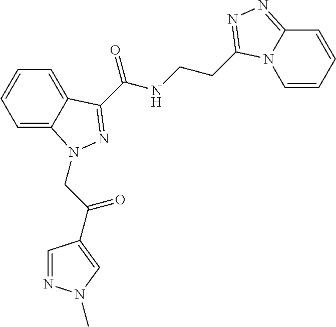 [SMILES: Cn1cc(C(=O)Cn2nc(C(=O)NCCc3nnc4ccccn34)c3ccccc32)cn1] |
| 468 | 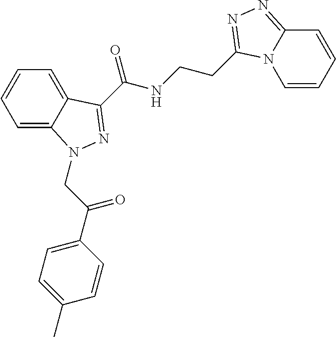 [SMILES: Cc1ccc(C(=O)Cn2nc(C(=O)NCCc3nnc4ccccn34)c3ccccc32)cc1] |
| 469 | 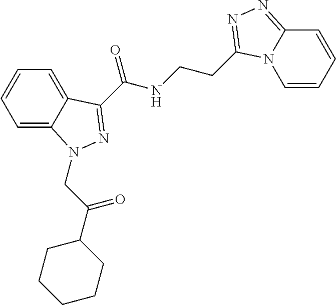 [SMILES: O=C(NCCc1nnc2ccccn12)c1nn(CC(=O)C2CCCCC2)c2ccccc12] |
| 470 | 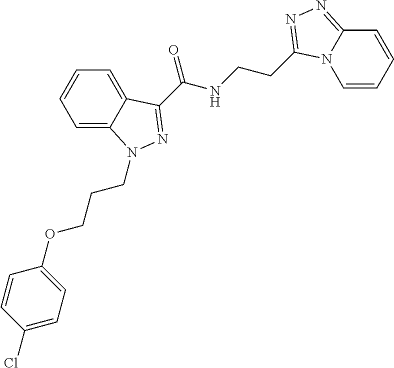 [SMILES: O=C(NCCc1nnc2ccccn12)c1nn(CCCOc2ccc(Cl)cc2)c2ccccc12] |
| 471 | 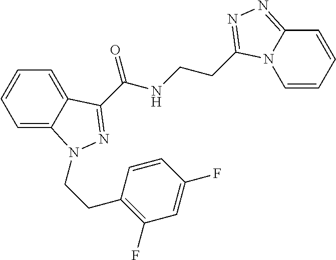 [SMILES: O=C(NCCc1nnc2ccccn12)c1nn(CCc2ccc(F)cc2F)c2ccccc12] |
| 472 | 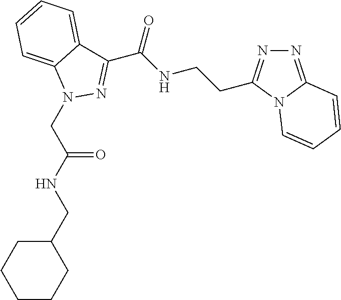 [SMILES: O=C(Cn1nc(C(=O)NCCc2nnc3ccccn23)c2ccccc21)NCC1CCCCC1] |
| 473 | 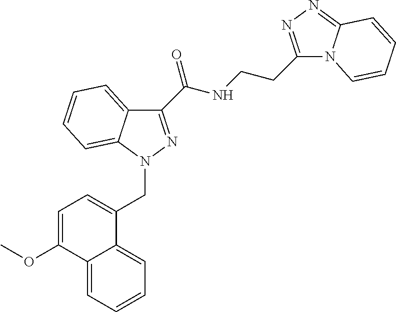 [SMILES: COc1ccc(Cn2nc(C(=O)NCCc3nnc4ccccn34)c3ccccc32)c2ccccc12] |
| 474 | 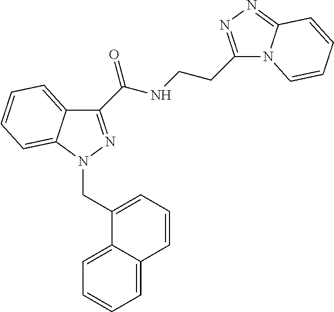 [SMILES: O=C(NCCc1nnc2ccccn12)c1nn(Cc2cccc3ccccc23)c2ccccc12] |
| 475 | 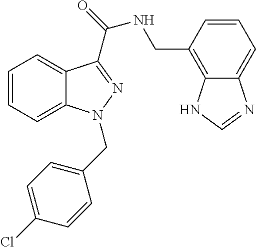 [SMILES: O=C(NCc1cccc2nc[nH]c12)c1nn(Cc2ccc(Cl)cc2)c2ccccc12] |
| 476 | 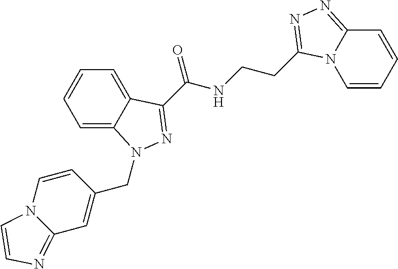 [SMILES: O=C(NCCc1nnc2ccccn12)c1nn(Cc2ccn3ccnc3c2)c2ccccc12] |
| 477 | 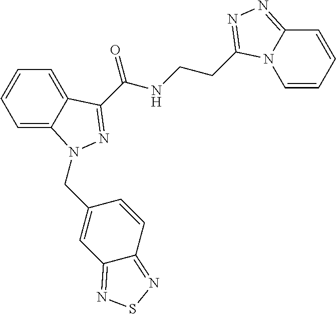 [SMILES: O=C(NCCc1nnc2ccccn12)c1nn(Cc2ccc3nsnc3c2)c2ccccc12] |
| 478 | 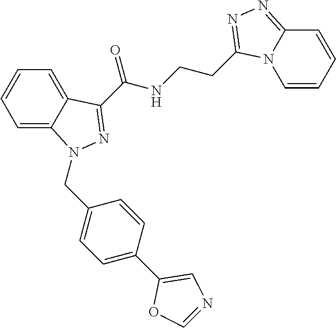 [SMILES: O=C(NCCc1nnc2ccccn12)c1nn(Cc2ccc(-c3cnco3)cc2)c2ccccc12] |
| 479 | 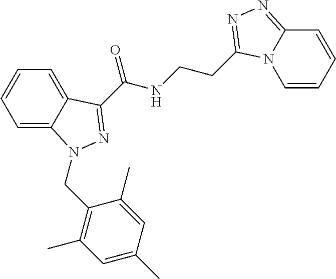 [SMILES: Cc1cc(C)c(Cn2nc(C(=O)NCCc3nnc4ccccn34)c3ccccc32)c(C)c1] |
| 480 | 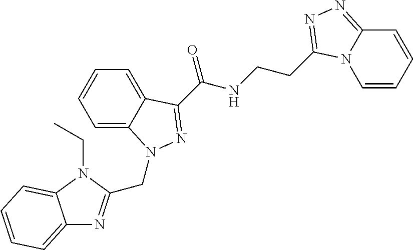 [SMILES: CCn1c(Cn2nc(C(=O)NCCc3nnc4ccccn34)c3ccccc32)nc2ccccc21] |
| 481 | 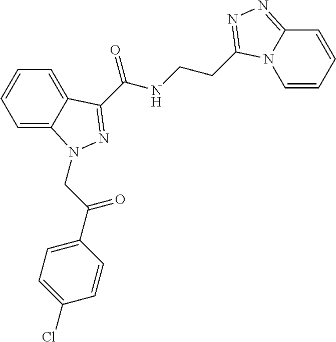 [SMILES: O=C(Cn1nc(C(=O)NCCc2nnc3ccccn23)c2ccccc21)c1ccc(Cl)cc1] |
| 482 | 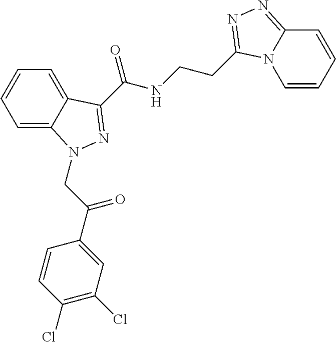 [SMILES: O=C(Cn1nc(C(=O)NCCc2nnc3ccccn23)c2ccccc21)c1ccc(Cl)c(Cl)c1] |
| 483 | 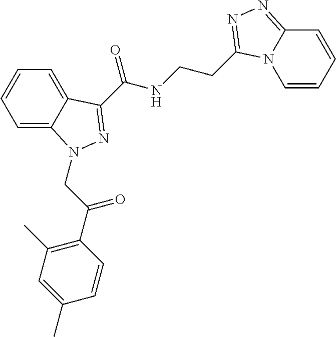 [SMILES: Cc1ccc(C(=O)Cn2nc(C(=O)NCCc3nnc4ccccn34)c3ccccc32)c(C)c1] |
| 484 | 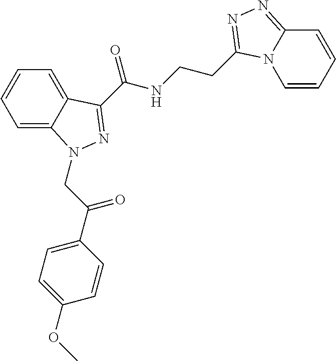 [SMILES: COc1ccc(C(=O)Cn2nc(C(=O)NCCc3nnc4ccccn34)c3ccccc32)cc1] |
| 485 | 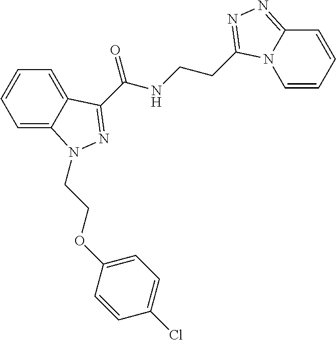 [SMILES: O=C(NCCc1nnc2ccccn12)c1nn(CCOc2ccc(Cl)cc2)c2ccccc12] |
| 486 | 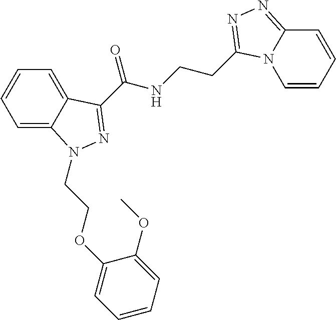 [SMILES: COc1ccccc1OCCn1nc(C(=O)NCCc2nnc3ccccn23)c2ccccc21] |
| 487 | 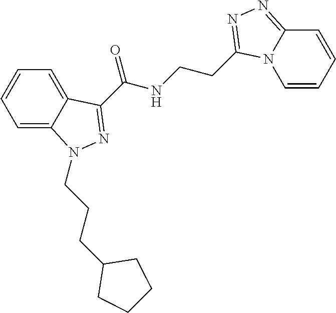 [SMILES: O=C(NCCc1nnc2ccccn12)c1nn(CCCC2CCCC2)c2ccccc12] |
| 488 | 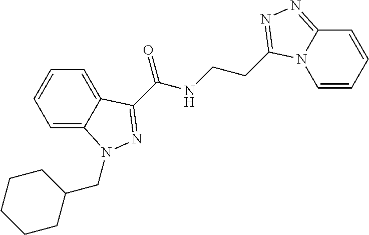 [SMILES: O=C(NCCc1nnc2ccccn12)c1nn(CC2CCCCC2)c2ccccc12] |
| 489 | 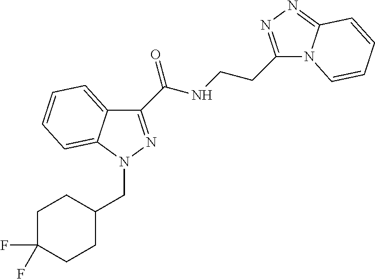 [SMILES: O=C(NCCc1nnc2ccccn12)c1nn(CC2CCC(F)(F)CC2)c2ccccc12] |
| 490 | 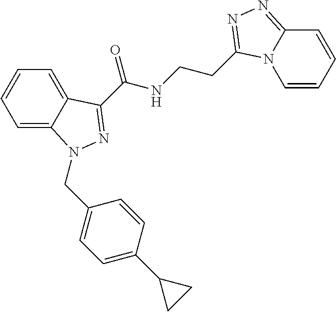 [SMILES: O=C(NCCc1nnc2ccccn12)c1nn(Cc2ccc(C3CC3)cc2)c2ccccc12] |
| 491 | 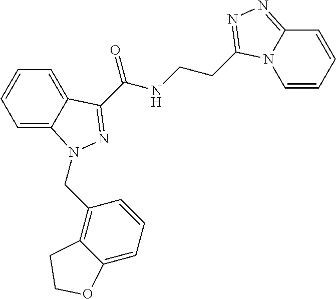 [SMILES: O=C(NCCc1nnc2ccccn12)c1nn(Cc2cccc3c2CCO3)c2ccccc12] |
| 492 | 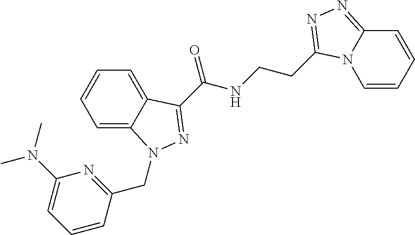 [SMILES: CN(C)c1cccc(Cn2nc(C(=O)NCCc3nnc4ccccn34)c3ccccc32)n1] |
| 493 | 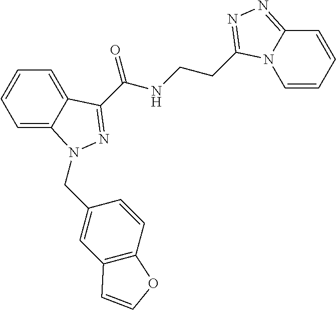 [SMILES: O=C(NCCc1nnc2ccccn12)c1nn(Cc2ccc3occc3c2)c2ccccc12] |
| 494 | 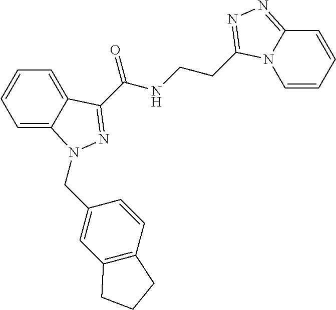 [SMILES: O=C(NCCc1nnc2ccccn12)c1nn(Cc2ccc3c(c2)CCC3)c2ccccc12] |
| 495 | 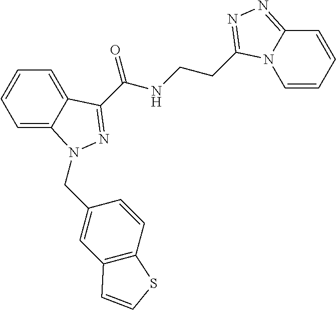 [SMILES: O=C(NCCc1nnc2ccccn12)c1nn(Cc2ccc3sccc3c2)c2ccccc12] |
| 496 | 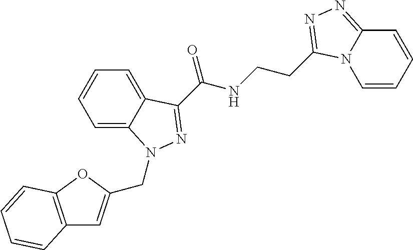 [SMILES: O=C(NCCc1nnc2ccccn12)c1nn(Cc2cc3ccccc3o2)c2ccccc12] |
| 497 | 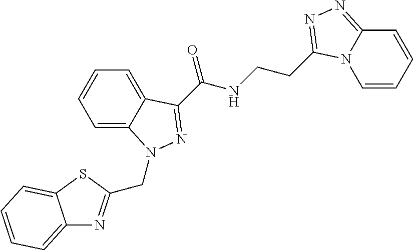 [SMILES: O=C(NCCc1nnc2ccccn12)c1nn(Cc2nc3ccccc3s2)c2ccccc12] |
| 498 | 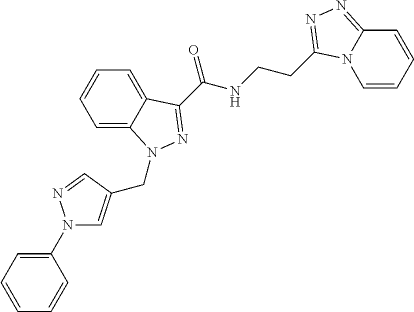 [SMILES: O=C(NCCc1nnc2ccccn12)c1nn(Cc2cnn(-c3ccccc3)c2)c2ccccc12] |
| 499 | 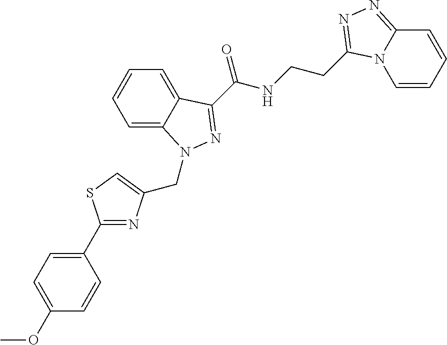 [SMILES: COc1ccc(-c2nc(Cn3nc(C(=O)NCCc4nnc5ccccn45)c4ccccc43)cs2)cc1] |
| 500 | 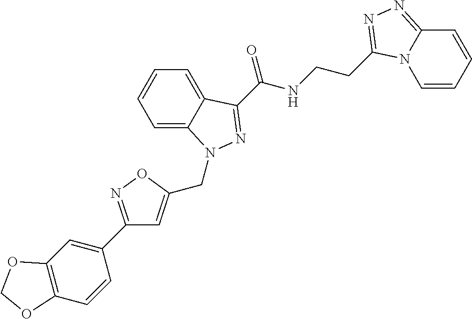 [SMILES: O=C(NCCc1nnc2ccccn12)c1nn(Cc2cc(-c3ccc4c(c3)OCO4)no2)c2ccccc12] |
| 501 | 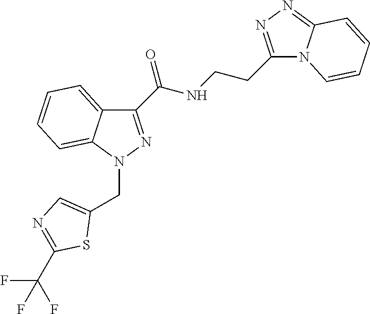 [SMILES: O=C(NCCc1nnc2ccccn12)c1nn(Cc2cnc(C(F)(F)F)s2)c2ccccc12] |
| 502 | 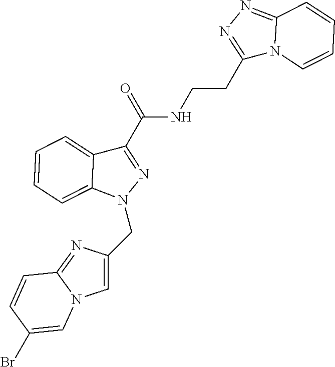 [SMILES: O=C(NCCc1nnc2ccccn12)c1nn(Cc2cn3cc(Br)ccc3n2)c2ccccc12] |
| 503 | 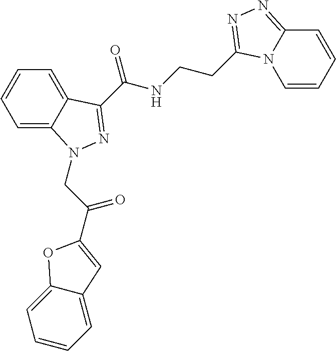 [SMILES: O=C(Cn1nc(C(=O)NCCc2nnc3ccccn23)c2ccccc21)c1cc2ccccc2o1] |
| 504 | 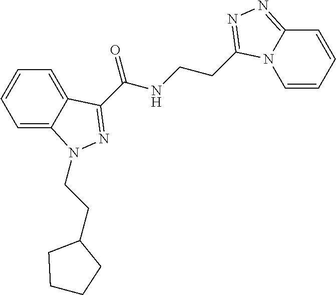 [SMILES: O=C(NCCc1nnc2ccccn12)c1nn(CCC2CCCC2)c2ccccc12] |
| 505 | 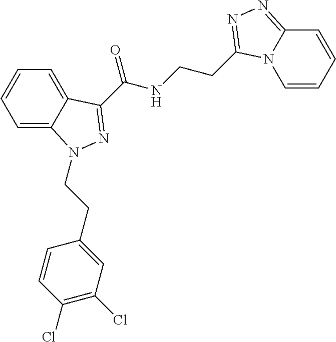 [SMILES: O=C(NCCc1nnc2ccccn12)c1nn(CCc2ccc(Cl)c(Cl)c2)c2ccccc12] |
| 506 | 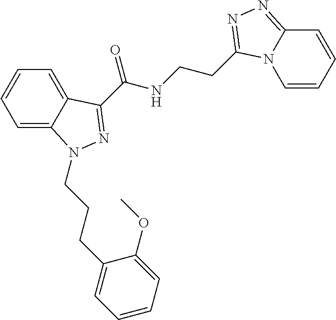 [SMILES: COc1ccccc1CCCn1nc(C(=O)NCCc2nnc3ccccn23)c2ccccc21] |
| 507 | 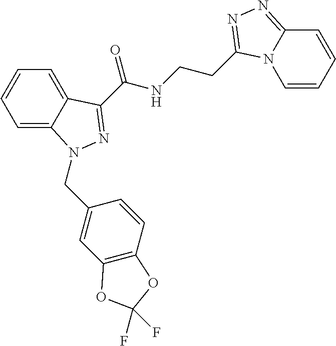 [SMILES: O=C(NCCc1nnc2ccccn12)c1nn(Cc2ccc3c(c2)OC(F)(F)O3)c2ccccc12] |
| 508 | 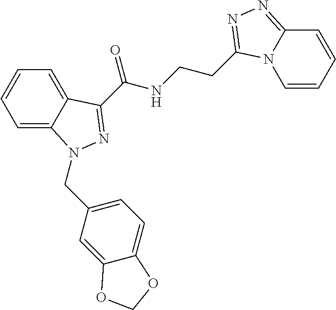 [SMILES: O=C(NCCc1nnc2ccccn12)c1nn(Cc2ccc3c(c2)OCO3)c2ccccc12] |
| 509 | 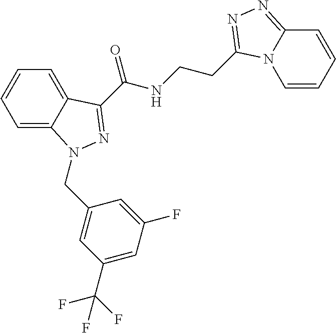 [SMILES: O=C(NCCc1nnc2ccccn12)c1nn(Cc2cc(F)cc(C(F)(F)F)c2)c2ccccc12] |
| 510 | 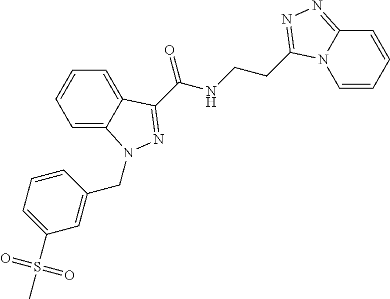 [SMILES: CS(=O)(=O)c1cccc(Cn2nc(C(=O)NCCc3nnc4ccccn34)c3ccccc32)c1] |
| 511 | 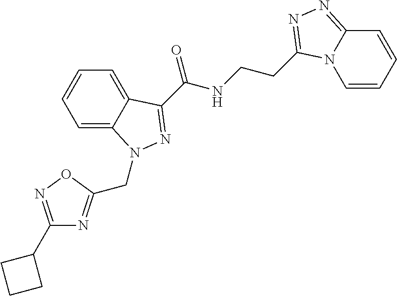 [SMILES: O=C(NCCc1nnc2ccccn12)c1nn(Cc2nc(C3CCC3)no2)c2ccccc12] |
| 512 | 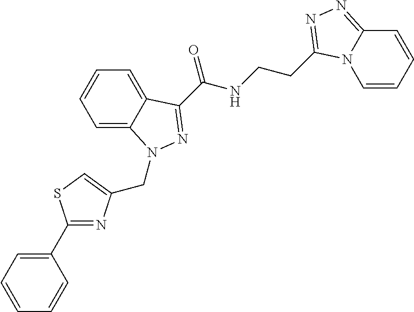 [SMILES: O=C(NCCc1nnc2ccccn12)c1nn(Cc2csc(-c3ccccc3)n2)c2ccccc12] |
| 513 | 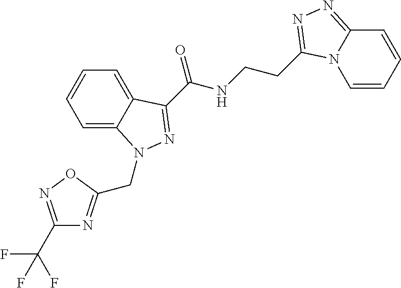 [SMILES: O=C(NCCc1nnc2ccccn12)c1nn(Cc2nc(C(F)(F)F)no2)c2ccccc12] |
| 514 | 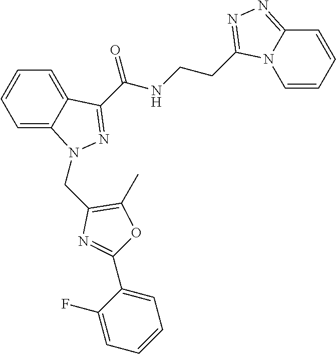 [SMILES: Cc1oc(-c2ccccc2F)nc1Cn1nc(C(=O)NCCc2nnc3ccccn23)c2ccccc21] |
| 515 | 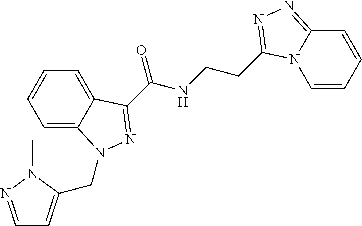 [SMILES: Cn1nccc1Cn1nc(C(=O)NCCc2nnc3ccccn23)c2ccccc21] |
| 516 | 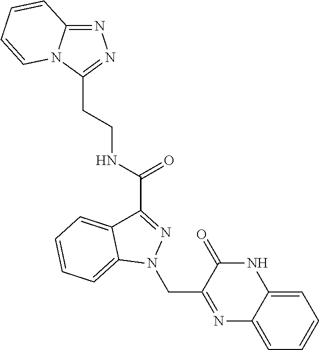 [SMILES: O=C(NCCc1nnc2ccccn12)c1nn(Cc2nc3ccccc3[nH]c2=O)c2ccccc12] |
| 517 | 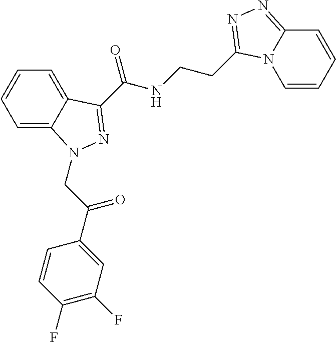 [SMILES: O=C(Cn1nc(C(=O)NCCc2nnc3ccccn23)c2ccccc21)c1ccc(F)c(F)c1] |
| 518 | 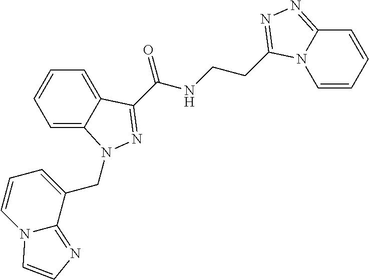 [SMILES: O=C(NCCc1nnc2ccccn12)c1nn(Cc2cccn3ccnc23)c2ccccc12] |
| 519 | 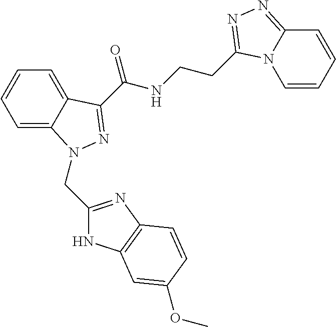 [SMILES: COc1ccc2nc(Cn3nc(C(=O)NCCc4nnc5ccccn45)c4ccccc43)[nH]c2c1] |
| 520 | 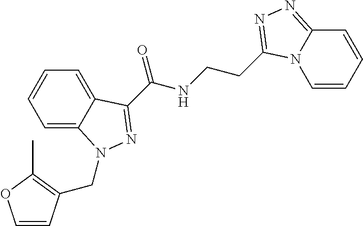 [SMILES: Cc1occc1Cn1nc(C(=O)NCCc2nnc3ccccn23)c2ccccc21] |
| 521 | 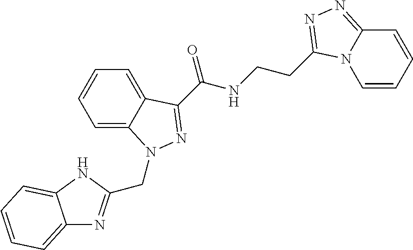 [SMILES: O=C(NCCc1nnc2ccccn12)c1nn(Cc2nc3ccccc3[nH]2)c2ccccc12] |
| 522 | 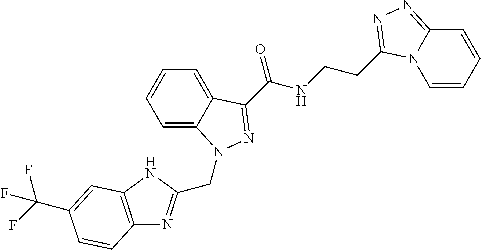 [SMILES: O=C(NCCc1nnc2ccccn12)c1nn(Cc2nc3ccc(C(F)(F)F)cc3[nH]2)c2ccccc12] |
| 523 | 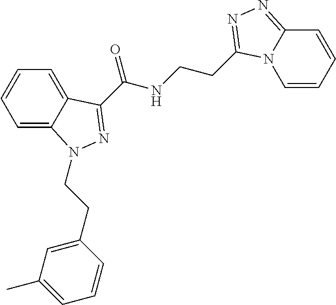 [SMILES: Cc1cccc(CCn2nc(C(=O)NCCc3nnc4ccccn34)c3ccccc32)c1] |
| 524 | 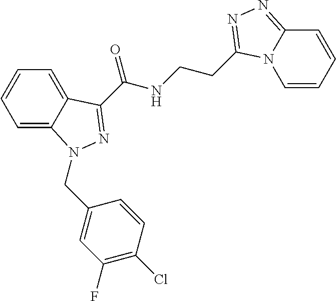 [SMILES: O=C(NCCc1nnc2ccccn12)c1nn(Cc2ccc(Cl)c(F)c2)c2ccccc12] |
| 525 | 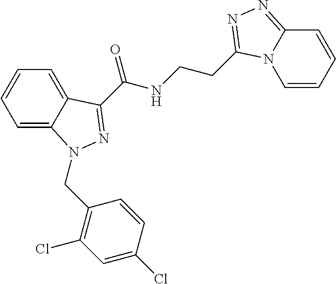 [SMILES: O=C(NCCc1nnc2ccccn12)c1nn(Cc2ccc(Cl)cc2Cl)c2ccccc12] |
| 526 | 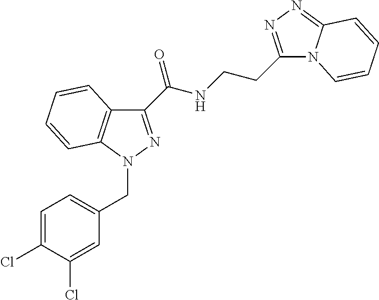 [SMILES: O=C(NCCc1nnc2ccccn12)c1nn(Cc2ccc(Cl)c(Cl)c2)c2ccccc12] |
| 527 | 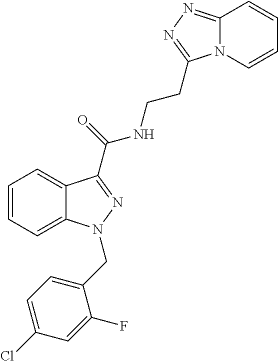 [SMILES: O=C(NCCc1nnc2ccccn12)c1nn(Cc2ccc(Cl)cc2F)c2ccccc12] |
| 528 | 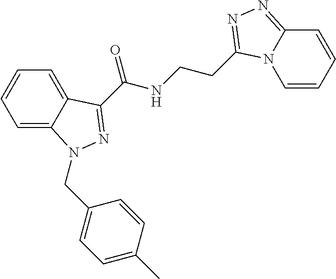 [SMILES: Cc1ccc(Cn2nc(C(=O)NCCc3nnc4ccccn34)c3ccccc32)cc1] |
| 529 | 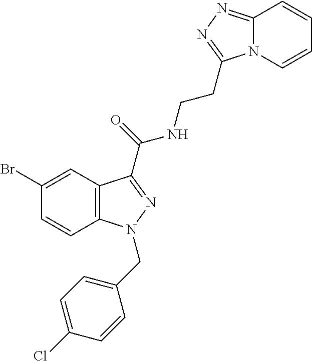 [SMILES: O=C(NCCc1nnc2ccccn12)c1nn(Cc2ccc(Cl)cc2)c2ccc(Br)cc12] |
| 530 | 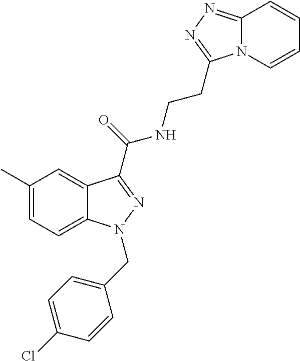 [SMILES: Cc1ccc2c(c1)c(C(=O)NCCc1nnc3ccccn13)nn2Cc1ccc(Cl)cc1] |
| 531 | 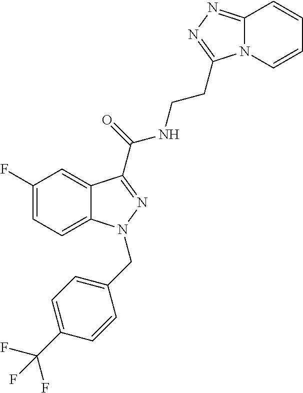 [SMILES: O=C(NCCc1nnc2ccccn12)c1nn(Cc2ccc(C(F)(F)F)cc2)c2ccc(F)cc12] |
| 532 | 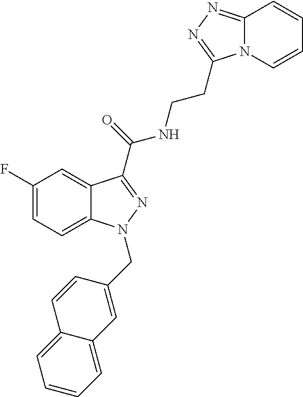 [SMILES: O=C(NCCc1nnc2ccccn12)c1nn(Cc2ccc3ccccc3c2)c2ccc(F)cc12] |
| 533 | 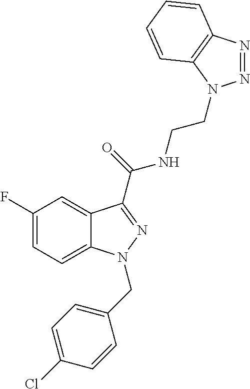 [SMILES: O=C(NCCn1nnc2ccccc21)c1nn(Cc2ccc(Cl)cc2)c2ccc(F)cc12] |
| 534 | 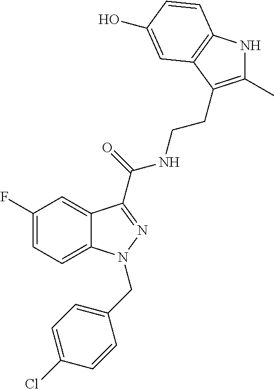 [SMILES: Cc1[nH]c2ccc(O)cc2c1CCNC(=O)c1nn(Cc2ccc(Cl)cc2)c2ccc(F)cc12] |
| 535 | 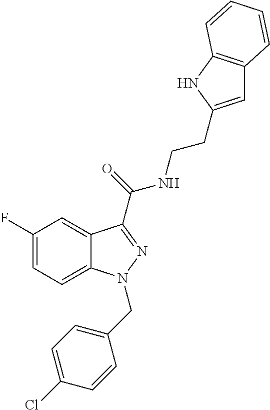 [SMILES: O=C(NCCc1cc2ccccc2[nH]1)c1nn(Cc2ccc(Cl)cc2)c2ccc(F)cc12] |
| 536 | 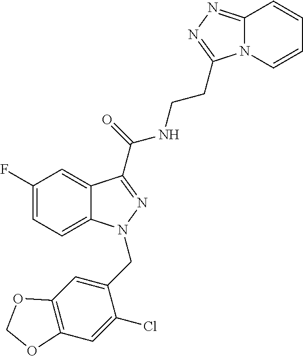 [SMILES: O=C(NCCc1nnc2ccccn12)c1nn(Cc2cc3c(cc2Cl)OCO3)c2ccc(F)cc12] |
| 537 | 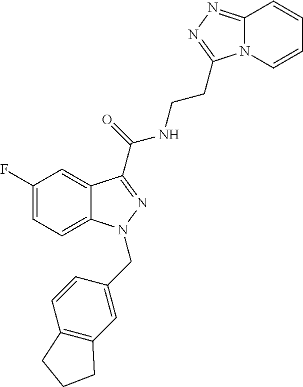 [SMILES: O=C(NCCc1nnc2ccccn12)c1nn(Cc2ccc3c(c2)CCC3)c2ccc(F)cc12] |
| 538 | 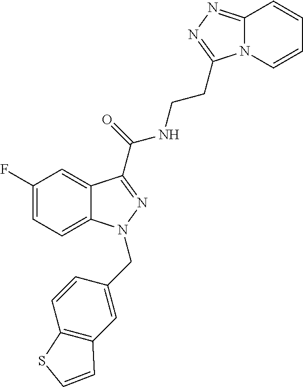 [SMILES: O=C(NCCc1nnc2ccccn12)c1nn(Cc2ccc3sccc3c2)c2ccc(F)cc12] |
| 539 | 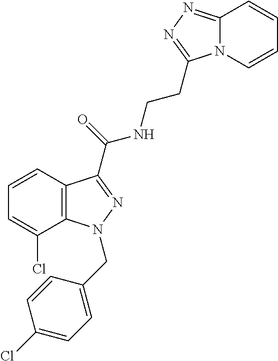 [SMILES: O=C(NCCc1nnc2ccccn12)c1nn(Cc2ccc(Cl)cc2)c2c(Cl)cccc12] |
| 540 | 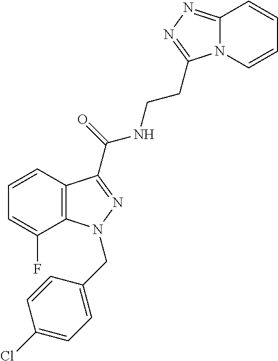 [SMILES: O=C(NCCc1nnc2ccccn12)c1nn(Cc2ccc(Cl)cc2)c2c(F)cccc12] |
| 541 | 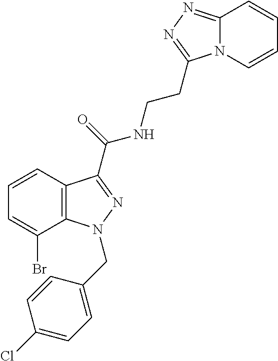 [SMILES: O=C(NCCc1nnc2ccccn12)c1nn(Cc2ccc(Cl)cc2)c2c(Br)cccc12] |
| 542 | 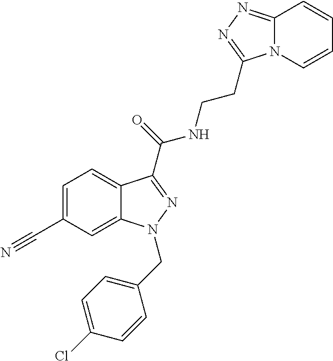 [SMILES: N#Cc1ccc2c(C(=O)NCCc3nnc4ccccn34)nn(Cc3ccc(Cl)cc3)c2c1] |
| 543 | 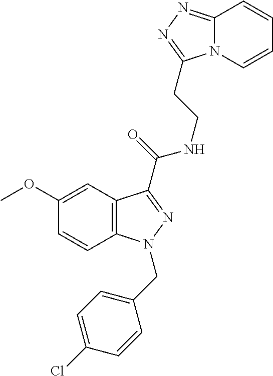 [SMILES: COc1ccc2c(c1)c(C(=O)NCCc1nnc3ccccn13)nn2Cc1ccc(Cl)cc1] |
| 544 | 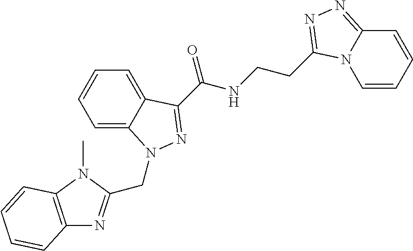 [SMILES: Cn1c(Cn2nc(C(=O)NCCc3nnc4ccccn34)c3ccccc32)nc2ccccc21] |
| 545 | 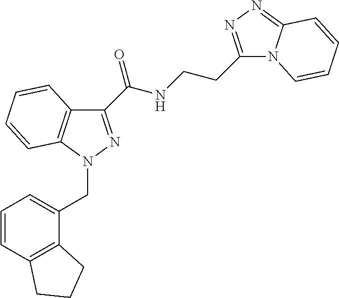 [SMILES: O=C(NCCc1nnc2ccccn12)c1nn(Cc2cccc3c2CCC3)c2ccccc12] |
| 546 | 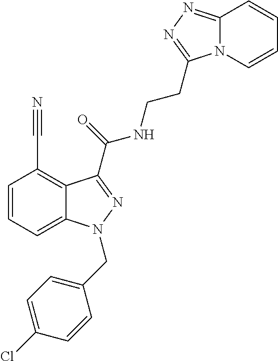 [SMILES: N#Cc1cccc2c1c(C(=O)NCCc1nnc3ccccn13)nn2Cc1ccc(Cl)cc1] |
| 547 | 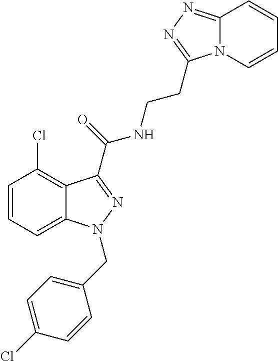 [SMILES: O=C(NCCc1nnc2ccccn12)c1nn(Cc2ccc(Cl)cc2)c2cccc(Cl)c12] |
| 548 | 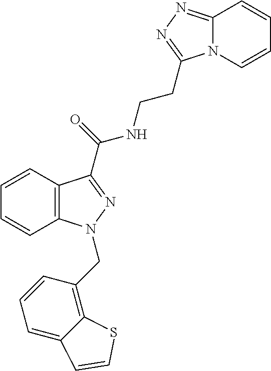 [SMILES: O=C(NCCc1nnc2ccccn12)c1nn(Cc2cccc3ccsc23)c2ccccc12] |
| 549 | 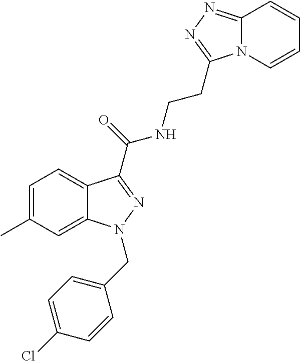 [SMILES: Cc1ccc2c(C(=O)NCCc3nnc4ccccn34)nn(Cc3ccc(Cl)cc3)c2c1] |
| 550 | 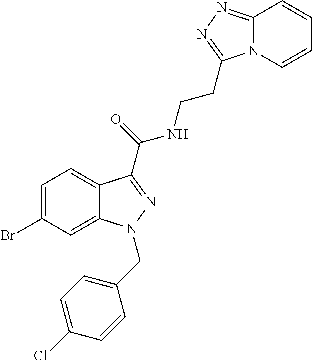 [SMILES: O=C(NCCc1nnc2ccccn12)c1nn(Cc2ccc(Cl)cc2)c2cc(Br)ccc12] |
| 551 | 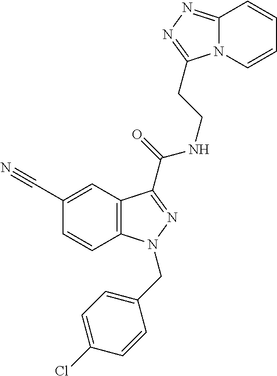 [SMILES: N#Cc1ccc2c(c1)c(C(=O)NCCc1nnc3ccccn13)nn2Cc1ccc(Cl)cc1] |
| 552 | 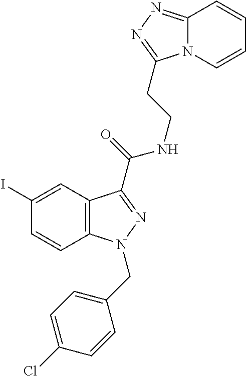 [SMILES: O=C(NCCc1nnc2ccccn12)c1nn(Cc2ccc(Cl)cc2)c2ccc(I)cc12] |
| 553 | 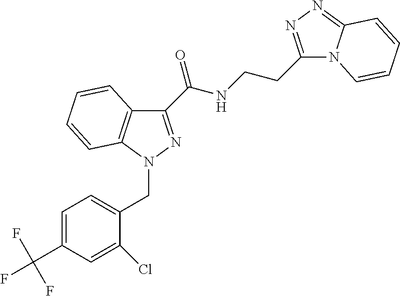 [SMILES: O=C(NCCc1nnc2ccccn12)c1nn(Cc2ccc(C(F)(F)F)cc2Cl)c2ccccc12] |
| 554 | 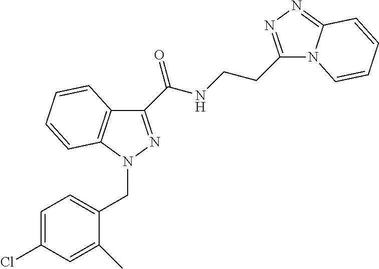 [SMILES: Cc1cc(Cl)ccc1Cn1nc(C(=O)NCCc2nnc3ccccn23)c2ccccc21] |
| 555 | 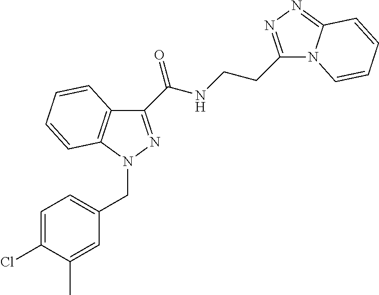 [SMILES: Cc1cc(Cn2nc(C(=O)NCCc3nnc4ccccn34)c3ccccc32)ccc1Cl] |
| 556 | 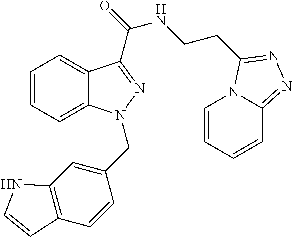 [SMILES: O=C(NCCc1nnc2ccccn12)c1nn(Cc2ccc3cc[nH]c3c2)c2ccccc12] |
| 557 | 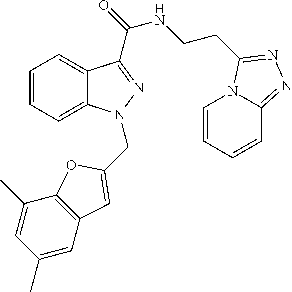 [SMILES: Cc1cc(C)c2oc(Cn3nc(C(=O)NCCc4nnc5ccccn45)c4ccccc43)cc2c1] |
| 558 | 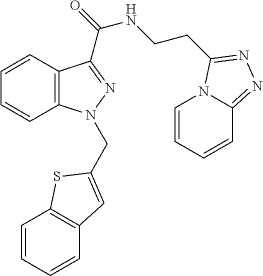 [SMILES: O=C(NCCc1nnc2ccccn12)c1nn(Cc2cc3ccccc3s2)c2ccccc12] |
| 559 | 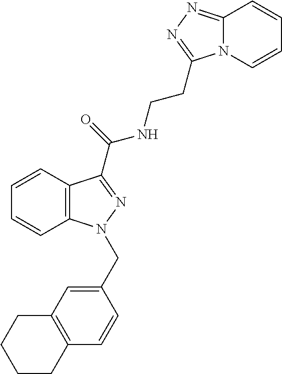 [SMILES: O=C(NCCc1nnc2ccccn12)c1nn(Cc2ccc3c(c2)CCCC3)c2ccccc12] |
| 560 | 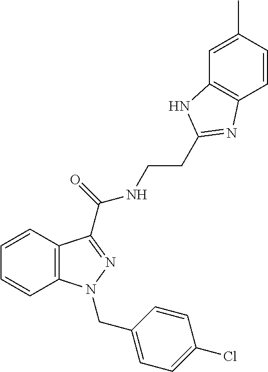 [SMILES: Cc1ccc2nc(CCNC(=O)c3nn(Cc4ccc(Cl)cc4)c4ccccc34)[nH]c2c1] |
| 561 | 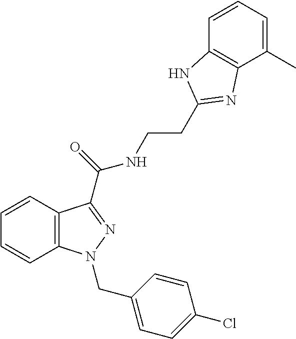 [SMILES: Cc1cccc2[nH]c(CCNC(=O)c3nn(Cc4ccc(Cl)cc4)c4ccccc34)nc12] |
| 562 | 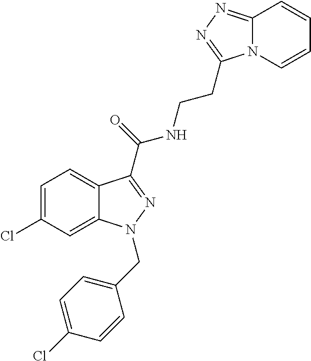 [SMILES: O=C(NCCc1nnc2ccccn12)c1nn(Cc2ccc(Cl)cc2)c2cc(Cl)ccc12] |
| 563 | 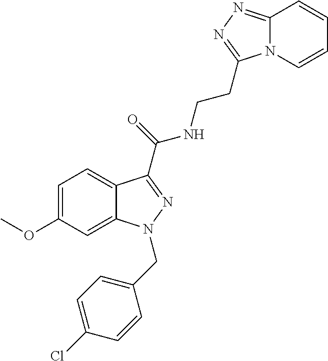 [SMILES: COc1ccc2c(C(=O)NCCc3nnc4ccccn34)nn(Cc3ccc(Cl)cc3)c2c1] |
| 564 | 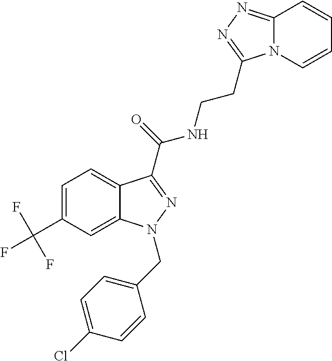 [SMILES: O=C(NCCc1nnc2ccccn12)c1nn(Cc2ccc(Cl)cc2)c2cc(C(F)(F)F)ccc12] |
| 565 | 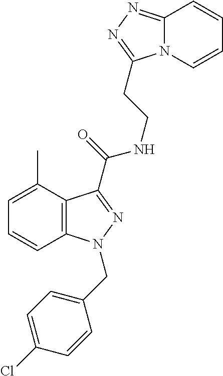 [SMILES: Cc1cccc2c1c(C(=O)NCCc1nnc3ccccn13)nn2Cc1ccc(Cl)cc1] |
| 566 | 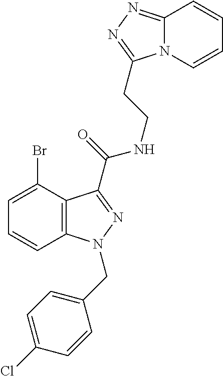 [SMILES: O=C(NCCc1nnc2ccccn12)c1nn(Cc2ccc(Cl)cc2)c2cccc(Br)c12] |
| 567 | 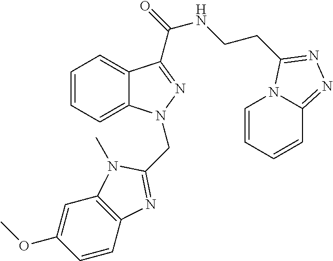 [SMILES: COc1ccc2nc(Cn3nc(C(=O)NCCc4nnc5ccccn45)c4ccccc43)n(C)c2c1] |
| 568 | 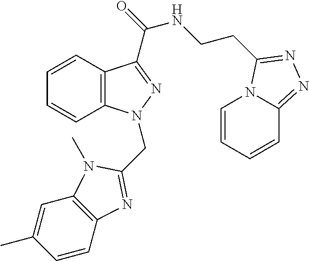 [SMILES: Cc1ccc2nc(Cn3nc(C(=O)NCCc4nnc5ccccn45)c4ccccc43)n(C)c2c1] |
| 569 | 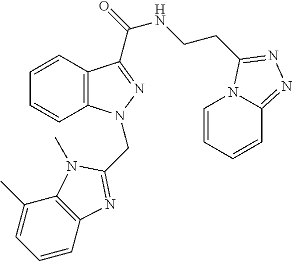 [SMILES: Cc1cccc2nc(Cn3nc(C(=O)NCCc4nnc5ccccn45)c4ccccc43)n(C)c12] |
| 570 | 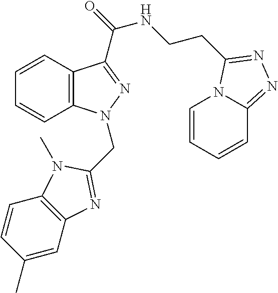 [SMILES: Cc1ccc2c(c1)nc(Cn1nc(C(=O)NCCc3nnc4ccccn34)c3ccccc31)n2C] |
| 571 | 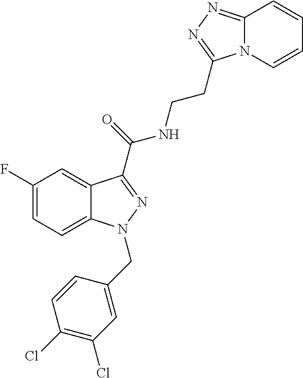 [SMILES: O=C(NCCc1nnc2ccccn12)c1nn(Cc2ccc(Cl)c(Cl)c2)c2ccc(F)cc12] |
| 572 | 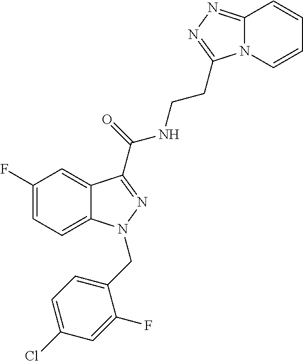 [SMILES: O=C(NCCc1nnc2ccccn12)c1nn(Cc2ccc(Cl)cc2F)c2ccc(F)cc12] |
| 573 | 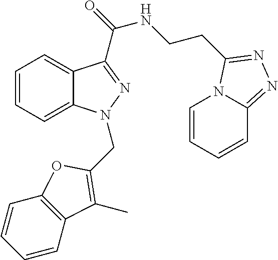 [SMILES: Cc1c(Cn2nc(C(=O)NCCc3nnc4ccccn34)c3ccccc32)oc2ccccc12] |
| 574 | 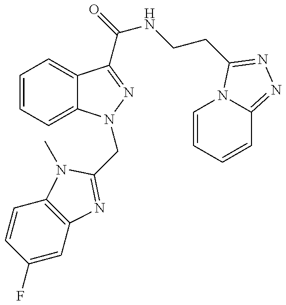 [SMILES: Cn1c(Cn2nc(C(=O)NCCc3nnc4ccccn34)c3ccccc32)nc2cc(F)ccc21] |

In certain embodiments, the invention provides a pharmaceutical composition comprising a compound of structure (I) or any one of formulas (IA) through (IH), or a pharmaceutically acceptable salt, isomer, hydrate, solvate or isotope thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, isotope or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for administration to a pediatric subject (e.g., solution, syrup, suspension, elixir, powder for reconstitution as suspension or solution, dispersible/effervescent tablet, chewable tablet, lollipop, freezer pops, troches, oral thin strips, orally disintegrating tablet, orally disintegrating strip, and sprinkle oral powder or granules); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In some embodiments, the pharmaceutical composition comprising a compound of structure (I) or any one of formulas (IA) through (IH), or a pharmaceutically acceptable salt, isomer, hydrate, solvate or isotope thereof, with at least one pharmaceutically acceptable carrier, diluent, or excipient further comprises a second therapeutic agent. In one embodiment, the second therapeutic agent is a liver disease therapeutic agent. In one embodiment, the liver disease therapeutic agent is ursodeoxycholic acid (UDCA), norUrsodeoxycholic acid, cholestyramine, stanozolol, naltrexone, rifampicin, Alisol B 23-acetate (AB23A), curcumin, dihydroartemisinin, fenofibrate, bezafibrate, metronidazole, methotrexate, colchicine, metformin, betaine, glucagon, naltrexone, a farnesoid X-receptor (FXR) agonist, a peroxisome proliferator-activated receptor (PPAR) agonist, a thyroid hormone receptor beta (TRO) agonist, or any combination thereof.

Examples of FXR agonists that may be used in the pharmaceutical compositions described herein include obeticholic acid, Turofexorate isopropyl (WAY-362450), 3-(2,6-dichlorophenyl)-4-(3′-carboxy-2-chlorostilben-4-yl)oxymethyl-5-isopropylisoxazole (GW4064), PX20606 (PX-102), PX-101, INT-767, INT-787, TERN-101, altenusin, tropifexor (LJN452), nidufexor, turofexorate isopropyl, fexaramine, silymarin, silybin, hedragonic acid, cafestol, Cilofexor (GS-9674 or Px-104), EDP-305, BAR704, BAR502, EYP-001, RDX-023, AGN-242266, HPG-1860, MET-409, AGN-242256, EP-024297, IOT-022, M-480, INV-33, RDX023-02, or any combination thereof.

In one embodiment, an FXR agonist is a bile acid or analog thereof (e.g., obeticholic acid, INT-767, INT-787, turofexorate isopropyl (WAY-362450), BAR502, hedragonic acid or BAR704) or a non-bile acid agonist (e.g., EDP-305, tropifexor, nidufexor, cilofexor, GW4064, Turofexorate isopropyl, fexaramine, PX20606 (PX-102), TERN-101, altenusin, silymarin, silybin, hedragonic acid, BAR502, EYP-001, RDX023-2, AGN-242266, HPG-1860, MET-409, EP-024297, M-480, or cafestol). In one embodiment, a PPAR agonist is a PPAR-alpha agonist, a PPAR-gamma agonist, a PPAR-delta agonist, a PPAR-alpha/gamma dual agonist, a PPAR alpha/delta dual agonist, a PPAR gamma/delta dual agonist, a PPAR alpha/gamma/delta pan agonist, or any combination thereof.

Examples of PPAR alpha agonists that may be used in the pharmaceutical compositions described herein include fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, and SRI 0171.

Examples of PPAR gamma agonists that may be used in the pharmaceutical compositions described herein include rosiglitazone, pioglitazone, deuterium-stabilized R-pioglitazone, efatutazone, ATx08-001, OMS-405, CHS-131, THR-0921, SER-150-DN, KDT-501, GED-0507-34-Levo, CLC-3001, and ALL-4.

Examples of PPAR delta agonists that may be used in the pharmaceutical compositions described herein include GW501516 (endurabol or ({4-[({4-methyl-2-[4-(trifluoromethyl)phenyl]-1,3-thiazol-5-yl}methyl)sulfanyl]-2-methylphenoxy} acetic acid)), MBX8025 (seladelpar or {2-methyl-4-[5-methyl-2-(4-trifluoromethyl-phenyl)-2H-[1,2,3]triazol-4-ylmethylsylfanyl]-phenoxy}-acetic acid), GW0742 ([4-[[[2-[3-fluoro-4-(trifluoromethyl)phenyl]-4-methyl-5-thiazolyl]methyl]thio]-2-methyl phenoxy]acetic acid), L165041, HPP-593, and NCP-1046.

Examples of PPAR alpha/gamma agonists that may be used in the pharmaceutical compositions described herein include saroglitazar, aleglitazar, muraglitazar, tesaglitazar, and DSP-8658.

Examples of PPAR alpha/delta agonists that may be used in the pharmaceutical compositions described herein include elafibranor and T913659.

Examples of PPAR gamma/delta agonists that may be used in the pharmaceutical compositions described herein include a conjugated linoleic acid (CLA) and T3D-959. Examples of PPAR alpha/gamma/delta agonists that may be used in the pharmaceutical compositions described herein include IVA337 (lanifibranor), TTA (tetradecylthioacetic acid), bavachinin, GW4148, GW9135, bezafibrate, lobeglitazone, 2-(4-(5,6-methylenedioxybenzo[d]thiazol-2-yl)-2-methylphenoxy)-2-methylpropanoic acid (MHY2013), and CS038.

Examples of thyroid hormone receptor beta agonists that may be used in the pharmaceutical compositions described herein include sobetirome, eprotirome, GC-24, MGL-3196, MGL-3745, VK-2809, KB141 [3,5-dichloro-4-(4-hydroxy-3-isopropylphenoxy) phenylacetic acid], and MB07811 (2R,4S)-4-(3-chlorophenyl)-2-[(3,5-dimethyl-4-(4′-hydroxy-3′-isopropylbenzyl)phenoxy)methyl]-2-oxido-[1,3,2]-dioxaphosphonane).

As used herein, the term “pharmaceutically acceptable carrier” refers to any ingredient other than the disclosed compounds, or a pharmaceutically acceptable isomer, racemate, hydrate, solvate, isotope or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal, or parenteral, including intravenous, subcutaneous and/or intramuscular. In one embodiment, the route of administration is oral. In another embodiment, the route of administration is topical.

Dosage forms can be administered once a day, or more than once a day, such as twice or thrice daily. Alternatively, dosage forms can be administered less frequently than daily, such as every other day, or weekly, if found to be advisable by a prescribing physician or drug's prescribing information. Dosing regimens include, for example, dose titration to the extent necessary or useful for the indication to be treated, thus allowing the patient's body to adapt to the treatment, to minimize or avoid unwanted side effects associated with the treatment, and/or to maximize the therapeutic effect of the present compounds. Other dosage forms include delayed or controlled-release forms. Suitable dosage regimens and/or forms include those set out, for example, in the latest edition of the Physicians' Desk Reference, incorporated herein by reference.

Proper dosages for pediatric patients can be determined using known methods, including weight, age, body surface area, and models such as Simcyp® Pediatric Simulation modeling (CERTARA, Princeton, N.J.) which can be used to establish a pharmacokinetic approach for dosing that takes into account patient age, ontogeny of the clearance pathways to eliminate a compound of any one of formulas (IA) through (IH), and body surface area (BSA). In one embodiment, the dosage form is formulated to provide a pediatric dose from about 30% to about 100% of an adult dose, or about 35%, 40%, 45%, 50%, 55%, 60%, 65%, 70%, 75%, 80%, 85%, 90%, 95%, or 100% of an adult dose.

In one embodiment, the invention provides an oral pharmaceutical composition comprising a compound of structure (I) or any one of formulas (IA) through (IH), or a pharmaceutically acceptable salt, isomer, hydrate, solvate or isotope thereof, together with at least one pharmaceutically acceptable oral carrier, diluent, or excipient. In another embodiment, the invention provides a topical pharmaceutical composition comprising a compound of structure (I) or any one of formulas (IA) through (IH), or a pharmaceutically acceptable salt, isomer, hydrate, solvate or isotope thereof, together with at least one pharmaceutically acceptable topical carrier, diluent, or excipient. For example, the oral pharmaceutical composition is provided to treat cholestatic pruritus, wherein the dosage regimen is, for example, once a day. In one embodiment, the topical pharmaceutical composition is provided to treat atopic dermatitis.

In another embodiment, there are provided methods of making a composition of a compound described herein including formulating a compound of the invention with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation. In some embodiments, the composition is formulated into a pediatric dosage form suitable for treating a pediatric subject.

In certain embodiments, the invention provides a compound having structure (I) or any one of formulas (IA) through (IH), or a pharmaceutically acceptable salt, isomer, hydrate, solvate or isotope thereof. Such compounds can be synthesized using standard synthetic techniques known to those skilled in the art. For example, compounds of the present invention can be synthesized using appropriately modified synthetic procedures set forth in the following Examples and Reaction Schemes.

To this end, the reactions, processes, and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention which is sufficiently acidic, an ammonium salt. A salt of this type can either be transformed into its free base or free acid form, respectively, by various methods known to a person skilled in the art, or be used as salts in subsequent biological assays. It is to be understood that the specific form of a compound of the present invention as isolated and as described herein is not necessarily the only form in which said compound can be applied to a biological assay in order to quantify the specific biological activity.

Chemical names were generated using the ChemDraw naming software (Version 17.0.0.206) by PerkinElmer Informatics, Inc. In some cases, generally accepted names of commercially available reagents were used in place of names generated by the naming software.

### EXAMPLES

General Methods ¹H NMR (400 MHz) spectra were obtained in solution of deuterochloroform (CDCl₃), deuteromethanol (CD₃OD) or dimethyl sulfoxide-D₆ (DMSO-D₆). HPLC retention times, purities and mass spectra (LCMS) were obtained using one of the following methods:

Method 1: Agilent 1260 Infinity II System equipped with an Agilent Poroshell 120 EC-18, 2.7 m, 4.6×100 mm column, using H₂O with 0.1% formic acid as mobile phase A, and MeCN with 0.1% formic acid as mobile phase B. The gradient was 10-95% mobile phase B over 12 min, held at 95% for 2 min, then returned to 10% mobile phase B over 1 min. The flow rate was 1 mL/min. An ESI detector in negative mode was used.

Method 2: Agilent 1260 Infinity II System equipped with an Agilent Poroshell 120 EC-18, 2.7 m, 4.6×100 mm column, using H₂O with 0.1% formic acid as mobile phase A, and MeCN with 0.1% formic acid as mobile phase B. The gradient was 10-95% mobile phase B over 12 min then held at 95% for 2 min, then return to 10% mobile phase B over 1 min. The flow rate was 1 mL/min. An ESI detector in positive mode was used.

Method 3: Agilent 1100 HPLC system equipped with an Agilent Eclipse XDB-C 18, 3.5μ, 4.6×150 mm column, using water with 0.05% TFA as mobile phase A, and methanol with 0.05% TFA as mobile phase B with a flow rate of 1 mL/minute. Using a gradient of 5% B (95% A) to 95% B over 12 minutes, held at 95% B for 3 minutes and then back to 5% B over 1 minute. An APCI detector in positive mode was used.

Method 4: Agilent 1100 HPLC system equipped with a BEH C18, 1.7 μM, 2.1×50 mm column using a low pH buffer gradient of 5% to 100% of MeCN in H₂O (10 mM NH₄HCO₂; pH 4) over 10 min at 0.7 mL/min. A Waters Micromass ZQ ESI detector was used.

Method 5: SHIMADZU LCMS-2020 equipped with Kinetex® EVO C18 2.1×30 mm 5 μm column, using H₂O with 0.0375% TFA as mobile phase A, and MeCN with 0.01875% TFA as mobile phase B. The gradient was 5-95% mobile phase B for 0.8 min, held at 95% for 0.15 min, then returned to 5% mobile phase B for 0.01 min, held at 5% for 0.04 min. The flow rate was 2 mL/min. An ESI detector in positive mode was used.

Method 6: SHIMADZU LCMS-2020 equipped with Kinetex® EVO C18 2.1×20 mm 2.6 μm column, using H₂O with 0.0375% TFA as mobile phase A, and MeCN with 0.01875% TFA as mobile phase B. The gradient was 5-95% mobile phase B for 0.8 min, held at 95% for 0.15 min, then returned to 5% mobile phase B for 0.01 min, held at 5% for 0.04 min. The flow rate was 2 mL/min. An ESI detector in positive mode was used.

The pyridine, dichloromethane (DCM), tetrahydrofuran (THF), and toluene used in the procedures were from Aldrich Sure-Seal bottles kept under nitrogen (N₂). All reactions were stirred magnetically, and temperatures are external reaction temperatures. Chromatographies were typically carried out using a Combiflash Rf flash purification system (Teledyne Isco) equipped with Redisep (Teledyne Isco) Rf Gold Normal-Phase silica gel (SiO₂) columns or by using a similar system.

Preparative HPLC purifications were typically performed using one of the following systems: 1) Waters System equipped with a Waters 2489 uv/vis detector, an Aquity QDA detector, a Waters xBridge Prep C18 5 μm OBD, 30×150 mm column, and eluting with various gradients of H₂O/MeCN (0.1% formic acid) at a 30 mL/min flow rate, 2) Teledyne Isco ACCQPrep® HP150 UV system equipped with a Waters xBridge Prep C18 5 μm OBD, 30×150 mm column, and eluting with various gradients of H₂O/MeCN (0.1% formic acid) at a 42.5 mL/min flow rate, or 3) column: Phenomenex Synergi C18 150×30 mm-4 m; mobile phase: [H₂O (0.225% formic acid)-MeCN]; B %: 55%-85%, 12 min) and were typically concentrated using a Genevac EZ-2.

The following additional abbreviations are used: ethyl acetate (EA), triethylamine (TEA), dimethyl sulfoxide (DMSO), silica gel (SiO₂), azobisisobutyronitrile (AIBN), diisobutylaluminium hydride (DIBAL), trifluoroacetic acid (TFA), 4-dimethylaminopyridine (DMAP), diphenylphosphoryl azide (DPPA), benzoyl peroxide (BPO), 1,1′-bis(diphenylphosphino)ferrocene (dppf), bis(pinacolato)diboron (B₂pin₂), tetrahydrofuran (THF), 1,4-diazabicyclo[2.2.2]octane bis(sulfur dioxide) adduct (DABSO), hexafluorophosphate azabenzotriazole tetramethyl uronium (HATU), hydroxybenzotriazole (HOBt), N-methyl morpholine (NMN), N-Bromosuccinimide (NBS), diisopropylethyl amine (DIPEA), diethyl azodicarboxylate (DEAD), diisopropyl azodicarboxylate (DIAD), 2-[2-(dicyclohexylphosphino)phenyl]-N-methylindole (CM-Phos), triflic acid (TfOH), 1-ethyl-3-(3-dimethylaminopropyl) carbodiimide hydrochloride (EDC), isopropanol (IPA), dimethylformamide (DMF), dimethyl acetamide (DMA), dichloromethane (DCM), 1,2-dichloroethane (DCE), acetonitrile (MeCN or ACN), 1,1′-thiocarbonyldiimidazole (TCDI), petroleum ether (PE), not determined (ND), retention time (RT), molecular weight (mw), room temperature (rt), hour (h), and not applicable (N/A).

### Example 1

### Synthesis of Compound 316

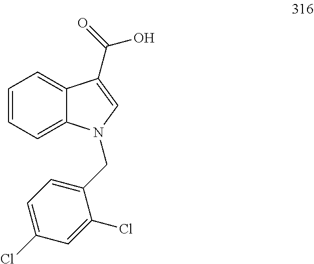 [SMILES: O=C(O)c1cn(Cc2ccc(Cl)cc2Cl)c2ccccc12]

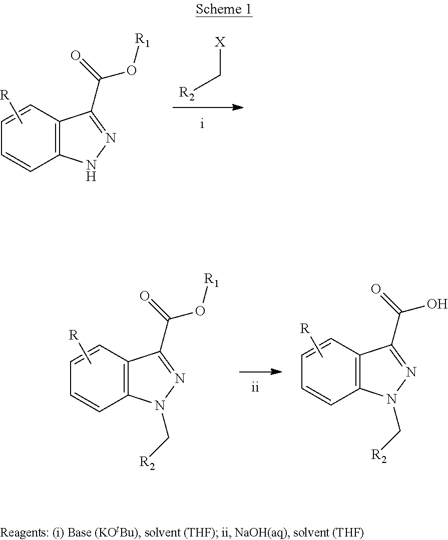

### Step 1-1. Synthesis of methyl 1-(2,4-dichlorobenzyl)-1H-indazole-3-carboxylate (INT-1A)

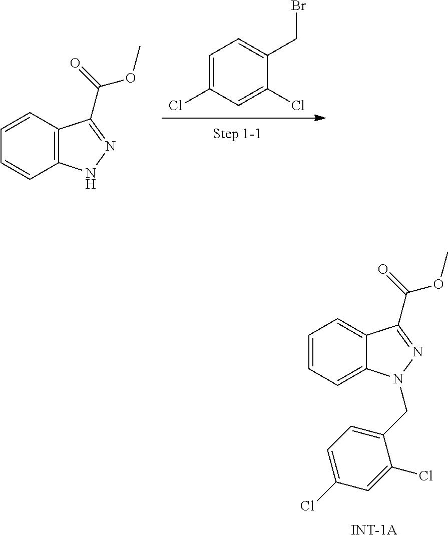

To a stirring solution of methyl 1H-indazole-3-carboxylate (300 mg, 1 Eq, 1.70 mmol) in THE (10 mL) at 0° C. was slowly added potassium tert-butoxide (210 mg, 1.2 Eq, 1.87 mmol) in two portions separated by 10 minutes. The reaction mixture was warmed to room temperature and stirred for 1 h. After 1 h, the reaction mixture was cooled back to 0° C. and 1-(bromomethyl)-2,4-dichlorobenzene (490 mg, 1.2 Eq., 2.04 mmol) was added. After 15 minutes, the reaction mixture was warmed to 50° C. After heating at 50° C. for 15 h, the reaction mixture was cooled to room temperature and H₂O (10 mL) was added. The aqueous layer was extracted with EtOAC (3×15 mL), dried over sodium sulfate, filtered, and concentrated in vacuo. The crude mixture was purified by SiO₂ gel chromatography (0->50% 10% MeOH in EtOAc and hexanes) to yield 443 mg (78%) of methyl 1-(2,4-dichlorobenzyl)-1H-indazole-3-carboxylate (INT-1A) as a white solid.

### Step 1-2. Synthesis of 1-(2,4-dichlorobenzyl)-1H-indazole-3-carboxylic acid (Compound 316)

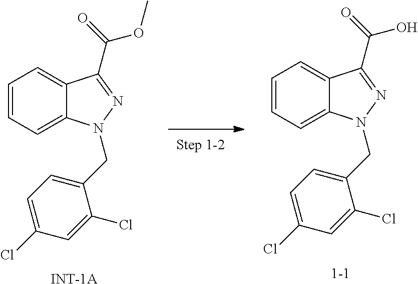

To a stirring solution of methyl 1-(2,4-dichlorobenzyl)-1H-indazole-3-carboxylate (INT-1A) (443 mg, 1.0 Eq., 1.32 mmol) in THE (5 mL) at room temperature was added 1M NaOH (aq.) (5.3 mL, 4.0 Eq., 5.3 mmol). After stirring for 14 h, the reaction mixture was concentrated in vacuo. To the crude residue was added H₂O (10 mL) followed by 3M HCl (3 mL). The aqueous layer was extracted with EtOAc (3×15 mL), dried over sodium sulfate, filtered, and concentrated in vacuo to afford 207 mg (49% yield) of 1-(2,4-dichlorobenzyl)-1H-indazole-3-carboxylic acid (Compound 316) as a white solid; LCMS (m/z) calculated for C₁₅H₁₀Cl₂N₂O₂: 320.0; found 321.0 [M+H]⁺, t_{R}=9.13 min (Method 2). ¹H NMR (400 MHz, DMSO-d6) δ 13.12 (br s, 1H), 8.12 (d, J=8 Hz, 1H), 7.81 (d, J=8 Hz, 1H), 7.70 (br s, 1H), 7.50 (t, J=8 Hz, 1H), 7.37 (m, 2H), 6.96 (d, J=8 Hz, 1H), 5.84 (s, 2H).

The compounds listed in Table 1 were made using the procedures of Scheme 1.

TABLE 1

| | Cpd | Purity | | Observed | | Purity |
|---|---|---|---|---|---|---|
| Structure | No. | RT (min) | MW | m/z | Ion | Method |
| 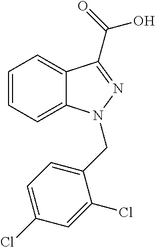 [SMILES: O=C(O)c1nn(Cc2ccc(Cl)cc2Cl)c2ccccc12] | 316 | 9.13 | 321.2 | 321.0 | [M + H]⁺ | 2 |
| 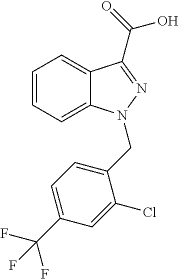 [SMILES: O=C(O)c1nn(Cc2ccc(C(F)(F)F)cc2Cl)c2ccccc12] | 317 | 9.851 | 354.7 | 353.0 | [M − H] | 1 |
| 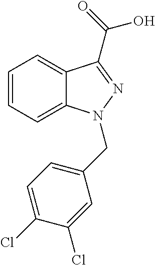 [SMILES: O=C(O)c1nn(Cc2ccc(Cl)c(Cl)c2)c2ccccc12] | 318 | 8.493 | 321.2 | 321.0 | [M + H]⁺ | 2 |
| 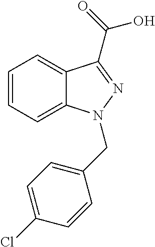 [SMILES: O=C(O)c1nn(Cc2ccc(Cl)cc2)c2ccccc12] | 319 | 8.744 | 286.72 | 287.1 | [M + H]⁺ | 2 |
| 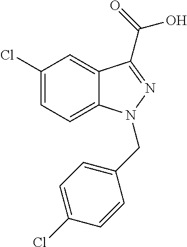 [SMILES: O=C(O)c1nn(Cc2ccc(Cl)cc2)c2ccc(Cl)cc12] | 320 | 9.139 | 321.16 | 321.0 | [M + H]⁺ | 2 |
| 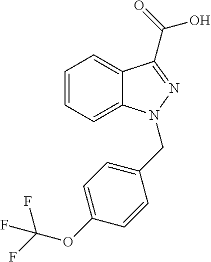 [SMILES: O=C(O)c1nn(Cc2ccc(OC(F)(F)F)cc2)c2ccccc12] | 321 | 8.292 | 336.3 | 334.8 | [M − H] | 1 |
| 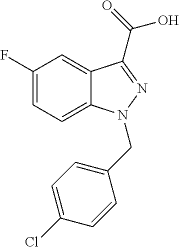 [SMILES: O=C(O)c1nn(Cc2ccc(Cl)cc2)c2ccc(F)cc12] | 322 | 8.438 | 304.71 | 305.1 | [M + H]⁺ | 2 |
| 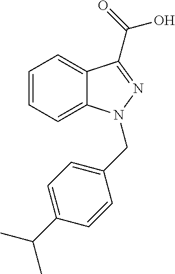 [SMILES: CC(C)c1ccc(Cn2nc(C(=O)O)c3ccccc32)cc1] | 323 | 4.791 | 294.4 | 295.2 | [M + H]⁺ | 2 |
| 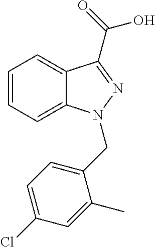 [SMILES: Cc1cc(Cl)ccc1Cn1nc(C(=O)O)c2ccccc21] | 324 | 9.52 | 300.74 | 301.1 | [M + H]⁺ | 2 |
| 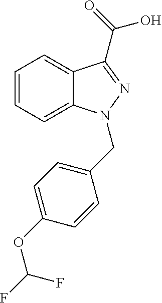 [SMILES: O=C(O)c1nn(Cc2ccc(OC(F)F)cc2)c2ccccc12] | 325 | 8.273 | 318.28 | 317.2 | [M − H] | 1 |
| 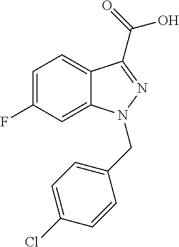 [SMILES: O=C(O)c1nn(Cc2ccc(Cl)cc2)c2cc(F)ccc12] | 326 | 8.572 | 304.71 | 305.1 | [M + H]⁺ | 2 |
| 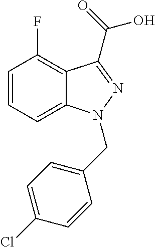 [SMILES: O=C(O)c1nn(Cc2ccc(Cl)cc2)c2cccc(F)c12] | 327 | 8.064 | 304.71 | 305.1 | [M + H]⁺ | 2 |
| 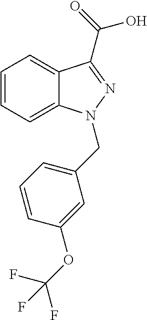 [SMILES: O=C(O)c1nn(Cc2cccc(OC(F)(F)F)c2)c2ccccc12] | 328 | 9.248 | 336.27 | 337.1 | [M + H]⁺ | 2 |
| 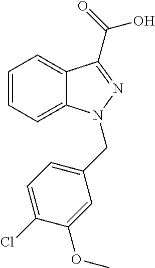 [SMILES: COc1cc(Cn2nc(C(=O)O)c3ccccc32)ccc1Cl] | 329 | 8.608 | 316.74 | 317.1 | [M + H]⁺ | 2 |
| 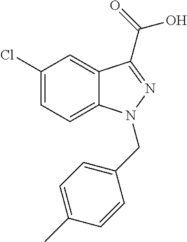 [SMILES: Cc1ccc(Cn2nc(C(=O)O)c3cc(Cl)ccc32)cc1] | 330 | 9.663 | 300.74 | 301.1 | [M + H]⁺ | 2 |
| 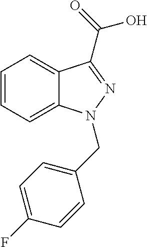 [SMILES: O=C(O)c1nn(Cc2ccc(F)cc2)c2ccccc12] | 331 | 8.059 | 270.26 | 271.1 | [M + H]⁺ | 2 |
| 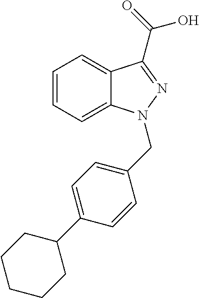 [SMILES: O=C(O)c1nn(Cc2ccc(C3CCCCC3)cc2)c2ccccc12] | 332 | 11.493 | 334.2 | 335.1 | [M + H]+ | 2 |
| 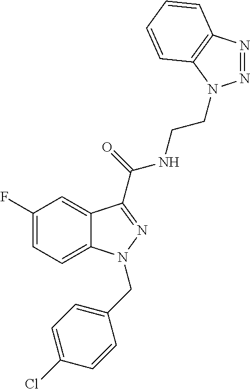 [SMILES: O=C(NCCn1nnc2ccccc21)c1nn(Cc2ccc(Cl)cc2)c2ccc(F)cc12] | 533 | 12.67 | 448.89 | 448.7 | [M + H]⁺ | 3 |
| 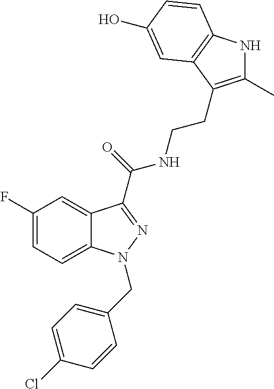 [SMILES: Cc1[nH]c2ccc(O)cc2c1CCNC(=O)c1nn(Cc2ccc(Cl)cc2)c2ccc(F)cc12] | 534 | 12.62 | 476.94 | 476.9 | [M + H]⁺ | 3 |
| 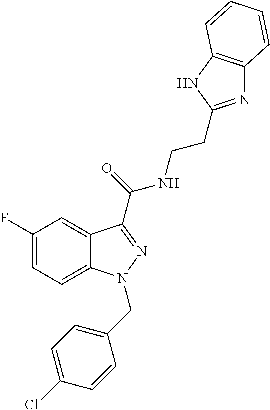 [SMILES: O=C(NCCc1nc2ccccc2[nH]1)c1nn(Cc2ccc(Cl)cc2)c2ccc(F)cc12] | 535 | 11.69 | 447.9 | 447.9 | [M + H]⁺ | 3 |
| 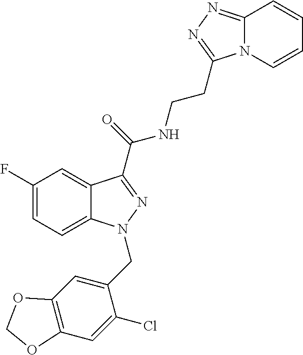 [SMILES: O=C(NCCc1nnc2ccccn12)c1nn(Cc2cc3c(cc2Cl)OCO3)c2ccc(F)cc12] | 536 | 11.86 | 492.9 | 493 | [M + H]⁺ | 3 |
| 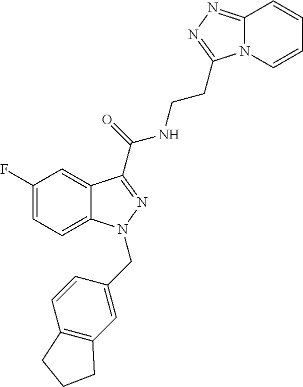 [SMILES: O=C(NCCc1nnc2ccccn12)c1nn(Cc2ccc3c(c2)CCC3)c2ccc(F)cc12] | 537 | 12.35 | 454.51 | 455.1 | [M + H]⁺ | 3 |
| 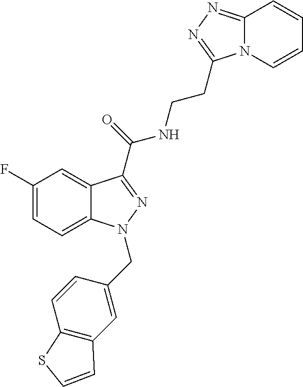 [SMILES: O=C(NCCc1nnc2ccccn12)c1nn(Cc2ccc3sccc3c2)c2ccc(F)cc12] | 538 | 11.72 | 470.53 | 471 | [M + H]⁺ | 3 |

### Example 2

### Synthesis of Compound 43

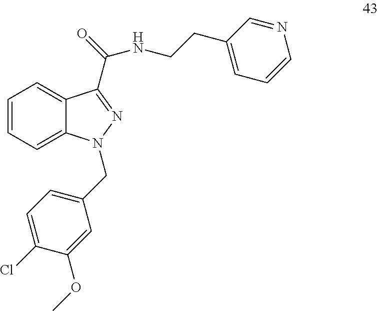 [SMILES: COc1cc(Cn2nc(C(=O)NCCc3cccnc3)c3ccccc32)ccc1Cl]

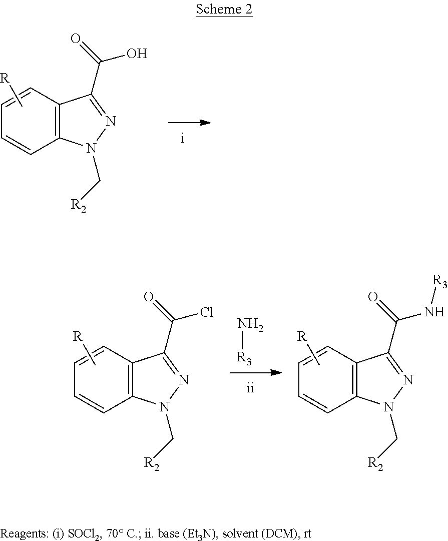

### Step 2-1. Synthesis of 1-(4-chloro-3-methoxybenzyl)-1H-indazole-3-carbonyl chloride (Compound 2-1)

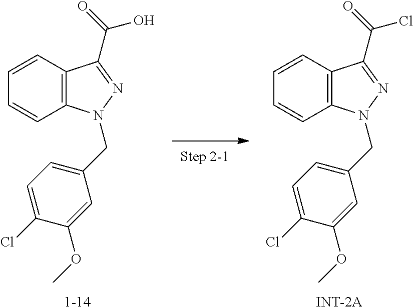

To 1-(4-chloro-3-methoxybenzyl)-1H-indazole-3-carboxylic acid (Compound 1-14) (25 mg, 1 Eq., 79 μmol) was added SOCl₂ in a vial. The vial was capped and heated at 70° C. After heating at 70° C. for 3 h, the reaction mixture was concentrated in vacuo.

To the crude residue was added toluene (2 mL) and the reaction mixture was concentrated in vacuo. This was repeated twice more with toluene (2 mL) to give 1-(4-chloro-3-methoxybenzyl)-1H-indazole-3-carbonyl chloride (INT-2A) (26 mg) yield (100%) as a light yellow oil that was carried on without further purification.

### Step 2-2. Synthesis of 1-(4-chloro-3-methoxybenzyl)-N-(2-(pyridin-3-yl)ethyl)-1H-indazole-3-carboxamide (Compound 43)

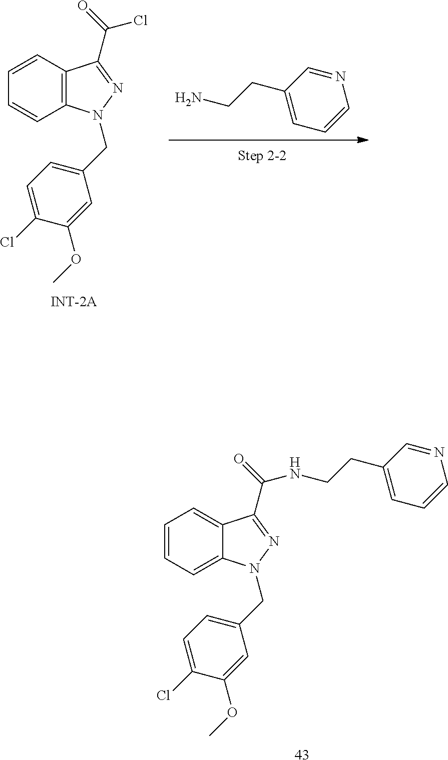

To a stirring solution of 1-(4-chloro-3-methoxybenzyl)-1H-indazole-3-carbonyl chloride (INT-2A) (26 mg, 1.0 Eq, 78 μmol) in DCM (1 mL) at room temperature was added Et₃N (54 μL, 5.0 Eq, 390 μmol) followed by a solution of 2-(pyridin-3-yl)ethan-1-amine (9.5 mg, 1.0 Eq, 78 μmol) in DCM (0.2 mL). After stirring for 14 h, the reaction mixture was concentrated in vacuo. The crude residue was purified by reversed phase preparatory HPLC (20->35% 0.1% formic acid in MeCN and 0.1% formic acid in H₂O) to yield 6.5 mg (20%) of 1-(2,4-dichlorobenzyl)-1H-indazole-3-carboxylic acid (Compound 43); LCMS (m/z) calculated for C₂₃H₁₁ClN₄O₂: 420.1; found 421.1 [M+H]⁺, t_{R}=7.073 min (Method 2). ¹H NMR (400 MHz, CDCl₃) δ 8.40 (d, J=8 Hz, 1H), 7.65 (d, J=8 Hz, 1H), 7.40 (m, 1H), 7.30 (m, 5H), 7.14 (m, 1H), 6.69 (m, 2H), 3.79 (m, 5H), 6.96 (d, J=8 Hz, 1H), 5.84 (s, 2H), 3.02 (t, J=8 Hz, 1H).

The compounds listed in Table 2 were made using the procedures of Scheme 2.

TABLE 2

| | Cpd | Purity RT | | Observed | | Purity |
|---|---|---|---|---|---|---|
| Structure | No. | (min) | MW | m/z | Ion | Method |
| 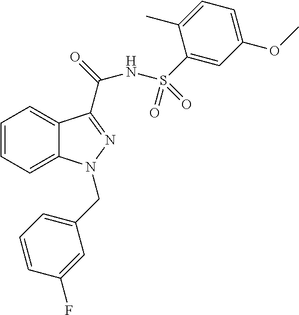 [SMILES: COc1ccc(C)c(S(=O)(=O)NC(=O)c2nn(Cc3cccc(F)c3)c3ccccc23)c1] | 257 | 10.804 | 453.5 | 454.1 | [M + H]⁺ | 2 |
| 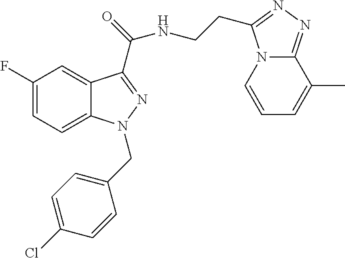 [SMILES: Cc1cccn2c(CCNC(=O)c3nn(Cc4ccc(Cl)cc4)c4ccc(F)cc34)nnc12] | 1 | 7.851 | 462.9 | 463.1 | [M + H]⁺ | 2 |
| 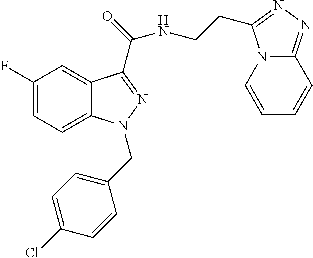 [SMILES: O=C(NCCc1nnc2ccccn12)c1nn(Cc2ccc(Cl)cc2)c2ccc(F)cc12] | 2 | 7.618 | 448.9 | 449.1 | [M + H]⁺ | 2 |
| 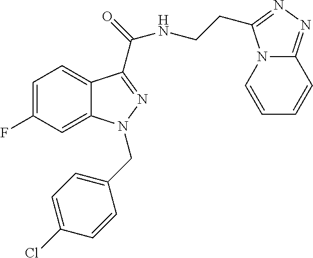 [SMILES: O=C(NCCc1nnc2ccccn12)c1nn(Cc2ccc(Cl)cc2)c2cc(F)ccc12] | 3 | 7.704 | 448.9 | 449.1 | [M + H]⁺ | 2 |
| 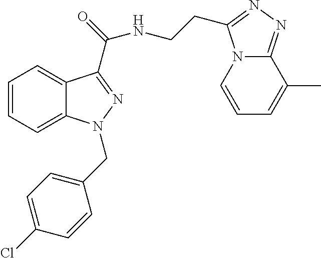 [SMILES: Cc1cccn2c(CCNC(=O)c3nn(Cc4ccc(Cl)cc4)c4ccccc34)nnc12] | 4 | 7.577 | 444.9 | 445.2 | [M + H]⁺ | 2 |
| 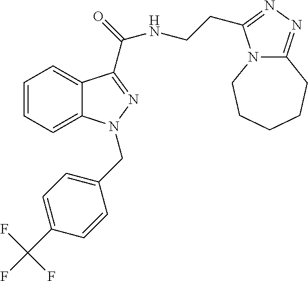 [SMILES: O=C(NCCc1nnc2n1CCCCC2)c1nn(Cc2ccc(C(F)(F)F)cc2)c2ccccc12] | 5 | 7.561 | 482.5 | 483.2 | [M + H]⁺ | 1 |
| 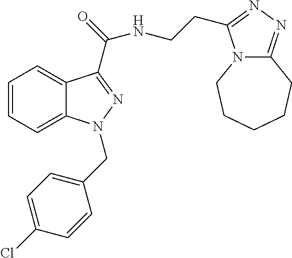 [SMILES: O=C(NCCc1nnc2n1CCCCC2)c1nn(Cc2ccc(Cl)cc2)c2ccccc12] | 6 | 7.937 | 449.0 | 449.2 | [M + H]⁺ | 2 |
| 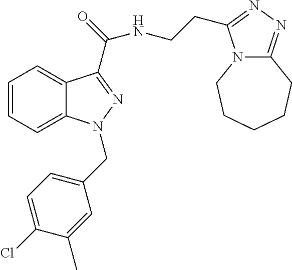 [SMILES: Cc1cc(Cn2nc(C(=O)NCCc3nnc4n3CCCCC4)c3ccccc32)ccc1Cl] | 7 | 8.044 | 463.0 | 463.2 | [M + H]⁺ | 2 |
| 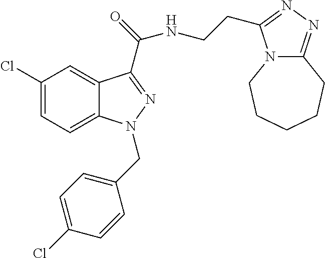 [SMILES: O=C(NCCc1nnc2n1CCCCC2)c1nn(Cc2ccc(Cl)cc2)c2ccc(Cl)cc12] | 8 | 8.069 | 483.4 | 483.1 | [M + H]⁺ | 2 |
| 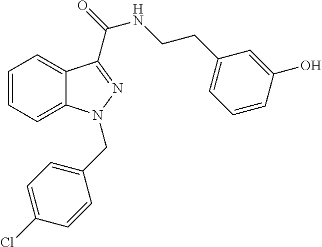 [SMILES: O=C(NCCc1cccc(O)c1)c1nn(Cc2ccc(Cl)cc2)c2ccccc12] | 9 | 9.654 | 405.9 | 406.1 | [M + H]⁺ | 2 |
| 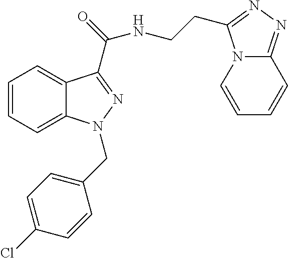 [SMILES: O=C(NCCc1nnc2ccccn12)c1nn(Cc2ccc(Cl)cc2)c2ccccc12] | 10 | 7.818 | 430.1 | 431.1 | [M + H]⁺ | 2 |
| 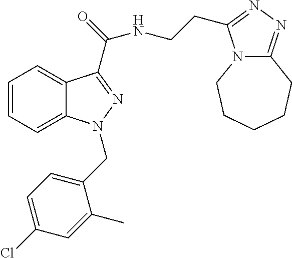 [SMILES: Cc1cc(Cl)ccc1Cn1nc(C(=O)NCCc2nnc3n2CCCCC3)c2ccccc21] | 11 | 8.00 | 462.2 | 463.2 | [M + H]⁺ | 2 |
| 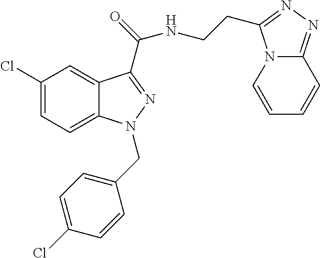 [SMILES: O=C(NCCc1nnc2ccccn12)c1nn(Cc2ccc(Cl)cc2)c2ccc(Cl)cc12] | 12 | 8.26 | 464.1 | 465.1 | [M + H]⁺ | 2 |
| 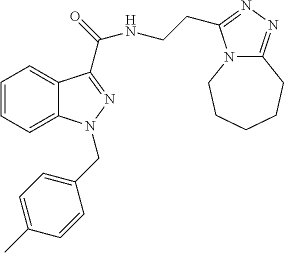 [SMILES: Cc1ccc(Cn2nc(C(=O)NCCc3nnc4n3CCCCC4)c3ccccc32)cc1] | 13 | 7.48 | 428.2 | 429.2 | [M + H]⁺ | 2 |
| 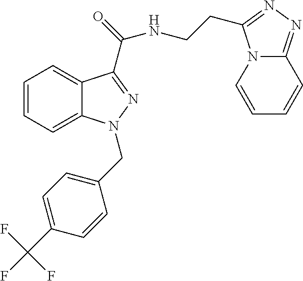 [SMILES: O=C(NCCc1nnc2ccccn12)c1nn(Cc2ccc(C(F)(F)F)cc2)c2ccccc12] | 14 | 7.68 | 464.2 | 465.1 | [M + H]⁺ | 2 |
| 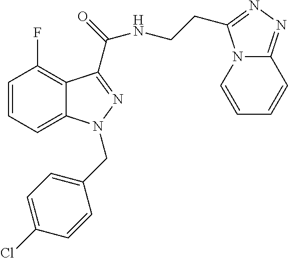 [SMILES: O=C(NCCc1nnc2ccccn12)c1nn(Cc2ccc(Cl)cc2)c2cccc(F)c12] | 15 | 6.998 | 448.1 | 449.1 | [M + H]⁺ | 2 |
| 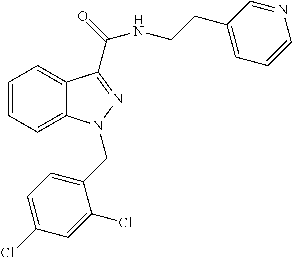 [SMILES: O=C(NCCc1cccnc1)c1nn(Cc2ccc(Cl)cc2Cl)c2ccccc12] | 16 | 7.787 | 424.1 | 425.1 | [M + H]⁺ | 2 |
| 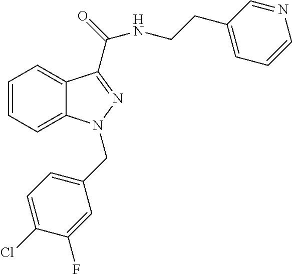 [SMILES: O=C(NCCc1cccnc1)c1nn(Cc2ccc(Cl)c(F)c2)c2ccccc12] | 17 | 7.173 | 408.1 | 409.1 | [M + H]⁺ | 2 |
| 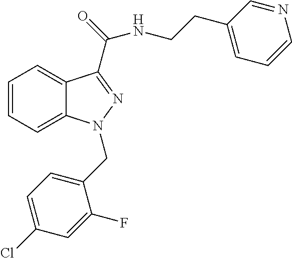 [SMILES: O=C(NCCc1cccnc1)c1nn(Cc2ccc(Cl)cc2F)c2ccccc12] | 18 | 7.228 | 408.1 | 409.1 | [M + H]⁺ | 2 |
| 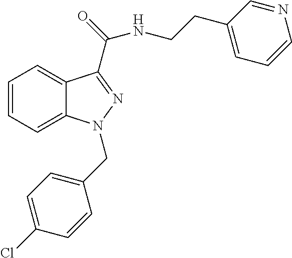 [SMILES: O=C(NCCc1cccnc1)c1nn(Cc2ccc(Cl)cc2)c2ccccc12] | 19 | 6.821 | 390.1 | 391.1 | [M + H]⁺ | 2 |
| 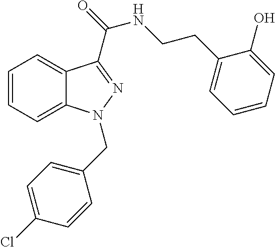 [SMILES: O=C(NCCc1ccccc1O)c1nn(Cc2ccc(Cl)cc2)c2ccccc12] | 20 | 10.573 | 405.1 | 406.1 | [M + H]⁺ | 2 |
| 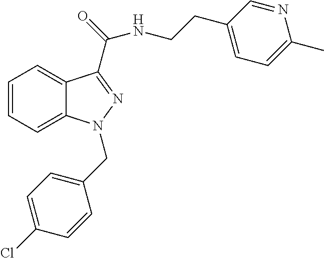 [SMILES: Cc1ccc(CCNC(=O)c2nn(Cc3ccc(Cl)cc3)c3ccccc23)cn1] | 21 | 6.166 | 404.1 | 405.2 | [M + H]⁺ | 2 |
| 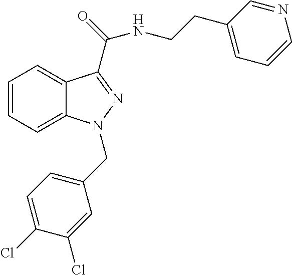 [SMILES: O=C(NCCc1cccnc1)c1nn(Cc2ccc(Cl)c(Cl)c2)c2ccccc12] | 22 | 7.594 | 424.1 | 425.1 | [M + H]⁺ | 2 |
| 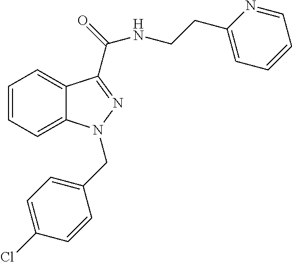 [SMILES: O=C(NCCc1ccccn1)c1nn(Cc2ccc(Cl)cc2)c2ccccc12] | 23 | 7.202 | 390.1 | 391.1 | [M + H]⁺ | 2 |
| 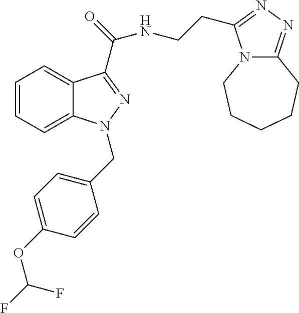 [SMILES: O=C(NCCc1nnc2n1CCCCC2)c1nn(Cc2ccc(OC(F)F)cc2)c2ccccc12] | 24 | 7.742 | 480.2 | 481.2 | [M + H]⁺ | 2 |
| 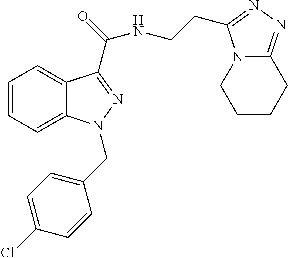 [SMILES: O=C(NCCc1nnc2n1CCCC2)c1nn(Cc2ccc(Cl)cc2)c2ccccc12] | 25 | 7.194 | 434.2 | 435.2 | [M + H]⁺ | 2 |
| 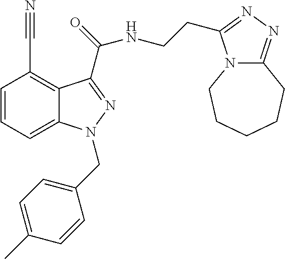 [SMILES: Cc1ccc(Cn2nc(C(=O)NCCc3nnc4n3CCCCC4)c3c(C#N)cccc32)cc1] | 26 | 6.986 | 453.2 | 454.2 | [M + H]⁺ | 2 |
| 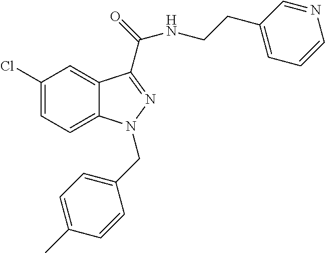 [SMILES: Cc1ccc(Cn2nc(C(=O)NCCc3cccnc3)c3cc(Cl)ccc32)cc1] | 27 | 7.792 | 404.1 | 405.2 | [M + H]⁺ | 2 |
| 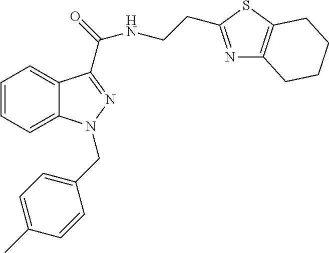 [SMILES: Cc1ccc(Cn2nc(C(=O)NCCc3nc4c(s3)CCCC4)c3ccccc32)cc1] | 28 | 11.27 | 430.2 | 431.2 | [M + H]⁺ | 2 |
| 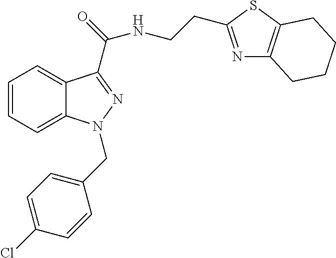 [SMILES: O=C(NCCc1nc2c(s1)CCCC2)c1nn(Cc2ccc(Cl)cc2)c2ccccc12] | 29 | 11.395 | 450.1 | 451.1 | [M + H]⁺ | 2 |
| 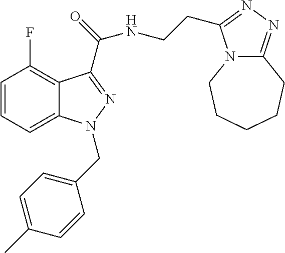 [SMILES: Cc1ccc(Cn2nc(C(=O)NCCc3nnc4n3CCCCC4)c3c(F)cccc32)cc1] | 30 | 7.171 | 446.2 | 447.2 | [M + H]⁺ | 2 |
| 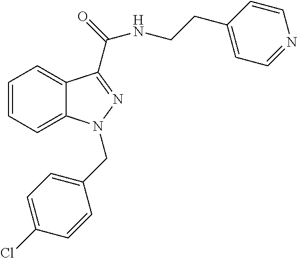 [SMILES: O=C(NCCc1ccncc1)c1nn(Cc2ccc(Cl)cc2)c2ccccc12] | 31 | 6.992 | 390.1 | 391.1 | [M + H]⁺ | 2 |
| 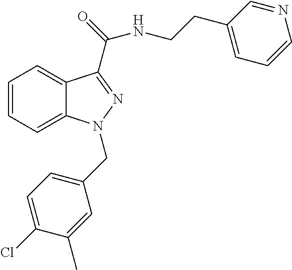 [SMILES: Cc1cc(Cn2nc(C(=O)NCCc3cccnc3)c3ccccc32)ccc1Cl] | 32 | 7.396 | 404.1 | 405.1 | [M + H]⁺ | 2 |
| 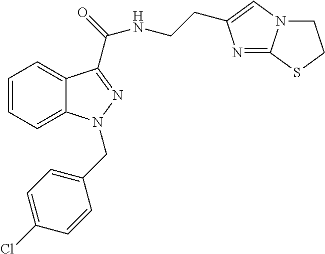 [SMILES: O=C(NCCc1cn2c(n1)SCC2)c1nn(Cc2ccc(Cl)cc2)c2ccccc12] | 33 | 7.044 | 437.1 | 438.1 | [M + H]⁺ | 2 |
| 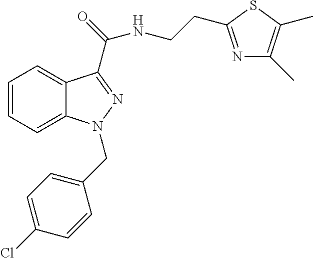 [SMILES: Cc1nc(CCNC(=O)c2nn(Cc3ccc(Cl)cc3)c3ccccc23)sc1C] | 34 | 10.249 | 424.1 | 425.1 | [M + H]⁺ | 2 |
| 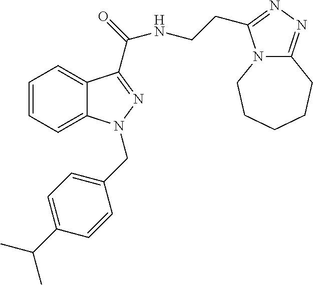 [SMILES: CC(C)c1ccc(Cn2nc(C(=O)NCCc3nnc4n3CCCCC4)c3ccccc32)cc1] | 35 | 8.381 | 456.3 | 457.2 | [M + H]⁺ | 2 |
| 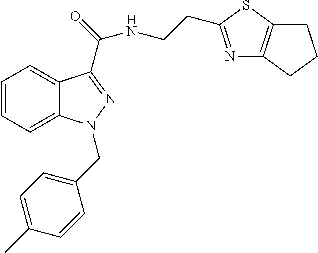 [SMILES: Cc1ccc(Cn2nc(C(=O)NCCc3nc4c(s3)CCC4)c3ccccc32)cc1] | 36 | 11.149 | 416.2 | 417.2 | [M + H]⁺ | 2 |
| 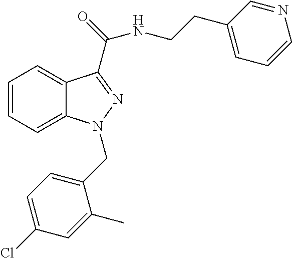 [SMILES: Cc1cc(Cl)ccc1Cn1nc(C(=O)NCCc2cccnc2)c2ccccc21] | 37 | 7.365 | 404.1 | 405.1 | [M + H]⁺ | 2 |
| 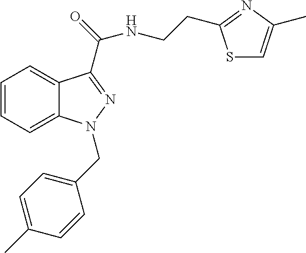 [SMILES: Cc1ccc(Cn2nc(C(=O)NCCc3nc(C)cs3)c3ccccc32)cc1] | 38 | 10.033 | 390.2 | 391.2 | [M + H]⁺ | 2 |
| 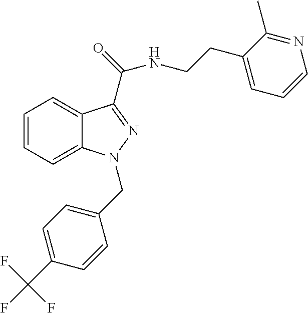 [SMILES: Cc1ncccc1CCNC(=O)c1nn(Cc2ccc(C(F)(F)F)cc2)c2ccccc12] | 39 | 6.766 | 438.2 | 439.2 | [M + H]⁺ | 2 |
| 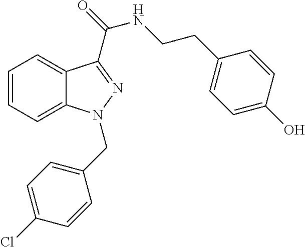 [SMILES: O=C(NCCc1ccc(O)cc1)c1nn(Cc2ccc(Cl)cc2)c2ccccc12] | 40 | 10.004 | 405.1 | 406.1 | [M + H]⁺ | 2 |
| 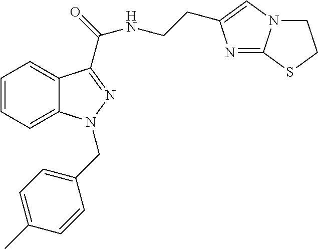 [SMILES: Cc1ccc(Cn2nc(C(=O)NCCc3cn4c(n3)SCC4)c3ccccc32)cc1] | 41 | 6.951 | 417.2 | 418.2 | [M + H]⁺ | 2 |
| 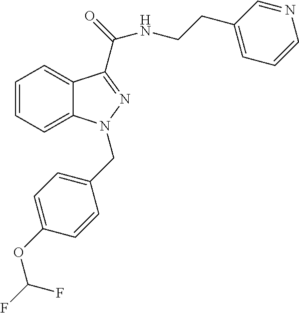 [SMILES: O=C(NCCc1cccnc1)c1nn(Cc2ccc(OC(F)F)cc2)c2ccccc12] | 42 | 6.723 | 422.2 | 423.2 | [M + H]⁺ | 2 |
| 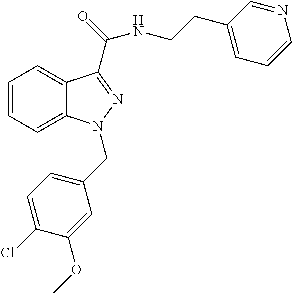 [SMILES: COc1cc(Cn2nc(C(=O)NCCc3cccnc3)c3ccccc32)ccc1Cl] | 43 | 7.073 | 420.1 | 421.1 | [M + H]⁺ | 2 |
| 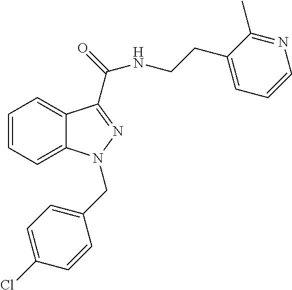 [SMILES: Cc1ncccc1CCNC(=O)c1nn(Cc2ccc(Cl)cc2)c2ccccc12] | 44 | 6.607 | 404.1 | 405.1 | [M + H]⁺ | 2 |
| 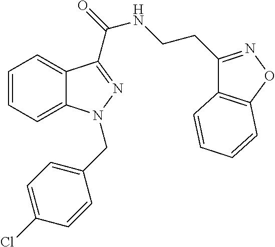 [SMILES: O=C(NCCc1noc2ccccc12)c1nn(Cc2ccc(Cl)cc2)c2ccccc12] | 45 | 10.77 | 430.1 | 431.1 | [M + H]⁺ | 2 |
| 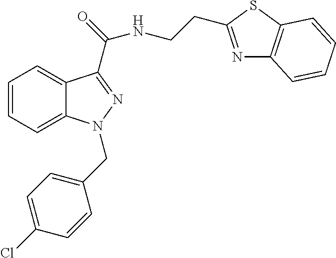 [SMILES: O=C(NCCc1nc2ccccc2s1)c1nn(Cc2ccc(Cl)cc2)c2ccccc12] | 46 | 11.476 | 446.1 | 447.1 | [M + H]⁺ | 2 |
| 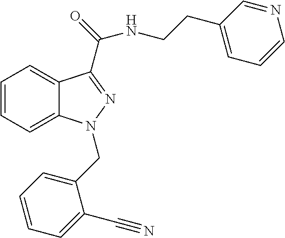 [SMILES: N#Cc1ccccc1Cn1nc(C(=O)NCCc2cccnc2)c2ccccc21] | 47 | 5.785 | 381.2 | 382.2 | [M + H]⁺ | 2 |
| 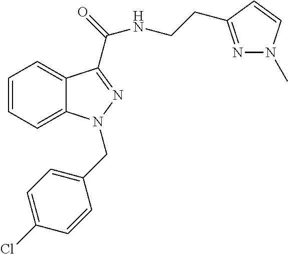 [SMILES: Cn1ccc(CCNC(=O)c2nn(Cc3ccc(Cl)cc3)c3ccccc23)n1] | 48 | 9.409 | 393.1 | 394.1 | [M + H]⁺ | 2 |
| 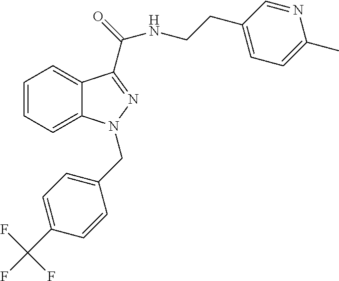 [SMILES: Cc1ccc(CCNC(=O)c2nn(Cc3ccc(C(F)(F)F)cc3)c3ccccc23)cn1] | 49 | 8.759 | 438.2 | 439.2 | [M + H]⁺ | 2 |
| 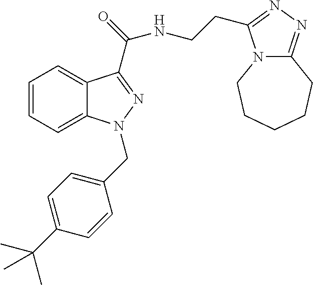 [SMILES: CC(C)(C)c1ccc(Cn2nc(C(=O)NCCc3nnc4n3CCCCC4)c3ccccc32)cc1] | 50 | 8.893 | 470.3 | 471.2 | [M + H]⁺ | 2 |
| 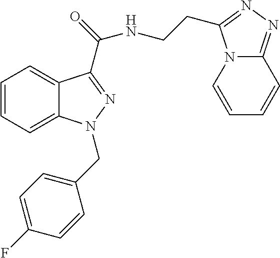 [SMILES: O=C(NCCc1nnc2ccccn12)c1nn(Cc2ccc(F)cc2)c2ccccc12] | 51 | 6.758 | 414.2 | 415.2 | [M + H]⁺ | 2 |
| 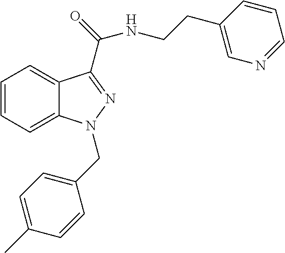 [SMILES: Cc1ccc(Cn2nc(C(=O)NCCc3cccnc3)c3ccccc32)cc1] | 52 | 6.898 | 370.2 | 371.2 | [M + H]⁺ | 2 |
| 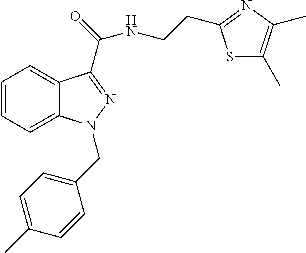 [SMILES: Cc1ccc(Cn2nc(C(=O)NCCc3nc(C)c(C)s3)c3ccccc32)cc1] | 53 | 10.116 | 404.2 | 405.2 | [M + H]⁺ | 2 |
| 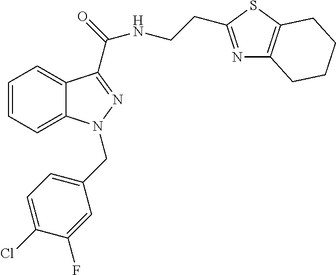 [SMILES: O=C(NCCc1nc2c(s1)CCCC2)c1nn(Cc2ccc(Cl)c(F)c2)c2ccccc12] | 54 | 11.548 | 468.1 | 469.1 | [M + H]⁺ | 2 |
| 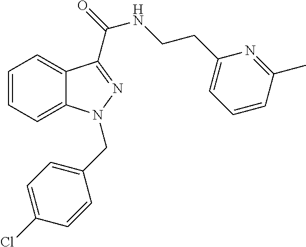 [SMILES: Cc1cccc(CCNC(=O)c2nn(Cc3ccc(Cl)cc3)c3ccccc23)n1] | 55 | 7.276 | 404.1 | 405.1 | [M + H]⁺ | 2 |
| 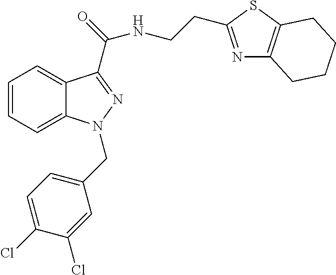 [SMILES: O=C(NCCc1nc2c(s1)CCCC2)c1nn(Cc2ccc(Cl)c(Cl)c2)c2ccccc12] | 56 | 12.196 | 484.1 | 485.1 | [M + H]⁺ | 2 |
| 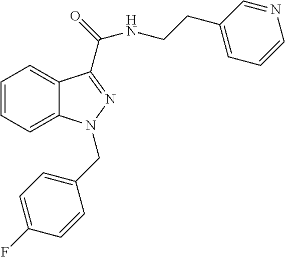 [SMILES: O=C(NCCc1cccnc1)c1nn(Cc2ccc(F)cc2)c2ccccc12] | 57 | 6.337 | 374.2 | 375.2 | [M + H]⁺ | 2 |
| 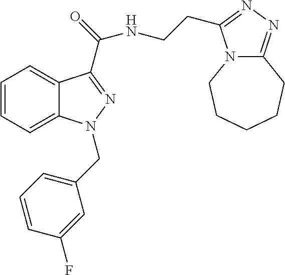 [SMILES: O=C(NCCc1nnc2n1CCCCC2)c1nn(Cc2cccc(F)c2)c2ccccc12] | 58 | 7.055 | 432.2 | 433.2 | [M + H]⁺ | 2 |
| 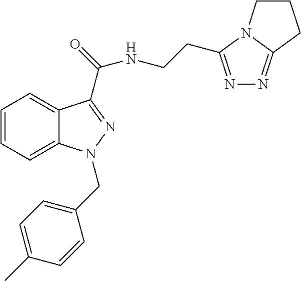 [SMILES: Cc1ccc(Cn2nc(C(=O)NCCc3nnc4n3CCC4)c3ccccc32)cc1] | 59 | 6.93 | 400.2 | 401.2 | [M + H]⁺ | 2 |
| 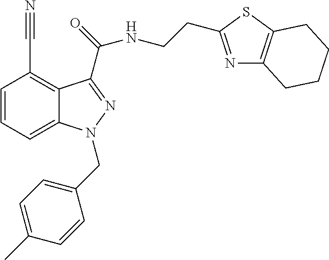 [SMILES: Cc1ccc(Cn2nc(C(=O)NCCc3nc4c(s3)CCCC4)c3c(C#N)cccc32)cc1] | 60 | 10.341 | 455.2 | 456.2 | [M + H]⁺ | 2 |
| 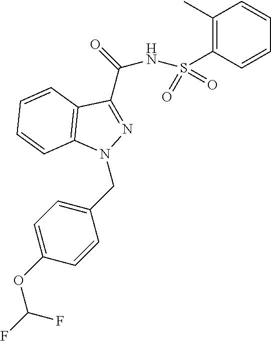 [SMILES: Cc1ccccc1S(=O)(=O)NC(=O)c1nn(Cc2ccc(OC(F)F)cc2)c2ccccc12] | 258 | 10.774 | 471.1 | 471.7 | [M + H]⁺ | 2 |
| 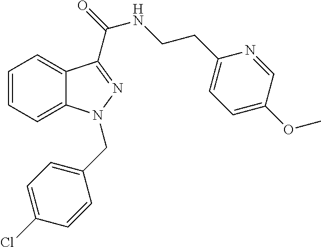 [SMILES: COc1ccc(CCNC(=O)c2nn(Cc3ccc(Cl)cc3)c3ccccc23)nc1] | 61 | 7.873 | 420.3 | 421.2 | [M + H]⁺ | 2 |
| 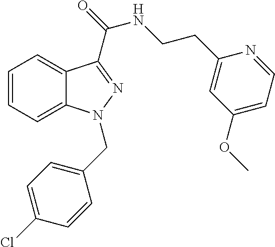 [SMILES: COc1ccnc(CCNC(=O)c2nn(Cc3ccc(Cl)cc3)c3ccccc23)c1] | 62 | 6.925 | 420.1 | 421.2 | [M + H]⁺ | 2 |
| 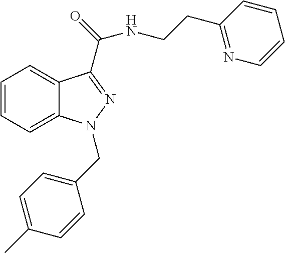 [SMILES: Cc1ccc(Cn2nc(C(=O)NCCc3ccccn3)c3ccccc32)cc1] | 63 | 7.023 | 370.2 | 371.1 | [M + H]⁺ | 2 |
| 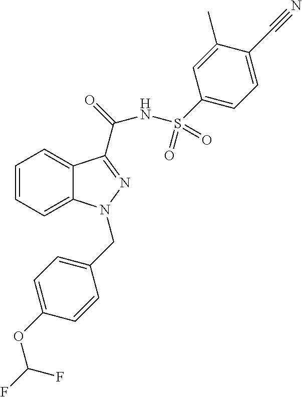 [SMILES: Cc1cc(S(=O)(=O)NC(=O)c2nn(Cc3ccc(OC(F)F)cc3)c3ccccc23)ccc1C#N] | 259 | 10.659 | 496.1 | 497.1 | [M + H]⁺ | 2 |
| 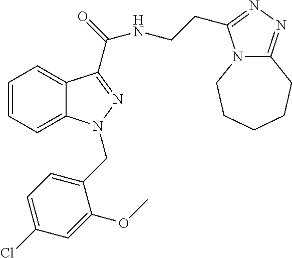 [SMILES: COc1cc(Cl)ccc1Cn1nc(C(=O)NCCc2nnc3n2CCCCC3)c2ccccc21] | 64 | 7.878 | 478.2 | 479.2 | [M + H]⁺ | 2 |
| 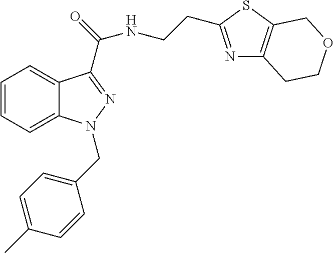 [SMILES: Cc1ccc(Cn2nc(C(=O)NCCc3nc4c(s3)COCC4)c3ccccc32)cc1] | 65 | 9.719 | 432.2 | 433.2 | [M + H]⁺ | 2 |
| 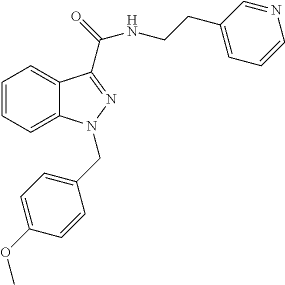 [SMILES: COc1ccc(Cn2nc(C(=O)NCCc3cccnc3)c3ccccc32)cc1] | 66 | 6.291 | 386.2 | 387.2 | [M + H]⁺ | 2 |
| 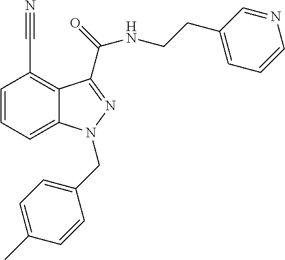 [SMILES: Cc1ccc(Cn2nc(C(=O)NCCc3cccnc3)c3c(C#N)cccc32)cc1] | 67 | 6.345 | 395.2 | 396.2 | [M + H]⁺ | 2 |
| 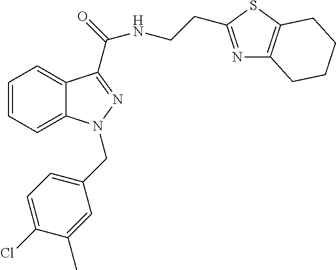 [SMILES: Cc1cc(Cn2nc(C(=O)NCCc3nc4c(s3)CCCC4)c3ccccc32)ccc1Cl] | 68 | 12.102 | 464.1 | 465.1 | [M + H]⁺ | 2 |
| 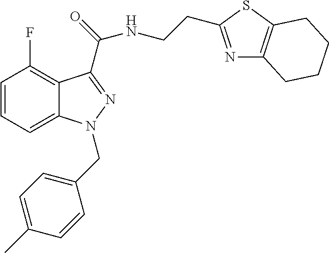 [SMILES: Cc1ccc(Cn2nc(C(=O)NCCc3nc4c(s3)CCCC4)c3c(F)cccc32)cc1] | 69 | 10.752 | 448.2 | 449.2 | [M + H]⁺ | 2 |
| 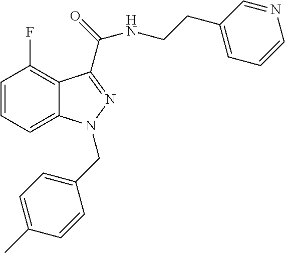 [SMILES: Cc1ccc(Cn2nc(C(=O)NCCc3cccnc3)c3c(F)cccc32)cc1] | 70 | 6.528 | 388.2 | 389.2 | [M + H]⁺ | 2 |
| 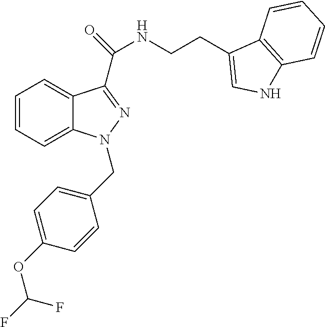 [SMILES: O=C(NCCc1c[nH]c2ccccc12)c1nn(Cc2ccc(OC(F)F)cc2)c2ccccc12] | 71 | 10.421 | 460.2 | 459.2 | [M − H] | 1 |
| 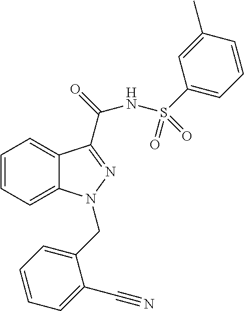 [SMILES: Cc1cccc(S(=O)(=O)NC(=O)c2nn(Cc3ccccc3C#N)c3ccccc23)c1] | 260 | 10.312 | 430.1 | 431.1 | [M + H]⁺ | 2 |
| 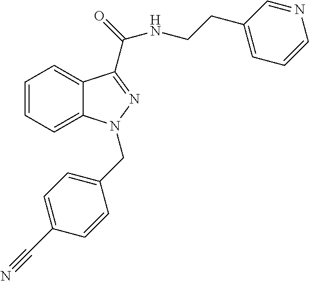 [SMILES: N#Cc1ccc(Cn2nc(C(=O)NCCc3cccnc3)c3ccccc32)cc1] | 72 | 5.681 | 381.2 | 382.2 | [M + H]⁺ | 2 |
| 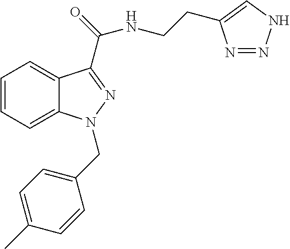 [SMILES: Cc1ccc(Cn2nc(C(=O)NCCc3c[nH]nn3)c3ccccc32)cc1] | 73 | 8.233 | 360.2 | 361.2 | [M + H]⁺ | 2 |
| 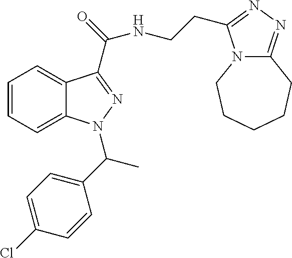 [SMILES: CC(c1ccc(Cl)cc1)n1nc(C(=O)NCCc2nnc3n2CCCCC3)c2ccccc21] | 74 | 8.031 | 462.2 | 463.2 | [M + H]⁺ | 2 |
| 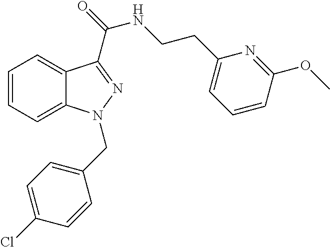 [SMILES: COc1cccc(CCNC(=O)c2nn(Cc3ccc(Cl)cc3)c3ccccc23)n1] | 75 | 11.50 | 420.1 | 421.2 | [M + H]⁺ | 2 |
| 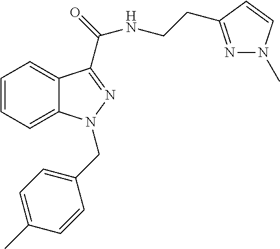 [SMILES: Cc1ccc(Cn2nc(C(=O)NCCc3ccn(C)n3)c3ccccc32)cc1] | 76 | 9.058 | 373.2 | 374.2 | [M + H]⁺ | 2 |
| 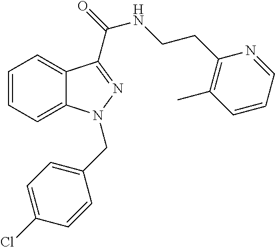 [SMILES: Cc1cccnc1CCNC(=O)c1nn(Cc2ccc(Cl)cc2)c2ccccc12] | 77 | 7.187 | 404.1 | 405.1 | [M + H]⁺ | 2 |
| 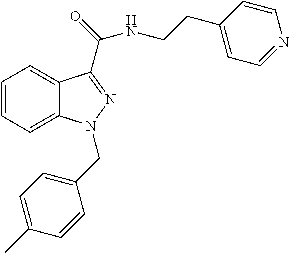 [SMILES: Cc1ccc(Cn2nc(C(=O)NCCc3ccncc3)c3ccccc32)cc1] | 78 | 6.887 | 370.2 | 371.2 | [M + H]⁺ | 2 |
| 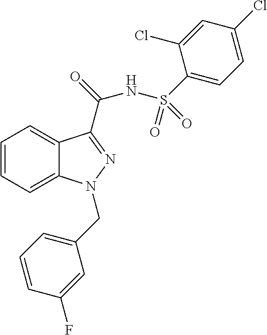 [SMILES: O=C(NS(=O)(=O)c1ccc(Cl)cc1Cl)c1nn(Cc2cccc(F)c2)c2ccccc12] | 261 | 11.728 | 477.0 | 478.0 | [M + H]⁺ | 2 |
| 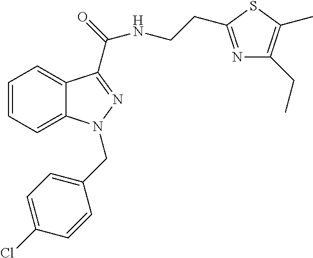 [SMILES: CCc1nc(CCNC(=O)c2nn(Cc3ccc(Cl)cc3)c3ccccc23)sc1C] | 79 | 11.14 | 438.1 | 439.1 | [M + H]⁺ | 2 |
| 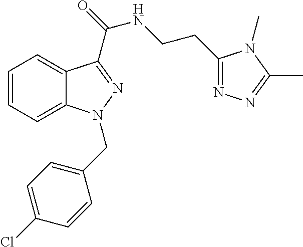 [SMILES: Cc1nnc(CCNC(=O)c2nn(Cc3ccc(Cl)cc3)c3ccccc23)n1C] | 80 | 6.97 | 408.1 | 409.1 | [M + H]⁺ | 2 |
| 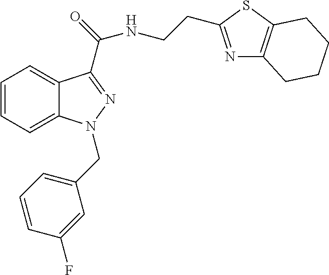 [SMILES: O=C(NCCc1nc2c(s1)CCCC2)c1nn(Cc2cccc(F)c2)c2ccccc12] | 81 | 10.648 | 434.2 | 435.2 | [M + H]⁺ | 2 |
| 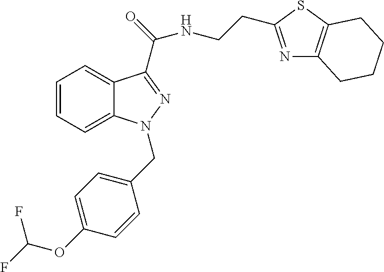 [SMILES: O=C(NCCc1nc2c(s1)CCCC2)c1nn(Cc2ccc(OC(F)F)cc2)c2ccccc12] | 82 | 10.899 | 482.2 | 483.1 | [M + H]⁺ | 2 |
| 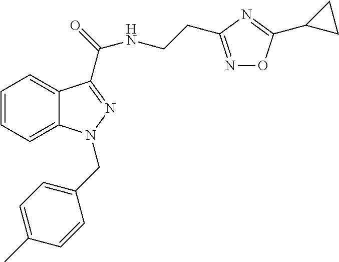 [SMILES: Cc1ccc(Cn2nc(C(=O)NCCc3noc(C4CC4)n3)c3ccccc32)cc1] | 83 | 10.351 | 401.2 | 402.2 | [M + H]⁺ | 2 |
| 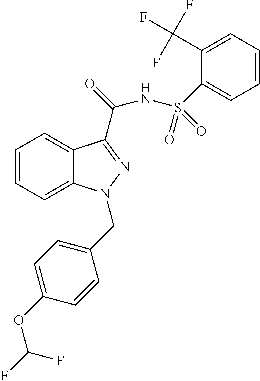 [SMILES: O=C(NS(=O)(=O)c1ccccc1C(F)(F)F)c1nn(Cc2ccc(OC(F)F)cc2)c2ccccc12] | 262 | 11.055 | 525.1 | 525.6 | [M + H]⁺ | 2 |
| 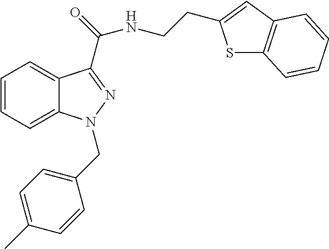 [SMILES: Cc1ccc(Cn2nc(C(=O)NCCc3cc4ccccc4s3)c3ccccc32)cc1] | 84 | 12.27 | 425.2 | 426.2 | [M + H]⁺ | 2 |
| 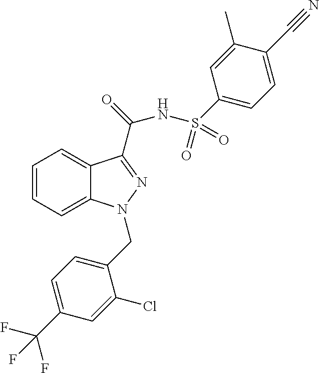 [SMILES: Cc1cc(S(=O)(=O)NC(=O)c2nn(Cc3ccc(C(F)(F)F)cc3Cl)c3ccccc23)ccc1C#N] | 263 | 11.879 | 532.1 | 533.0 | [M + H]⁺ | 2 |
| 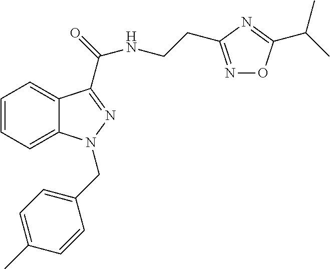 [SMILES: Cc1ccc(Cn2nc(C(=O)NCCc3noc(C(C)C)n3)c3ccccc32)cc1] | 85 | 10.871 | 403.2 | 404.2 | [M + H]⁺ | 2 |
| 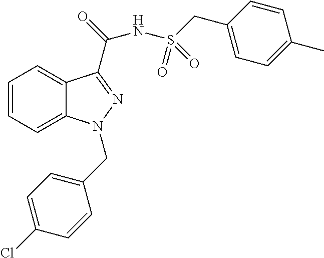 [SMILES: Cc1ccc(CS(=O)(=O)NC(=O)c2nn(Cc3ccc(Cl)cc3)c3ccccc23)cc1] | 264 | 11.661 | 453.1 | 454.1 | [M + H]⁺ | 2 |
| 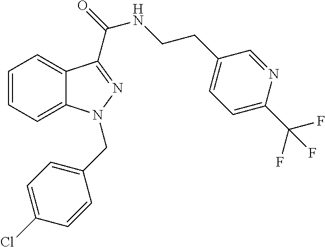 [SMILES: O=C(NCCc1ccc(C(F)(F)F)nc1)c1nn(Cc2ccc(Cl)cc2)c2ccccc12] | 86 | 10.635 | 458.1 | 459.1 | [M + H]⁺ | 2 |
| 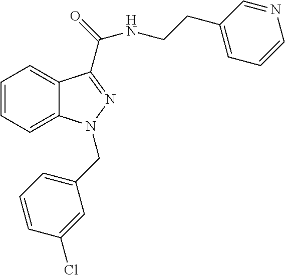 [SMILES: O=C(NCCc1cccnc1)c1nn(Cc2cccc(Cl)c2)c2ccccc12] | 87 | 6.771 | 390.1 | 391.1 | [M + H]⁺ | 2 |
| 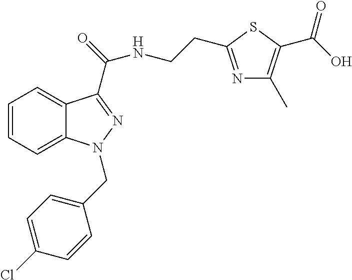 [SMILES: Cc1nc(CCNC(=O)c2nn(Cc3ccc(Cl)cc3)c3ccccc23)sc1C(=O)O] | 88 | 9.134 | 454.1 | 455.1 | [M + H]⁺ | 2 |
| 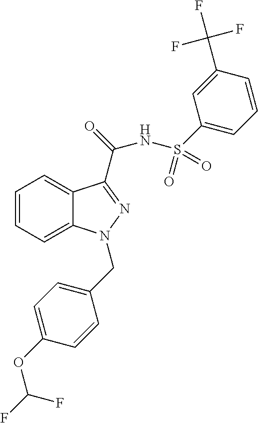 [SMILES: O=C(NS(=O)(=O)c1cccc(C(F)(F)F)c1)c1nn(Cc2ccc(OC(F)F)cc2)c2ccccc12] | 265 | 11.308 | 525.1 | 525.6 | [M + H]⁺ | 2 |
| 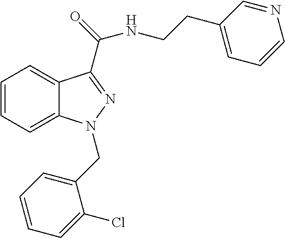 [SMILES: O=C(NCCc1cccnc1)c1nn(Cc2ccccc2Cl)c2ccccc12] | 89 | 6.768 | 390.1 | 391.1 | [M + H]⁺ | 2 |
| 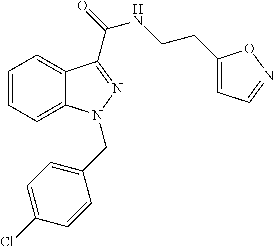 [SMILES: O=C(NCCc1ccno1)c1nn(Cc2ccc(Cl)cc2)c2ccccc12] | 90 | 9.300 | 380.1 | 381.1 | [M + H]⁺ | 2 |
| 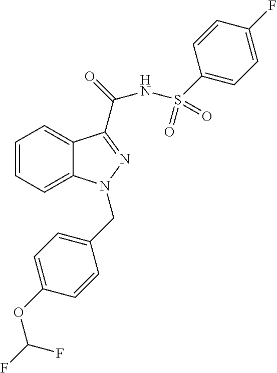 [SMILES: O=C(NS(=O)(=O)c1ccc(F)cc1)c1nn(Cc2ccc(OC(F)F)cc2)c2ccccc12] | 266 | 10.697 | 475.1 | 475.7 | [M + H]⁺ | 2 |
| 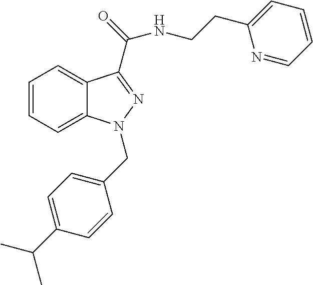 [SMILES: CC(C)c1ccc(Cn2nc(C(=O)NCCc3ccccn3)c3ccccc32)cc1] | 91 | 7.919 | 398.2 | 399.2 | [M + H]⁺ | 2 |
| 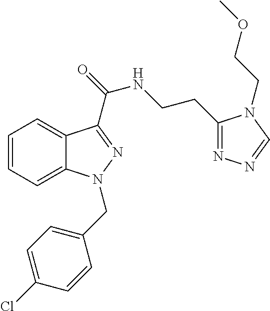 [SMILES: COCCn1cnnc1CCNC(=O)c1nn(Cc2ccc(Cl)cc2)c2ccccc12] | 92 | 7.167 | 438.2 | 439.1 | [M + H]⁺ | 2 |
| 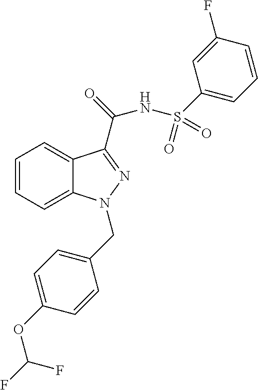 [SMILES: O=C(NS(=O)(=O)c1cccc(F)c1)c1nn(Cc2ccc(OC(F)F)cc2)c2ccccc12] | 267 | 10.697 | 475.1 | 475.7 | [M + H]⁺ | 2 |
| 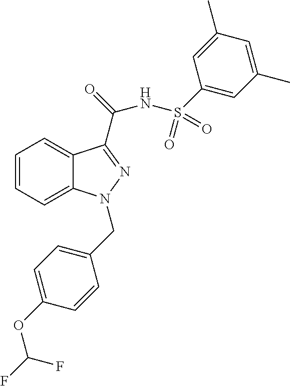 [SMILES: Cc1cc(C)cc(S(=O)(=O)NC(=O)c2nn(Cc3ccc(OC(F)F)cc3)c3ccccc23)c1] | 268 | 11.352 | 485.1 | 486.1 | [M + H]⁺ | 2 |
| 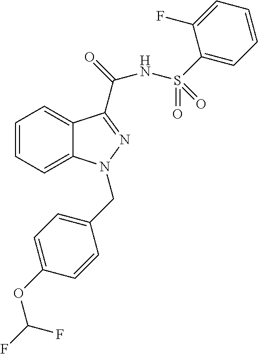 [SMILES: O=C(NS(=O)(=O)c1ccccc1F)c1nn(Cc2ccc(OC(F)F)cc2)c2ccccc12] | 269 | 10.405 | 475.1 | 475.7 | [M + H]⁺ | 2 |
| 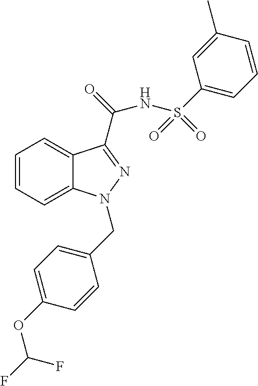 [SMILES: Cc1cccc(S(=O)(=O)NC(=O)c2nn(Cc3ccc(OC(F)F)cc3)c3ccccc23)c1] | 270 | 10.886 | 471.1 | 471.7 | [M + H]⁺ | 2 |
| 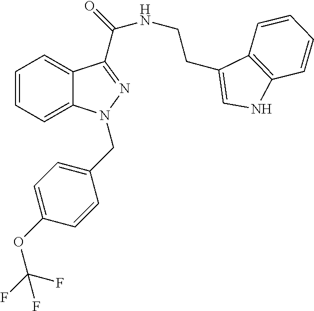 [SMILES: O=C(NCCc1c[nH]c2ccccc12)c1nn(Cc2ccc(OC(F)(F)F)cc2)c2ccccc12] | 93 | 11.435 | 478.2 | 478.8 | [M + H]⁺ | 2 |
| 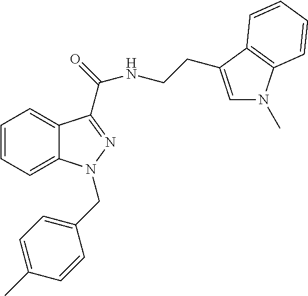 [SMILES: Cc1ccc(Cn2nc(C(=O)NCCc3cn(C)c4ccccc34)c3ccccc32)cc1] | 94 | 12.054 | 422.2 | 423.2 | [M + H]⁺ | 2 |
| 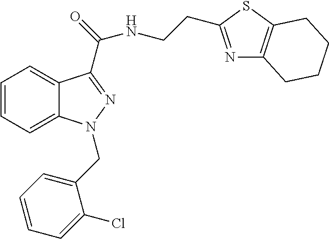 [SMILES: O=C(NCCc1nc2c(s1)CCCC2)c1nn(Cc2ccccc2Cl)c2ccccc12] | 95 | 11.604 | 450.1 | 451.1 | [M + H]⁺ | 2 |
| 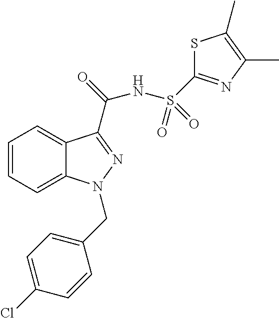 [SMILES: Cc1nc(S(=O)(=O)NC(=O)c2nn(Cc3ccc(Cl)cc3)c3ccccc23)sc1C] | 271 | 10.865 | 460.0 | 461.1 | [M + H]⁺ | 2 |
| 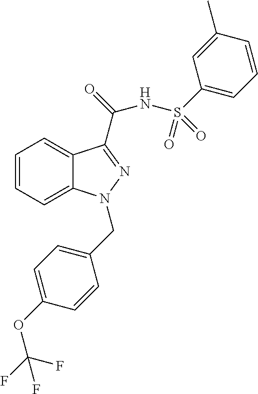 [SMILES: Cc1cccc(S(=O)(=O)NC(=O)c2nn(Cc3ccc(OC(F)(F)F)cc3)c3ccccc23)c1] | 272 | 11.675 | 489.1 | 489.7 | [M + H]⁺ | 2 |
| 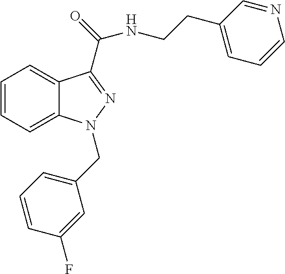 [SMILES: O=C(NCCc1cccnc1)c1nn(Cc2cccc(F)c2)c2ccccc12] | 96 | 6.27 | 374.2 | 375.2 | [M + H]⁺ | 2 |
| 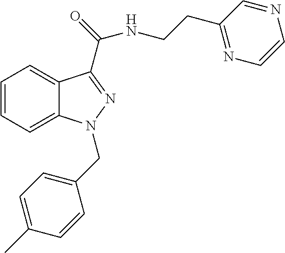 [SMILES: Cc1ccc(Cn2nc(C(=O)NCCc3cnccn3)c3ccccc32)cc1] | 97 | 8.94 | 371.2 | 372.2 | [M + H]⁺ | 2 |
| 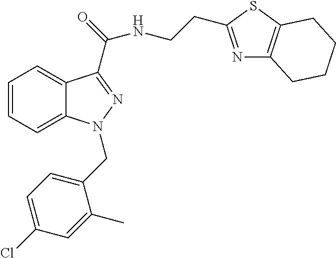 [SMILES: Cc1cc(Cl)ccc1Cn1nc(C(=O)NCCc2nc3c(s2)CCCC3)c2ccccc21] | 98 | 11.999 | 464.1 | 465.1 | [M + H]⁺ | 2 |
| 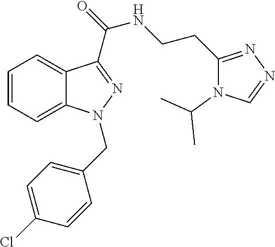 [SMILES: CC(C)n1cnnc1CCNC(=O)c1nn(Cc2ccc(Cl)cc2)c2ccccc12] | 99 | 7.493 | 422.2 | 423.1 | [M + H]⁺ | 2 |
| 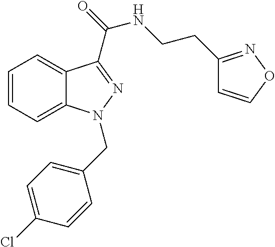 [SMILES: O=C(NCCc1ccon1)c1nn(Cc2ccc(Cl)cc2)c2ccccc12] | 100 | 9.274 | 380.1 | 381.1 | [M + H]⁺ | 2 |
| 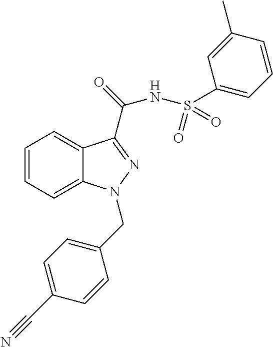 [SMILES: Cc1cccc(S(=O)(=O)NC(=O)c2nn(Cc3ccc(C#N)cc3)c3ccccc23)c1] | 273 | 10.158 | 430.1 | 431.1 | [M + H]⁺ | 2 |
| 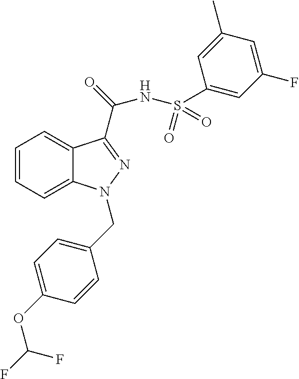 [SMILES: Cc1cc(F)cc(S(=O)(=O)NC(=O)c2nn(Cc3ccc(OC(F)F)cc3)c3ccccc23)c1] | 274 | 11.167 | 489.1 | 490.1 | [M + H]⁺ | 2 |
| 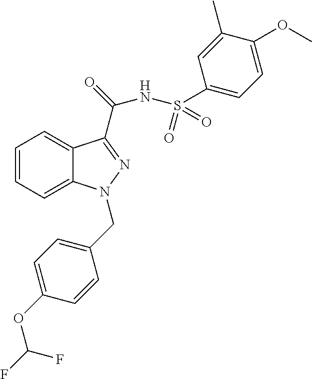 [SMILES: COc1ccc(S(=O)(=O)NC(=O)c2nn(Cc3ccc(OC(F)F)cc3)c3ccccc23)cc1C] | 275 | 11.033 | 501.1 | 502.1 | [M + H]⁺ | 2 |
| 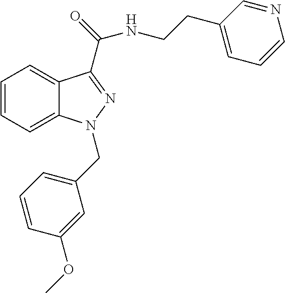 [SMILES: COc1cccc(Cn2nc(C(=O)NCCc3cccnc3)c3ccccc32)c1] | 101 | 6.320 | 386.2 | 387.2 | [M + H]⁺ | 2 |
| 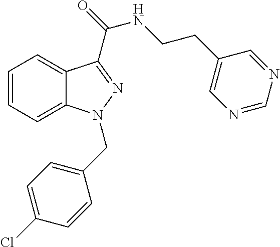 [SMILES: O=C(NCCc1cncnc1)c1nn(Cc2ccc(Cl)cc2)c2ccccc12] | 102 | 8.597 | 391.1 | 392.1 | [M + H]⁺ | 2 |
| 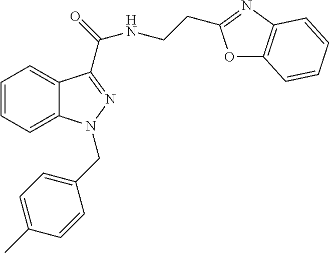 [SMILES: Cc1ccc(Cn2nc(C(=O)NCCc3nc4ccccc4o3)c3ccccc32)cc1] | 103 | 10.649 | 410.2 | 411.2 | [M + H]⁺ | 2 |
| 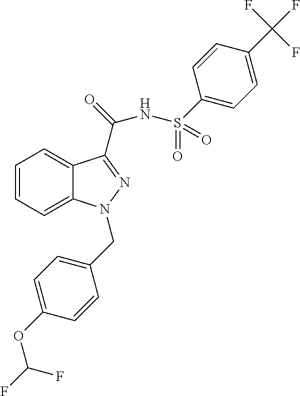 [SMILES: O=C(NS(=O)(=O)c1ccc(C(F)(F)F)cc1)c1nn(Cc2ccc(OC(F)F)cc2)c2ccccc12] | 276 | 11.411 | 525.1 | 525.6 | [M + H]⁺ | 2 |
| 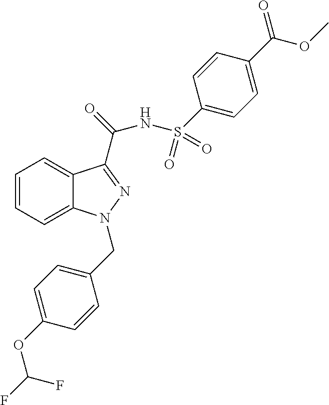 [SMILES: COC(=O)c1ccc(S(=O)(=O)NC(=O)c2nn(Cc3ccc(OC(F)F)cc3)c3ccccc23)cc1] | 277 | 10.594 | 515.1 | 515.7 | [M + H]⁺ | 2 |
| 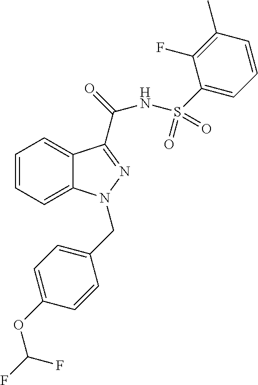 [SMILES: Cc1cccc(S(=O)(=O)NC(=O)c2nn(Cc3ccc(OC(F)F)cc3)c3ccccc23)c1F] | 278 | 10.811 | 489.1 | 490.0 | [M + H]⁺ | 2 |
| 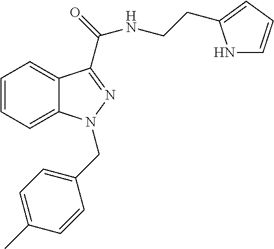 [SMILES: Cc1ccc(Cn2nc(C(=O)NCCc3ccc[nH]3)c3ccccc32)cc1] | 104 | 10.37 | 358.2 | 359.2 | [M + H]⁺ | 2 |
| 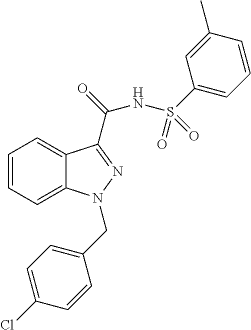 [SMILES: Cc1cccc(S(=O)(=O)NC(=O)c2nn(Cc3ccc(Cl)cc3)c3ccccc23)c1] | 279 | 11.441 | 439.1 | 440.1 | [M + H]⁺ | 2 |
| 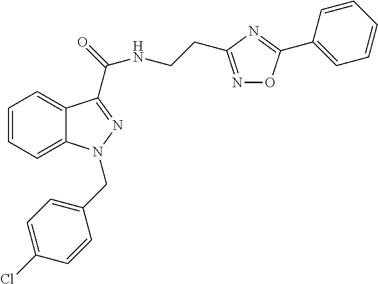 [SMILES: O=C(NCCc1noc(-c2ccccc2)n1)c1nn(Cc2ccc(Cl)cc2)c2ccccc12] | 105 | 11.776 | 457.1 | 458.1 | [M + H]⁺ | 2 |
| 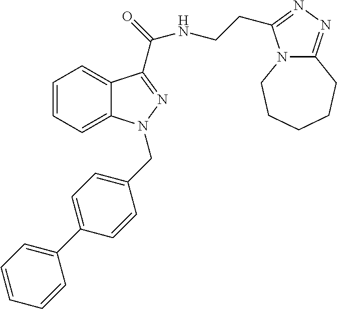 [SMILES: O=C(NCCc1nnc2n1CCCCC2)c1nn(Cc2ccc(-c3ccccc3)cc2)c2ccccc12] | 106 | 8.617 | 490.2 | 491.2 | [M + H]⁺ | 2 |
| 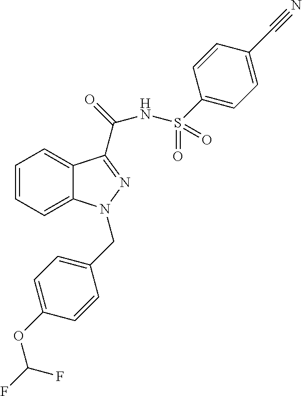 [SMILES: N#Cc1ccc(S(=O)(=O)NC(=O)c2nn(Cc3ccc(OC(F)F)cc3)c3ccccc23)cc1] | 280 | 10.305 | 482.1 | 482.7 | [M + H]⁺ | 2 |
| 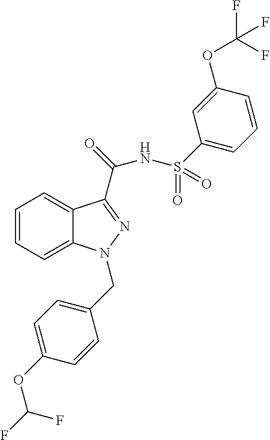 [SMILES: O=C(NS(=O)(=O)c1cccc(OC(F)(F)F)c1)c1nn(Cc2ccc(OC(F)F)cc2)c2ccccc12] | 281 | 11.484 | 541.1 | 541.6 | [M + H]⁺ | 2 |
| 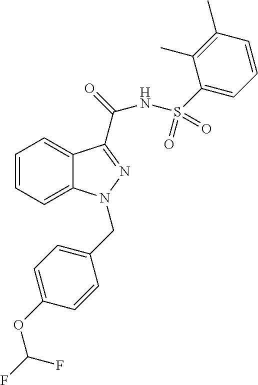 [SMILES: Cc1cccc(S(=O)(=O)NC(=O)c2nn(Cc3ccc(OC(F)F)cc3)c3ccccc23)c1C] | 282 | 11.085 | 485.1 | 486.1 | [M + H]⁺ | 2 |
| 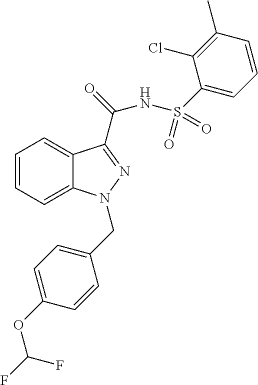 [SMILES: Cc1cccc(S(=O)(=O)NC(=O)c2nn(Cc3ccc(OC(F)F)cc3)c3ccccc23)c1Cl] | 283 | 11.157 | 505.1 | 506.0 | [M + H]⁺ | 2 |
| 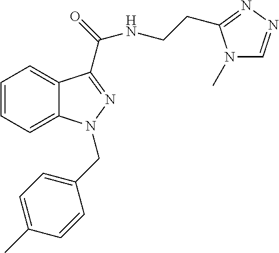 [SMILES: Cc1ccc(Cn2nc(C(=O)NCCc3nncn3C)c3ccccc32)cc1] | 107 | 7.154 | 374.2 | 375.2 | [M + H]⁺ | 2 |
| 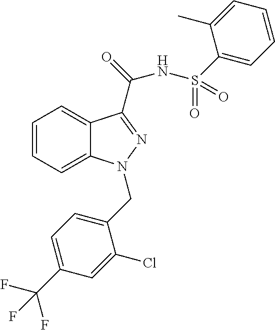 [SMILES: Cc1ccccc1S(=O)(=O)NC(=O)c1nn(Cc2ccc(C(F)(F)F)cc2Cl)c2ccccc12] | 284 | 12.076 | 507.9 | 508.0 | [M + H]⁺ | 2 |
| 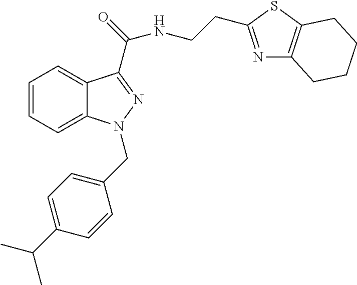 [SMILES: CC(C)c1ccc(Cn2nc(C(=O)NCCc3nc4c(s3)CCCC4)c3ccccc32)cc1] | 108 | 12.46 | 458.6 | 459.2 | [M + H]⁺ | 2 |
| 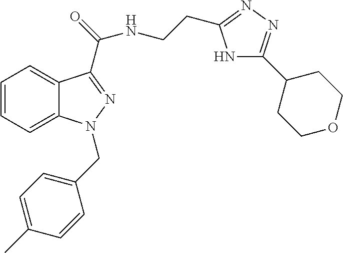 [SMILES: Cc1ccc(Cn2nc(C(=O)NCCc3nnc(C4CCOCC4)[nH]3)c3ccccc32)cc1] | 109 | 7.59 | 444.2 | 445.2 | [M + H]⁺ | 2 |
| 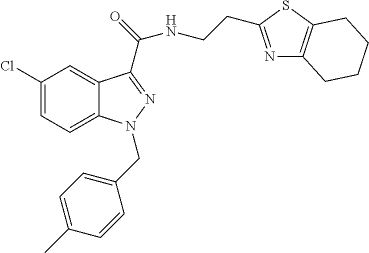 [SMILES: Cc1ccc(Cn2nc(C(=O)NCCc3nc4c(s3)CCCC4)c3cc(Cl)ccc32)cc1] | 110 | 12.445 | 464.1 | 465.1 | [M + H]⁺ | 2 |
| 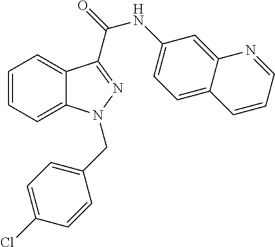 [SMILES: O=C(Nc1ccc2cccnc2c1)c1nn(Cc2ccc(Cl)cc2)c2ccccc12] | 111 | 8.413 | 412.1 | 413.1 | [M + H]⁺ | 2 |
| 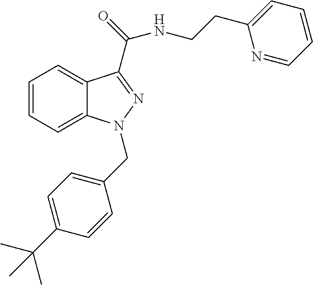 [SMILES: CC(C)(C)c1ccc(Cn2nc(C(=O)NCCc3ccccn3)c3ccccc32)cc1] | 112 | 8.436 | 412.2 | 413.2 | [M + H]⁺ | 2 |
| 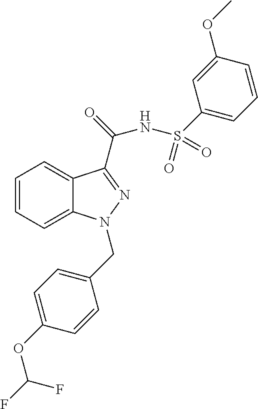 [SMILES: COc1cccc(S(=O)(=O)NC(=O)c2nn(Cc3ccc(OC(F)F)cc3)c3ccccc23)c1] | 285 | 10.602 | 487.1 | 487.7 | [M + H]⁺ | 2 |
| 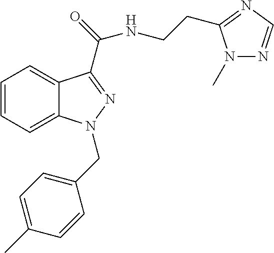 [SMILES: Cc1ccc(Cn2nc(C(=O)NCCc3ncnn3C)c3ccccc32)cc1] | 113 | 8.138 | 374.2 | 375.2 | [M + H]⁺ | 2 |
| 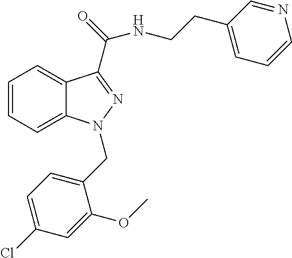 [SMILES: COc1cc(Cl)ccc1Cn1nc(C(=O)NCCc2cccnc2)c2ccccc21] | 114 | 7.222 | 420.1 | 421.1 | [M + H]⁺ | 2 |
| 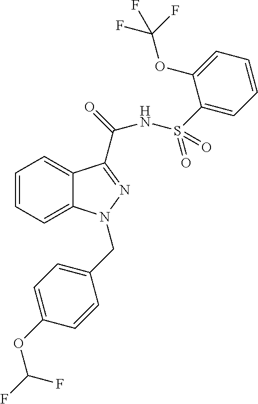 [SMILES: O=C(NS(=O)(=O)c1ccccc1OC(F)(F)F)c1nn(Cc2ccc(OC(F)F)cc2)c2ccccc12] | 286 | 11.079 | 541.1 | 541.6 | [M + H]⁺ | 2 |
| 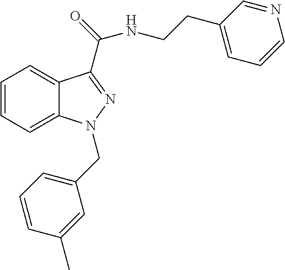 [SMILES: Cc1cccc(Cn2nc(C(=O)NCCc3cccnc3)c3ccccc32)c1] | 115 | 6.737 | 370.2 | 371.2 | [M + H]⁺ | 2 |
| 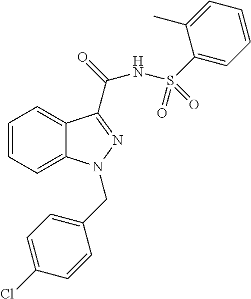 [SMILES: Cc1ccccc1S(=O)(=O)NC(=O)c1nn(Cc2ccc(Cl)cc2)c2ccccc12] | 287 | 11.304 | 439.1 | 440.1 | [M + H]⁺ | 2 |
| 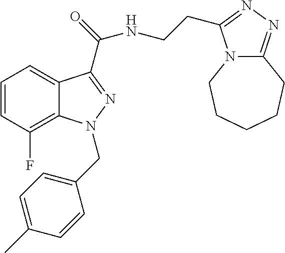 [SMILES: Cc1ccc(Cn2nc(C(=O)NCCc3nnc4n3CCCCC4)c3cccc(F)c32)cc1] | 116 | 7.963 | 446.2 | 447.2 | [M + H]⁺ | 2 |
| 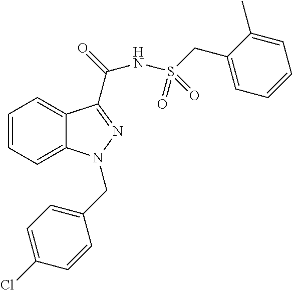 [SMILES: Cc1ccccc1CS(=O)(=O)NC(=O)c1nn(Cc2ccc(Cl)cc2)c2ccccc12] | 288 | 11.677 | 453.1 | 454.1 | [M + H]⁺ | 2 |
| 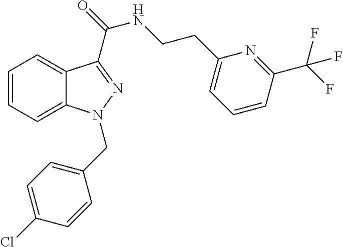 [SMILES: O=C(NCCc1cccc(C(F)(F)F)n1)c1nn(Cc2ccc(Cl)cc2)c2ccccc12] | 117 | 11.476 | 458.1 | 459.1 | [M + H]⁺ | 2 |
| 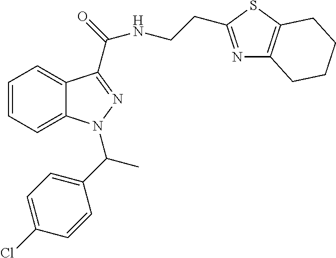 [SMILES: CC(c1ccc(Cl)cc1)n1nc(C(=O)NCCc2nc3c(s2)CCCC3)c2ccccc21] | 118 | 11.969 | 464.1 | 465.1 | [M + H]⁺ | 2 |
| 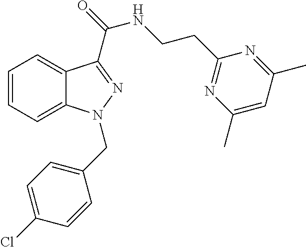 [SMILES: Cc1cc(C)nc(CCNC(=O)c2nn(Cc3ccc(Cl)cc3)c3ccccc23)n1] | 119 | 9.227 | 419.2 | 420.1 | [M + H]⁺ | 2 |
| 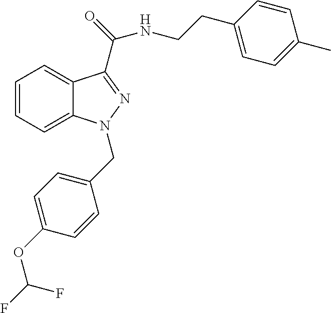 [SMILES: Cc1ccc(CCNC(=O)c2nn(Cc3ccc(OC(F)F)cc3)c3ccccc23)cc1] | 120 | 11.38 | 435.2 | 436.2 | [M + H]⁺ | 2 |
| 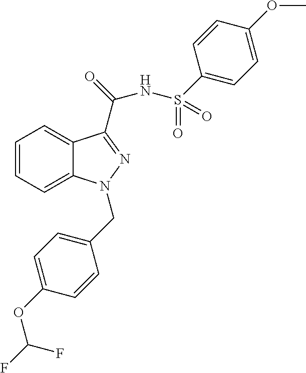 [SMILES: COc1ccc(S(=O)(=O)NC(=O)c2nn(Cc3ccc(OC(F)F)cc3)c3ccccc23)cc1] | 289 | 10.479 | 487.1 | 487.7 | [M + H]⁺ | 2 |
| 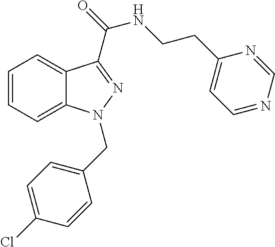 [SMILES: O=C(NCCc1ccncn1)c1nn(Cc2ccc(Cl)cc2)c2ccccc12] | 121 | 8.674 | 391.1 | 392.1 | [M + H]⁺ | 2 |
| 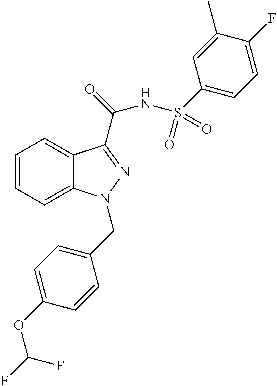 [SMILES: Cc1cc(S(=O)(=O)NC(=O)c2nn(Cc3ccc(OC(F)F)cc3)c3ccccc23)ccc1F] | 290 | 11.168 | 489.1 | 490.1 | [M + H]⁺ | 2 |
| 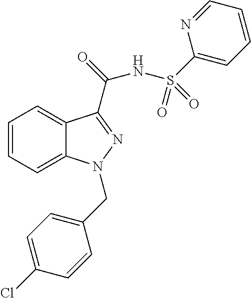 [SMILES: O=C(NS(=O)(=O)c1ccccn1)c1nn(Cc2ccc(Cl)cc2)c2ccccc12] | 291 | 9.916 | 426.1 | 427.0 | [M + H]⁺ | 2 |
| 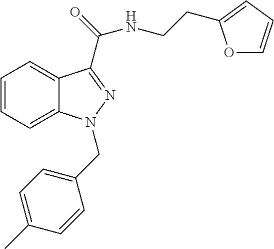 [SMILES: Cc1ccc(Cn2nc(C(=O)NCCc3ccco3)c3ccccc32)cc1] | 122 | 11.022 | 359.2 | 360.2 | [M + H]⁺ | 2 |
| 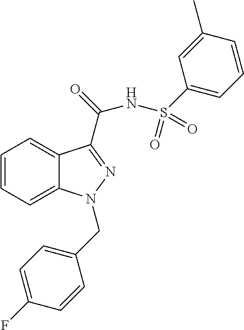 [SMILES: Cc1cccc(S(=O)(=O)NC(=O)c2nn(Cc3ccc(F)cc3)c3ccccc23)c1] | 292 | 10.807 | 423.1 | 424.1 | [M + H]⁺ | 2 |
| 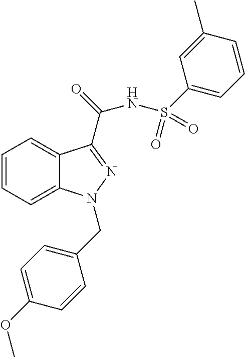 [SMILES: COc1ccc(Cn2nc(C(=O)NS(=O)(=O)c3cccc(C)c3)c3ccccc32)cc1] | 293 | 10.723 | 435.1 | 436.1 | [M + H]⁺ | 2 |
| 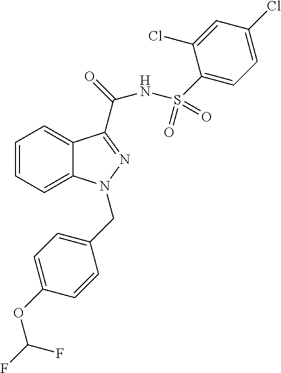 [SMILES: O=C(NS(=O)(=O)c1ccc(Cl)cc1Cl)c1nn(Cc2ccc(OC(F)F)cc2)c2ccccc12] | 294 | 11.865 | 525 | 526 | [M + H]⁺ | 2 |
| 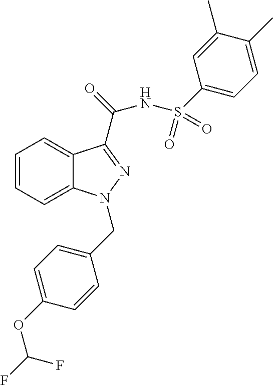 [SMILES: Cc1ccc(S(=O)(=O)NC(=O)c2nn(Cc3ccc(OC(F)F)cc3)c3ccccc23)cc1C] | 295 | 11.243 | 485.1 | 486.1 | [M + H]⁺ | 2 |
| 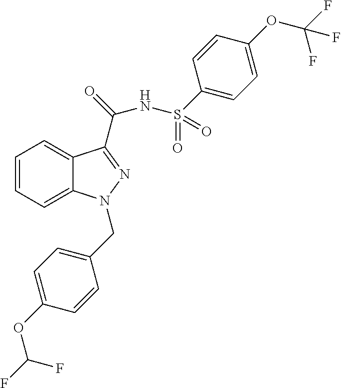 [SMILES: O=C(NS(=O)(=O)c1ccc(OC(F)(F)F)cc1)c1nn(Cc2ccc(OC(F)F)cc2)c2ccccc12] | 296 | 11.532 | 541.1 | 541.6 | [M + H]⁺ | 2 |
| 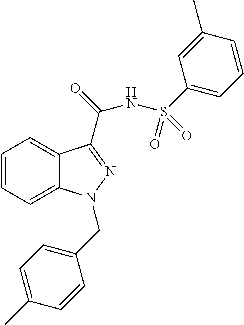 [SMILES: Cc1ccc(Cn2nc(C(=O)NS(=O)(=O)c3cccc(C)c3)c3ccccc32)cc1] | 297 | 11.361 | 419.1 | 420.1 | [M + H]⁺ | 2 |
| 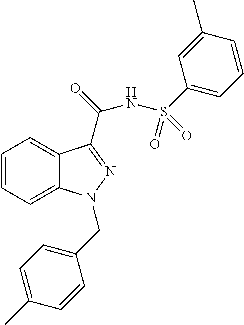 [SMILES: Cc1ccc(Cn2nc(C(=O)NS(=O)(=O)c3cccc(C)c3)c3ccccc32)cc1] | 298 | 11.353 | 419.1 | 420.1 | [M + H]⁺ | 2 |
| 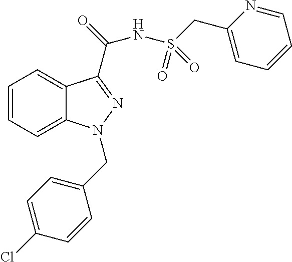 [SMILES: O=C(NS(=O)(=O)Cc1ccccn1)c1nn(Cc2ccc(Cl)cc2)c2ccccc12] | 299 | 9.626 | 440.1 | 441.1 | [M + H]⁺ | 2 |
| 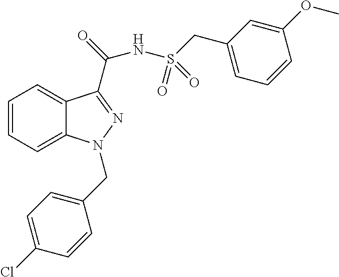 [SMILES: COc1cccc(CS(=O)(=O)NC(=O)c2nn(Cc3ccc(Cl)cc3)c3ccccc23)c1] | 300 | 11.181 | 469.1 | 470.1 | [M + H]⁺ | 2 |
| 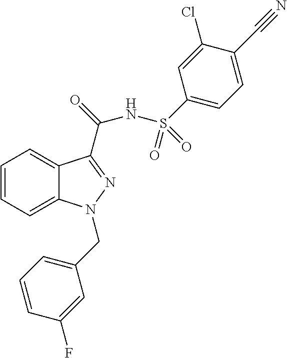 [SMILES: N#Cc1ccc(S(=O)(=O)NC(=O)c2nn(Cc3cccc(F)c3)c3ccccc23)cc1Cl] | 301 | 10.878 | 468.0 | 469.0 | [M + H]⁺ | 2 |
| 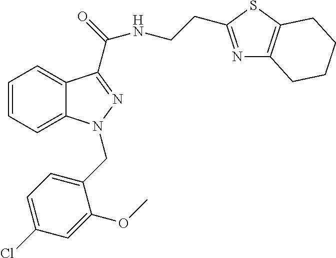 [SMILES: COc1cc(Cl)ccc1Cn1nc(C(=O)NCCc2nc3c(s2)CCCC3)c2ccccc21] | 123 | 11.764 | 480.1 | 481.2 | [M + H]⁺ | 2 |
| 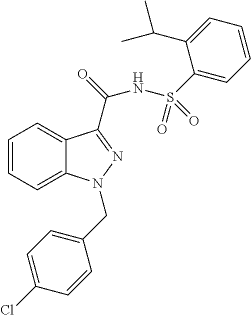 [SMILES: CC(C)c1ccccc1S(=O)(=O)NC(=O)c1nn(Cc2ccc(Cl)cc2)c2ccccc12] | 302 | 11.993 | 467.1 | 468.1 | [M + H]⁺ | 2 |
| 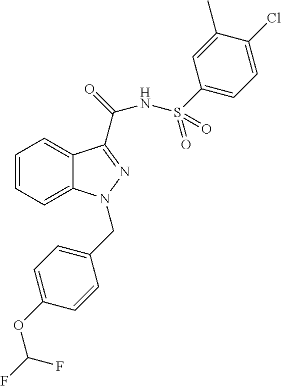 [SMILES: Cc1cc(S(=O)(=O)NC(=O)c2nn(Cc3ccc(OC(F)F)cc3)c3ccccc23)ccc1Cl] | 303 | 11.767 | 505.1 | 506.1 | [M + H]⁺ | 2 |
| 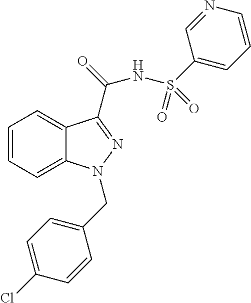 [SMILES: O=C(NS(=O)(=O)c1cccnc1)c1nn(Cc2ccc(Cl)cc2)c2ccccc12] | 304 | 9.676 | 426.1 | 427.0 | [M + H]⁺ | 2 |
| 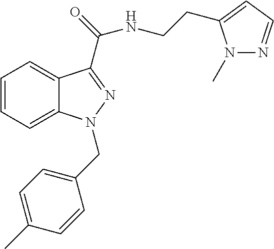 [SMILES: Cc1ccc(Cn2nc(C(=O)NCCc3ccnn3C)c3ccccc32)cc1] | 124 | 8.034 | 373.2 | 374.2 | [M + H]⁺ | 2 |
| 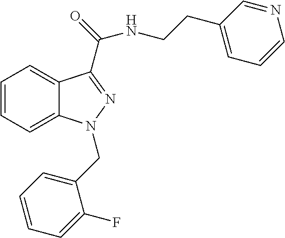 [SMILES: O=C(NCCc1cccnc1)c1nn(Cc2ccccc2F)c2ccccc12] | 125 | 6.224 | 374.2 | 375.2 | [M + H]⁺ | 2 |
| 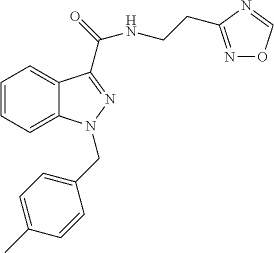 [SMILES: Cc1ccc(Cn2nc(C(=O)NCCc3ncon3)c3ccccc32)cc1] | 126 | 9.341 | 361.2 | 362.1 | [M + H]⁺ | 2 |
| 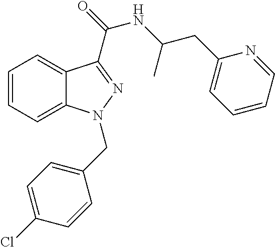 [SMILES: CC(Cc1ccccn1)NC(=O)c1nn(Cc2ccc(Cl)cc2)c2ccccc12] | 127 | 7.33 | 404.1 | 405.1 | [M + H]⁺ | 2 |
| 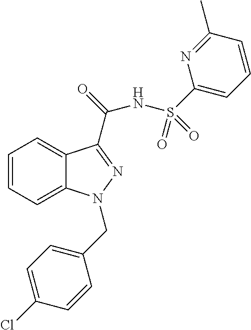 [SMILES: Cc1cccc(S(=O)(=O)NC(=O)c2nn(Cc3ccc(Cl)cc3)c3ccccc23)n1] | 305 | 10.345 | 440.1 | 441.1 | [M + H]⁺ | 2 |
| 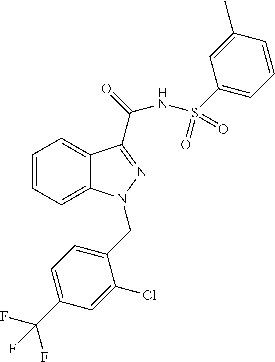 [SMILES: Cc1cccc(S(=O)(=O)NC(=O)c2nn(Cc3ccc(C(F)(F)F)cc3Cl)c3ccccc23)c1] | 306 | 12.168 | 507.1 | 508.0 | [M + H]⁺ | 2 |
| 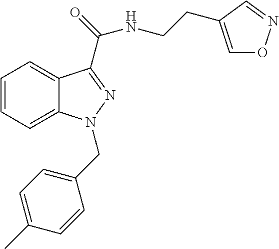 [SMILES: Cc1ccc(Cn2nc(C(=O)NCCc3cnoc3)c3ccccc32)cc1] | 128 | 9.697 | 360.2 | 361.1 | [M + H]⁺ | 2 |
| 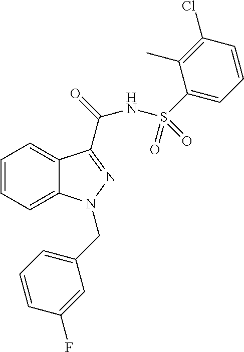 [SMILES: Cc1c(Cl)cccc1S(=O)(=O)NC(=O)c1nn(Cc2cccc(F)c2)c2ccccc12] | 307 | 11.60 | 457.1 | 458.0 | [M + H]⁺ | 2 |
| 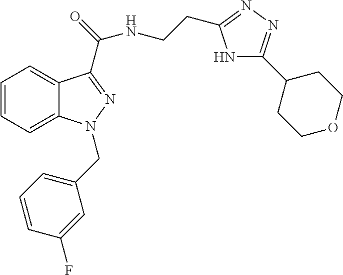 [SMILES: O=C(NCCc1nnc(C2CCOCC2)[nH]1)c1nn(Cc2cccc(F)c2)c2ccccc12] | 129 | 7.126 | 448.2 | 449.2 | [M + H]⁺ | 2 |
| 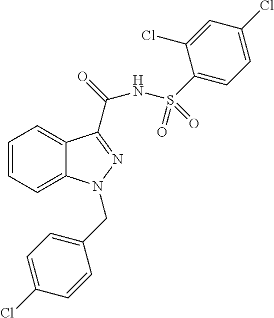 [SMILES: O=C(NS(=O)(=O)c1ccc(Cl)cc1Cl)c1nn(Cc2ccc(Cl)cc2)c2ccccc12] | 308 | 12.268 | 493.0 | 493.9 | [M + H]⁺ | 2 |
| 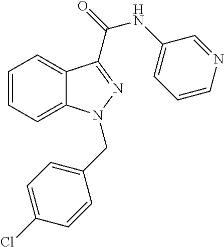 [SMILES: O=C(Nc1cccnc1)c1nn(Cc2ccc(Cl)cc2)c2ccccc12] | 130 | 8.203 | 362.1 | 363.1 | [M + H]⁺ | 2 |
| 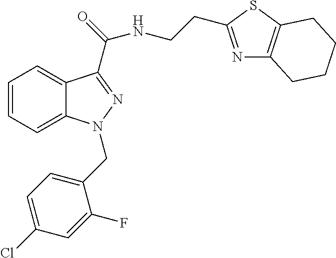 [SMILES: O=C(NCCc1nc2c(s1)CCCC2)c1nn(Cc2ccc(Cl)cc2F)c2ccccc12] | 131 | 11.733 | 468.1 | 469.1 | [M + H]⁺ | 2 |

### Example 3

### Synthesis of Compound 136

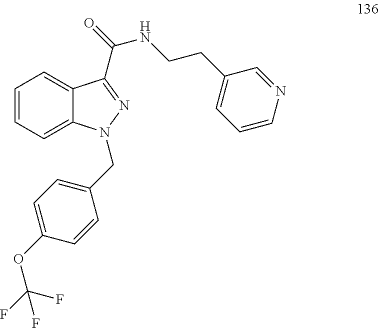 [SMILES: O=C(NCCc1cccnc1)c1nn(Cc2ccc(OC(F)(F)F)cc2)c2ccccc12]

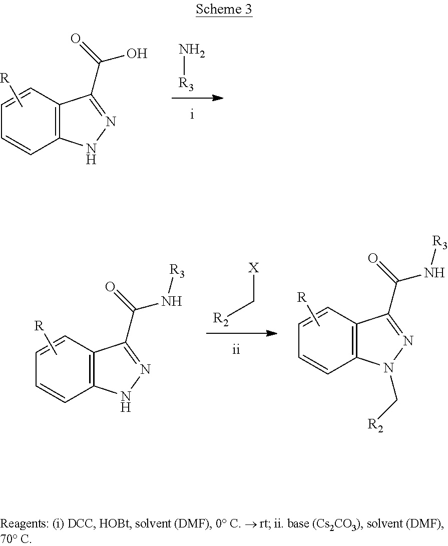

### Step 3-1. Synthesis of N-(2-(pyridin-3-yl)ethyl)-1H-indazole-3-carboxamide

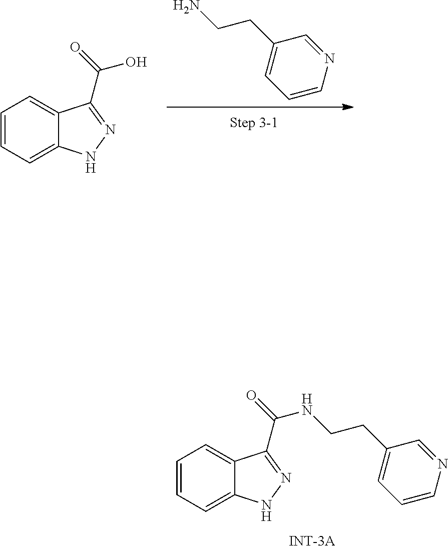

To a stirring solution of 1H-indazole-3-carboxylic acid (100 mg, 1 Eq, 617 μmol) in DMF (1 mL) at 0° C. was added 1-Hydroxybenzotriazole—hydrate (HOBt) (116 mg, 1.1 Eq, 678 μmol) and Dicyclohexylcarbodiimide (DCC) (134 mg, 1.05 Eq, 648 μmol). The reaction mixture was stirred at 0° C. for 1 h. To the reaction mixture was added a precooled solution (0° C.) of 2-(pyridin-3-yl)ethan-1-amine (86.6 mg, 1.15 Eq, 709 μmol) in DMF (1 mL). The reaction mixture was stirred for 2 h at 0° C. and then warmed to room temperature overnight. The reaction mixture was diluted with EtOAc (5 mL), filtered, and extracted with 1M HCl (10 mL). The aqueous phase was basified to a pH ˜13 with 1M NaOH. The mixture was extracted with DCM (3×10 mL), dried over sodium sulfate, filtered, and concentrated in vacuo to afford 130 mg (79% yield) of N-(2-(pyridin-3-yl)ethyl)-1H-indazole-3-carboxamide (INT-3A) that was used without further purification. ¹H NMR (400 MHz, DMSO-d6) δ 8.44 (m, 3H), 8.15 (d, J=8 Hz, 1H), 7.69 (d, J=8 Hz, 1H), 7.60 (d, J=8 Hz, 1H), 7.41 (t, J=8 Hz, 1H), 7.31 (dd, J=8, 4 Hz, 1H), 7.23, (t, J=8 Hz, 1H), 3.57 (m, 2H), 2.93 (m, 2H).

### Step 3-2. Synthesis of N-(2-(pyridin-3-yl)ethyl)-1-(4-(trifluoromethoxy)benzyl)-1H-indazole-3-carboxamide (Compound 136)

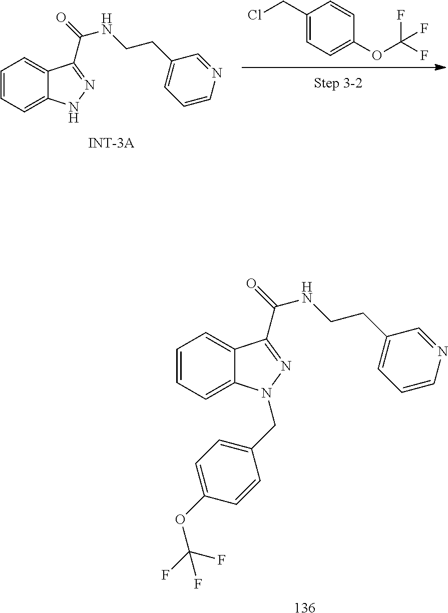

To a stirring solution of crude N-(2-(pyridin-3-yl)ethyl)-1H-indazole-3-carboxamide (INT-3A) (30 mg, 1 Eq, 0.11 mmol) in DMF (1.5 mL) in a vial was added Cs₂CO₃ (0.11 g, 3 Eq, 0.34 mmol) and 1-(chloromethyl)-4-(trifluoromethoxy) benzene (26 mg, 1.1 Eq, 0.12 mmol). The vial was capped and heated at 70° C. for 14 h. After heating for 14 h, the reaction mixture was concentrated in vacuo to afford crude solid. The crude solid was purified by ISCO reversed phase prep (25-35% 0.1% formic acid in MeCN and 0.1% formic acid in water) to afford 16 mg (32% yield) of N-(2-(pyridin-3-yl)ethyl)-1-(4-(trifluoromethoxy)benzyl)-1H-indazole-3-carboxamide (Compound 136) as a white solid; LCMS (m/z) calculated for C₂₃H₁₉F₃N₄O₂: 440.2; found 440.8 [M+H]⁺, t_{R}=7.443 min (Method 2). ¹H NMR (400 MHz, DMSO-d6) δ 8.54 (d, J=16 Hz, 2H), 8.42 (d, J=8 Hz, 1H), 7.63 (d, J=8 Hz, 1H), 7.30 (m, 9H), 5.59 (s, 2H), 3.77 (q, J=8 Hz, 1H), 3.02 (t, J=8 Hz, 1H).

The compounds listed in Table 3 were made using the procedures of Scheme 3.

TABLE 3

| | Cpd. | Purity | | Observed | | Purity |
|---|---|---|---|---|---|---|
| Structure | No. | RT (min) | MW | m/z | Ion | Method |
| 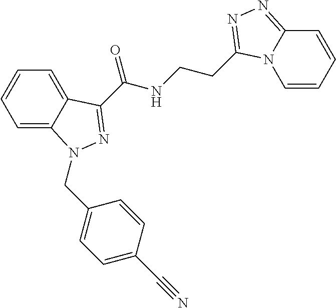 [SMILES: N#Cc1ccc(Cn2nc(C(=O)NCCc3nnc4ccccn34)c3ccccc32)cc1] | 408 | 0.39 | 421.464 | 422.2 | [M + H]⁺ | 6 |
| 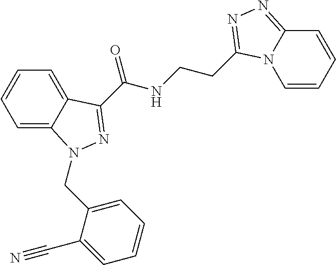 [SMILES: N#Cc1ccccc1Cn1nc(C(=O)NCCc2nnc3ccccn23)c2ccccc21] | 409 | 0.386 | 421.464 | 422.2 | [M + H]⁺ | 6 |
| 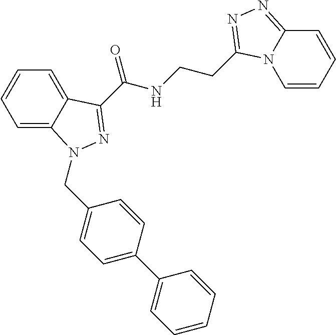 [SMILES: O=C(NCCc1nnc2ccccn12)c1nn(Cc2ccc(-c3ccccc3)cc2)c2ccccc12] | 410 | 0.529 | 472.552 | 473.3 | [M + H]⁺ | 6 |
| 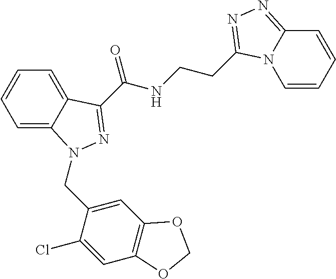 [SMILES: O=C(NCCc1nnc2ccccn12)c1nn(Cc2cc3c(cc2Cl)OCO3)c2ccccc12] | 411 | 0.412 | 474.91 | 475.2 | [M + H]⁺ | 5 |
| 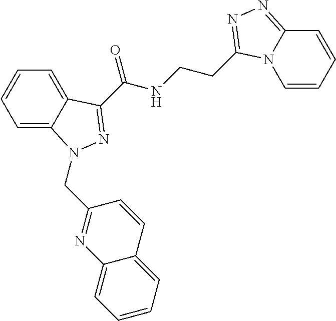 [SMILES: O=C(NCCc1nnc2ccccn12)c1nn(Cc2ccc3ccccc3n2)c2ccccc12] | 412 | 0.37 | 447.502 | 448.2 | [M + H]⁺ | 5 |
| 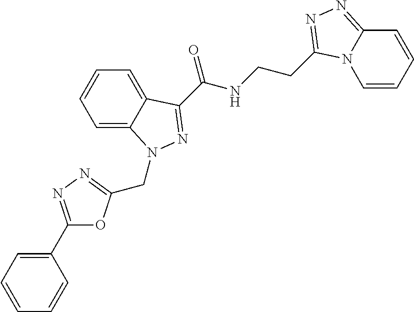 [SMILES: O=C(NCCc1nnc2ccccn12)c1nn(Cc2nnc(-c3ccccc3)o2)c2ccccc12] | 414 | 0.369 | 464.489 | 465.2 | [M + H]⁺ | 5 |
| 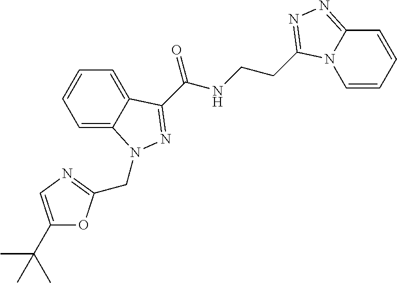 [SMILES: CC(C)(C)c1cnc(Cn2nc(C(=O)NCCc3nnc4ccccn34)c3ccccc32)o1] | 415 | 0.398 | 443.511 | 444.2 | [M + H]⁺ | 5 |
| 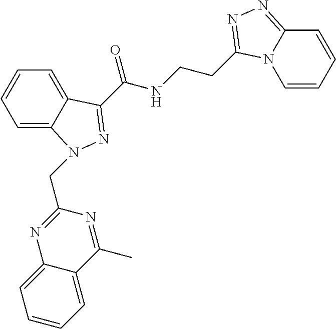 [SMILES: Cc1nc(Cn2nc(C(=O)NCCc3nnc4ccccn34)c3ccccc32)nc2ccccc12] | 431 | 0.371 | 462.517 | 463.3 | [M + H]⁺ | 5 |
| 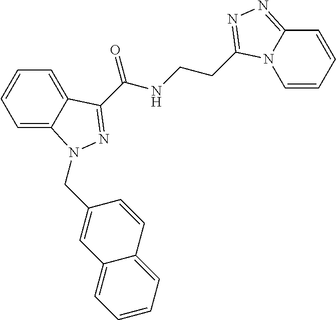 [SMILES: O=C(NCCc1nnc2ccccn12)c1nn(Cc2ccc3ccccc3c2)c2ccccc12] | 432 | 0.432 | 446.514 | 447.3 | [M + H]⁺ | 5 |
| 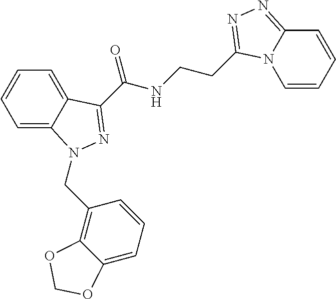 [SMILES: O=C(NCCc1nnc2ccccn12)c1nn(Cc2cccc3c2OCO3)c2ccccc12] | 433 | 0.385 | 440.463 | 441.2 | [M + H]⁺ | 5 |
| 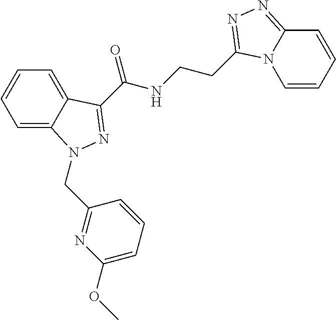 [SMILES: COc1cccc(Cn2nc(C(=O)NCCc3nnc4ccccn34)c3ccccc32)n1] | 434 | 0.372 | 427.468 | 428.3 | [M + H]⁺ | 5 |
| 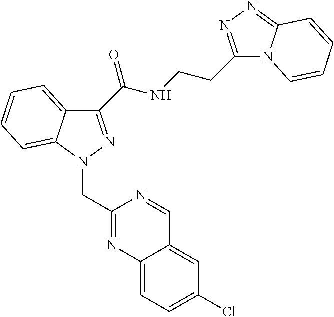 [SMILES: O=C(NCCc1nnc2ccccn12)c1nn(Cc2ncc3cc(Cl)ccc3n2)c2ccccc12] | 436 | 0.447 | 482.93 | 483.1 | [M + H]⁺ | 5 |
| 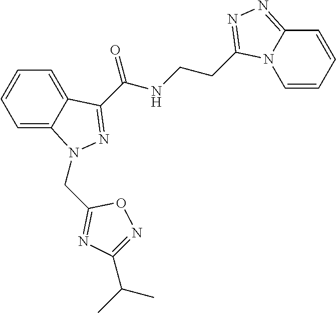 [SMILES: CC(C)c1noc(Cn2nc(C(=O)NCCc3nnc4ccccn34)c3ccccc32)n1] | 437 | 0.36 | 430.472 | 431.2 | [M + H]⁺ | 5 |
| 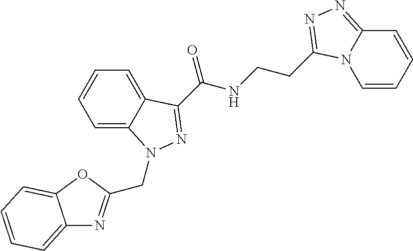 [SMILES: O=C(NCCc1nnc2ccccn12)c1nn(Cc2nc3ccccc3o2)c2ccccc12] | 438 | 0.373 | 437.463 | 438.2 | [M + H]⁺ | 5 |
| 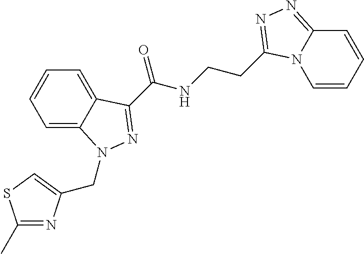 [SMILES: Cc1nc(Cn2nc(C(=O)NCCc3nnc4ccccn34)c3ccccc32)cs1] | 439 | 0.344 | 417.49 | 418.1 | [M + H]⁺ | 5 |
| 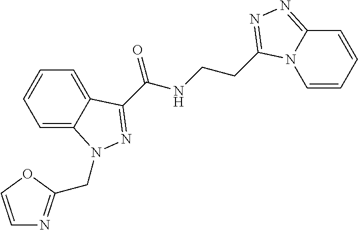 [SMILES: O=C(NCCc1nnc2ccccn12)c1nn(Cc2ncco2)c2ccccc12] | 440 | 0.308 | 387.403 | 388.1 | [M + H]⁺ | 5 |
| 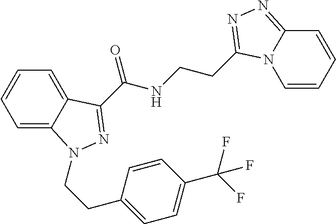 [SMILES: O=C(NCCc1nnc2ccccn12)c1nn(CCc2ccc(C(F)(F)F)cc2)c2ccccc12] | 441 | 0.502 | 478.479 | 479.1 | [M + H]⁺ | 5 |
| 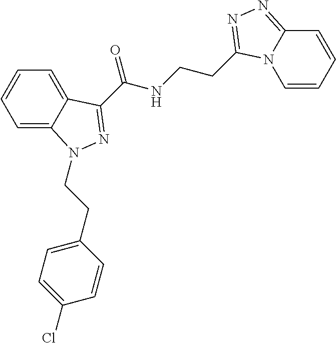 [SMILES: O=C(NCCc1nnc2ccccn12)c1nn(CCc2ccc(Cl)cc2)c2ccccc12] | 442 | 0.419 | 444.92 | 445.2 | [M + H]⁺ | 5 |
| 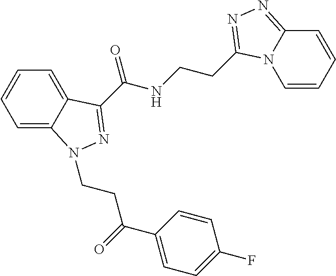 [SMILES: O=C(CCn1nc(C(=O)NCCc2nnc3ccccn23)c2ccccc21)c1ccc(F)cc1] | 443 | 0.387 | 456.481 | 457.2 | [M + H]⁺ | 5 |
| 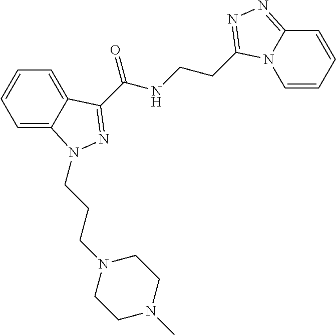 [SMILES: CN1CCN(CCCn2nc(C(=O)NCCc3nnc4ccccn34)c3ccccc32)CC1] | 444 | 0.263 | 446.559 | 447.4 | [M + H]⁺ | 5 |
| 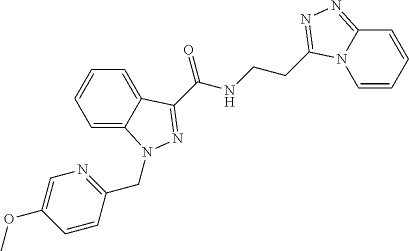 [SMILES: COc1ccc(Cn2nc(C(=O)NCCc3nnc4ccccn34)c3ccccc32)nc1] | 448 | 0.279 | 427.468 | 428.2 | [M + H]⁺ | 5 |
| 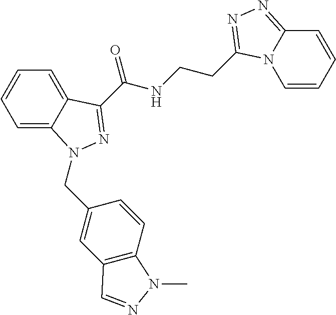 [SMILES: Cn1ncc2cc(Cn3nc(C(=O)NCCc4nnc5ccccn45)c4ccccc43)ccc21] | 452 | 0.433 | 450.506 | 451.2 | [M + H]⁺ | 5 |
| 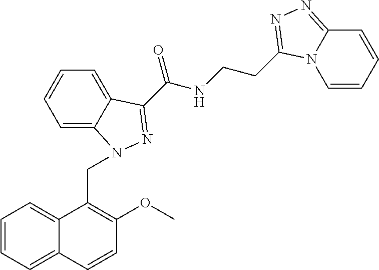 [SMILES: COc1ccc2ccccc2c1Cn1nc(C(=O)NCCc2nnc3ccccn23)c2ccccc21] | 453 | 0.506 | 476.54 | 477.1 | [M + H]⁺ | 5 |
| 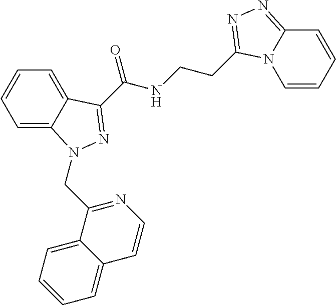 [SMILES: O=C(NCCc1nnc2ccccn12)c1nn(Cc2nccc3ccccc23)c2ccccc12] | 454 | 0.288 | 474.502 | 448.2 | [M + H]⁺ | 5 |
| 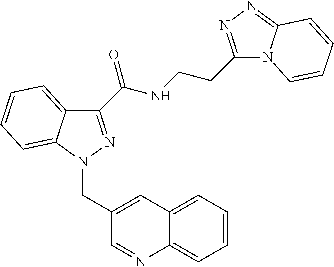 [SMILES: O=C(NCCc1nnc2ccccn12)c1nn(Cc2cnc3ccccc3c2)c2ccccc12] | 455 | 0.396 | 447.502 | 448.2 | [M + H]⁺ | 5 |
| 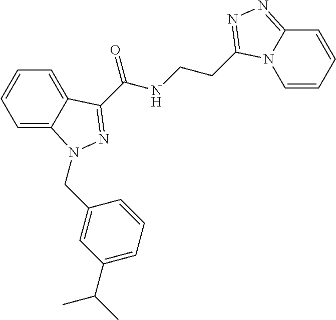 [SMILES: CC(C)c1cccc(Cn2nc(C(=O)NCCc3nnc4ccccn34)c3ccccc32)c1] | 456 | 0.515 | 438.535 | 439.2 | [M + H]⁺ | 5 |
| 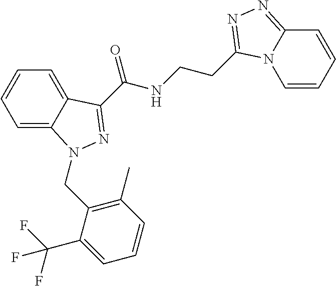 [SMILES: Cc1cccc(C(F)(F)F)c1Cn1nc(C(=O)NCCc2nnc3ccccn23)c2ccccc21] | 457 | 0.497 | 478.479 | 479.1 | [M + H]⁺ | 5 |
| 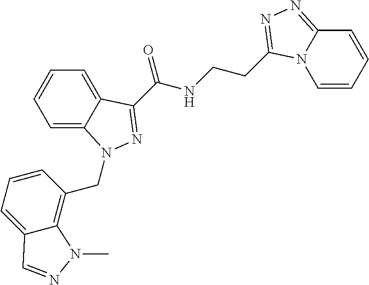 [SMILES: Cn1ncc2cccc(Cn3nc(C(=O)NCCc4nnc5ccccn45)c4ccccc43)c21] | 460 | 0.375 | 450.506 | 451.2 | [M + H]⁺ | 5 |
| 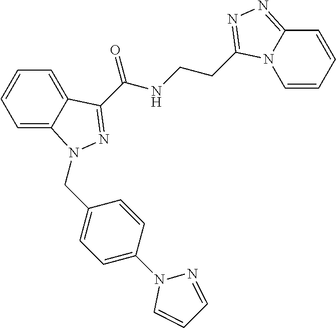 [SMILES: O=C(NCCc1nnc2ccccn12)c1nn(Cc2ccc(-n3cccn3)cc2)c2ccccc12] | 461 | 0.38 | 462.517 | 463.3 | [M + H]⁺ | 5 |
| 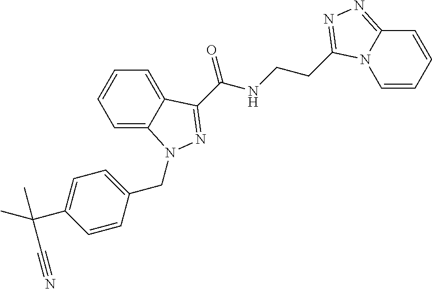 [SMILES: CC(C)(C#N)c1ccc(Cn2nc(C(=O)NCCc3nnc4ccccn34)c3ccccc32)cc1] | 462 | 0.398 | 463.545 | 464.3 | [M + H]⁺ | 5 |
| 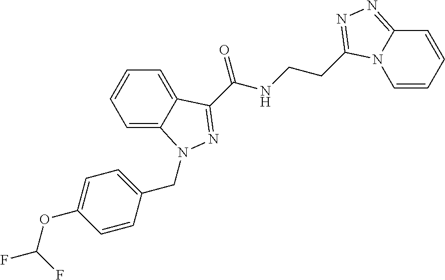 [SMILES: O=C(NCCc1nnc2ccccn12)c1nn(Cc2ccc(OC(F)F)cc2)c2ccccc12] | 463 | 0.344 | 462.461 | 463.2 | [M + H]⁺ | 5 |
| 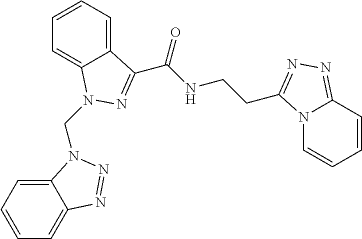 [SMILES: O=C(NCCc1nnc2ccccn12)c1nn(Cn2nnc3ccccc32)c2ccccc12] | 465 | 0.357 | 437.467 | 438.3 | [M + H]⁺ | 5 |
| 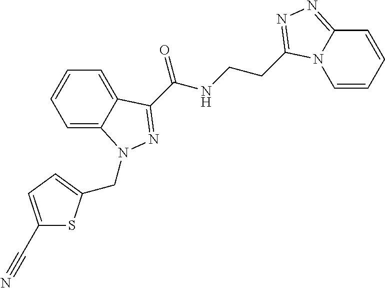 [SMILES: N#Cc1ccc(Cn2nc(C(=O)NCCc3nnc4ccccn34)c3ccccc32)s1] | 466 | 0.306 | 427.49 | 428.1 | [M + H]⁺ | 5 |
| 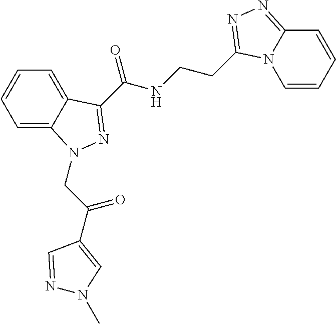 [SMILES: Cn1cc(C(=O)Cn2nc(C(=O)NCCc3nnc4ccccn34)c3ccccc32)cn1] | 467 | 0.317 | 428.456 | 429.3 | [M + H]⁺ | 5 |
| 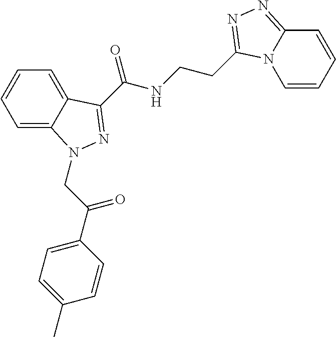 [SMILES: Cc1ccc(C(=O)Cn2nc(C(=O)NCCc3nnc4ccccn34)c3ccccc32)cc1] | 468 | 0.329 | 438.491 | 439.1 | [M + H]⁺ | 5 |
| 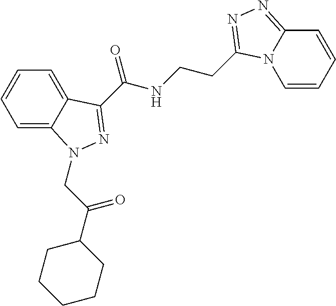 [SMILES: O=C(NCCc1nnc2ccccn12)c1nn(CC(=O)C2CCCCC2)c2ccccc12] | 469 | 0.389 | 430.512 | 431.3 | [M + H]⁺ | 5 |
| 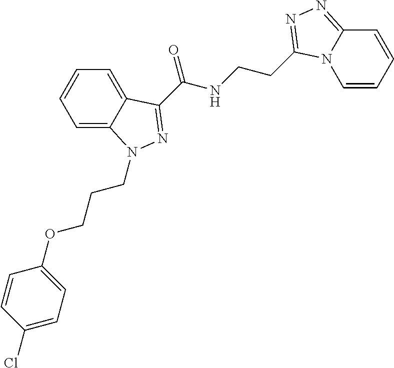 [SMILES: O=C(NCCc1nnc2ccccn12)c1nn(CCCOc2ccc(Cl)cc2)c2ccccc12] | 470 | 0.438 | 474.95 | 475.2 | [M + H]⁺ | 5 |
| 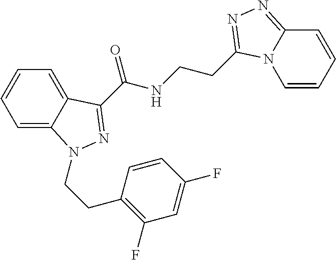 [SMILES: O=C(NCCc1nnc2ccccn12)c1nn(CCc2ccc(F)cc2F)c2ccccc12] | 471 | 0.348 | 446.462 | 447.2 | [M + H]⁺ | 5 |
| 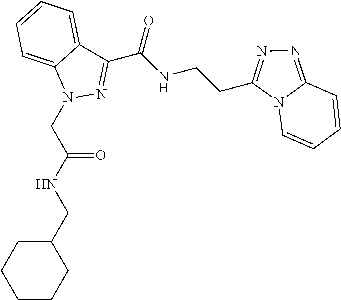 [SMILES: O=C(Cn1nc(C(=O)NCCc2nnc3ccccn23)c2ccccc21)NCC1CCCCC1] | 472 | 0.39 | 459.554 | 460.3 | [M + H]⁺ | 5 |
| 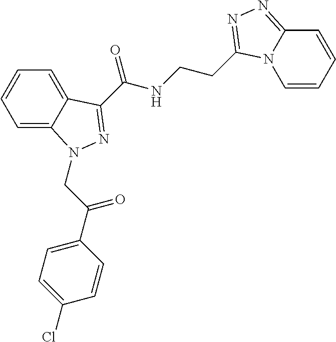 [SMILES: O=C(Cn1nc(C(=O)NCCc2nnc3ccccn23)c2ccccc21)c1ccc(Cl)cc1] | 481 | 0.333 | 458.91 | 459.1 | [M + H]⁺ | 5 |
| 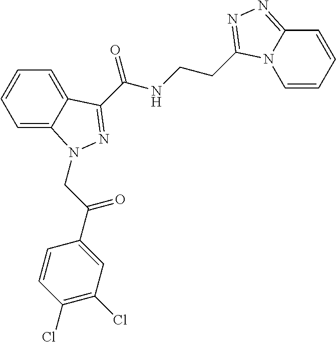 [SMILES: O=C(Cn1nc(C(=O)NCCc2nnc3ccccn23)c2ccccc21)c1ccc(Cl)c(Cl)c1] | 482 | 0.363 | 493.35 | 493.1 | [M + H]⁺ | 5 |
| 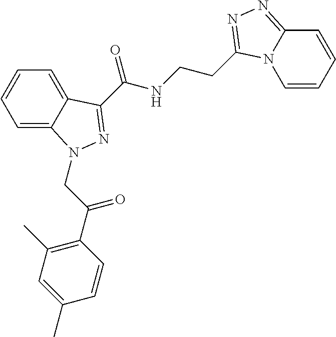 [SMILES: Cc1ccc(C(=O)Cn2nc(C(=O)NCCc3nnc4ccccn34)c3ccccc32)c(C)c1] | 483 | 0.411 | 452.518 | 453.3 | [M + H]⁺ | 5 |
| 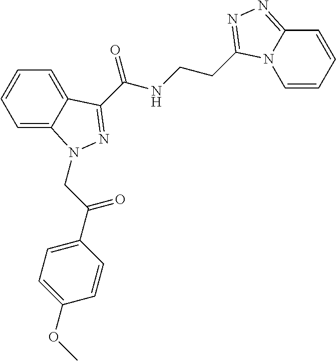 [SMILES: COc1ccc(C(=O)Cn2nc(C(=O)NCCc3nnc4ccccn34)c3ccccc32)cc1] | 484 | 0.373 | 454.49 | 455.2 | [M + H]⁺ | 5 |
| 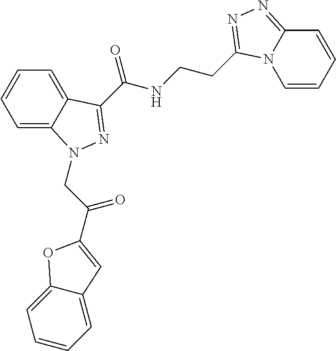 [SMILES: O=C(Cn1nc(C(=O)NCCc2nnc3ccccn23)c2ccccc21)c1cc2ccccc2o1] | 503 | 0.46 | 464.485 | 465.3 | [M + H]⁺ | 5 |
| 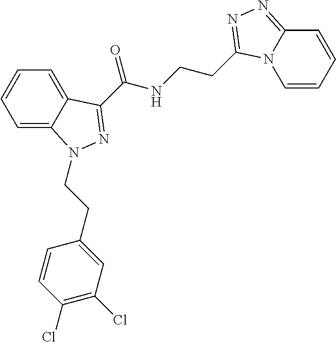 [SMILES: O=C(NCCc1nnc2ccccn12)c1nn(CCc2ccc(Cl)c(Cl)c2)c2ccccc12] | 505 | 0.483 | 479.37 | 479.2 | [M + H]⁺ | 5 |
| 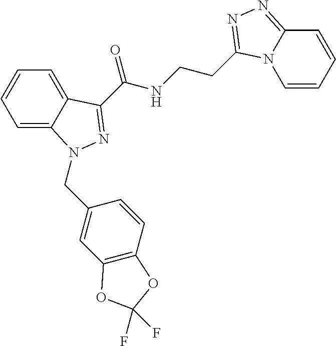 [SMILES: O=C(NCCc1nnc2ccccn12)c1nn(Cc2ccc3c(c2)OC(F)(F)O3)c2ccccc12] | 507 | 0.478 | 476.444 | 477.5 | [M + H]⁺ | 5 |
| 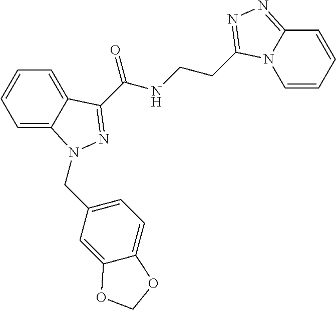 [SMILES: O=C(NCCc1nnc2ccccn12)c1nn(Cc2ccc3c(c2)OCO3)c2ccccc12] | 508 | 0.417 | 440.463 | 441.5 | [M + H]⁺ | 5 |
| 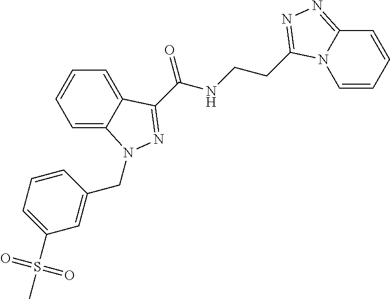 [SMILES: CS(=O)(=O)c1cccc(Cn2nc(C(=O)NCCc3nnc4ccccn34)c3ccccc32)c1] | 510 | 0.375 | 474.54 | 475.2 | [M + H]⁺ | 5 |
| 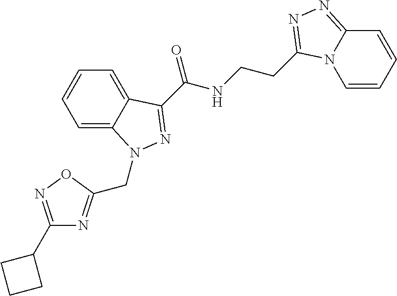 [SMILES: O=C(NCCc1nnc2ccccn12)c1nn(Cc2nc(C3CCC3)no2)c2ccccc12] | 511 | 0.432 | 442.483 | 443.3 | [M + H]⁺ | 5 |
| 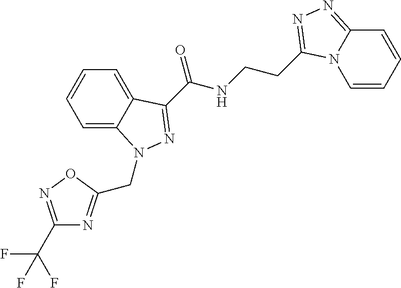 [SMILES: O=C(NCCc1nnc2ccccn12)c1nn(Cc2nc(C(F)(F)F)no2)c2ccccc12] | 513 | 0.45 | 456.389 | 457.2 | [M + H]⁺ | 5 |
| 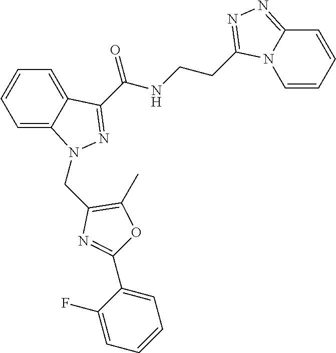 [SMILES: Cc1oc(-c2ccccc2F)nc1Cn1nc(C(=O)NCCc2nnc3ccccn23)c2ccccc21] | 514 | 0.461 | 495.518 | 496.3 | [M + H]⁺ | 5 |
| 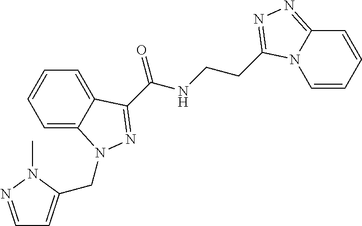 [SMILES: Cn1nccc1Cn1nc(C(=O)NCCc2nnc3ccccn23)c2ccccc21] | 515 | 0.352 | 400.446 | 401.1 | [M + H]⁺ | 5 |
| 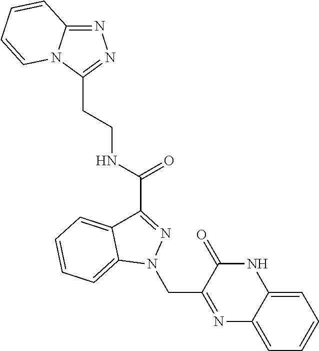 [SMILES: O=C(NCCc1nnc2ccccn12)c1nn(Cc2nc3ccccc3[nH]c2=O)c2ccccc12] | 516 | 0.376 | 464.489 | 465.2 | [M + H]⁺ | 5 |
| 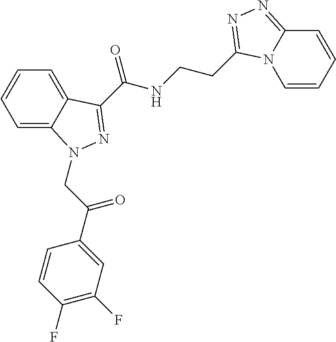 [SMILES: O=C(Cn1nc(C(=O)NCCc2nnc3ccccn23)c2ccccc21)c1ccc(F)c(F)c1] | 517 | 0.452 | 460.445 | 461.1 | [M + H]⁺ | 5 |
| 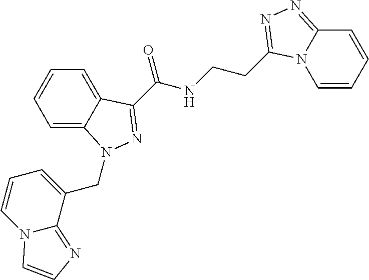 [SMILES: O=C(NCCc1nnc2ccccn12)c1nn(Cc2cccn3ccnc23)c2ccccc12] | 518 | 0.303 | 436.479 | 437.3 | [M + H]⁺ | 5 |
| 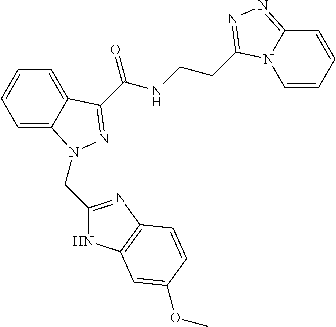 [SMILES: COc1ccc2nc(Cn3nc(C(=O)NCCc4nnc5ccccn45)c4ccccc43)[nH]c2c1] | 519 | 0.349 | 466.505 | 467.2 | [M + H]⁺ | 5 |
| 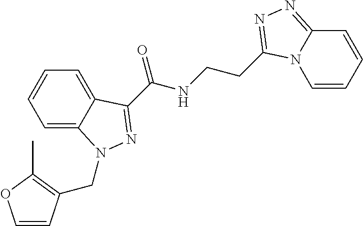 [SMILES: Cc1occc1Cn1nc(C(=O)NCCc2nnc3ccccn23)c2ccccc21] | 520 | 0.425 | 400.442 | 401.2 | [M + H]⁺ | 5 |
| 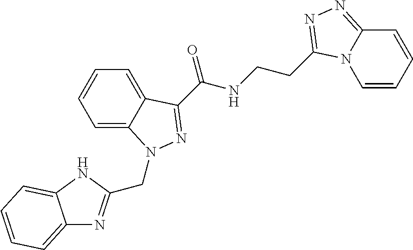 [SMILES: O=C(NCCc1nnc2ccccn12)c1nn(Cc2nc3ccccc3[nH]2)c2ccccc12] | 521 | 0.333 | 436.479 | 437.3 | [M + H]⁺ | 5 |
| 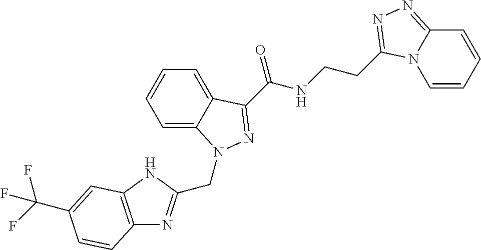 [SMILES: O=C(NCCc1nnc2ccccn12)c1nn(Cc2nc3ccc(C(F)(F)F)cc3[nH]2)c2ccccc12] | 522 | 0.433 | 504.477 | 505.4 | [M + H]⁺ | 5 |
| 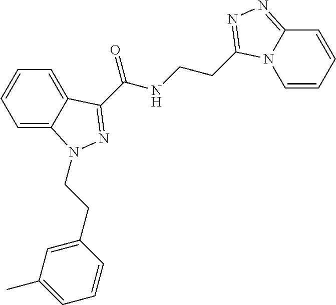 [SMILES: Cc1cccc(CCn2nc(C(=O)NCCc3nnc4ccccn34)c3ccccc32)c1] | 523 | 0.495 | 424.508 | 425.3 | [M + H]⁺ | 5 |
| 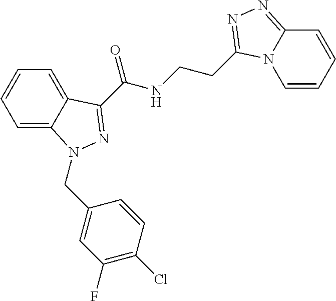 [SMILES: O=C(NCCc1nnc2ccccn12)c1nn(Cc2ccc(Cl)c(F)c2)c2ccccc12] | 524 | 11.93 | 448.89 | 448.8 | [M + H]⁺ | 3 |
| 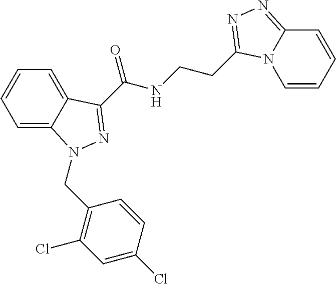 [SMILES: O=C(NCCc1nnc2ccccn12)c1nn(Cc2ccc(Cl)cc2Cl)c2ccccc12] | 525 | 12.72 | 465.34 | 464.6 | [M + H]⁺ | 3 |
| 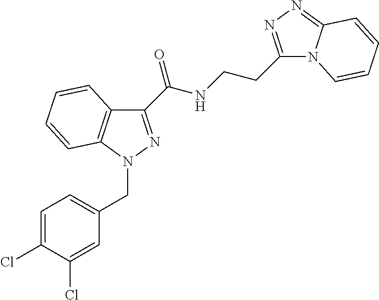 [SMILES: O=C(NCCc1nnc2ccccn12)c1nn(Cc2ccc(Cl)c(Cl)c2)c2ccccc12] | 526 | 12.36 | 465.34 | 464.9 | [M + H]⁺ | 3 |
| 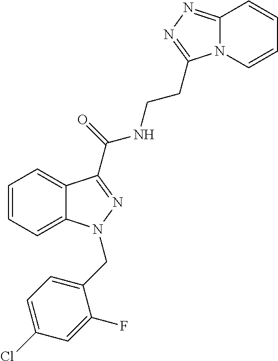 [SMILES: O=C(NCCc1nnc2ccccn12)c1nn(Cc2ccc(Cl)cc2F)c2ccccc12] | 527 | 12.03 | 448.89 | 448.8 | [M + H]⁺ | 3 |
| 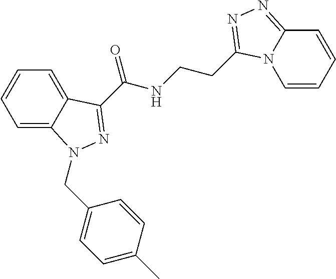 [SMILES: Cc1ccc(Cn2nc(C(=O)NCCc3nnc4ccccn34)c3ccccc32)cc1] | 528 | 11.7 | 410.481 | 410.8 | [M + H]⁺ | 3 |
| 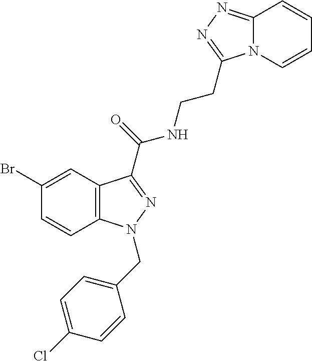 [SMILES: O=C(NCCc1nnc2ccccn12)c1nn(Cc2ccc(Cl)cc2)c2ccc(Br)cc12] | 529 | 12.73 | 509.79 | 508.8 | [M + H]⁺ | 3 |
| 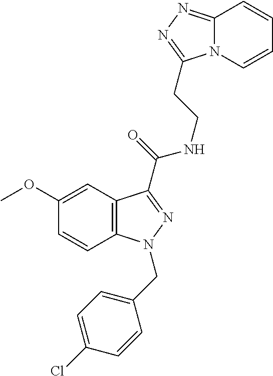 [SMILES: COc1ccc2c(c1)c(C(=O)NCCc1nnc3ccccn13)nn2Cc1ccc(Cl)cc1] | 543 | 11.76 | 460.92 | 461.1 | [M + H]⁺ | 3 |
| 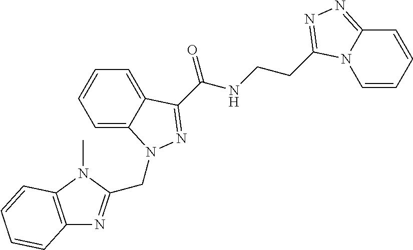 [SMILES: Cn1c(Cn2nc(C(=O)NCCc3nnc4ccccn34)c3ccccc32)nc2ccccc21] | 544 | 8.56 | 450.506 | 450.9 | [M + H]⁺ | 3 |
| 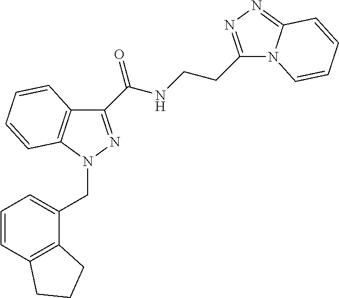 [SMILES: O=C(NCCc1nnc2ccccn12)c1nn(Cc2cccc3c2CCC3)c2ccccc12] | 545 | 12.21 | 436.519 | 436.9 | [M + H]⁺ | 3 |
| 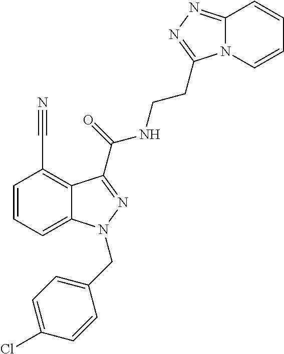 [SMILES: N#Cc1cccc2c1c(C(=O)NCCc1nnc3ccccn13)nn2Cc1ccc(Cl)cc1] | 546 | 10.67 | 455.91 | 456.2 | [M + H]⁺ | 3 |
| 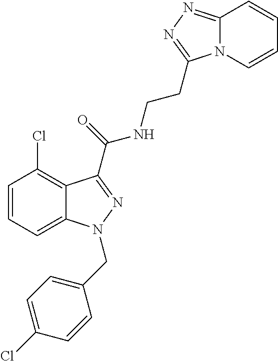 [SMILES: O=C(NCCc1nnc2ccccn12)c1nn(Cc2ccc(Cl)cc2)c2cccc(Cl)c12] | 547 | 11.14 | 465.34 | 464.6 | [M + H]⁺ | 3 |
| 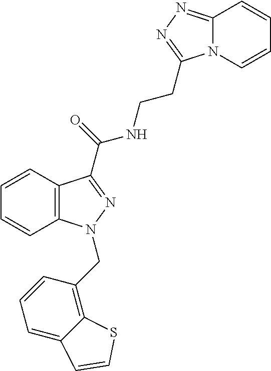 [SMILES: O=C(NCCc1nnc2ccccn12)c1nn(Cc2cccc3ccsc23)c2ccccc12] | 548 | 11.52 | 452.54 | 452.9 | [M + H]⁺ | 3 |
| 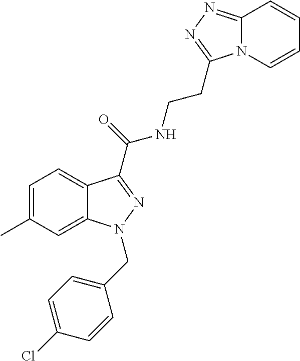 [SMILES: Cc1ccc2c(C(=O)NCCc3nnc4ccccn34)nn(Cc3ccc(Cl)cc3)c2c1] | 549 | 12.18 | 444.92 | 444.6 | [M + H]⁺ | 3 |
| 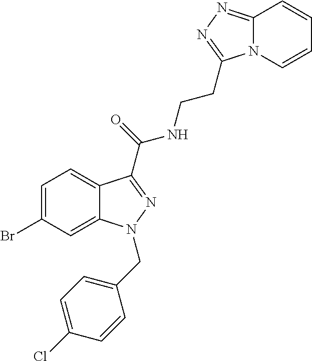 [SMILES: O=C(NCCc1nnc2ccccn12)c1nn(Cc2ccc(Cl)cc2)c2cc(Br)ccc12] | 550 | 12.61 | 509.79 | 508.5 | [M + H]⁺ | 3 |
| 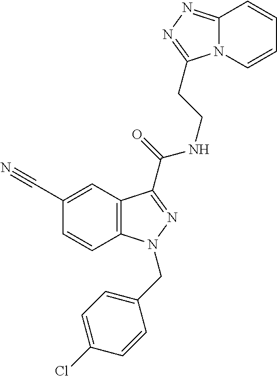 [SMILES: N#Cc1ccc2c(c1)c(C(=O)NCCc1nnc3ccccn13)nn2Cc1ccc(Cl)cc1] | 551 | 11.36 | 455.91 | 455.9 | [M + H]⁺ | 3 |
| 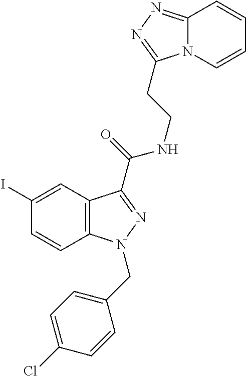 [SMILES: O=C(NCCc1nnc2ccccn12)c1nn(Cc2ccc(Cl)cc2)c2ccc(I)cc12] | 552 | 12.69 | 556.79 | 556.5 | [M + H]⁺ | 3 |
| 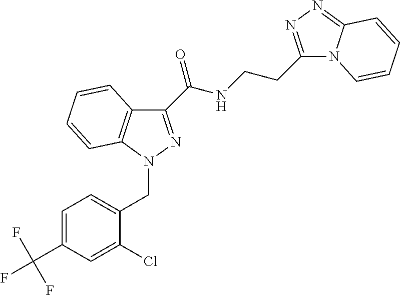 [SMILES: O=C(NCCc1nnc2ccccn12)c1nn(Cc2ccc(C(F)(F)F)cc2Cl)c2ccccc12] | 553 | 12.34 | 498.89 | 498.6 | [M + H]⁺ | 3 |
| 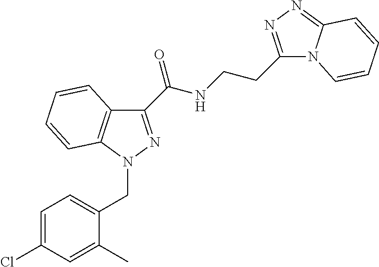 [SMILES: Cc1cc(Cl)ccc1Cn1nc(C(=O)NCCc2nnc3ccccn23)c2ccccc21] | 554 | 11.91 | 444.92 | 444.9 | [M + H]⁺ | 3 |
| 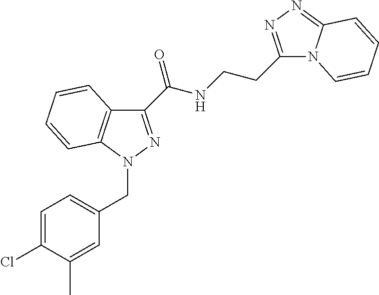 [SMILES: Cc1cc(Cn2nc(C(=O)NCCc3nnc4ccccn34)c3ccccc32)ccc1Cl] | 555 | 12.1 | 444.92 | 445 | [M + H]⁺ | 3 |
| 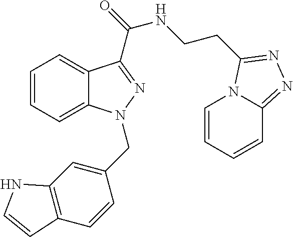 [SMILES: O=C(NCCc1nnc2ccccn12)c1nn(Cc2ccc3cc[nH]c3c2)c2ccccc12] | 556 | 10.88 | 435.491 | 436 | [M + H]⁺ | 3 |
| 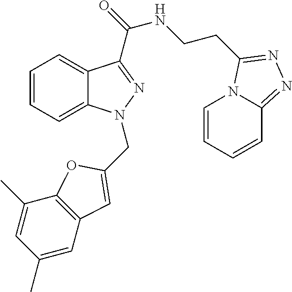 [SMILES: Cc1cc(C)c2oc(Cn3nc(C(=O)NCCc4nnc5ccccn45)c4ccccc43)cc2c1] | 557 | 12.53 | 464.529 | 464.9 | [M + H]⁺ | 3 |
| 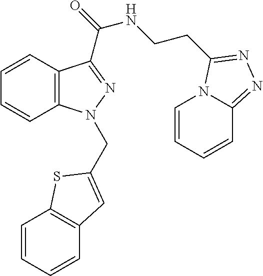 [SMILES: O=C(NCCc1nnc2ccccn12)c1nn(Cc2cc3ccccc3s2)c2ccccc12] | 558 | 13.4 | 452.54 | 452.8 | [M + H]⁺ | 3 |
| 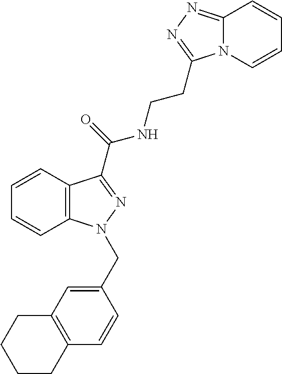 [SMILES: O=C(NCCc1nnc2ccccn12)c1nn(Cc2ccc3c(c2)CCCC3)c2ccccc12] | 559 | 12.7 | 450.546 | 450.8 | [M + H]⁺ | 3 |
| 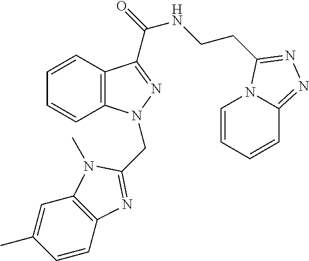 [SMILES: Cc1ccc2nc(Cn3nc(C(=O)NCCc4nnc5ccccn45)c4ccccc43)n(C)c2c1] | 568 | 8.11 | 464.533 | 464.9 | [M + H]⁺ | 3 |
| 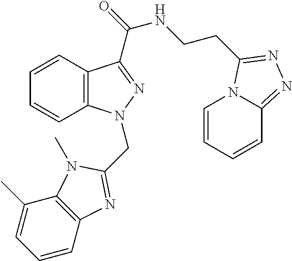 [SMILES: Cc1cccc2nc(Cn3nc(C(=O)NCCc4nnc5ccccn45)c4ccccc43)n(C)c12] | 569 | 9.17 | 464.533 | 464.5 | [M + H]⁺ | 3 |
| 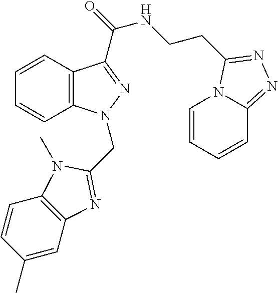 [SMILES: Cc1ccc2c(c1)nc(Cn1nc(C(=O)NCCc3nnc4ccccn34)c3ccccc31)n2C] | 570 | 9.19 | 464.533 | 465.2 | [M + H]⁺ | 3 |
| 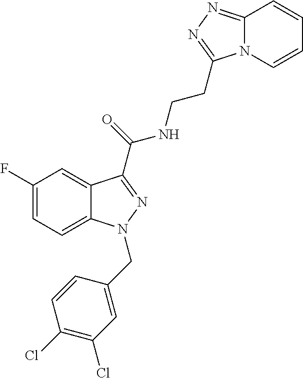 [SMILES: O=C(NCCc1nnc2ccccn12)c1nn(Cc2ccc(Cl)c(Cl)c2)c2ccc(F)cc12] | 571 | 12.2 | 483.33 | 483 | [M + H]⁺ | 3 |
| 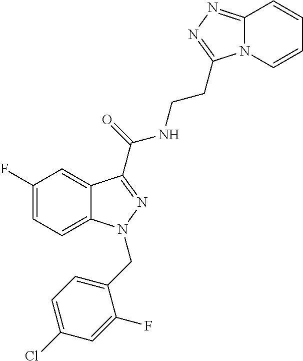 [SMILES: O=C(NCCc1nnc2ccccn12)c1nn(Cc2ccc(Cl)cc2F)c2ccc(F)cc12] | 572 | 11.91 | 466.88 | 467 | [M + H]⁺ | 3 |
| 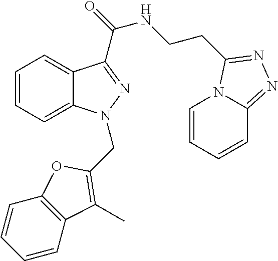 [SMILES: Cc1c(Cn2nc(C(=O)NCCc3nnc4ccccn34)c3ccccc32)oc2ccccc12] | 573 | 11.91 | 450.502 | 451.4 | [M + H]⁺ | 3 |
| 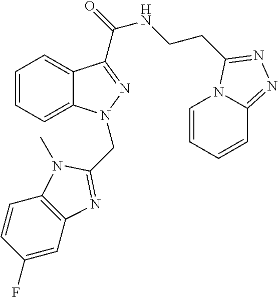 [SMILES: Cn1c(Cn2nc(C(=O)NCCc3nnc4ccccn34)c3ccccc32)nc2cc(F)ccc21] | 574 | 9.63 | 468.496 | 468.9 | [M + H]⁺ | 3 |

### Example 4

### Synthesis of Compound 135

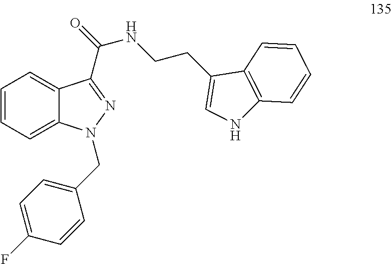 [SMILES: O=C(NCCc1c[nH]c2ccccc12)c1nn(Cc2ccc(F)cc2)c2ccccc12]

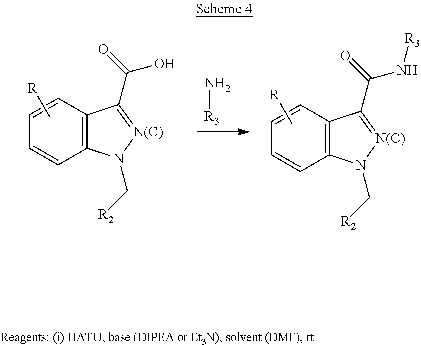

### Step 4-1. Synthesis of N-(2-(1H-indol-3-yl)ethyl)-1-(4-methylbenzyl)-1H-indazole-3-carboxamide (Compound 135)

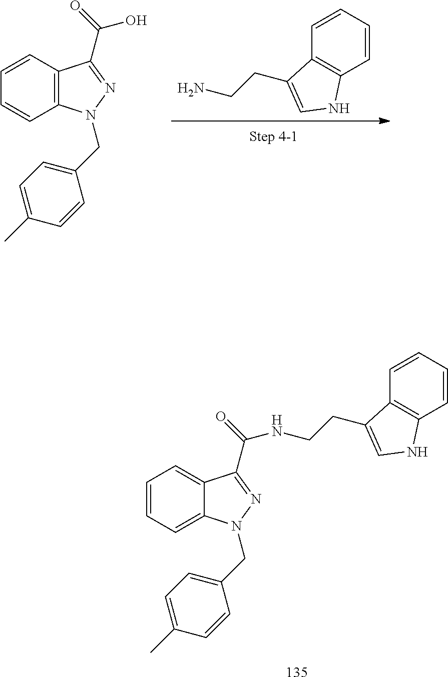 [SMILES: Cc1ccc(Cn2nc(C(=O)O)c3ccccc32)cc1 ; NCCc1c[nH]c2ccccc12 ; Cc1ccc(Cn2nc(C(=O)NCCc3c[nH]c4ccccc34)c3ccccc32)cc1 ; "135"]

To a vial containing a stirred solution of 1-(4-methylbenzyl)-1H-indazole-3-carboxylica acid (100 mg, 1 Eq, 376 μmol), and DIPEA (194 mg, 0.26 mL, 4 Eq, 1.50 mmol), in DMF (2 mL) was added HATU (157 mg, 1.1 Eq, 413 μmol) and the resulting yellow solution was stirred at room temperature for 5 minutes. Tryptamine (72 mg, 1.2 Eq, 451 μmol) was added, and the resulting yellow-orange solution was stirred overnight at room temperature. After stirring for 17 hours, the reaction mixture (orange solution) was diluted with EtOAc (5 mL) and washed with saturated aqueous NaHCO₃ (10 mL). The aqueous layer was extracted with EtOAc (2×10 mL). The combined organics were washed with brine (10 mL), dried over Na₂SO₄, filtered, and concentrated in vacuo to give crude residue. The crude residue (orange oil) was purified by silica gel column chromatography (0-70% EtOAc and hexanes) to give 131 mg (85% yield) of N-(2-(1H-indol-3-yl)ethyl)-1-(4-methylbenzyl)-1H-indazole-3-carboxamide (Compound 135) as a pale-yellow foam; LCMS (m/z) calculated for C₂₆H₂₄N₄O: 408.2; found 409.3 [M+H]⁺, t_{R}=10.911 min (Method 2).

The compounds listed in Table 4 were made using the procedures of Scheme 4.

TABLE 4

| | Cpd | Purity | | Observed | | Purity |
|---|---|---|---|---|---|---|
| Structure | No. | RT (min) | MW | m/z | Ion | Method |
| 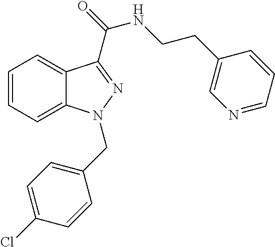 [SMILES: O=C(NCCc1cccnc1)c1nn(Cc2ccc(Cl)cc2)c2ccccc12] | 149 | 11.26 | 389.1 | 390.6 | [M + H]⁺ | 3 |
| 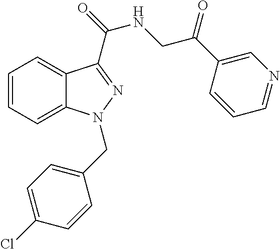 [SMILES: O=C(CNC(=O)c1nn(Cc2ccc(Cl)cc2)c2ccccc12)c1cccnc1] | 132 | 12.77 | 404.1 | 405.4 | [M + H]⁺ | 3 |
| 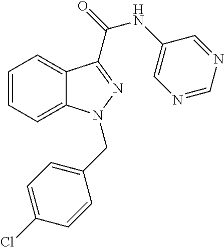 [SMILES: O=C(Nc1cncnc1)c1nn(Cc2ccc(Cl)cc2)c2ccccc12] | 133 | 13.34 | 363.1 | 364.4 | [M + H]⁺ | 3 |
| 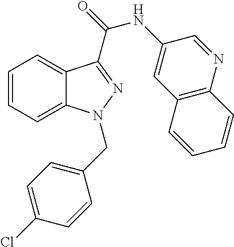 [SMILES: O=C(Nc1cnc2ccccc2c1)c1nn(Cc2ccc(Cl)cc2)c2ccccc12] | 134 | 13.99 | 412.1 | 413.2 | [M + H]⁺ | 3 |
| 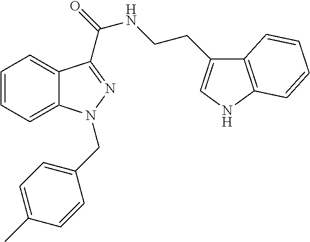 [SMILES: Cc1ccc(Cn2nc(C(=O)NCCc3c[nH]c4ccccc34)c3ccccc32)cc1] | 135 | 10.911 | 408.2 | 409.3 | [M + H]⁺ | 2 |
| 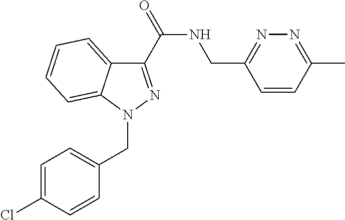 [SMILES: Cc1ccc(CNC(=O)c2nn(Cc3ccc(Cl)cc3)c3ccccc23)nn1] | 334 | 0.501 | 391.86 | 391.9 | [M + H]⁺ | 5 |
| 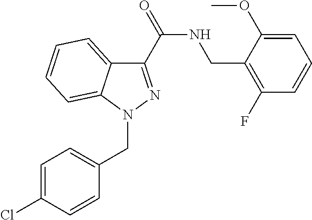 [SMILES: COc1cccc(F)c1CNC(=O)c1nn(Cc2ccc(Cl)cc2)c2ccccc12] | 335 | 0.628 | 423.87 | 423.9 | [M + H]⁺ | 5 |
| 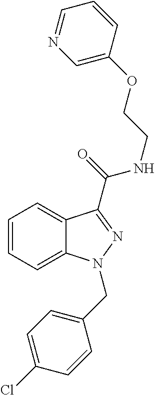 [SMILES: O=C(NCCOc1cccnc1)c1nn(Cc2ccc(Cl)cc2)c2ccccc12] | 336 | 0.469 | 406.87 | 407 | [M + H]⁺ | 5 |
| 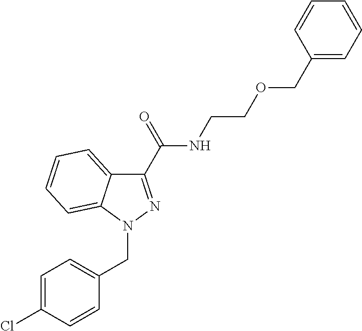 [SMILES: O=C(NCCOCc1ccccc1)c1nn(Cc2ccc(Cl)cc2)c2ccccc12] | 337 | 0.622 | 419.91 | 420 | [M + H]⁺ | 5 |
| 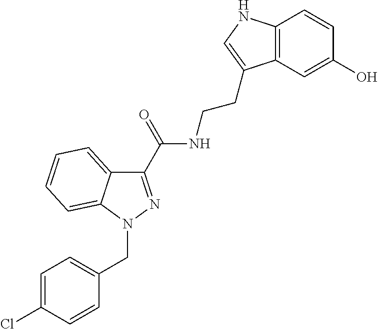 [SMILES: O=C(NCCc1c[nH]c2ccc(O)cc12)c1nn(Cc2ccc(Cl)cc2)c2ccccc12] | 338 | 0.491 | 444.92 | 445.2 | [M + H]⁺ | 5 |
| 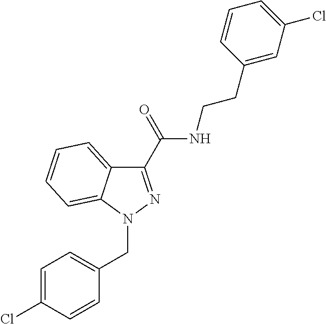 [SMILES: O=C(NCCc1cccc(Cl)c1)c1nn(Cc2ccc(Cl)cc2)c2ccccc12] | 339 | 0.58 | 424.33 | 424.1 | [M + H]⁺ | 5 |
| 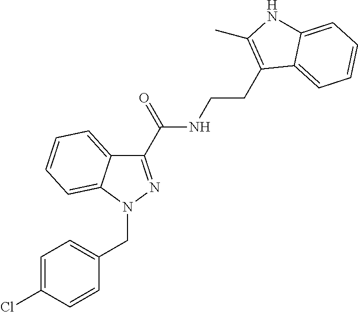 [SMILES: Cc1[nH]c2ccccc2c1CCNC(=O)c1nn(Cc2ccc(Cl)cc2)c2ccccc12] | 340 | 0.554 | 442.95 | 443.2 | [M + H]⁺ | 5 |
| 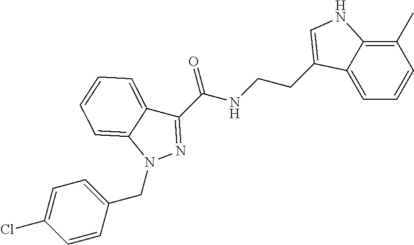 [SMILES: Cc1cccc2c(CCNC(=O)c3nn(Cc4ccc(Cl)cc4)c4ccccc34)c[nH]c12] | 341 | 0.561 | 442.95 | 443.2 | [M + H]⁺ | 5 |
| 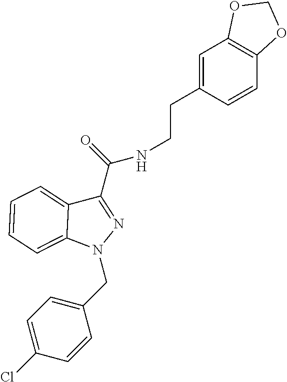 [SMILES: O=C(NCCc1ccc2c(c1)OCO2)c1nn(Cc2ccc(Cl)cc2)c2ccccc12] | 342 | 0.553 | 433.89 | 434.1 | [M + H]⁺ | 5 |
| 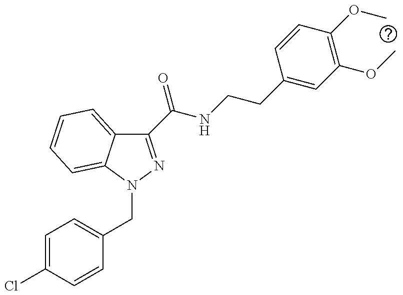 [SMILES: COc1ccc(CCNC(=O)c2nn(Cc3ccc(Cl)cc3)c3ccccc23)cc1OC] | 343 | 0.534 | 449.94 | 450.1 | [M + H]⁺ | 5 |
| 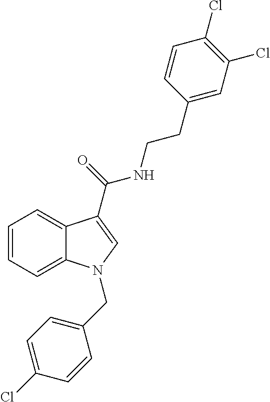 [SMILES: O=C(NCCc1ccc(Cl)c(Cl)c1)c1cn(Cc2ccc(Cl)cc2)c2ccccc12] | 344 | 0.596 | 458.77 | 458 | [M + H]⁺ | 5 |
| 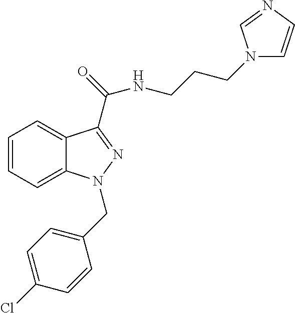 [SMILES: O=C(NCCCn1ccnc1)c1nn(Cc2ccc(Cl)cc2)c2ccccc12] | 345 | 0.379 | 393.88 | 394.2 | [M + H]⁺ | 5 |
| 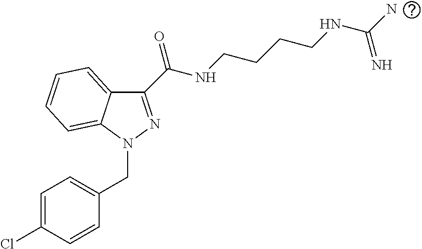 [SMILES: N=C(N)NCCCCNC(=O)c1nn(Cc2ccc(Cl)cc2)c2ccccc12] | 346 | 0.396 | 398.9 | 399.2 | [M + H]⁺ | 5 |
| 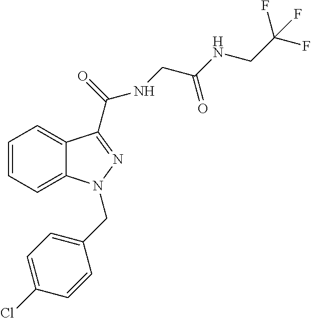 [SMILES: O=C(CNC(=O)c1nn(Cc2ccc(Cl)cc2)c2ccccc12)NCC(F)(F)F] | 347 | 0.487 | 424.81 | 425.1 | [M + H]⁺ | 5 |
| 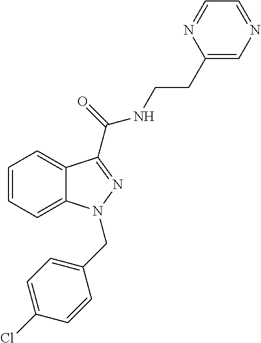 [SMILES: O=C(NCCc1cnccn1)c1nn(Cc2ccc(Cl)cc2)c2ccccc12] | 348 | 0.473 | 391.86 | 392.3 | [M + H]⁺ | 5 |
| 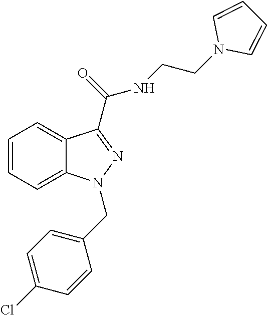 [SMILES: O=C(NCCn1cccc1)c1nn(Cc2ccc(Cl)cc2)c2ccccc12] | 349 | 0.52 | 378.86 | 379.2 | [M + H]⁺ | 5 |
| 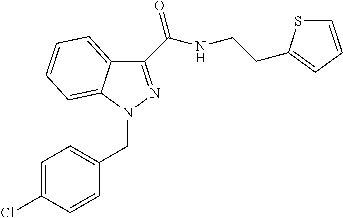 [SMILES: O=C(NCCc1cccs1)c1nn(Cc2ccc(Cl)cc2)c2ccccc12] | 350 | 0.551 | 395.91 | 396.1 | [M + H]⁺ | 5 |
| 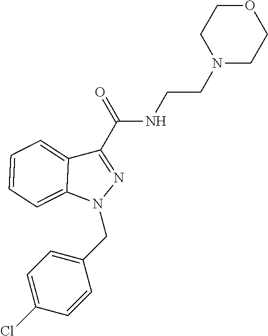 [SMILES: O=C(NCCN1CCOCC1)c1nn(Cc2ccc(Cl)cc2)c2ccccc12] | 351 | 0.38 | 398.89 | 399.3 | [M + H]⁺ | 5 |
| 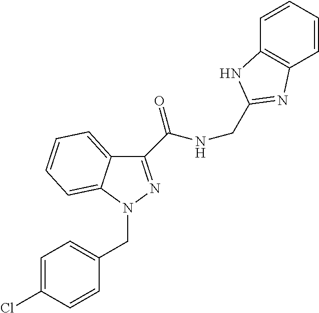 [SMILES: O=C(NCc1nc2ccccc2[nH]1)c1nn(Cc2ccc(Cl)cc2)c2ccccc12] | 352 | 0.402 | 415.88 | 4A.1 | [M + H]⁺ | 5 |
| 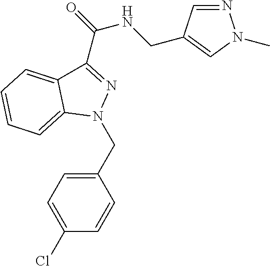 [SMILES: Cn1cc(CNC(=O)c2nn(Cc3ccc(Cl)cc3)c3ccccc23)cn1] | 353 | 0.468 | 379.85 | 380.1 | [M + H]⁺ | 5 |
| 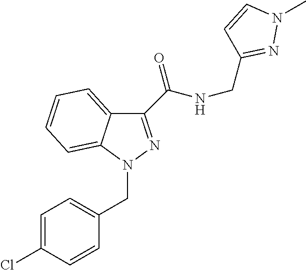 [SMILES: Cn1ccc(CNC(=O)c2nn(Cc3ccc(Cl)cc3)c3ccccc23)n1] | 354 | 0.543 | 379.85 | 380.1 | [M + H]⁺ | 6 |
| 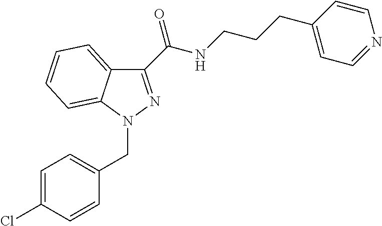 [SMILES: O=C(NCCCc1ccncc1)c1nn(Cc2ccc(Cl)cc2)c2ccccc12] | 355 | 0.387 | 404.9 | 405.1 | [M + H]⁺ | 5 |
| 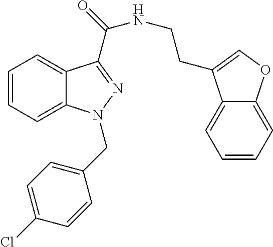 [SMILES: O=C(NCCc1coc2ccccc12)c1nn(Cc2ccc(Cl)cc2)c2ccccc12] | 356 | 0.566 | 429.9 | 430.2 | [M + H]⁺ | 5 |
| 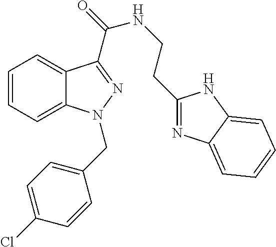 [SMILES: O=C(NCCc1nc2ccccc2[nH]1)c1nn(Cc2ccc(Cl)cc2)c2ccccc12] | 357 | 0.405 | 429.91 | 430.2 | [M + H]⁺ | 5 |
| 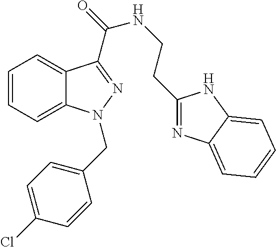 [SMILES: O=C(NCCc1nc2ccccc2[nH]1)c1nn(Cc2ccc(Cl)cc2)c2ccccc12] | 358 | 0.411 | 492.91 | 430.2 | [M + H]⁺ | 5 |
| 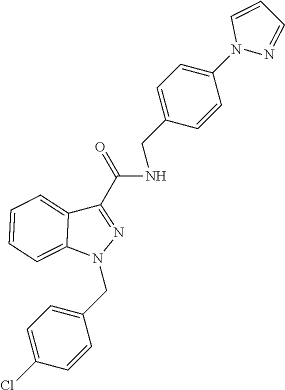 [SMILES: O=C(NCc1ccc(-n2cccn2)cc1)c1nn(Cc2ccc(Cl)cc2)c2ccccc12] | 359 | 0.533 | 441.92 | 442.2 | [M + H]⁺ | 5 |
| 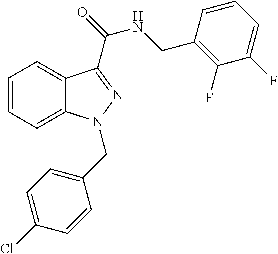 [SMILES: O=C(NCc1cccc(F)c1F)c1nn(Cc2ccc(Cl)cc2)c2ccccc12] | 360 | 0.557 | 411.84 | 412.1 | [M + H]⁺ | 5 |
| 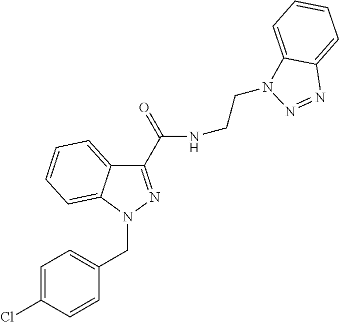 [SMILES: O=C(NCCn1nnc2ccccc21)c1nn(Cc2ccc(Cl)cc2)c2ccccc12] | 361 | 0.507 | 430.9 | 431.1 | [M + H]⁺ | 5 |
| 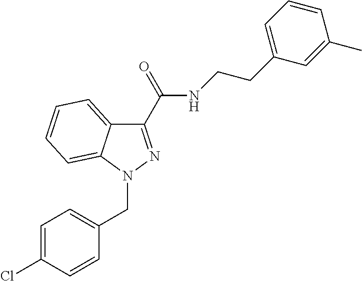 [SMILES: Cc1cccc(CCNC(=O)c2nn(Cc3ccc(Cl)cc3)c3ccccc23)c1] | 362 | 0.58 | 403.91 | 404.1 | [M + H]⁺ | 5 |
| 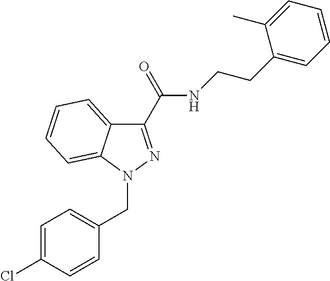 [SMILES: Cc1ccccc1CCNC(=O)c1nn(Cc2ccc(Cl)cc2)c2ccccc12] | 363 | 0.57 | 403.91 | 404.3 | [M + H]⁺ | 5 |
| 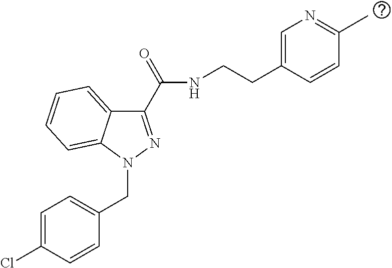 | 364 | 0.491 | 420.9 | 421.2 | [M + H]⁺ | 5 |
| 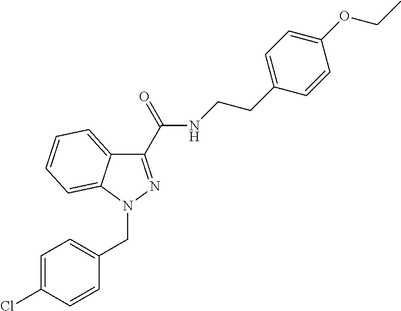 [SMILES: CCOc1ccc(CCNC(=O)c2nn(Cc3ccc(Cl)cc3)c3ccccc23)cc1] | 365 | 0.57 | 433.94 | 434.1 | [M + H]⁺ | 5 |
| 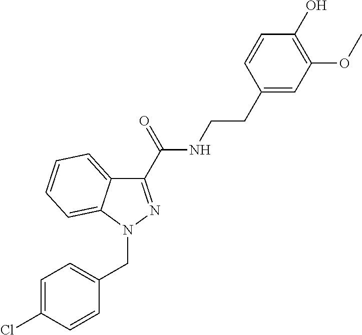 [SMILES: COc1cc(CCNC(=O)c2nn(Cc3ccc(Cl)cc3)c3ccccc23)ccc1O] | 366 | 0.53 | 435.91 | 436.2 | [M + H]⁺ | 6 |
| 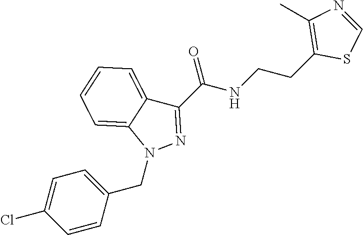 [SMILES: Cc1ncsc1CCNC(=O)c1nn(Cc2ccc(Cl)cc2)c2ccccc12] | 367 | 0.468 | 410.92 | 411.1 | [M + H]⁺ | 5 |
| 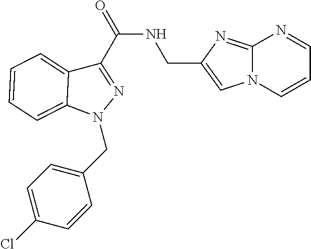 [SMILES: O=C(NCc1cn2cccnc2n1)c1nn(Cc2ccc(Cl)cc2)c2ccccc12] | 368 | 0.381 | 4A.87 | 417.1 | [M + H]⁺ | 5 |
| 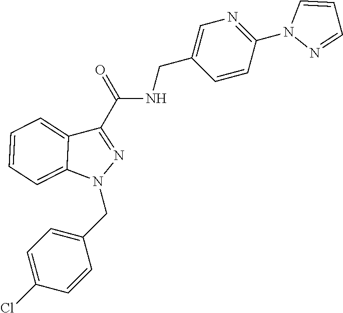 [SMILES: O=C(NCc1ccc(-n2cccn2)nc1)c1nn(Cc2ccc(Cl)cc2)c2ccccc12] | 369 | 0.527 | 442.91 | 443.2 | [M + H]⁺ | 5 |
| 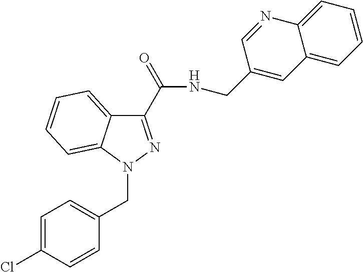 [SMILES: O=C(NCc1cnc2ccccc2c1)c1nn(Cc2ccc(Cl)cc2)c2ccccc12] | 370 | 0.432 | 426.9 | 427.1 | [M + H]⁺ | 5 |
| 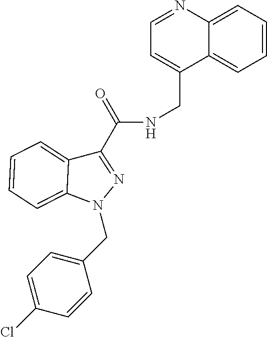 [SMILES: O=C(NCc1ccnc2ccccc12)c1nn(Cc2ccc(Cl)cc2)c2ccccc12] | 371 | 0.418 | 426.9 | 427.1 | [M + H]⁺ | 5 |
| 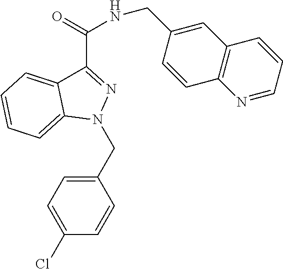 [SMILES: O=C(NCc1ccc2ncccc2c1)c1nn(Cc2ccc(Cl)cc2)c2ccccc12] | 372 | 0.435 | 426.9 | 427.2 | [M + H]⁺ | 6 |
| 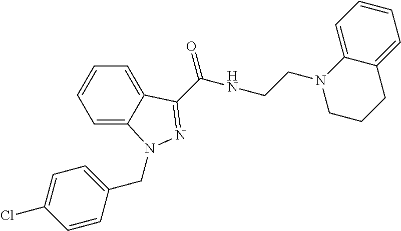 [SMILES: O=C(NCCN1CCCc2ccccc21)c1nn(Cc2ccc(Cl)cc2)c2ccccc12] | 373 | 0.568 | 444.96 | 445.2 | [M + H]⁺ | 5 |
| 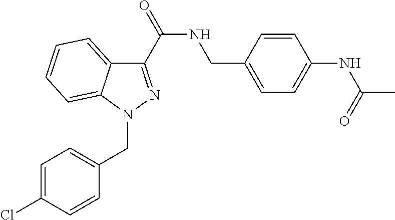 [SMILES: CC(=O)Nc1ccc(CNC(=O)c2nn(Cc3ccc(Cl)cc3)c3ccccc23)cc1] | 374 | 0.487 | 432.91 | 433.1 | [M + H]⁺ | 5 |
| 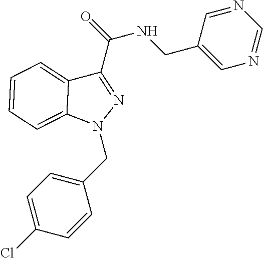 [SMILES: O=C(NCc1cncnc1)c1nn(Cc2ccc(Cl)cc2)c2ccccc12] | 375 | 0.459 | 377.83 | 378.1 | [M + H]⁺ | 5 |
| 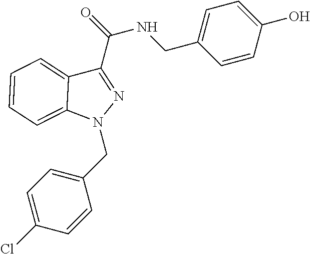 [SMILES: O=C(NCc1ccc(O)cc1)c1nn(Cc2ccc(Cl)cc2)c2ccccc12] | 376 | 0.494 | 391.86 | 392.1 | [M + H]⁺ | 5 |
| 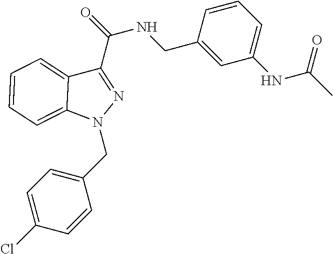 [SMILES: CC(=O)Nc1cccc(CNC(=O)c2nn(Cc3ccc(Cl)cc3)c3ccccc23)c1] | 377 | 0.498 | 432.91 | 433.3 | [M + H]⁺ | 5 |
| 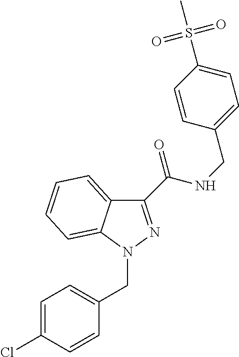 [SMILES: CS(=O)(=O)c1ccc(CNC(=O)c2nn(Cc3ccc(Cl)cc3)c3ccccc23)cc1] | 378 | 0.497 | 453.94 | 454.2 | [M + H]⁺ | 5 |
| 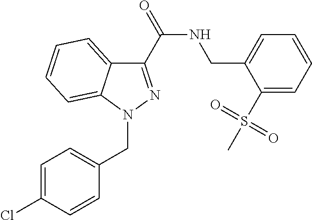 [SMILES: CS(=O)(=O)c1ccccc1CNC(=O)c1nn(Cc2ccc(Cl)cc2)c2ccccc12] | 379 | 0.524 | 453.94 | 454.1 | [M + H]⁺ | 5 |
| 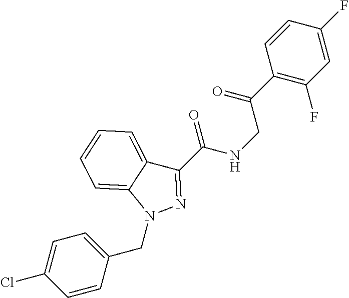 [SMILES: O=C(CNC(=O)c1nn(Cc2ccc(Cl)cc2)c2ccccc12)c1ccc(F)cc1F] | 380 | 0.541 | 439.85 | 440 | [M + H]⁺ | 5 |
| 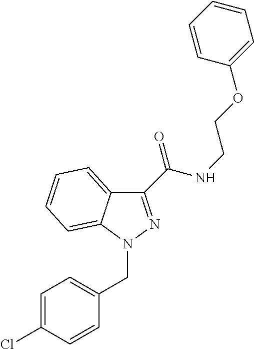 [SMILES: O=C(NCCOc1ccccc1)c1nn(Cc2ccc(Cl)cc2)c2ccccc12] | 381 | 0.549 | 405.88 | 406.3 | [M + H]⁺ | 5 |
| 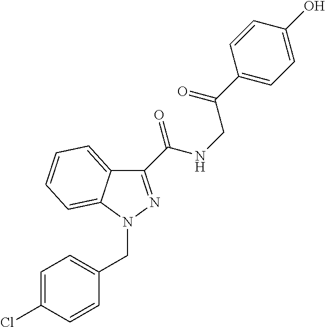 [SMILES: O=C(CNC(=O)c1nn(Cc2ccc(Cl)cc2)c2ccccc12)c1ccc(O)cc1] | 382 | 0.505 | 419.87 | 420.1 | [M + H]⁺ | 5 |
| 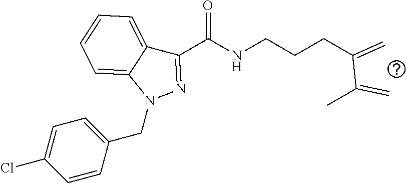 | 383 | 0.526 | 422.91 | 423.2 | [M + H]⁺ | 5 |
| 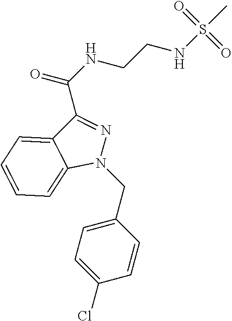 [SMILES: CS(=O)(=O)NCCNC(=O)c1nn(Cc2ccc(Cl)cc2)c2ccccc12] | 384 | 0.448 | 406.89 | 407.1 | [M + H]⁺ | 5 |
| 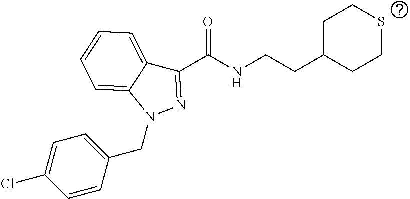 [SMILES: O=C(NCCC1CCSCC1)c1nn(Cc2ccc(Cl)cc2)c2ccccc12] | 385 | 0.48 | 445.96 | 446.1 | [M + H]⁺ | 5 |
| 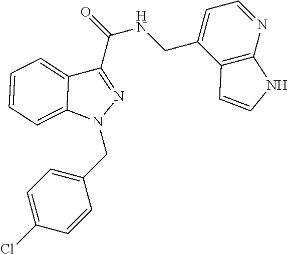 [SMILES: O=C(NCc1ccnc2[nH]ccc12)c1nn(Cc2ccc(Cl)cc2)c2ccccc12] | 386 | 0.418 | 415.88 | 4A.1 | [M + H]⁺ | 5 |
| 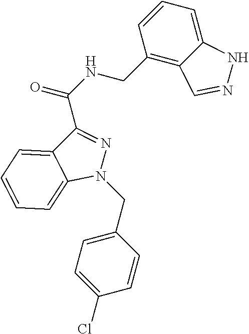 [SMILES: O=C(NCc1cccc2[nH]ncc12)c1nn(Cc2ccc(Cl)cc2)c2ccccc12] | 387 | 0.499 | 415.88 | 4A.1 | [M + H]⁺ | 5 |
| 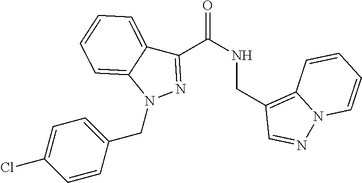 [SMILES: O=C(NCc1cnn2ccccc12)c1nn(Cc2ccc(Cl)cc2)c2ccccc12] | 388 | 0.513 | 415.88 | 4A.2 | [M + H]⁺ | 5 |
| 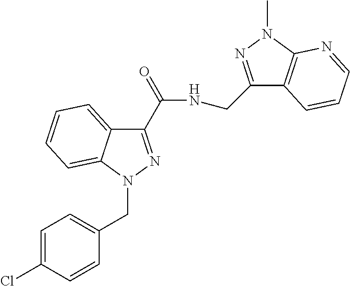 [SMILES: Cn1nc(CNC(=O)c2nn(Cc3ccc(Cl)cc3)c3ccccc23)c2cccnc21] | 389 | 0.515 | 430.9 | 431.2 | [M + H]⁺ | 5 |
| 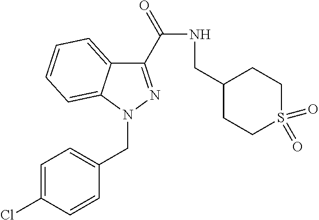 [SMILES: O=C(NCC1CCS(=O)(=O)CC1)c1nn(Cc2ccc(Cl)cc2)c2ccccc12] | 390 | 0.53 | 431.94 | 432.1 | [M + H]⁺ | 5 |
| 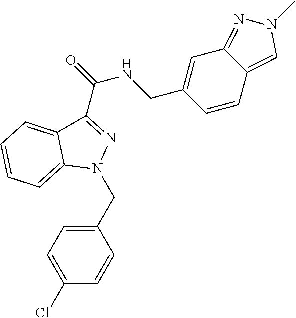 [SMILES: Cn1cc2ccc(CNC(=O)c3nn(Cc4ccc(Cl)cc4)c4ccccc34)cc2n1] | 391 | 0.499 | 429.91 | 430.1 | [M + H]⁺ | 5 |
| 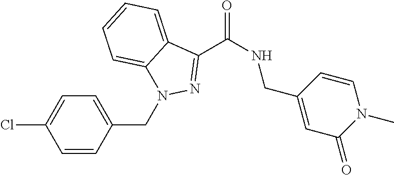 [SMILES: Cn1ccc(CNC(=O)c2nn(Cc3ccc(Cl)cc3)c3ccccc23)cc1=O] | 392 | 0.454 | 406.87 | 407.1 | [M + H]⁺ | 5 |
| 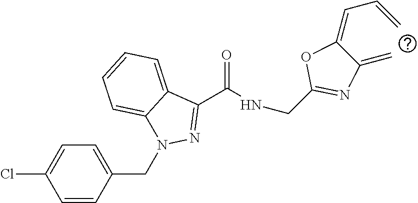 [SMILES: C=C/C=c1/oc(CNC(=O)c2nn(Cc3ccc(Cl)cc3)c3ccccc23)nc1=C] | 393 | 0.555 | 416.87 | 417.2 | [M + H]⁺ | 6 |
| 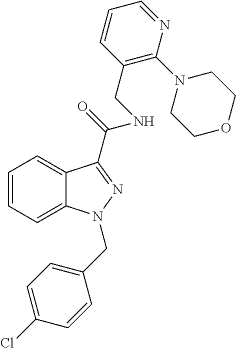 [SMILES: O=C(NCc1cccnc1N1CCOCC1)c1nn(Cc2ccc(Cl)cc2)c2ccccc12] | 395 | 0.452 | 461.95 | 462.2 | [M + H]⁺ | 6 |
| 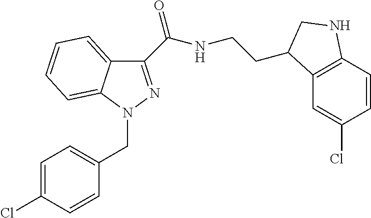 [SMILES: O=C(NCCC1CNc2ccc(Cl)cc21)c1nn(Cc2ccc(Cl)cc2)c2ccccc12] | 396 | 0.594 | 463.36 | 463.1 | [M + H]⁺ | 6 |
| 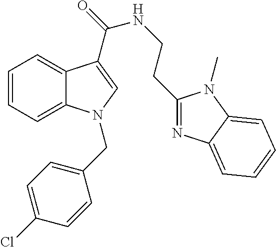 [SMILES: Cn1c(CCNC(=O)c2cn(Cc3ccc(Cl)cc3)c3ccccc23)nc2ccccc21] | 397 | 0.433 | 443.94 | 444.2 | [M + H]⁺ | 6 |
| 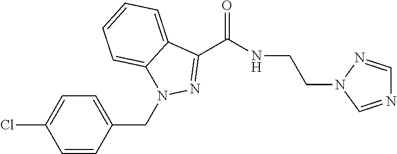 [SMILES: O=C(NCCn1cncn1)c1nn(Cc2ccc(Cl)cc2)c2ccccc12] | 398 | 0.456 | 380.84 | 381.2 | [M + H]⁺ | 6 |
| 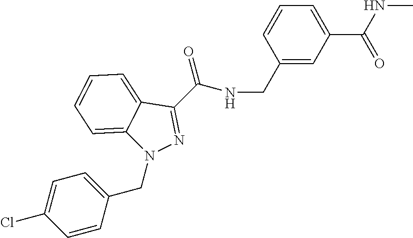 [SMILES: CNC(=O)c1cccc(CNC(=O)c2nn(Cc3ccc(Cl)cc3)c3ccccc23)c1] | 399 | 0.508 | 432.91 | 433.1 | [M + H]⁺ | 6 |
| 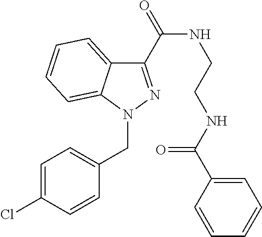 [SMILES: O=C(NCCNC(=O)c1nn(Cc2ccc(Cl)cc2)c2ccccc12)c1ccccc1] | 400 | 0.501 | 432.91 | 433.1 | [M + H]⁺ | 5 |
| 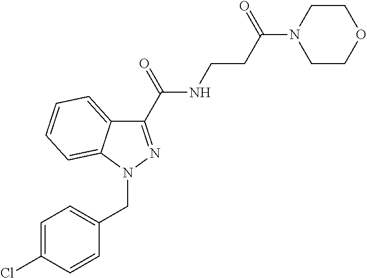 [SMILES: O=C(NCCC(=O)N1CCOCC1)c1nn(Cc2ccc(Cl)cc2)c2ccccc12] | 401 | 0.465 | 426.9 | 427.2 | [M + H]⁺ | 5 |
| 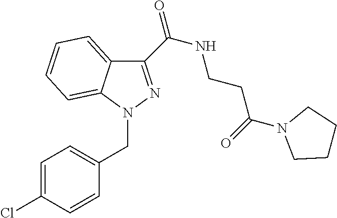 [SMILES: O=C(NCCC(=O)N1CCCC1)c1nn(Cc2ccc(Cl)cc2)c2ccccc12] | 403 | 0.488 | 410.9 | 411.2 | [M + H]⁺ | 5 |
| 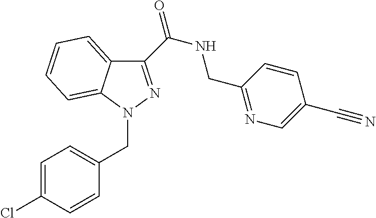 [SMILES: N#Cc1ccc(CNC(=O)c2nn(Cc3ccc(Cl)cc3)c3ccccc23)nc1] | 404 | 0.497 | 401.85 | 402.1 | [M + H]⁺ | 5 |
| 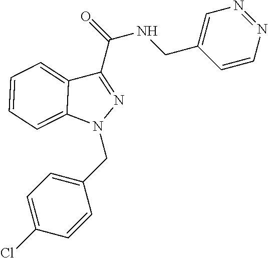 [SMILES: O=C(NCc1ccnnc1)c1nn(Cc2ccc(Cl)cc2)c2ccccc12] | 405 | 0.441 | 377.83 | 378.1 | [M + H]⁺ | 5 |
| 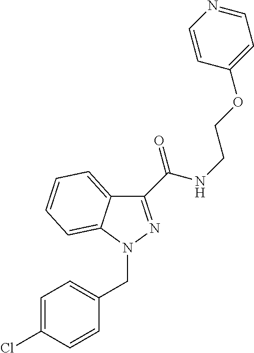 [SMILES: O=C(NCCOc1ccncc1)c1nn(Cc2ccc(Cl)cc2)c2ccccc12] | 406 | 0.386 | 406.87 | 407.2 | [M + H]⁺ | 5 |
| 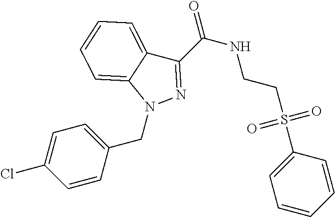 [SMILES: O=C(NCCS(=O)(=O)c1ccccc1)c1nn(Cc2ccc(Cl)cc2)c2ccccc12] | 407 | 0.532 | 453.94 | 454.1 | [M + H]⁺ | 6 |
| 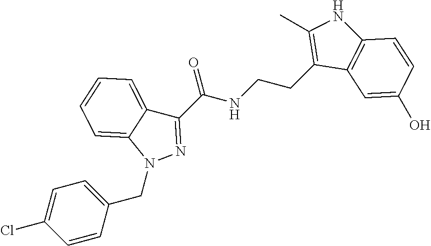 [SMILES: Cc1[nH]c2ccc(O)cc2c1CCNC(=O)c1nn(Cc2ccc(Cl)cc2)c2ccccc12] | 416 | 0.563 | 458.95 | 459.1 | [M + H]⁺ | 5 |
| 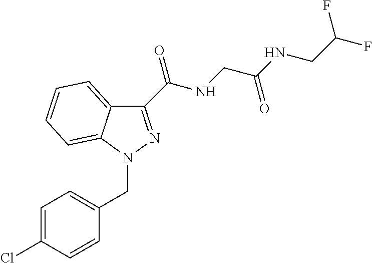 [SMILES: O=C(CNC(=O)c1nn(Cc2ccc(Cl)cc2)c2ccccc12)NCC(F)F] | 418 | 0.467 | 406.82 | 407.2 | [M + H]⁺ | 5 |
| 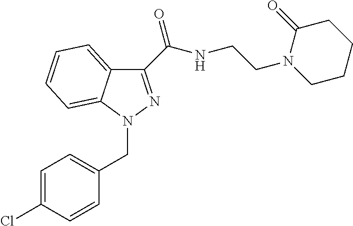 [SMILES: O=C(NCCN1CCCCC1=O)c1nn(Cc2ccc(Cl)cc2)c2ccccc12] | 419 | 0.534 | 410.9 | 411.1 | [M + H]⁺ | 6 |
| 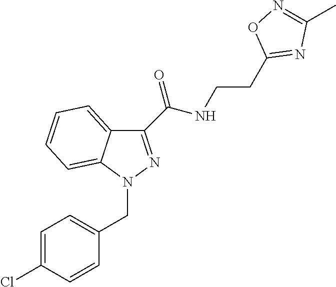 [SMILES: Cc1noc(CCNC(=O)c2nn(Cc3ccc(Cl)cc3)c3ccccc23)n1] | 420 | 0.484 | 395.85 | 396.2 | [M + H]⁺ | 5 |
| 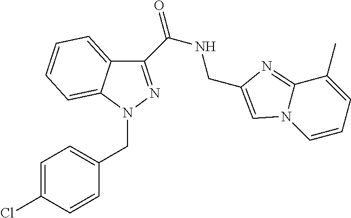 [SMILES: Cc1cccn2cc(CNC(=O)c3nn(Cc4ccc(Cl)cc4)c4ccccc34)nc12] | 421 | 0.403 | 429.91 | 430.1 | [M + H]⁺ | 5 |
| 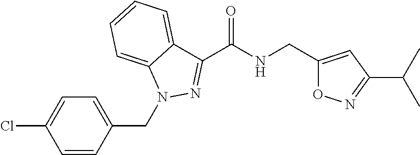 [SMILES: CC(C)c1cc(CNC(=O)c2nn(Cc3ccc(Cl)cc3)c3ccccc23)on1] | 422 | 0.525 | 408.89 | 409.1 | [M + H]⁺ | 5 |
| 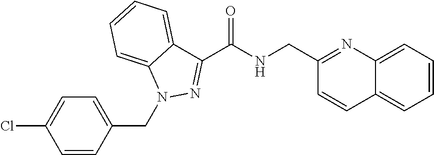 [SMILES: O=C(NCc1ccc2ccccc2n1)c1nn(Cc2ccc(Cl)cc2)c2ccccc12] | 423 | 0.457 | 426.9 | 427.1 | [M + H]⁺ | 5 |
| 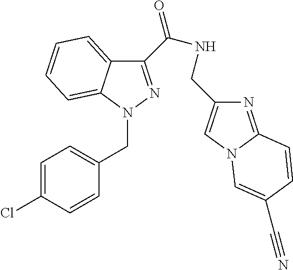 [SMILES: N#Cc1ccc2nc(CNC(=O)c3nn(Cc4ccc(Cl)cc4)c4ccccc34)cn2c1] | 424 | 0.425 | 440.89 | 441.2 | [M + H]⁺ | 5 |
| 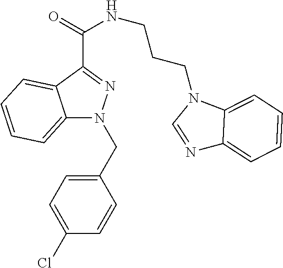 [SMILES: O=C(NCCCn1cnc2ccccc21)c1nn(Cc2ccc(Cl)cc2)c2ccccc12] | 425 | 0.409 | 443.94 | 444.3 | [M + H]⁺ | 5 |
| 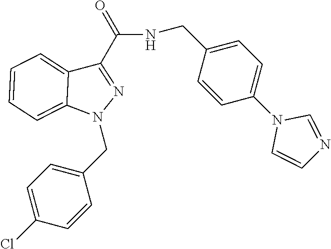 [SMILES: O=C(NCc1ccc(-n2ccnc2)cc1)c1nn(Cc2ccc(Cl)cc2)c2ccccc12] | 426 | 0.408 | 441.92 | 442.2 | [M + H]⁺ | 5 |
| 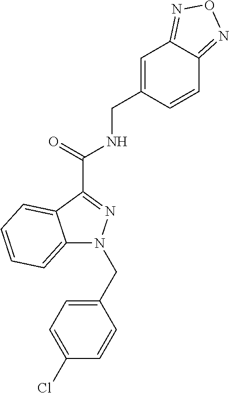 [SMILES: O=C(NCc1ccc2nonc2c1)c1nn(Cc2ccc(Cl)cc2)c2ccccc12] | 427 | 0.533 | 417.85 | 418.2 | [M + H]⁺ | 5 |
| 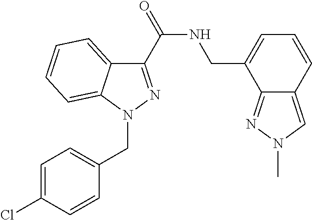 [SMILES: Cn1cc2cccc(CNC(=O)c3nn(Cc4ccc(Cl)cc4)c4ccccc34)c2n1] | 428 | 0.5A | 429.91 | 430.2 | [M + H]⁺ | 5 |
| 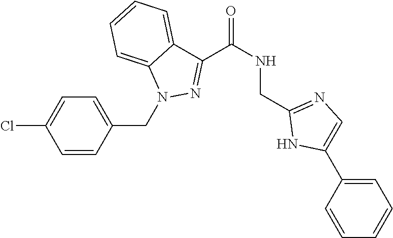 [SMILES: O=C(NCc1ncc(-c2ccccc2)[nH]1)c1nn(Cc2ccc(Cl)cc2)c2ccccc12] | 429 | 0.415 | 441.92 | 442.3 | [M + H]⁺ | 5 |
| 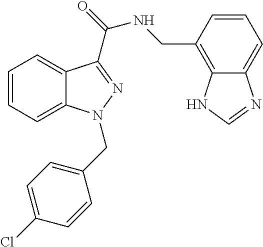 [SMILES: O=C(NCc1cccc2nc[nH]c12)c1nn(Cc2ccc(Cl)cc2)c2ccccc12] | 475 | 0.455 | 415.88 | 416.2 | [M + H]⁺ | 5 |

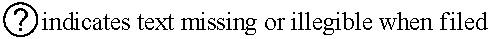

### Example 5

### Synthesis of Representative Compound 309

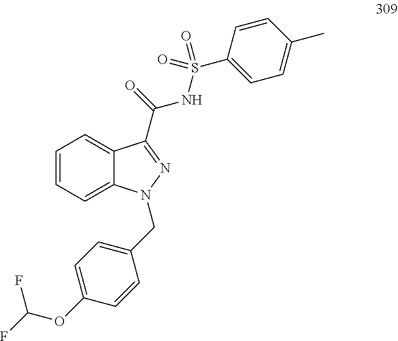 [SMILES: Cc1ccc(S(=O)(=O)NC(=O)c2nn(Cc3ccc(OC(F)F)cc3)c3ccccc23)cc1]

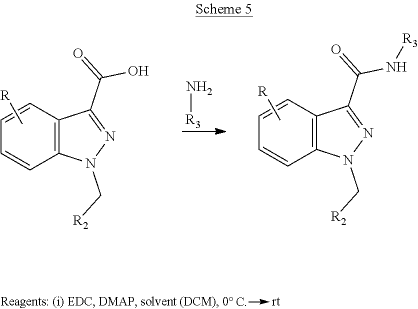

### Step 5-1. Synthesis of 1-(4-(difluoromethoxy)benzyl)-N-tosyl-1H-indazole-3-carboxamide (Compound 309)

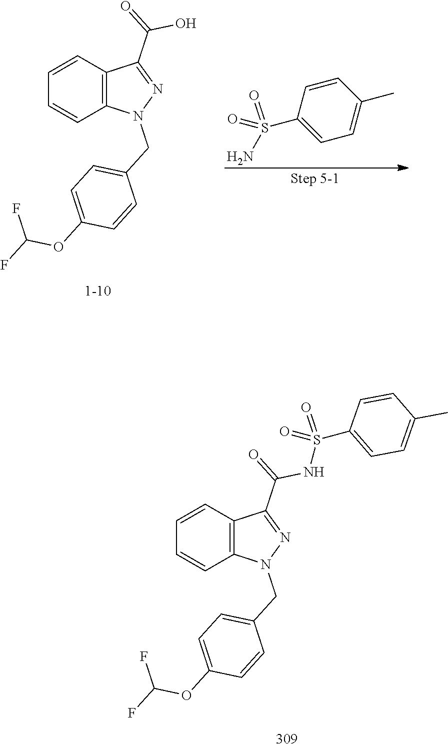 [SMILES: O=C(O)c1nn(Cc2ccc(OC(F)F)cc2)c2ccccc12 ; Cc1ccc(S(N)(=O)=O)cc1 ; Cc1ccc(S(=O)(=O)NC(=O)c2nn(Cc3ccc(OC(F)F)cc3)c3ccccc23)cc1]

To a solution of 1-Ethyl-3-(3-dimethylaminopropyl)carbodiimide (EDC) (78 mg, 1.3 Eq, 408 μmol and 4-dimethlaminopyridine (DMAP) (54 mg, 1.4 Eq, 440 μmol) in DCM (1 mL) at 0° C. was added 1-(4-(difluoromethoxy)benzyl)-1H-indazole-3-carboxylic acid (1-10) (100 mg, 1 Eq, 314 μmol). After stirring for 5 minutes, p-toluenesulfonamide (65 mg, 1.2 Eq, 377 μmol) was added. The reaction mixture was warmed to room temperature and stirred overnight. After stirring for 17 hours, The reaction mixture was purified by silica gel column chromatography (0->10% MeOH in DCM) and subsequently lyophilized in MeOH/H₂O (4 mL 1:1) to afford 71 mg (48% yield) of 1-(4-(difluoromethoxy)benzyl)-N-tosyl-1H-indazole-3-carboxamide (Compound 309); LCMS (m/z) calculated for C₂₃H₁₉F₂N₃O₄S: 471.1; found 472.2 [M+H]⁺, t_{R}=10.718 min (Method 2).

The compounds listed in Table 5 were made using the procedures of Scheme 5.

TABLE 5

| | Cpd | Purity | | Observed | | Purity |
|---|---|---|---|---|---|---|
| Structure | No. | RT (min) | MW | m/z | Ion | Method |
| 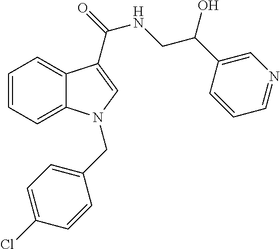 [SMILES: O=C(NCC(O)c1cccnc1)c1cn(Cc2ccc(Cl)cc2)c2ccccc12] | 150 | 11.37 | 405.1 | 406.4 | [M + H]⁺ | 3 |
| 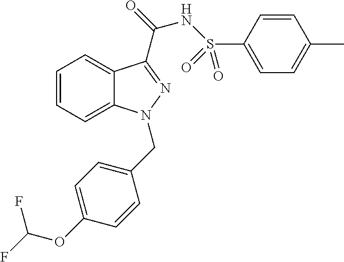 [SMILES: Cc1ccc(S(=O)(=O)NC(=O)c2nn(Cc3ccc(OC(F)F)cc3)c3ccccc23)cc1] | 309 | 10.718 | 471.1 | 472.2 | [M + H]⁺ | 2 |
| 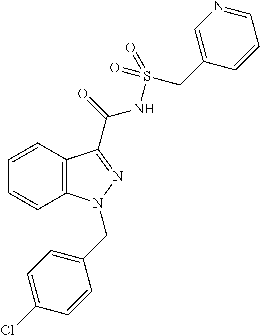 [SMILES: O=C(NS(=O)(=O)Cc1cccnc1)c1nn(Cc2ccc(Cl)cc2)c2ccccc12] | 310 | 3.63 | 440.1 | 440.1 | [M] | 4 |

### Example 6

### Synthesis of Representative Compound 152

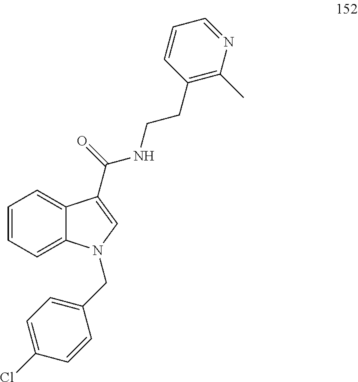 [SMILES: Cc1ncccc1CCNC(=O)c1cn(Cc2ccc(Cl)cc2)c2ccccc12]

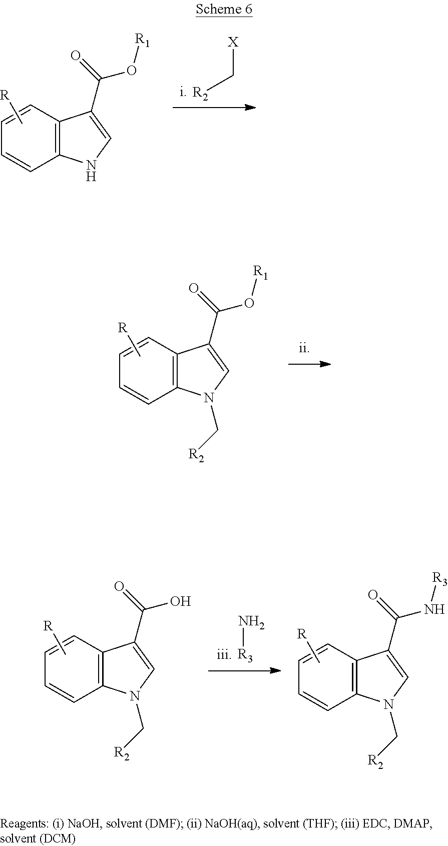

### Step 6-1. methyl 1-(4-chlorobenzyl)-1H-indole-3-carboxylate (INT-6A)

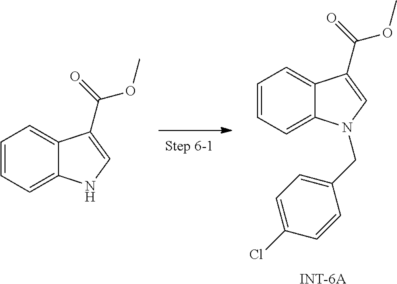

A 250 mL round bottom flask containing methyl 1H-indole-3-carboxylate (4.39 g, 1.0 equiv., 37.4 mmol) in DMF (30 mL) was added NaH (60% dispersion in mineral oil, 2.04 g, 1.4 equiv., 51.1 mmol) and then the suspension was stirred at rt for 5 minutes, 1-(bromomethyl)-4-chlorobenzene (7.0 g, 1.3 equiv., 34.1 mmol) was slowly added.

Stirred at rt for 20 minutes. Monitored by LCMS. 20 min: Coupling complete. Quenched with NH₄Cl (100 mL) slowly. Product was extracted with EtOAc (300 mL), washed with brine (200 mL), dried over Na₂SO₄, filtered, and concentrated in vacuo to afford 8.0 g (78% yield) of methyl 1-(4-chlorobenzyl)-1H-indole-3-carboxylate (INT-6A); LCMS (m/z) calculated for C₁₇H₁₄ClNO₂: 299.07; found 300.2 [M+H]⁺, t_{R}=5.37 min (Method 3).

### Step 6-2. 1-(4-chlorobenzyl)-1H-indole-3-carboxylic acid (INT-6B)

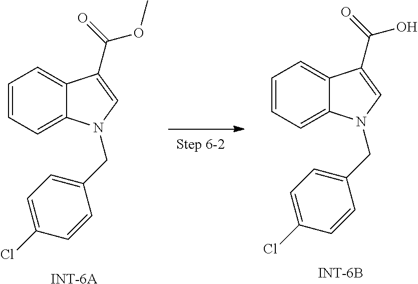

To a 250 mL round bottom flask containing INT-6A (8.0 g, 1.0 equiv., 26.7 mmol) in MeOH (100 mL) was added NaOH (3.2 g, 1.0 equiv., 80.1 mmol) dissolved in H₂O (10 mL). The mixture was stirred at rt for 20 h. Monitored by LCMS. No reaction occurred. Additional NaOH (1 g, 0.94 equiv., 25.0 mmol) was added to the reaction and the reaction was heated to 80° C. for 20 h. Concentrated to remove solvent and diluted with H₂O (200 mL). The aqueous layer was acidified with 1M HCl. Product was extracted with EtOAc (300 mL), washed with brine (100 mL), dried over Na₂SO₄, filtered, and concentrated. Diluted with DCM and filtered, collecting the solid to afford 4.0 g (53% yield) 1-(4-chlorobenzyl)-1H-indole-3-carboxylic acid (INT-6B); LCMS (m/z) calculated for C₁₆H₁₂ClNO₂: 285.06; found 286.1 [M+H]⁺, t_{R}=4.82 min (Method 3). ¹H NMR (400 MHz, DMSO-d₆) δ 12.81 (s, 1H), δ 8.24 (s, 1H), 8.03 (t, J=4 Hz, 1H), 7.52 (t, J=4 Hz, 1H), 7.40 (d, J=8 Hz, 2H), 7.30 (d, J=8 Hz, 2H), 7.20 (t, J=4 Hz, 2H), 5.51 (s, 2H).

### Step 6-3. 1-(4-chlorobenzyl)-N-(2-(2-methylpyridin-3-yl)ethyl)-1H-indole-3-carboxamide (Compound 152)

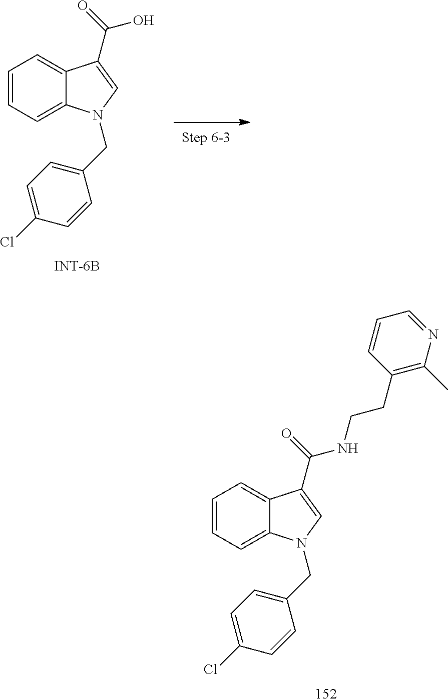

To a solution of INT-6B (30 mg, 1.0 equiv., 0.1 mmol) in DCM (4 ml) was added 2-(2-methylpyridin-3-yl)ethan-1-amine (15.7 mg, 0.12 equiv., 0.12 mmol), EDCI (17.9 mg, 0.12 equiv., 0.0115 mmol) and DMAP (14.2 mg, 0.12 equiv., 0.12 mmol) at rt for overnight. The reaction mixture was diluted with DCM and water. The DCM layer was separated and dried, concentrated and purified by silica gel column chromatography (0-100% EtOAc/Hexane first and then 0-10% MeOH/DCM) to afford 27 mg (64% yield) of 1-(4-chlorobenzyl)-N-(2-(2-methylpyridin-3-yl)ethyl)-1H-indole-3-carboxamide (Compound 152) as a white solid; LCMS (m/z) calculated for C₂₄H₂₂ClN₃O: 403.15; found 404.5 [M+H]⁺, t_{R}=11.27 min (Method 3). ¹H NMR (400 MHz, DMSO-d6) δ 8.29 (s, 1H), 8.13 (d, J=4 Hz, 1H), 8.0 (m, 2H), 7.52 (m, 2H), 7.41 (d, J=8 Hz, 2H), 7.23 (d, J=8 Hz, 2H), 7.13 (m, 3H), 5.46 (s, 2H), 3.47 (m, 2H), 2.87 (m, 2H), 2.53 (s, 3H).

The compounds listed in Table 6 were made using the procedures of Scheme 6.

TABLE 6

| | | Purity | | Observed | | Purity |
|---|---|---|---|---|---|---|
| Structure | Cpd No. | RT (min) | NM | m/z | Ion | Method |
| 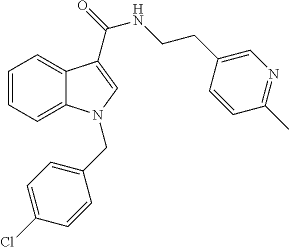 [SMILES: Cc1ccc(CCNC(=O)c2cn(Cc3ccc(Cl)cc3)c3ccccc23)cn1] | 151 | 11.5 | 403.91 | 404.6 | [M + H]⁺ | 3 |
| 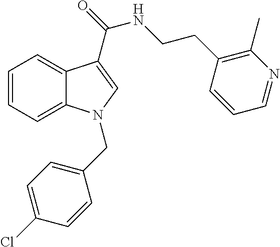 [SMILES: Cc1ncccc1CCNC(=O)c1cn(Cc2ccc(Cl)cc2)c2ccccc12] | 152 | 11.27 | 403.91 | 404.5 | [M + H]⁺ | 3 |
| 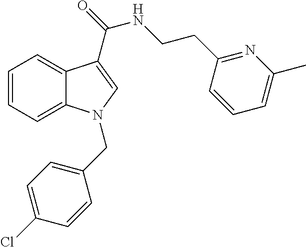 [SMILES: Cc1cccc(CCNC(=O)c2cn(Cc3ccc(Cl)cc3)c3ccccc23)n1] | 153 | 11.63 | 403.91 | 404.5 | [M + H]⁺ | 3 |
| 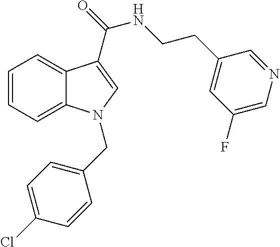 [SMILES: O=C(NCCc1cncc(F)c1)c1cn(Cc2ccc(Cl)cc2)c2ccccc12] | 154 | 12.95 | 407.87 | 408.3 | [M + H]⁺ | 3 |
| 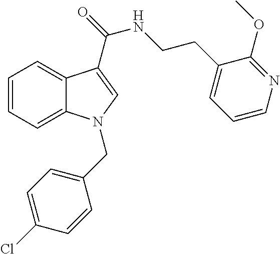 [SMILES: COc1ncccc1CCNC(=O)c1cn(Cc2ccc(Cl)cc2)c2ccccc12] | 155 | 13.36 | 419.91 | 420.2 | [M + H]⁺ | 3 |
| 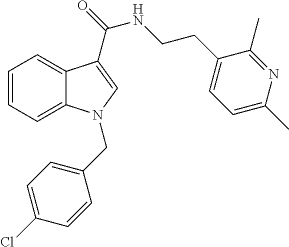 [SMILES: Cc1ccc(CCNC(=O)c2cn(Cc3ccc(Cl)cc3)c3ccccc23)c(C)n1] | 156 | 11.4 | 417.94 | 418.5 | [M + H]⁺ | 3 |
| 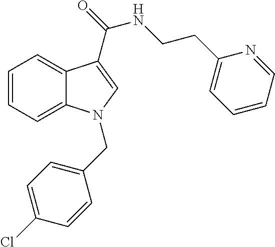 [SMILES: O=C(NCCc1ccccn1)c1cn(Cc2ccc(Cl)cc2)c2ccccc12] | 157 | 11.25 | 389.88 | 390.5 | [M + H]⁺ | 3 |
| 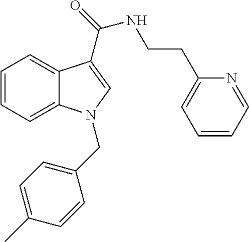 [SMILES: Cc1ccc(Cn2cc(C(=O)NCCc3ccccn3)c3ccccc32)cc1] | 158 | 95 | 369.468 | 370.3 | [M + H]⁺ | 3 |
| 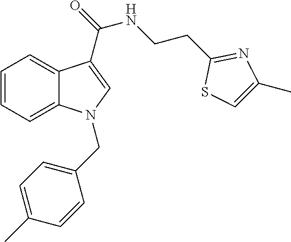 [SMILES: Cc1ccc(Cn2cc(C(=O)NCCc3nc(C)cs3)c3ccccc32)cc1] | 159 | 12.92 | 389.52 | 390.5 | [M + H]⁺ | 3 |
| 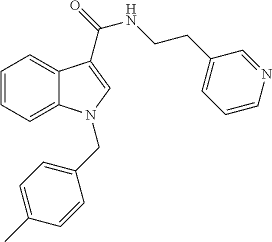 [SMILES: Cc1ccc(Cn2cc(C(=O)NCCc3cccnc3)c3ccccc32)cc1] | 160 | 11.29 | 369.468 | 370.4 | [M + H]⁺ | 3 |
| 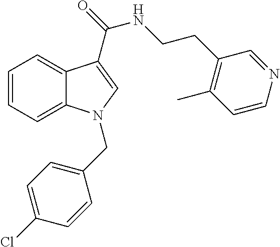 [SMILES: Cc1ccncc1CCNC(=O)c1cn(Cc2ccc(Cl)cc2)c2ccccc12] | 161 | 11.58 | 403.91 | 404.5 | [M + H]⁺ | 3 |
| 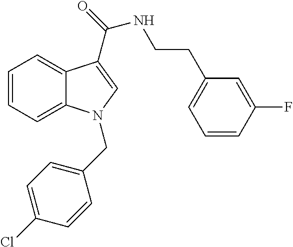 [SMILES: O=C(NCCc1cccc(F)c1)c1cn(Cc2ccc(Cl)cc2)c2ccccc12] | 162 | 13.66 | 406.89 | 407.3 | [M + H]⁺ | 3 |
| 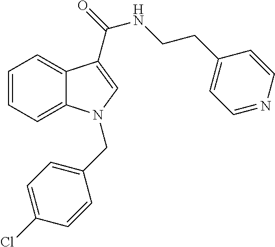 [SMILES: O=C(NCCc1ccncc1)c1cn(Cc2ccc(Cl)cc2)c2ccccc12] | 163 | 11.33 | 389.88 | 390.5 | [M + H]⁺ | 3 |
| 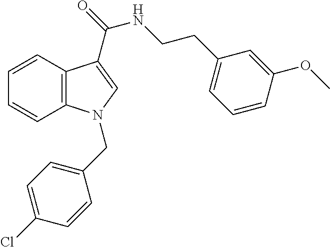 [SMILES: COc1cccc(CCNC(=O)c2cn(Cc3ccc(Cl)cc3)c3ccccc23)c1] | 164 | 13.6 | 418.92 | 419.4 | [M + H]⁺ | 3 |
| 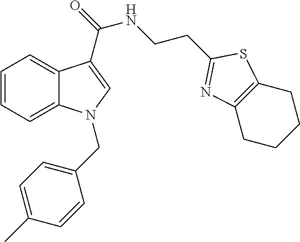 [SMILES: Cc1ccc(Cn2cc(C(=O)NCCc3nc4c(s3)CCCC4)c3ccccc32)cc1] | 165 | 13.63 | 429.58 | 430.7 | [M + H]⁺ | 3 |
| 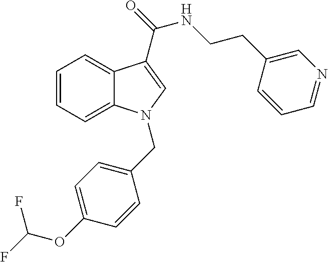 [SMILES: O=C(NCCc1cccnc1)c1cn(Cc2ccc(OC(F)F)cc2)c2ccccc12] | 166 | 11.83 | 421.448 | 422.2 | [M + H]⁺ | 3 |
| 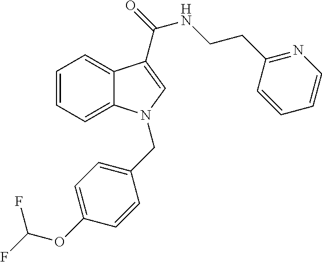 [SMILES: O=C(NCCc1ccccn1)c1cn(Cc2ccc(OC(F)F)cc2)c2ccccc12] | 167 | 10.91 | 457.908 | 422.2 | [M + H]⁺ | 3 |
| 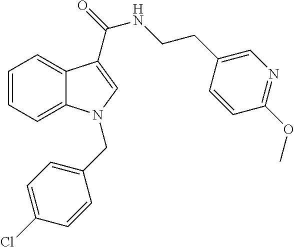 [SMILES: COc1ccc(CCNC(=O)c2cn(Cc3ccc(Cl)cc3)c3ccccc23)cn1] | 168 | 12.93 | 419.91 | 420.4 | [M + H]⁺ | 3 |
| 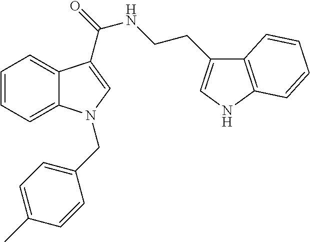 [SMILES: Cc1ccc(Cn2cc(C(=O)NCCc3c[nH]c4ccccc34)c3ccccc32)cc1] | 169 | 13.43 | 407.517 | 408.4 | [M + H]⁺ | 3 |
| 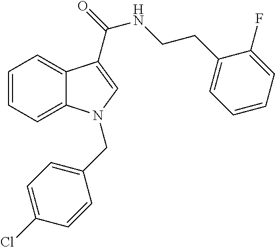 [SMILES: O=C(NCCc1ccccc1F)c1cn(Cc2ccc(Cl)cc2)c2ccccc12] | 170 | 13.56 | 406.89 | 407.5 | [M + H]⁺ | 3 |
| 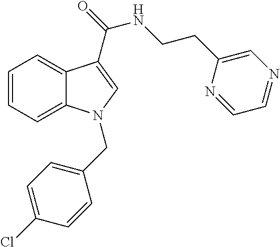 [SMILES: O=C(NCCc1cnccn1)c1cn(Cc2ccc(Cl)cc2)c2ccccc12] | 171 | 12.64 | 390.87 | 391.5 | [M + H]⁺ | 3 |
| 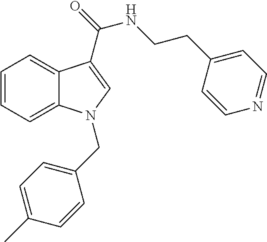 [SMILES: Cc1ccc(Cn2cc(C(=O)NCCc3ccncc3)c3ccccc32)cc1] | 172 | 11.17 | 369.468 | 370.4 | [M + H]⁺ | 3 |
| 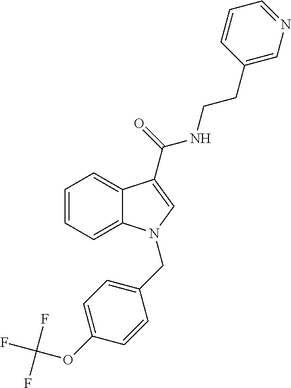 [SMILES: O=C(NCCc1cccnc1)c1cn(Cc2ccc(OC(F)(F)F)cc2)c2ccccc12] | 173 | 11.8 | 439.438 | 440.2 | [M + H]⁺ | 3 |
| 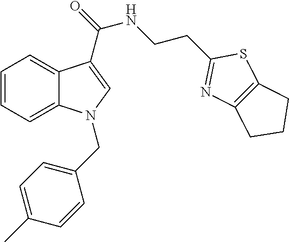 [SMILES: Cc1ccc(Cn2cc(C(=O)NCCc3nc4c(s3)CCC4)c3ccccc32)cc1] | 174 | 13.78 | 415.56 | 416.6 | [M + H]⁺ | 3 |
| 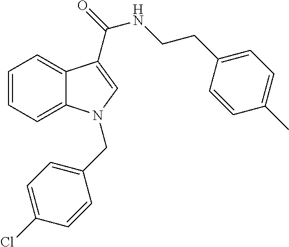 [SMILES: Cc1ccc(CCNC(=O)c2cn(Cc3ccc(Cl)cc3)c3ccccc23)cc1] | 175 | 14.01 | 402.92 | 403.5 | [M + H]⁺ | 3 |
| 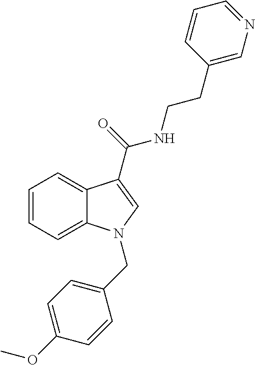 [SMILES: COc1ccc(Cn2cc(C(=O)NCCc3cccnc3)c3ccccc32)cc1] | 176 | 11.16 | 385.467 | 386.3 | [M + H]⁺ | 3 |
| 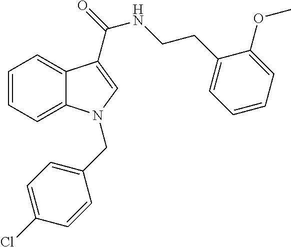 [SMILES: COc1ccccc1CCNC(=O)c1cn(Cc2ccc(Cl)cc2)c2ccccc12] | 177 | 13.84 | 418.92 | 419.3 | [M + H]⁺ | 3 |
| 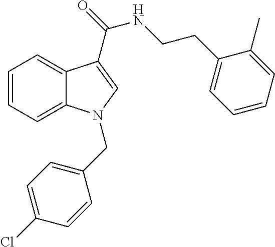 [SMILES: Cc1ccccc1CCNC(=O)c1cn(Cc2ccc(Cl)cc2)c2ccccc12] | 178 | 13.97 | 402.92 | 403.6 | [M + H]⁺ | 3 |
| 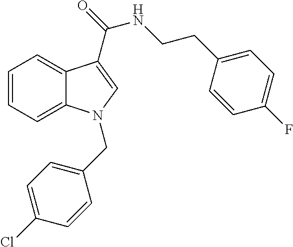 [SMILES: O=C(NCCc1ccc(F)cc1)c1cn(Cc2ccc(Cl)cc2)c2ccccc12] | 179 | 13.67 | 406.89 | 407.3 | [M + H]⁺ | 3 |
| 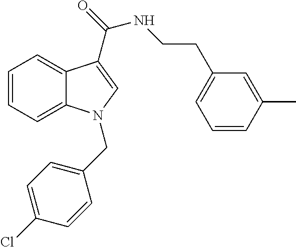 [SMILES: Cc1cccc(CCNC(=O)c2cn(Cc3ccc(Cl)cc3)c3ccccc23)c1] | 180 | 14.08 | 402.92 | 403.6 | [M + H]⁺ | 3 |
| 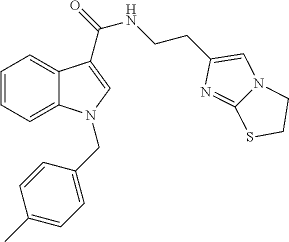 [SMILES: Cc1ccc(Cn2cc(C(=O)NCCc3cn4c(n3)SCC4)c3ccccc32)cc1] | 181 | 11.49 | 416.54 | 417.5 | [M + H]⁺ | 3 |
| 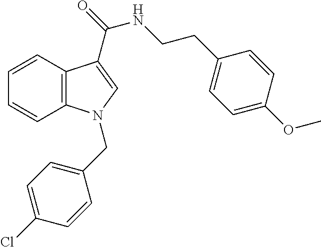 [SMILES: COc1ccc(CCNC(=O)c2cn(Cc3ccc(Cl)cc3)c3ccccc23)cc1] | 182 | 13.6 | 418.92 | 419 | [M + H]⁺ | 3 |
| 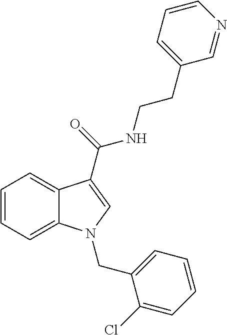 [SMILES: O=C(NCCc1cccnc1)c1cn(Cc2ccccc2Cl)c2ccccc12] | 183 | 11.65 | 389.88 | 390.5 | [M + H]⁺ | 3 |
| 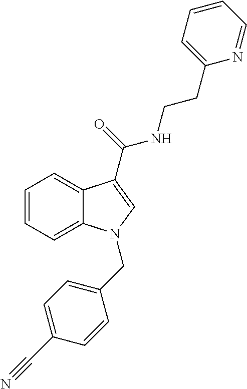 [SMILES: N#Cc1ccc(Cn2cc(C(=O)NCCc3ccccn3)c3ccccc32)cc1] | 184 | 9.93 | 380.45 | 381.3 | [M + H]⁺ | 3 |
| 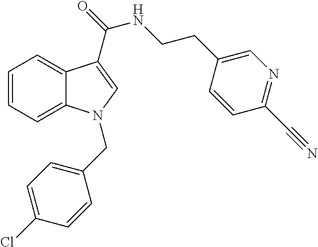 [SMILES: N#Cc1ccc(CCNC(=O)c2cn(Cc3ccc(Cl)cc3)c3ccccc23)cn1] | 185 | 12.84 | 414.89 | 415.4 | [M + H]⁺ | 3 |
| 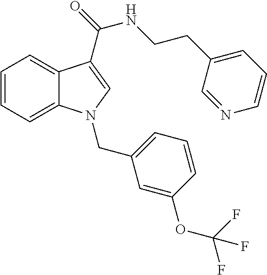 [SMILES: O=C(NCCc1cccnc1)c1cn(Cc2cccc(OC(F)(F)F)c2)c2ccccc12] | 186 | 11.58 | 439.43 | 440.3 | [M + H]⁺ | 3 |
| 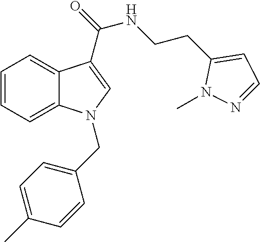 [SMILES: Cc1ccc(Cn2cc(C(=O)NCCc3ccnn3C)c3ccccc32)cc1] | 187 | 12.6 | 372.472 | 373.3 | [M + H]⁺ | 3 |
| 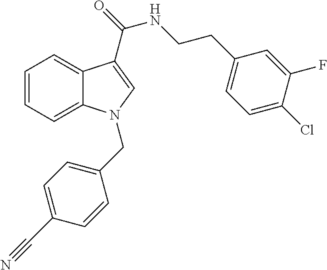 [SMILES: N#Cc1ccc(Cn2cc(C(=O)NCCc3ccc(Cl)c(F)c3)c3ccccc32)cc1] | 188 | 13.01 | 431.9 | 432.5 | [M + H]⁺ | 3 |
| 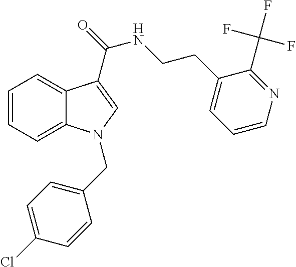 [SMILES: O=C(NCCc1cccnc1C(F)(F)F)c1cn(Cc2ccc(Cl)cc2)c2ccccc12] | 189 | 13.39 | 457.88 | 458.6 | [M + H]⁺ | 3 |
| 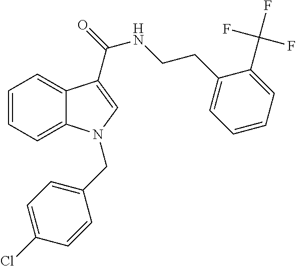 [SMILES: O=C(NCCc1ccccc1C(F)(F)F)c1cn(Cc2ccc(Cl)cc2)c2ccccc12] | 190 | 13.98 | 456.89 | 457.6 | [M + H]⁺ | 3 |
| 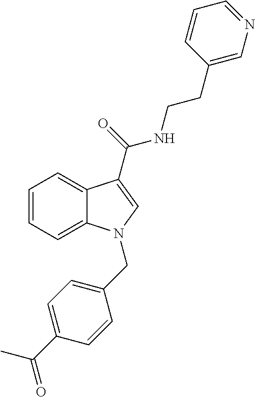 [SMILES: CC(=O)c1ccc(Cn2cc(C(=O)NCCc3cccnc3)c3ccccc32)cc1] | 191 | 10.17 | 397.478 | 398.4 | [M + H]⁺ | 3 |
| 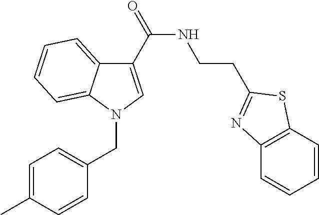 [SMILES: Cc1ccc(Cn2cc(C(=O)NCCc3nc4ccccc4s3)c3ccccc32)cc1] | 192 | 13.89 | 425.55 | 426.3 | [M + H]⁺ | 3 |
| 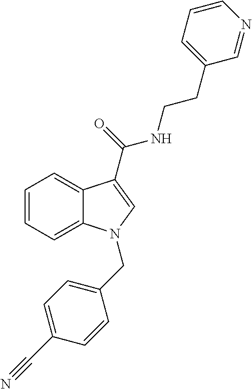 [SMILES: N#Cc1ccc(Cn2cc(C(=O)NCCc3cccnc3)c3ccccc32)cc1] | 193 | 10.07 | 380.451 | 381.3 | [M + H]⁺ | 3 |
| 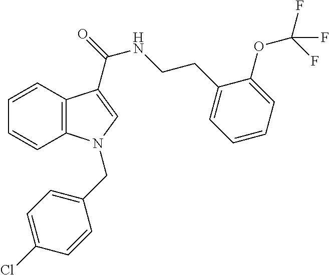 [SMILES: O=C(NCCc1ccccc1OC(F)(F)F)c1cn(Cc2ccc(Cl)cc2)c2ccccc12] | 194 | 14.1 | 472.89 | 473.3 | [M + H]⁺ | 3 |
| 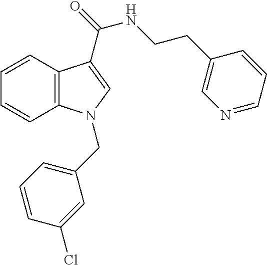 [SMILES: O=C(NCCc1cccnc1)c1cn(Cc2cccc(Cl)c2)c2ccccc12] | 195 | 11.14 | 389.88 | 390.5 | [M + H]⁺ | 3 |
| 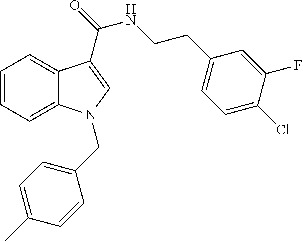 [SMILES: Cc1ccc(Cn2cc(C(=O)NCCc3ccc(Cl)c(F)c3)c3ccccc32)cc1] | 196 | 14.03 | 420.91 | 421.3 | [M + H]⁺ | 3 |
| 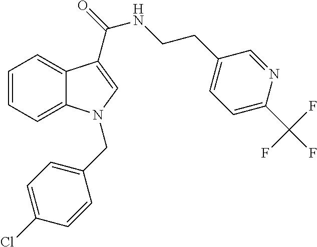 [SMILES: O=C(NCCc1ccc(C(F)(F)F)nc1)c1cn(Cc2ccc(Cl)cc2)c2ccccc12] | 197 | 13.38 | 457.88 | 458.5 | [M + H]⁺ | 3 |
| 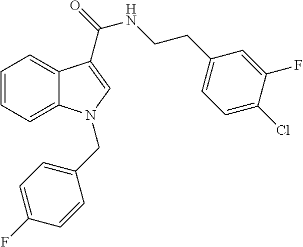 [SMILES: O=C(NCCc1ccc(Cl)c(F)c1)c1cn(Cc2ccc(F)cc2)c2ccccc12] | 198 | 13.66 | 424.88 | 425.3 | [M + H]⁺ | 3 |
| 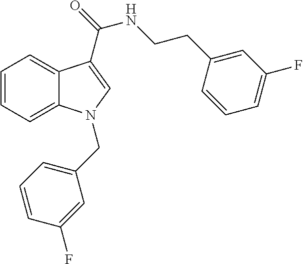 [SMILES: O=C(NCCc1cccc(F)c1)c1cn(Cc2cccc(F)c2)c2ccccc12] | 199 | 13.34 | 390.434 | 391.3 | [M + H]⁺ | 3 |
| 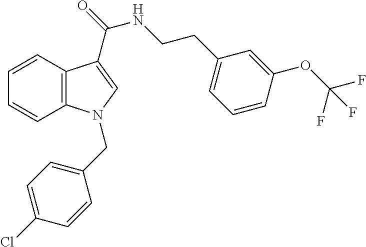 [SMILES: O=C(NCCc1cccc(OC(F)(F)F)c1)c1cn(Cc2ccc(Cl)cc2)c2ccccc12] | 200 | 14.1 | 472.89 | 473.3 | [M + H]⁺ | 3 |
| 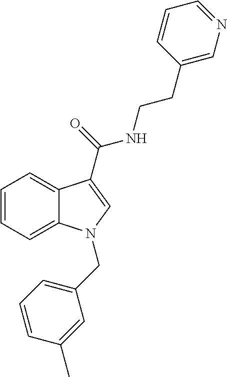 [SMILES: Cc1cccc(Cn2cc(C(=O)NCCc3cccnc3)c3ccccc32)c1] | 201 | 13.79 | 369.468 | 370.4 | [M + H]⁺ | 3 |
| 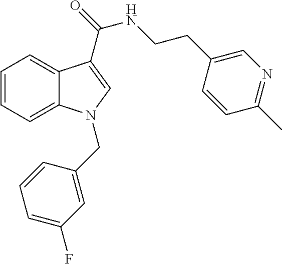 [SMILES: Cc1ccc(CCNC(=O)c2cn(Cc3cccc(F)c3)c3ccccc23)cn1] | 202 | 10.82 | 387.458 | 388.3 | [M + H]⁺ | 3 |
| 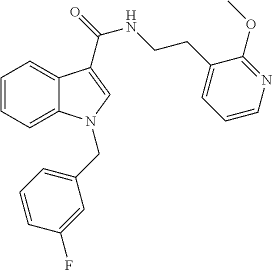 [SMILES: COc1ncccc1CCNC(=O)c1cn(Cc2cccc(F)c2)c2ccccc12] | 203 | 12.82 | 403.457 | 404.6 | [M + H]⁺ | 3 |
| 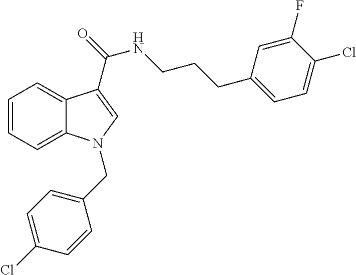 [SMILES: O=C(NCCCc1ccc(Cl)c(F)c1)c1cn(Cc2ccc(Cl)cc2)c2ccccc12] | 204 | 14.22 | 455.35 | 455.4 | [M + H]⁺ | 3 |
| 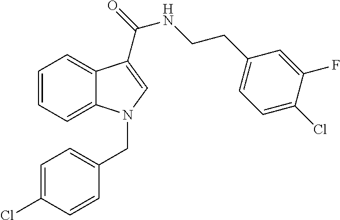 [SMILES: O=C(NCCc1ccc(Cl)c(F)c1)c1cn(Cc2ccc(Cl)cc2)c2ccccc12] | 205 | 14.13 | 441.33 | 441.1 | [M + H]⁺ | 3 |
| 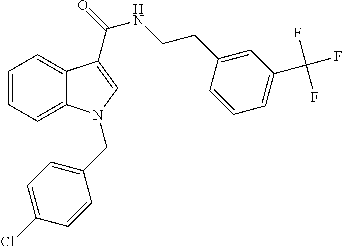 [SMILES: O=C(NCCc1cccc(C(F)(F)F)c1)c1cn(Cc2ccc(Cl)cc2)c2ccccc12] | 206 | 13.94 | 456.89 | 457.6 | [M + H]⁺ | 3 |
| 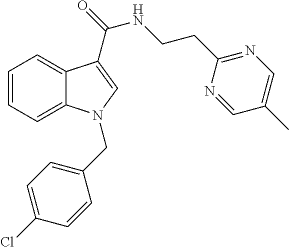 [SMILES: Cc1cnc(CCNC(=O)c2cn(Cc3ccc(Cl)cc3)c3ccccc23)nc1] | 207 | 12.85 | 404.9 | 405.4 | [M + H]⁺ | 3 |
| 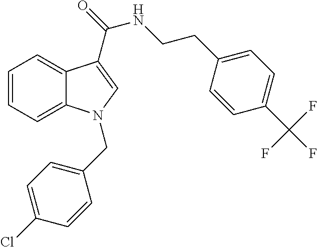 [SMILES: O=C(NCCc1ccc(C(F)(F)F)cc1)c1cn(Cc2ccc(Cl)cc2)c2ccccc12] | 208 | 13.96 | 456.89 | 457.5 | [M + H]⁺ | 3 |
| 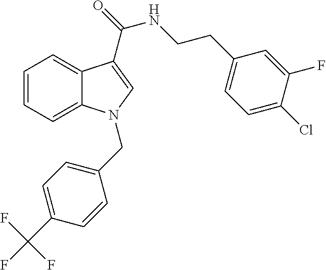 [SMILES: O=C(NCCc1ccc(Cl)c(F)c1)c1cn(Cc2ccc(C(F)(F)F)cc2)c2ccccc12] | 209 | 14 | 474.88 | 475.3 | [M + H]⁺ | 3 |
| 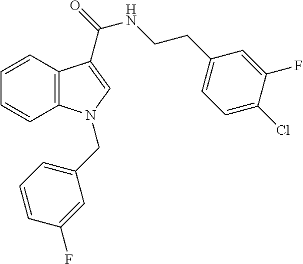 [SMILES: O=C(NCCc1ccc(Cl)c(F)c1)c1cn(Cc2cccc(F)c2)c2ccccc12] | 210 | 13.64 | 424.88 | 425.3 | [M + H]⁺ | 3 |
| 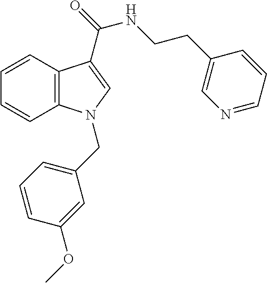 [SMILES: COc1cccc(Cn2cc(C(=O)NCCc3cccnc3)c3ccccc32)c1] | 211 | 10.72 | 385.467 | 386.4 | [M + H]⁺ | 3 |
| 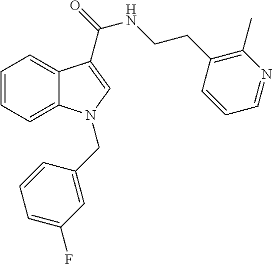 [SMILES: Cc1ncccc1CCNC(=O)c1cn(Cc2cccc(F)c2)c2ccccc12] | 212 | 10.79 | 387.458 | 388.2 | [M + H]⁺ | 3 |
| 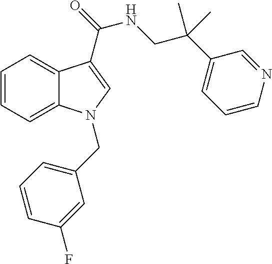 [SMILES: CC(C)(CNC(=O)c1cn(Cc2cccc(F)c2)c2ccccc12)c1cccnc1] | 213 | 11.14 | 401.485 | 402.5 | [M + H]⁺ | 3 |
| 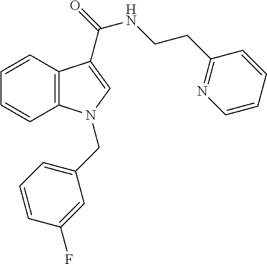 [SMILES: O=C(NCCc1ccccn1)c1cn(Cc2cccc(F)c2)c2ccccc12] | 214 | 10.73 | 373.431 | 374 | [M + H]⁺ | 3 |
| 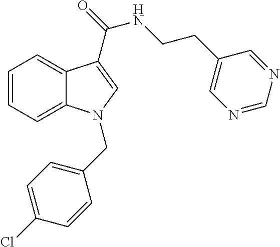 [SMILES: O=C(NCCc1cncnc1)c1cn(Cc2ccc(Cl)cc2)c2ccccc12] | 215 | 12.53 | 390.87 | 391.4 | [M + H]⁺ | 3 |
| 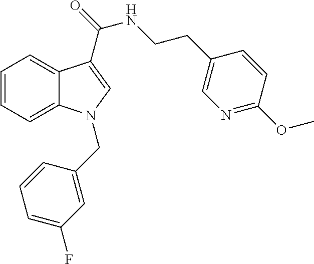 [SMILES: COc1ccc(CCNC(=O)c2cn(Cc3cccc(F)c3)c3ccccc23)cn1] | 216 | 12.3 | 403.457 | 404.6 | [M + H]⁺ | 3 |
| 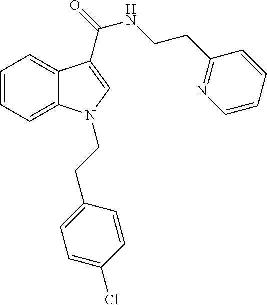 [SMILES: O=C(NCCc1ccccn1)c1cn(CCc2ccc(Cl)cc2)c2ccccc12] | 217 | 11.06 | 403.91 | 404.5 | [M + H]⁺ | 3 |
| 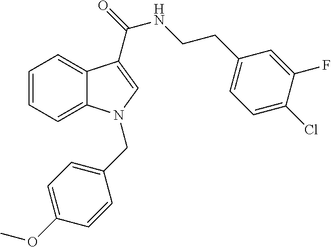 [SMILES: COc1ccc(Cn2cc(C(=O)NCCc3ccc(Cl)c(F)c3)c3ccccc32)cc1] | 218 | 13.65 | 436.91 | 437.3 | [M + H]⁺ | 3 |
| 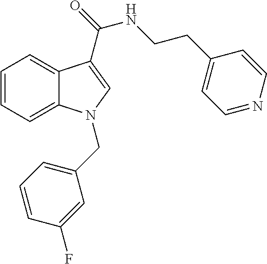 [SMILES: O=C(NCCc1ccncc1)c1cn(Cc2cccc(F)c2)c2ccccc12] | 219 | 10.69 | 373.431 | 374.4 | [M + H]⁺ | 3 |
| 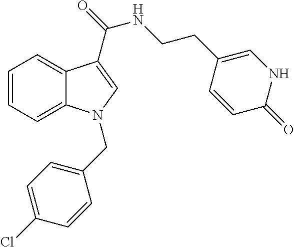 [SMILES: O=C(NCCc1ccc(=O)[nH]c1)c1cn(Cc2ccc(Cl)cc2)c2ccccc12] | 220 | 12.3 | 405.88 | 406.5 | [M + H]⁺ | 3 |
| 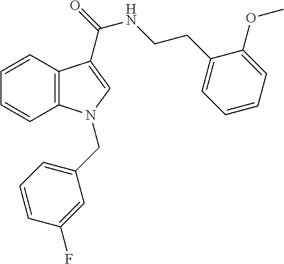 [SMILES: COc1ccccc1CCNC(=O)c1cn(Cc2cccc(F)c2)c2ccccc12] | 221 | 13.51 | 402.469 | 403.4 | [M + H]⁺ | 3 |
| 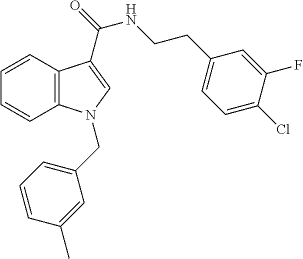 [SMILES: Cc1cccc(Cn2cc(C(=O)NCCc3ccc(Cl)c(F)c3)c3ccccc32)c1] | 222 | 14.05 | 420.91 | 421.3 | [M + H]⁺ | 3 |
| 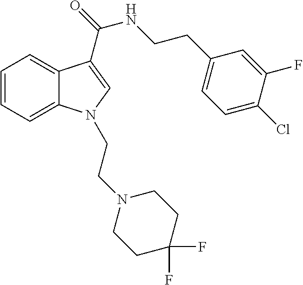 [SMILES: O=C(NCCc1ccc(Cl)c(F)c1)c1cn(CCN2CCC(F)(F)CC2)c2ccccc12] | 223 | 7.429 | 463.93 | 464.2 | [M + H]⁺ | 3 |
| 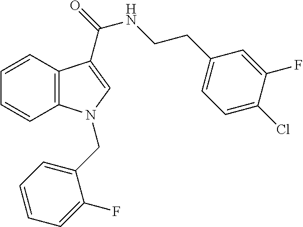 [SMILES: O=C(NCCc1ccc(Cl)c(F)c1)c1cn(Cc2ccccc2F)c2ccccc12] | 224 | 13.72 | 424.88 | 425.1 | [M + H]⁺ | 3 |
| 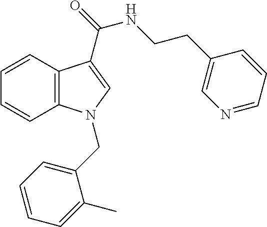 [SMILES: Cc1ccccc1Cn1cc(C(=O)NCCc2cccnc2)c2ccccc21] | 225 | 11.29 | 369.468 | 370.4 | [M + H]⁺ | 3 |
| 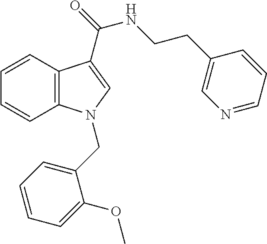 [SMILES: COc1ccccc1Cn1cc(C(=O)NCCc2cccnc2)c2ccccc21] | 226 | 11.52 | 385.467 | 386.4 | [M + H]⁺ | 3 |
| 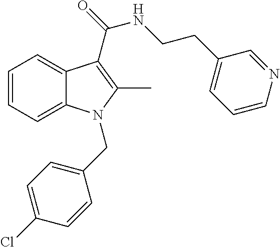 [SMILES: Cc1c(C(=O)NCCc2cccnc2)c2ccccc2n1Cc1ccc(Cl)cc1] | 227 | 11.59 | 403.91 | 404.6 | [M + H]⁺ | 3 |
| 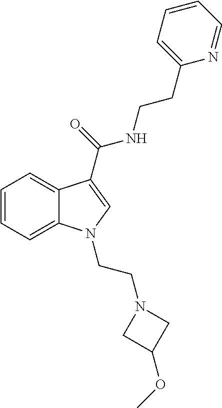 [SMILES: COC1CN(CCn2cc(C(=O)NCCc3ccccn3)c3ccccc32)C1] | 228 | 6.54 | 378.476 | 379.4 | [M + H]⁺ | 3 |
| 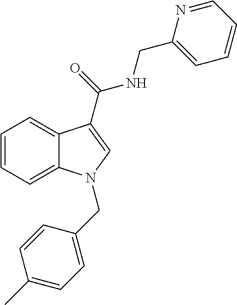 [SMILES: Cc1ccc(Cn2cc(C(=O)NCc3ccccn3)c3ccccc32)cc1] | 229 | 11.75 | 355.441 | 356.4 | [M + H]⁺ | 3 |
| 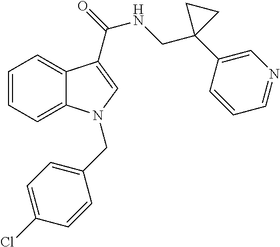 [SMILES: O=C(NCC1(c2cccnc2)CC1)c1cn(Cc2ccc(Cl)cc2)c2ccccc12] | 230 | 11.71 | 415.92 | 416.5 | [M + H]⁺ | 3 |
| 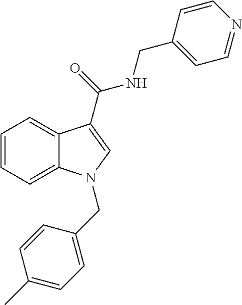 [SMILES: Cc1ccc(Cn2cc(C(=O)NCc3ccncc3)c3ccccc32)cc1] | 231 | 11.51 | 355.441 | 356.4 | [M + H]⁺ | 3 |
| 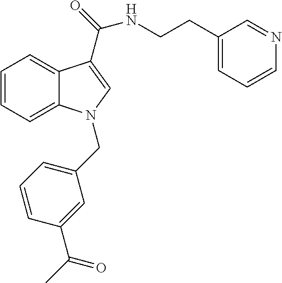 [SMILES: CC(=O)c1cccc(Cn2cc(C(=O)NCCc3cccnc3)c3ccccc32)c1] | 232 | 10.06 | 397.478 | 398.3 | [M + H]⁺ | 3 |
| 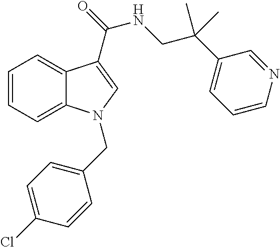 [SMILES: CC(C)(CNC(=O)c1cn(Cc2ccc(Cl)cc2)c2ccccc12)c1cccnc1] | 233 | 11.58 | 417.94 | 418.4 | [M + H]⁺ | 3 |
| 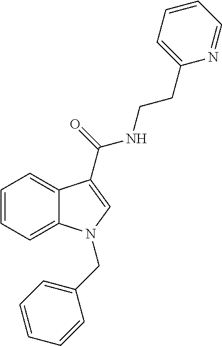 [SMILES: O=C(NCCc1ccccn1)c1cn(Cc2ccccc2)c2ccccc12] | 234 | 10.53 | 355.441 | 356.2 | [M + H]⁺ | 3 |
| 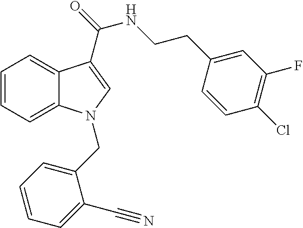 [SMILES: N#Cc1ccccc1Cn1cc(C(=O)NCCc2ccc(Cl)c(F)c2)c2ccccc21] | 235 | 13.24 | 431.9 | 432.4 | [M + H]⁺ | 3 |
| 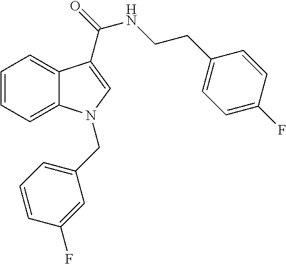 [SMILES: O=C(NCCc1ccc(F)cc1)c1cn(Cc2cccc(F)c2)c2ccccc12] | 236 | 13.33 | 390.434 | 391.4 | [M + H]⁺ | 3 |

### Example 7

### Synthesis of Representative Compound 311

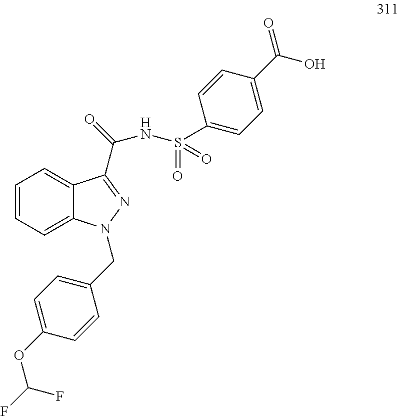 [SMILES: O=C(O)c1ccc(S(=O)(=O)NC(=O)c2nn(Cc3ccc(OC(F)F)cc3)c3ccccc23)cc1]

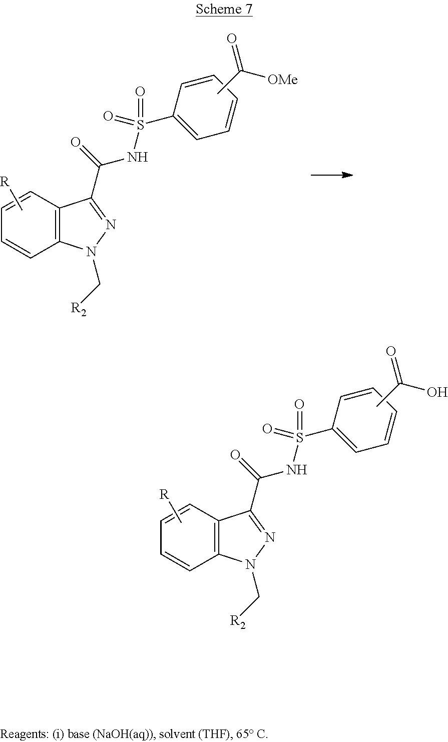

### Step 7-1. Synthesis of 4-(N-(1-(4-(difluoromethoxy)benzyl)-1H-indazole-3-carbonyl)-sulfamoyl)benzoic acid (311)

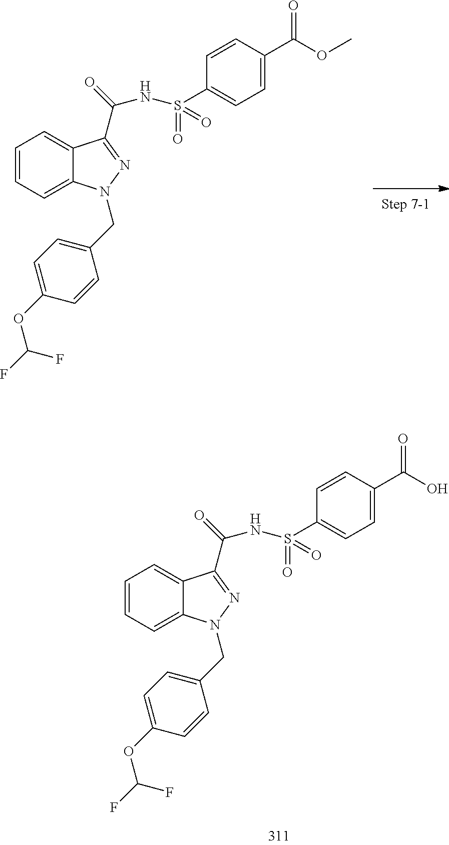 [SMILES: COC(=O)c1ccc(S(=O)(=O)NC(=O)c2nn(Cc3ccc(OC(F)F)cc3)c3ccccc23)cc1 ; O=C(O)c1ccc(S(=O)(=O)NC(=O)c2nn(Cc3ccc(OC(F)F)cc3)c3ccccc23)cc1]

To a stirring solution of methyl 4-(N-(1-(4-(difluoromethoxy)benzyl)-1H-indazole-3-carbonyl)sulfamoyl)benzoate (15 mg, 1 Eq, 29 μmol) in THF (2 mL) was added 1M sodium hydroxide (0.29 mL, 10 Eq, 0.29 mmol). The vial was capped and heated at 65° C. overnight. After heating at 65° C. for 14 h, the reaction mixture was cooled to room temperature. The THF layer was removed. To the aqueous layer was added 3M HCl (1 mL). The aqueous layer was extracted with EtOAc (3×5 mL), dried over sodium sulfate, filtered, and concentrated in vacuo to afford crude material. The crude product was purified by ISCO reversed phase prep system (25-35% formic acid in MeCN and 0.1% formic acid in water) to afford 7 mg (48% yield) of 4-(N-(1-(4-(difluoromethoxy)benzyl)-1H-indazole-3-carbonyl)sulfamoyl)benzoic acid (Compound 311) as a white solid; LCMS (m/z) calculated for C₂₃H₁₇F₂N₃O₄S: 501.1; found 499.8 [M+H]⁺, t_{R}=9.267 min (Method 1). ¹H NMR (400 MHz, DMSO-d6) δ 8.17 (m, 4H), 7.99 (d, J=8 Hz, 1H), 7.85 (d, J=8 Hz, 1H), 7.45 (m, 3H), 7.31 (t, J=8 Hz, 1H), 7.15 (m, 3H), 5.80 (s, 2H).

The compounds listed in Table 7 were made using the procedures of Scheme 7.

TABLE 7

| | Cpd | Purity | | Observed | | Purity |
|---|---|---|---|---|---|---|
| Structure | No. | RT (min) | MW | m/z | Ion | Method |
| 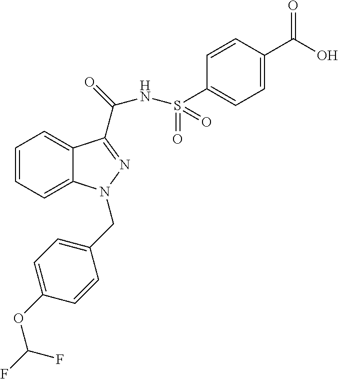 [SMILES: O=C(O)c1ccc(S(=O)(=O)NC(=O)c2nn(Cc3ccc(OC(F)F)cc3)c3ccccc23)cc1] | 311 | 9.267 | 501.1 | 499.8 | [M − H]⁻ | 1 |

### Example 8

### Synthesis of Representative Compound 137

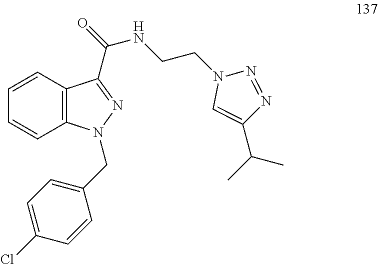 [SMILES: CC(C)c1cn(CCNC(=O)c2nn(Cc3ccc(Cl)cc3)c3ccccc23)nn1]

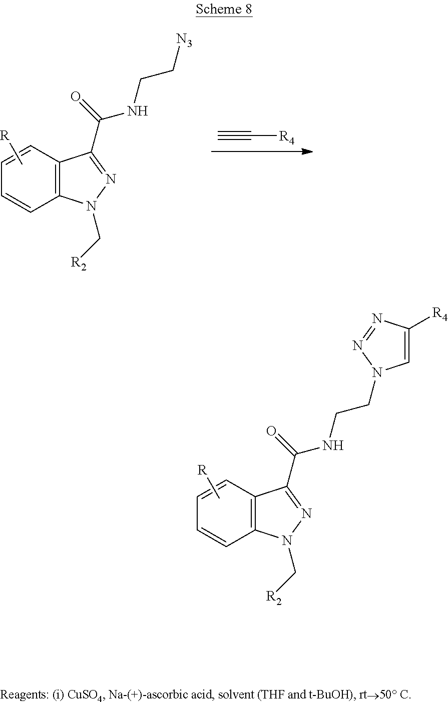

### Step 8-1. Synthesis of 1-(4-chlorobenzyl)-N-(2-(4-isopropyl-1H-1,2,3-triazol-1-yl)ethyl)-1H-indazole-3-carboxamide (Compound 137)

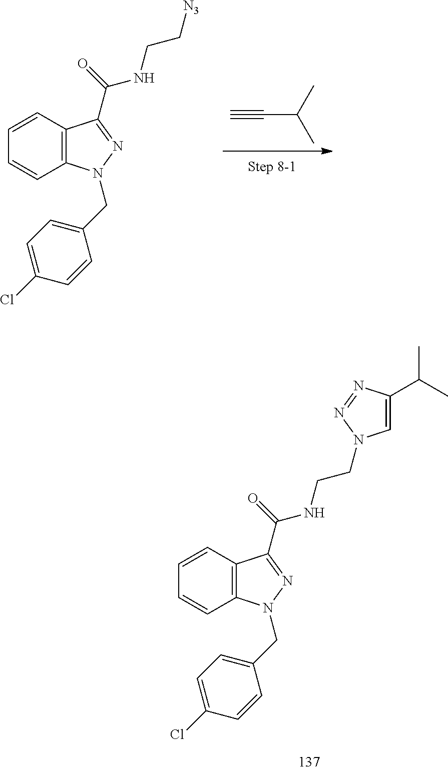

A solution of CuSO₄·5H₂O (50.0 μL, 2.82 μmol, 0.02 Eq, 0.0564 M in water) and a solution of sodium L-ascorbic acid (50.0 μL, 14.1 μmol, 0.1 Eq, 0.282 M in water) were added to a mixture of N-(2-azidoethyl)-1-[(4-chlorophenyl)methyl]indazole-3-carboxamide (50.0 mg, 1 Eq, 0.141 mmol) and 3-methylbut-1-yne (15.9 μL, 1.1 Eq, 0.155 mmol) in THE (0.62 mL), t-BuOH (0.38 mL) and H₂O (1.90 mL) at room temperature under nitrogen. The mixture was then heated at 50° C. for 72 h. After heating at 50° C. for 72 h, the reaction mixture was cooled to room temperature. The mixture was diluted with aqueous NaHCO₃ (50 mL), and the aqueous phase was extracted with DCM (3×75 mL). The combined organic phases were washed with brine (25 mL), dried with sodium sulfate, filtered, and concentrated in vacuo. The crude residue was purified by silica gel chromatography (0->20% MeOH and DCM) to provide 40 mg (63% yield) of 1-(4-chlorobenzyl)-N-(2-(4-isopropyl-1H-1,2,3-triazol-1-yl)ethyl)-1H-indazole-3-carboxamide (Compound 137) as a white solid; LCMS (m/z) calculated for C₂₂H₂₃ClN₆O: 422.2; found 422.2 [M+H]⁺, t_{R}=2.52 min (Method 4). ¹H NMR (400 MHz, DMSO-d₆) δ 8.54 (t, J=5.9 Hz, 1H), 8.16 (dt, J=8.2, 1.1 Hz, 1H), 7.82 (d, J=0.7 Hz, 1H), 7.79 (d, J=8.6 Hz, 1H), 7.45 (ddd, J=8.5, 6.9, 1.1 Hz, 1H), 7.42-7.22 (m, 5H), 5.75 (s, 2H), 4.53 (t, J=6.4 Hz, 2H), 3.73 (q, J=6.2 Hz, 2H), 2.92 (pd, J=6.9, 0.7 Hz, 1H), 1.17 (d, J=6.9 Hz, 6H).

The compounds listed in Table 8 were made using the procedures of Scheme 8.

TABLE 8

| | Cpd | Purity | | Observed | | Purity |
|---|---|---|---|---|---|---|
| Structure | No. | RT (min) | MW | m/z | Ion | Method |
| 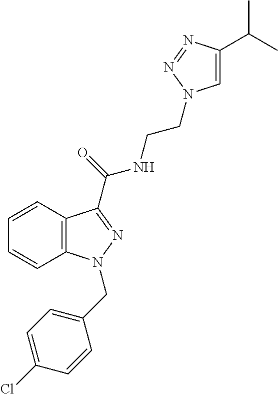 [SMILES: CC(C)c1cn(CCNC(=O)c2nn(Cc3ccc(Cl)cc3)c3ccccc23)nn1] | 137 | 4.66 | 422.2 | 422.2 | [M] | 4 |
| 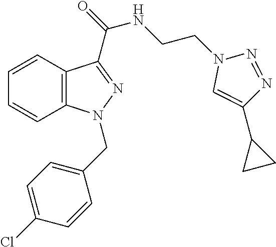 [SMILES: O=C(NCCn1cc(C2CC2)nn1)c1nn(Cc2ccc(Cl)cc2)c2ccccc12] | 138 | 4.50 | 420.2 | 420.2 | [M] | 4 |
| 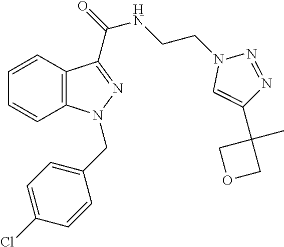 [SMILES: CC1(c2cn(CCNC(=O)c3nn(Cc4ccc(Cl)cc4)c4ccccc34)nn2)COC1] | 139 | 4.09 | 450.2 | 450.2 | [M] | 4 |
| 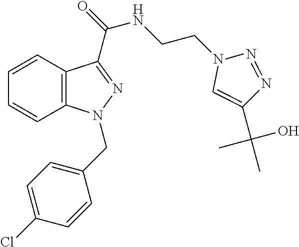 [SMILES: CC(C)(O)c1cn(CCNC(=O)c2nn(Cc3ccc(Cl)cc3)c3ccccc23)nn1] | 140 | 3.86 | 438.2 | 438.2 | [M] | 4 |
| 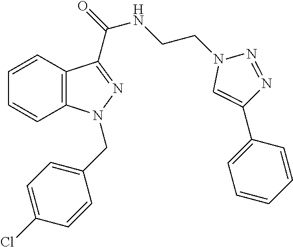 [SMILES: O=C(NCCn1cc(-c2ccccc2)nn1)c1nn(Cc2ccc(Cl)cc2)c2ccccc12] | 141 | 5.05 | 456.2 | 456.2 | [M] | 4 |
| 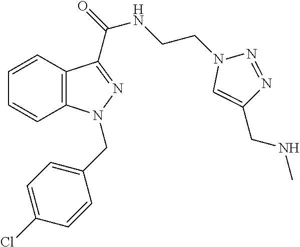 [SMILES: CNCc1cn(CCNC(=O)c2nn(Cc3ccc(Cl)cc3)c3ccccc23)nn1] | 142 | 3.23 | 423.2 | 423.2 | [M] | 4 |

### Example 9

### Synthesis of Representative Compound 143

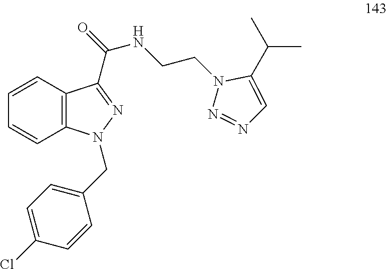 [SMILES: CC(C)c1cnnn1CCNC(=O)c1nn(Cc2ccc(Cl)cc2)c2ccccc12]

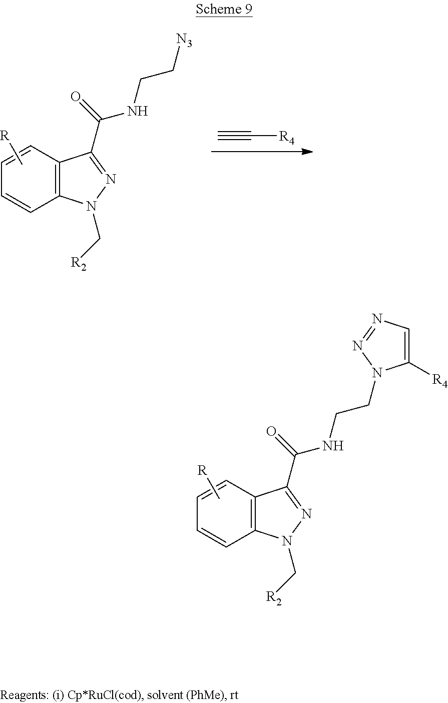

### Step 9-1. Synthesis of 1-(4-chlorobenzyl)-N-(2-(5-isopropyl-1H-1,2,3-triazol-1-yl)ethyl)-1H-indazole-3-carboxamide (Compound 143)

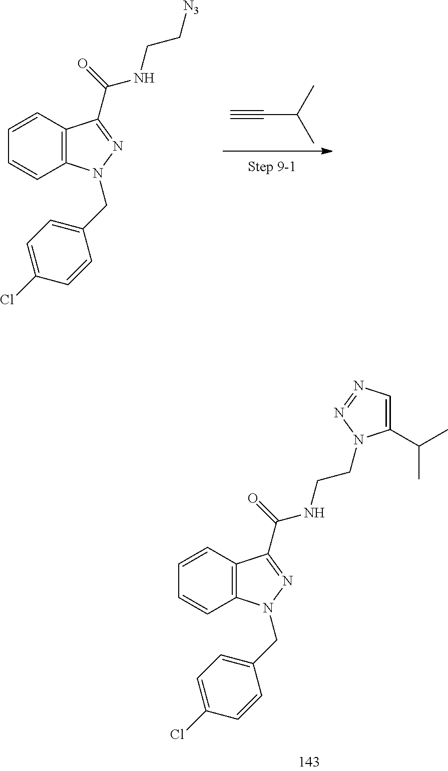

Cp*RuCl(cod) (10.7 mg, 0.0282 mmol) was added to a mixture of N-(2-azidoethyl)-1-[(4-chlorophenyl)methyl]indazole-3-carboxamide (50.0 mg, 1 Eq, 0.141 mmol) and 3-methylbut-1-yne (72 μL, 5 Eq, 0.71 mmol) in PhMe (4 mL) at room temperature under nitrogen. The mixture was stirred at room temperature for 24 h and concentrated in vacuo. The crude residue was purified by reverse phase chromatography (C18) (10-100% with 10 mM ammonium formate (aq) and MeCN) to afford 19 mg (32% yield) of 1-(4-chlorobenzyl)-N-(2-(5-isopropyl-1H-1,2,3-triazol-1-yl)ethyl)-1H-indazole-3-carboxamide (143) as a white solid. LCMS (m/z) calculated for C₂₂H₂₃ClN₆O: 422.2; found 422.2 [M], t_{R}=4.7 min (Method 4). ¹H NMR (400 MHz, DMSO-d₆) δ 8.60 (t, J=6.0 Hz, 1H), 8.17 (dt, J=8.2, 1.0 Hz, 1H), 7.79 (dt, J=8.6, 0.9 Hz, 1H), 7.52 (d, J=0.5 Hz, 1H), 7.50-7.41 (m, 1H), 7.39 (d, J=8.5 Hz, 2H), 7.28 (ddd, J=7.9, 6.9, 0.9 Hz, 1H), 7.25 (d, J=8.5 Hz, 2H), 5.75 (s, 2H), 4.48 (t, J=6.6 Hz, 2H), 3.71 (q, J=6.4 Hz, 2H), 3.11 (p, J=6.8 Hz, 1H), 1.16 (d, J=6.9 Hz, 6H).

The compound listed in Table 9 was made using the procedures of Scheme 9.

TABLE 9

| | Cpd | Purity | | Observed | | Purity |
|---|---|---|---|---|---|---|
| Structure | No. | RT (min) | MW | m/z | Ion | Method |
| 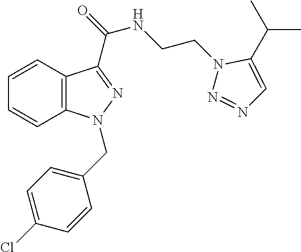 [SMILES: CC(C)c1cnnn1CCNC(=O)c1nn(Cc2ccc(Cl)cc2)c2ccccc12] | 143 | 4.7 | 422.2 | 422.2 | [M] | 4 |

### Example 10

### Synthesis of Representative Compound 144

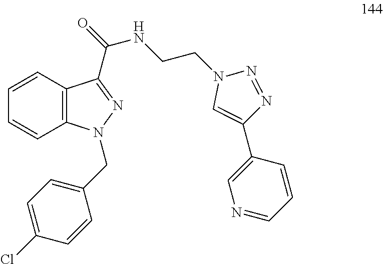 [SMILES: O=C(NCCn1cc(-c2cccnc2)nn1)c1nn(Cc2ccc(Cl)cc2)c2ccccc12]

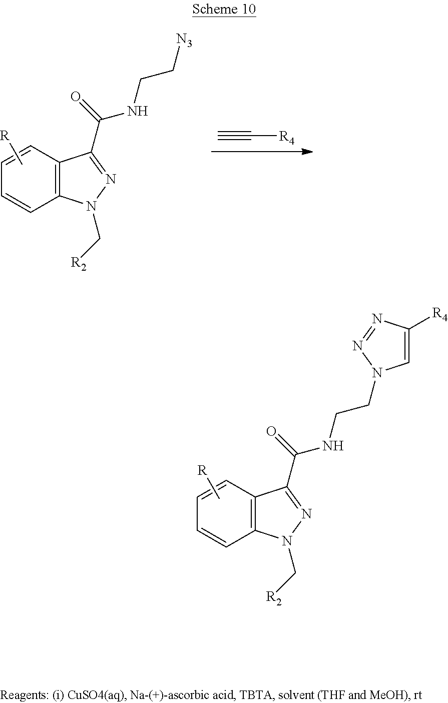

### Step 10-1. Synthesis of 1-(4-chlorobenzyl)-N-(2-(4-(pyridin-3-yl)-1H-1,2,3-triazol-1-yl)ethyl)-1H-indazole-3-carboxamide (Compound 144)

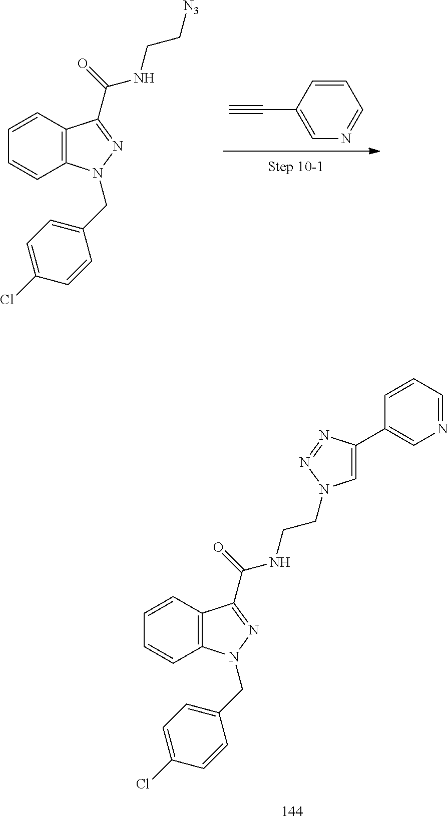

A stirring solution of CuSO₄·5H₂O (750 μL, 0.0423 mmol, 0.2 Eq, 0.0564 M in water) and a solution of sodium L-ascorbic acid (750 μL, 0.211 mmol, 1 Eq, 0.282 M in water) were added to a stirring mixture of N-(2-azidoethyl)-1-[(4-chlorophenyl)methyl]indazole-3-carboxamide (75.0 mg, 1 Eq, 0.211 mmol), tris[(1-benzyl-1H-1,2,3-triazol-4-yl)methyl]amine (TBTA) (22.4 mg, 0.2 Eq, 0.0423 mmol) and 3-ethynylpyridine (109 mg, 5 Eq, 1.06 mmol) in THE (1.5 mL) and MeOH (1.5 mL) at room temperature under nitrogen. The mixture was stirred at room temperature for 18 h. The mixture was diluted with NaOH (1M in H₂O, 20 mL), and the aqueous phase was extracted with EtOAc (3×20 mL). The combined organics were washed with brine (25 mL), dried over sodium sulfate (Na₂SO₄), filtered, and concentrated in vacuo. The crude residue was purified by silica gel chromatography (0→7% MeOH in EtOAc) to afford 73 mg (75% yield) of 1-(4-chlorobenzyl)-N— (2-(4-(pyridin-3-yl)-1H-1,2,3-triazol-1-yl)ethyl)-1H-indazole-3-carboxamide (Compound 144) as a solid; LCMS (m/z) calculated for C₂₄H₂₀ClN₇O: 457.1; found 457.1 [M], t_{R}=4.13 mi (Method 4). ¹H NR (500 MHz, DMSO-d₆) δ 9.03 (dd, J=2.2, 0.8 Hz, 1H), 8.73 (s, 1H), 8.62 (t, J=5.9 Hz, 1H), 8.53 (dd, J=4.8, 1.6 Hz, 1H), 8.21-8.17 (m, 1H), 8.16 (d, J=8.2 Hz, 1H), 7.77 (d, J=8.6 Hz, 1H), 7.48-7.43 (m, 2H), 7.38-7.34 (m, 2H), 7.29-7.23 (m, 3H), 5.74 (s, 2H), 4.67 (t, J=6.2 Hz, 2H), 3.81 (q, J=6.0 Hz, 2H).

The compound listed in Table 10 was made using the procedures of Scheme 10.

TABLE 10

| | Cpd | Purity RT | | Observed | | Purity |
|---|---|---|---|---|---|---|
| Structure | No. | (min) | MW | m/z | Ion | Method |
| 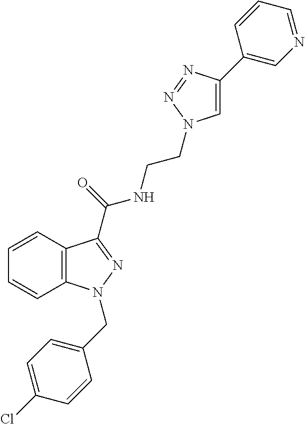 [SMILES: O=C(NCCn1cc(-c2cccnc2)nn1)c1nn(Cc2ccc(Cl)cc2)c2ccccc12] | 144 | 4.13 | 457.1 | 457.1 | [M] | 4 |
| 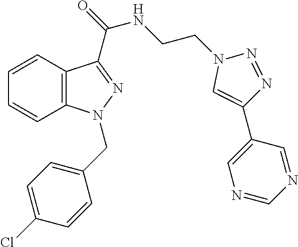 [SMILES: O=C(NCCn1cc(-c2cncnc2)nn1)c1nn(Cc2ccc(Cl)cc2)c2ccccc12] | 145 | 3.94 | 458.1 | 458.1 | [M] | 4 |
| 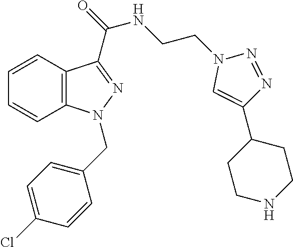 [SMILES: O=C(NCCn1cc(C2CCNCC2)nn1)c1nn(Cc2ccc(Cl)cc2)c2ccccc12] | 146 | 3.32 | 463.2 | 463.2 | [M] | 4 |
| 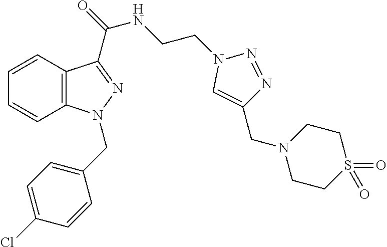 [SMILES: O=C(NCCn1cc(CN2CCS(=O)(=O)CC2)nn1)c1nn(Cc2ccc(Cl)cc2)c2ccccc12] | 147 | 3.75 | 527.2 | 527.2 | [M] | 4 |

### Example 11

### Synthesis of Representative Compound 333

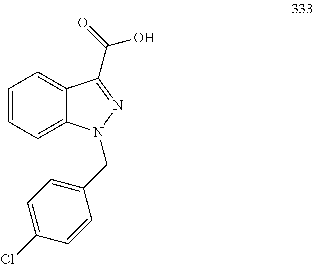 [SMILES: O=C(O)c1nn(Cc2ccc(Cl)cc2)c2ccccc12]

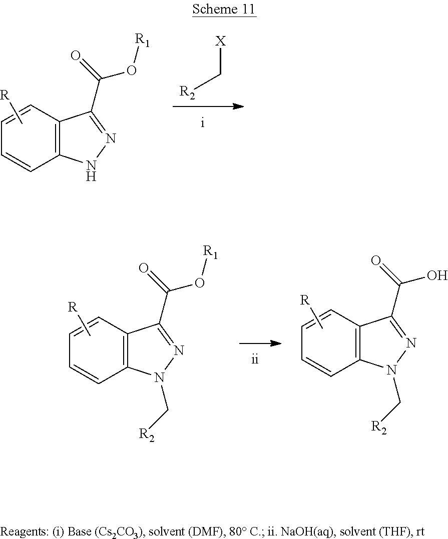

### Step 11-1. Synthesis of methyl 1-(4-chlorobenzyl)-1H-indazole-3-carboxylate (INT-11A)

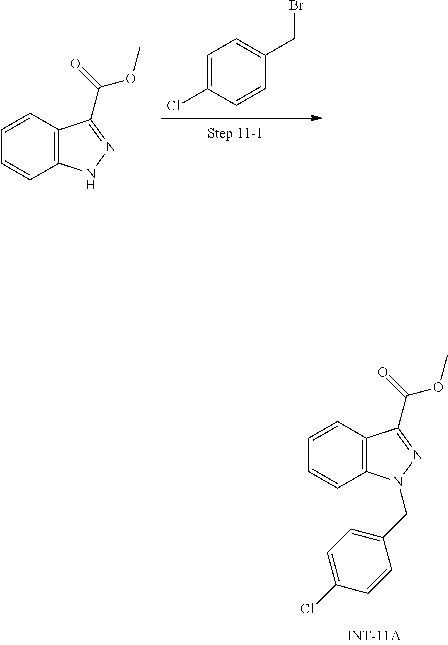 [SMILES: COC(=O)c1n[nH]c2ccccc12 ; Clc1ccc(CBr)cc1 ; COC(=O)c1nn(Cc2ccc(Cl)cc2)c2ccccc12]

1-(Bromomethyl)-4-chloro-benzene (1.40 g, 1.2 Eq, 6.81 mmol) was added to a mixture of methyl 1H-indazole-3-carboxylate (1.00 g, 1 Eq, 5.68 mmol) and Cs₂CO₃ (3.70 g, 2 Eq, 11.4 mmol) in DMF (6 mL) at room temperature under nitrogen. The mixture was heated at 80° C. for 18 h. After heating the reaction mixture at 80° C. for 18 h, the reaction mixture was cooled to room temperature and diluted with EtOAc (100 mL) and water (100 mL). The aqueous phase was extracted with EtOAc (3×75 mL). The combined organics were washed with brine (50 mL), dried with sodium sulfate, filtered, and concentrated in vacuo. The crude residue was purified by silica gel chromatography (0->75% EtOAc and hexanes) to afford 1.06 g (62% yield) of methyl 1-(4-chlorobenzyl)-1H-indazole-3-carboxylate (INT-11A) as a white solid; LCMS (m/z) calculated for C₁₆H₁₃ClN₂O₂: 300.1; found 301.2 [M+H]⁺, t_{R}=2.59 min (Method 4). ¹H NMR (400 MHz, CDCl₃) δ 8.25 (dt, J=8.1, 1.1 Hz, 1H), 7.40-7.37 (m, 1H), 7.34 (dd, J=1.8, 1.1 Hz, 1H), 7.32 (q, J=1.2 Hz, 1H), 7.29-7.26 (m, 2H), 7.18-7.12 (m, 2H), 5.67 (s, 2H), 4.06 (s, 3H).

### Step 11-2. Synthesis of 1-(4-chlorobenzyl)-1H-indazole-3-carboxylic acid (Compound 333)

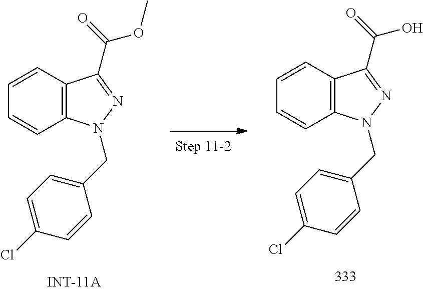 [SMILES: COC(=O)c1nn(Cc2ccc(Cl)cc2)c2ccccc12 ; O=C(O)c1nn(Cc2ccc(Cl)cc2)c2ccccc12]

To a mixture of methyl 1-[(4-chlorophenyl)methyl]indazole-3-carboxylate (INT-11A) (3.95 g, 13.1 mmol) in THF (20 mL) and H₂O (20 mL) was added NaOH (1.58 g, 39.4 mmol). The reaction mixture was heated at 60° C. for 18 h. After heating for 18 h at 60° C., the reaction mixture was cooled to room temperature. The mixture was acidified with 12 M HCl (pH ˜ 1). The precipitate was filtered, washed with water (100 mL), and dried to afford 3.60 g (96% yield) of 1-(4-chlorobenzyl)-1H-indazole-3-carboxylic acid (Compound 333) as a white solid; LCMS (m/z) calculated for C₁₅H₁₁ClN₂O₂: 286.1; found 285.1 [M−H]⁻, t_{R}=2.39 min (Method 4). ¹H NMR (400 MHz, DMSO-d₆) δ 13.10 (s, 1H), 8.10 (dt, J=8.2, 1.0 Hz, 1H), 7.84 (dt, J=8.6, 0.8 Hz, 1H), 7.51-7.44 (m, 1H), 7.42-7.37 (m, 2H), 7.35-7.30 (m, 1H), 7.30-7.25 (m, 2H), 5.78 (s, 2H).

The compound listed in Table 11 was made using the procedures of Scheme 11.

TABLE 11

| | Cpd | Purity RT | | Observed | | Purity |
|---|---|---|---|---|---|---|
| Structure | No. | (min) | MW | m/z | Ion | Method |
| 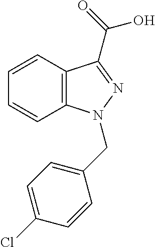 [SMILES: O=C(O)c1nn(Cc2ccc(Cl)cc2)c2ccccc12] | 333 | 2.39 | 286.1 | 285.1 | [M − H]⁻ | 4 |

### Example 12

### Synthesis of Representative Compound 148

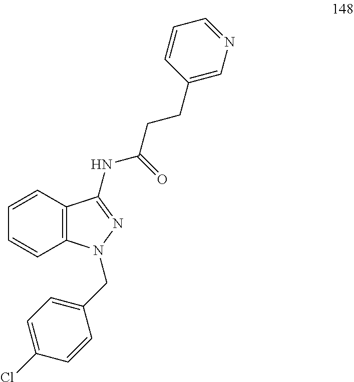 [SMILES: O=C(CCc1cccnc1)Nc1nn(Cc2ccc(Cl)cc2)c2ccccc12]

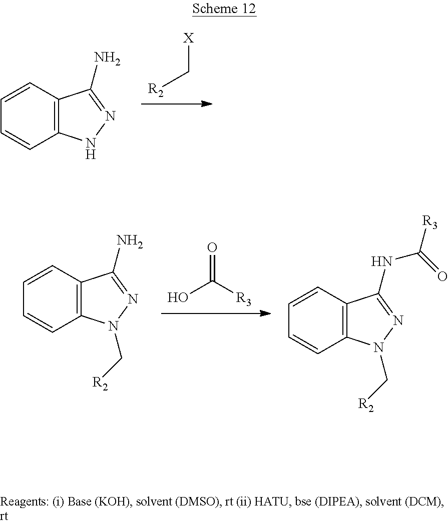

### Step 12-1. Synthesis of 1-(4-chlorobenzyl)-1H-indazol-3-amine (INT-12A)

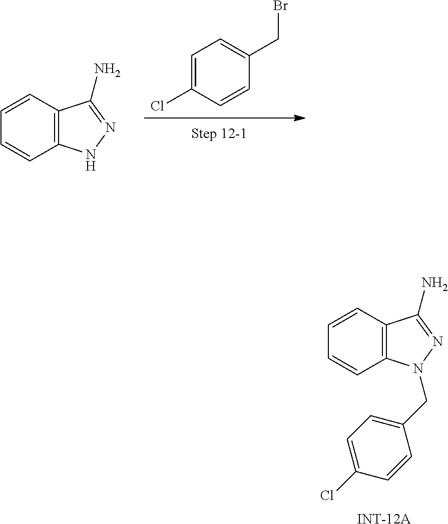

1H-Indazol-3-amine (1.00 g, 7.51 mmol) was added to a stirring solution of KOH (843 mg, 15.0 mmol) in DMSO (6.0 mL) at room temperature. After stirring the mixture for 30 min at room temperature, 1-(Bromomethyl)-4-chloro-benzene (1.62 g, 7.89 mmol) was added to the mixture. After stirring at room temperature for 24 h, the mixture was diluted with DCM (100 mL) and water (100 mL). The aqueous phase was extracted with DCM (3×75 mL). The combined organics were washed with brine (50 mL), dried over sodium sulfate, filtered, and concentrated in vacuo. The residue was purified by silica gel chromatography (0->15% MeOH in DCM) to provide 1.04 g (54% yield) of 1-(4-chlorobenzyl)-1H-indazol-3-amine (INT-12A) as a white solid; LCMS (m/z) calculated for C₂₄H₁₂ClN₃: 257.1; found 259.2 [M+H]⁺, t_{R}=2.33 min (Method 4). ¹H NMR (500 MHz, CDCl₃) δ 7.55 (dt, J=8.1, 1.0 Hz, 1H), 7.36-7.29 (m, 1H), 7.24 (d, J=8.5 Hz, 2H), 7.18 (dt, J=8.5, 0.9 Hz, 1H), 7.11 (d, J=8.3 Hz, 2H), 7.04 (ddd, J=8.0, 6.9, 0.9 Hz, 1H), 5.32 (s, 2H), 4.08 (s, 2H).

### Step 12-2. Synthesis of N-(1-(4-chlorobenzyl)-1H-indazol-3-yl)-3-(pyridin-3-yl)-propanamide (Compound 148)

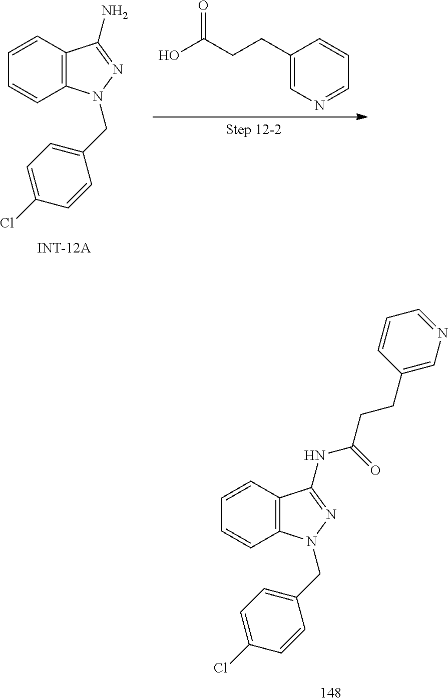

To a stirring mixture of 3-(3-pyridyl)propanoic acid (INT-12A) (100 mg, 0.662 mmol) in DCM (5 mL) at room temperature was added DIPEA (230 μL, 1.32 mmol) followed by HATU (377 mg, 0.992 mmol) and 1-[(4-chlorophenyl)methyl]indazol-3-amine (205 mg, 0.794 mmol). After stirring at room temperature for 18 h, the reaction mixture was concentrated in vacuo. The residue was purified by reverse phase chromatography (C18) (10->100% 0.1% formic acid in MeCN and 0.1% formic acid in H₂O) to afford 27 mg (10% yield) of N-(1-(4-chlorobenzyl)-1H-indazol-3-yl)-3-(pyridin-3-yl)propenamide (Compound 148) as a white solid; LCMS (m/z) calculated for C₂₂H₁₉ClN₃: 390.1; found 392.6 [M+H]⁺, t_{R}=3.91 min (Method 4). ¹H NMR (400 MHz, DMSO-d₆) δ 10.48 (s, 1H), 8.58 (s, 1H), 8.48 (d, J=4.0 Hz, 1H), 7.86 (d, J=7.9 Hz, 1H), 7.72 (dt, J=8.2, 1.0 Hz, 1H), 7.69-7.62 (m, 1H), 7.45 (dd, J=7.9, 4.9 Hz, 1H), 7.41-7.31 (m, 3H), 7.23 (d, J=8.5 Hz, 2H), 7.07 (dd, J=8.3, 6.8 Hz, 1H), 5.54 (s, 2H), 3.00 (t, J=7.5 Hz, 2H), 2.76 (t, J=7.5 Hz, 2H).

The compound listed in Table 12 was made using the procedures of Scheme 12.

TABLE 12

| | Cpd | Purity | | Observed | | Purity |
|---|---|---|---|---|---|---|
| Structure | No. | RT (min) | MW | m/z | Ion | Method |
| 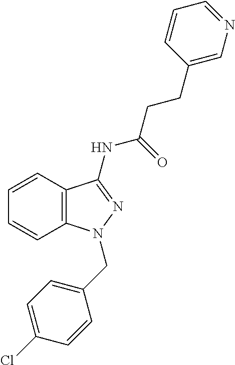 [SMILES: O=C(CCc1cccnc1)Nc1nn(Cc2ccc(Cl)cc2)c2ccccc12] | 148 | 3.91 | 390.1 | 392.6 | [M + H]⁺ | 4 |

### Example 13

### Synthesis of Representative Compound 237

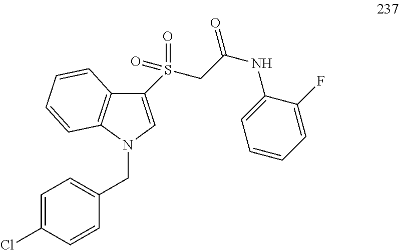 [SMILES: O=C(CS(=O)(=O)c1cn(Cc2ccc(Cl)cc2)c2ccccc12)Nc1ccccc1F]

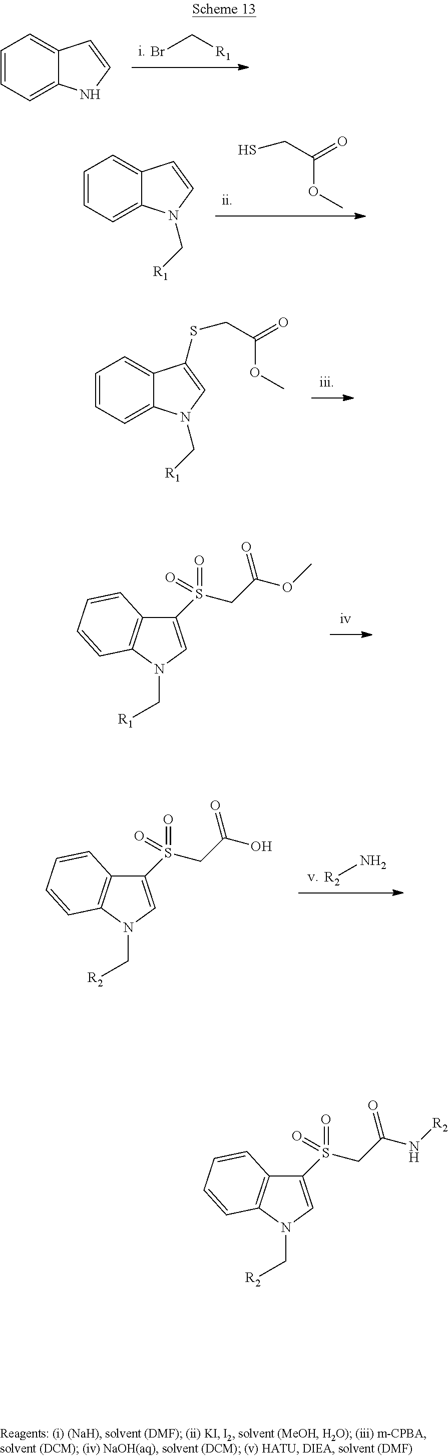

### Step 13-1. 1-(4-chlorobenzyl)-1H-indole (INT 13-A)

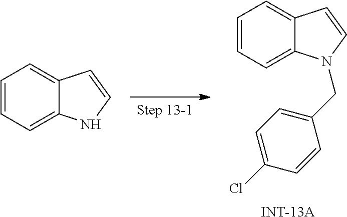

A 250 mL round bottom flask containing methyl 1H-indole (5.0 g, 1.0 equiv., 42.7 mmol) in DMF (15 mL) was added 60% NaH in oil (1.79 g, 1.0 equiv., 42.7 mmol) and then stirred at rt for 5 min. 1-(bromomethyl)-4-chlorobenzene (8.77 g, 1.0 equiv., 42.7 mmol) in DMF (5 mL) was slowly added. Stirred at rt for 20 min. Monitored by LCMS. 20 min: Coupling complete. Quenched with water (100 mL) slowly at 1 h. Product was extracted with DCM (300 mL), washed with brine (200 mL), dried over Na₂SO₄, filtered, and concentrated. The resulting crude residue was purified by silica gel chromatography using ISCO eluting with (0-10% EtOAc/hexanes) to yield 7.0 g (68%) of 1-(4-chlorobenzyl)-1H-indole (INT-13A). LCMS (m/z) calculated for C₁₅H₁₂ClN: 241.07; found 242.3 [M+H]⁺, t_{R}=5.70 min (Method 3).

### Step 13-2. Methyl 2-((1-(4-chlorobenzyl)-1H-indol-3-yl)thio)acetate (INT 13-B)

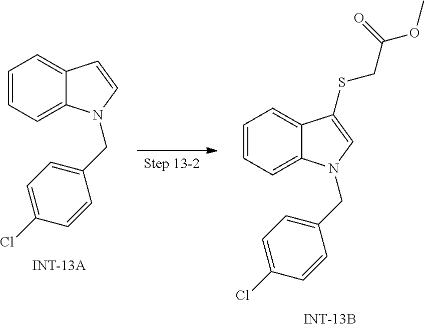

A 250 mL round bottom flask containing 1-(4-chlorobenzyl)-1H-indole (INT-13A) (3.0 g, 1.0 equiv., 1.24 mmol), and methyl 2-mercaptoacetate (1.32 g, 1.0 equiv., 1.24 mmol) in MeOH (80 mL) and distilled water (20 mL) was added iodine (3.15 g, 2.5 equiv., 3.15 mmol), followed by KI (2.06 g, 1.0 equiv., 1.24 mmol). The mixture was stirred at rt for 16 h and then concentrated in vacuo. The remaining aqueous solution was diluted with saturated aqueous NaHCO₃ (100 mL) and product was extracted with EtOAc (200 mL). The organic solution was dried over Na₂SO₄, filtered and concentrated. The resulting crude residue was purified by silica gel chromatography using ISCO eluting with 0-100% EtOAc/hexanes to yield 2.51 g (59% yield) of methyl 2-((1-(4-chlorobenzyl)-1H-indol-3-yl)thio)acetate (INT-13B). LCMS (m/z) calculated for C₁₈H₁₆ClNO₂S: 345.06; found 346.4 [M+H]⁺, t_{R}=5.52 min (Method 3).

### Step 13-3. Methyl 2-((1-(4-chlorobenzyl)-1H-indol-3-yl)sulfonyl)acetate (INT-13C)

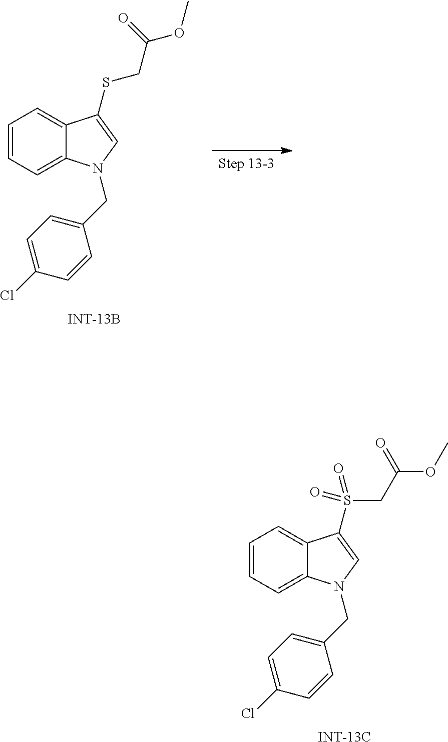

A 100-mL round bottom flask containing methyl 2-((1-(4-chlorobenzyl)-1H-indol-3-yl)thio)acetate (INT-13B) (2.1 g, 1.0 equiv., 6.07 mmol) in DCM (20 ml) was added m-CPBA (2.79 g, 2.0 equiv., 12.1 mmol). The mixture was stirred at rt for 1 h at rt which point LCMS analysis indicated reaction reached completion. The reaction was quenched with saturated aqueous NaHCO₃ (100 mL) and extracted with DCM (150 mL). The organic phase was dried over Na₂SO₄, filtered, and concentrated under reduced pressure. The resulting crude residue was purified by silica gel chromatography using ISCO eluting with 0-100% EtOAc/hexanes to yield 1.52 g (66%) of methyl 2-((1-(4-chlorobenzyl)-1H-indol-3-yl)sulfonyl)acetate (INT-13C). LCMS (m/z) calculated for C₁₈H₁₆ClNO₄S: 377.05; found 378.3 [M+H]⁺, t_{R}=4.40 min (Method 3).

### Step 13-4. 2-((1-(4-chlorobenzyl)-1H-indol-3-yl)sulfonyl)acetic acid (INT-13D)

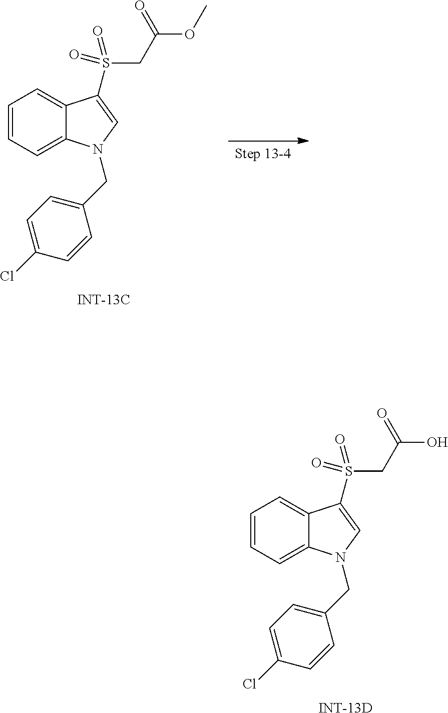

A 250 mL round bottom flask containing methyl 2-((1-(4-chlorobenzyl)-1H-indol-3-yl)sulfonyl)acetate (INT-13C) (1.52 g, 1.0 equiv., 4.0 mmol) in MeOH (20 ml) was added NaOH (0.81 g, 5 equiv., 20 mmol) in water (9 mL). The mixture was stirred at 80° C. for 30 minutes at which point LCMS analysis indicated consumption of starting material. PH of the reaction mixture was adjusted to 1 with the addition of 2 N HCl(aq).

The product was extracted with EtOAc (100 mL) and concentrated under vacuo. The crude residue was purified by recrystallization from EtOAc/hexanes to yield 1.3 g (89% yield) of chlorobenzyl)-1H-indol-3-yl)sulfonyl)acetic acid (INT-13D). LCMS (m/z) calculated for C₁₇H₁₄ClNO₄S: 363.03; found 364.4 [M+H]⁺, t_{R}=11.57 min (method 3).

### Step 13-5. 2-((1-(4-chlorobenzyl)-1H-indol-3-yl)sulfonyl)-N-(2-fluorophenyl)acetamide (Compound 237)

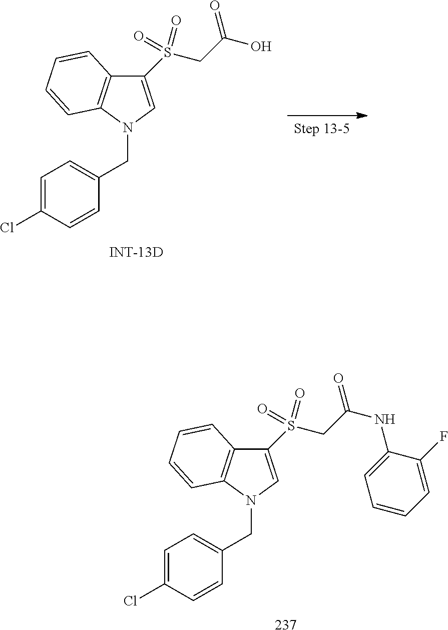

A 25-mL round bottom flask containing 2-((1-(4-chlorobenzyl)-1H-indol-3-yl)sulfonyl)acetic acid, (INT-13D), (75 mg, 1.0 equiv., 0.21 mmol) and HATU (86 mg, 1.1 equiv., 2.3 mmol) was added DMF (6 mL). The mixture was stirred at rt and 2-fluoroaniline (25 mg, 1.1 equiv., 23 mmol) was added followed by DIEA (83 mg, 3.1 equiv., 64 mmol). The reaction mixture was stirred at rt for 16 h at which point LCMS analysis indicated consumption of starting material. The solution was diluted with EtOAc (100 mL) and washed consecutively with 75 mL each 1 M NaOH (aq), 1 M HCl(aq), and brine. The organic solution was dried over Na₂SO₄, filtered and concentrated. The resulting crude residue was purified by silica gel chromatography eluting with 0-100% EtOAc/hexanes to afford 33 mg (35% yield) of 2-((1-(4-chlorobenzyl)-1H-indol-3-yl)sulfonyl)—N-(2-fluorophenyl) acetamide (Compound 237). LCMS (m/z) calculated for C₂₃H₁₈ClFN₂O₃S: 456.07; found 457.5 [M+H]⁺, t_{R}=11.74 min (Method 3). ¹H NMR (400 MHz, DMSO-d6) δ 10.03 (s, 1H), 8.31 (s, 1H), 7.84 (d, J=8 Hz, 1H), 7.77 (d, J=4 Hz, 1H), 7.57 (d, J=8 Hz, 1H), 7.34-7.12 (m, 9H), 5.56 (s, 2H), 4.53 (s, 2H).

The compounds listed in Table 13 were made using the procedures of Scheme 13.

TABLE 13

| | Cpd. | Purity | | Observed | | Purity |
|---|---|---|---|---|---|---|
| Structure | No. | RT (min) | MW | m/z | Ion | Method |
| 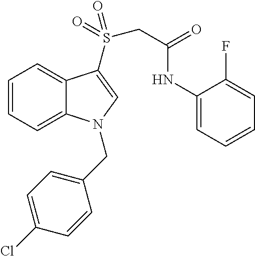 [SMILES: O=C(CS(=O)(=O)c1cn(Cc2ccc(Cl)cc2)c2ccccc12)Nc1ccccc1F] | 237 | 12.64 | 456.92 | 457.4 | [M + H]⁺ | 3 |
| 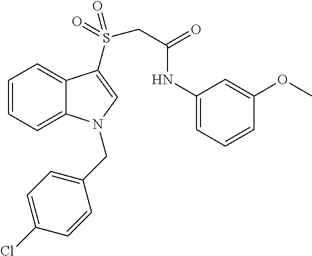 [SMILES: COc1cccc(NC(=O)CS(=O)(=O)c2cn(Cc3ccc(Cl)cc3)c3ccccc23)c1] | 238 | 12.73 | 468.95 | 469.4 | [M + H]⁺ | 3 |
| 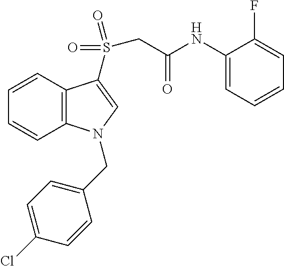 [SMILES: O=C(CS(=O)(=O)c1cn(Cc2ccc(Cl)cc2)c2ccccc12)Nc1ccccc1F] | 239 | 11.26 | 439.91 | 440.3 | [M + H]⁺ | 3 |
| 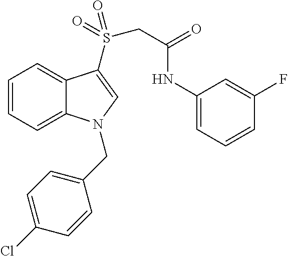 [SMILES: O=C(CS(=O)(=O)c1cn(Cc2ccc(Cl)cc2)c2ccccc12)Nc1cccc(F)c1] | 240 | 12.88 | 456.92 | 457.6 | [M + H]⁺ | 3 |
| 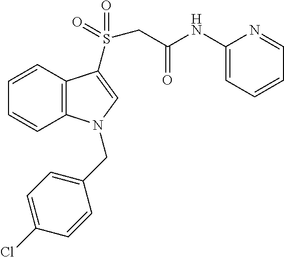 [SMILES: O=C(CS(=O)(=O)c1cn(Cc2ccc(Cl)cc2)c2ccccc12)Nc1ccccn1] | 241 | 12.32 | 439.91 | 440.2 | [M + H]⁺ | 3 |
| 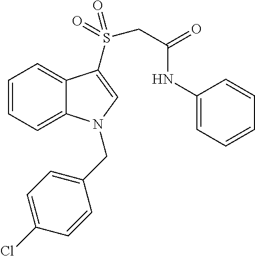 [SMILES: O=C(CS(=O)(=O)c1cn(Cc2ccc(Cl)cc2)c2ccccc12)Nc1ccccc1] | 242 | 12.66 | 438.93 | 439.1 | [M + H]⁺ | 3 |
| 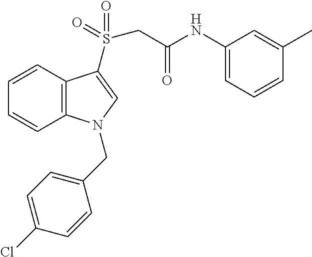 [SMILES: Cc1cccc(NC(=O)CS(=O)(=O)c2cn(Cc3ccc(Cl)cc3)c3ccccc23)c1] | 243 | 10.401 | 452.95 | 453.2 | [M + H]⁺ | 3 |
| 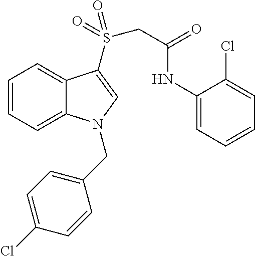 [SMILES: O=C(CS(=O)(=O)c1cn(Cc2ccc(Cl)cc2)c2ccccc12)Nc1ccccc1Cl] | 244 | 13.01 | 473.37 | 473.3 | [M + H]⁺ | 3 |
| 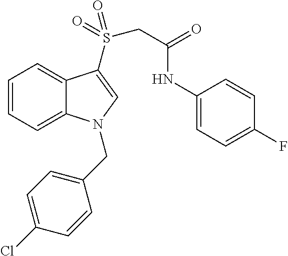 [SMILES: O=C(CS(=O)(=O)c1cn(Cc2ccc(Cl)cc2)c2ccccc12)Nc1ccc(F)cc1] | 245 | 13.91 | 456.92 | 457.6 | [M + H ]⁺ | 3 |
| 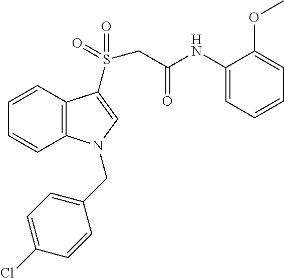 [SMILES: COc1ccccc1NC(=O)CS(=O)(=O)c1cn(Cc2ccc(Cl)cc2)c2ccccc12] | 246 | 12.87 | 468.95 | 469.4 | [M + H]⁺ | 3 |
| 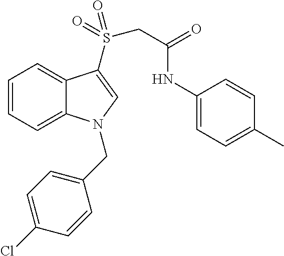 [SMILES: Cc1ccc(NC(=O)CS(=O)(=O)c2cn(Cc3ccc(Cl)cc3)c3ccccc23)cc1] | 247 | 13.16 | 452.95 | 453.4 | [M + H]⁺ | 3 |
| 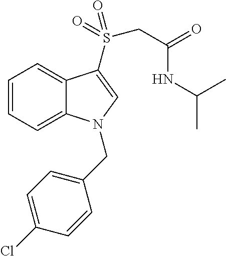 [SMILES: CC(C)NC(=O)CS(=O)(=O)c1cn(Cc2ccc(Cl)cc2)c2ccccc12] | 248 | 12.21 | 404.91 | 405.6 | [M + H]⁺ | 3 |
| 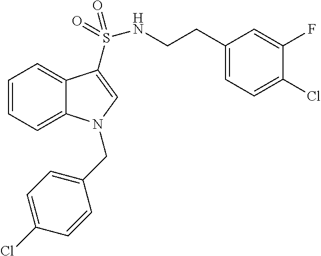 [SMILES: O=S(=O)(NCCc1ccc(Cl)c(F)c1)c1cn(Cc2ccc(Cl)cc2)c2ccccc12] | 314 | 13.92 | 477.38 | 477.1 | [M + H]⁺ | 3 |
| 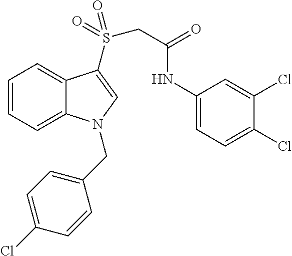 [SMILES: O=C(CS(=O)(=O)c1cn(Cc2ccc(Cl)cc2)c2ccccc12)Nc1ccc(Cl)c(Cl)c1] | 249 | 13.76 | 507.81 | 509.5 | [M + H]⁺ | 3 |
| 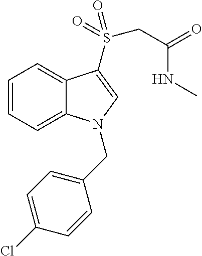 [SMILES: CNC(=O)CS(=O)(=O)c1cn(Cc2ccc(Cl)cc2)c2ccccc12] | 250 | 11.58 | 376.86 | 377.3 | [M + H]⁺ | 3 |
| 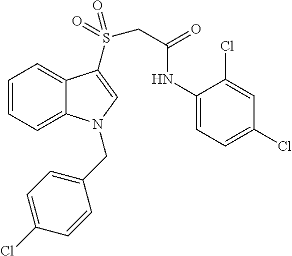 [SMILES: O=C(CS(=O)(=O)c1cn(Cc2ccc(Cl)cc2)c2ccccc12)Nc1ccc(Cl)cc1Cl] | 251 | 13.65 | 507.81 | 507.1 | [M + H]⁺ | 3 |
| 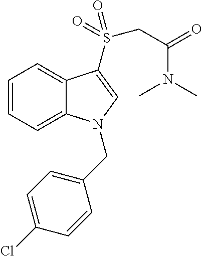 [SMILES: CN(C)C(=O)CS(=O)(=O)c1cn(Cc2ccc(Cl)cc2)c2ccccc12] | 252 | 13.09 | 390.88 | 391.4 | [M + H]⁺ | 3 |
| 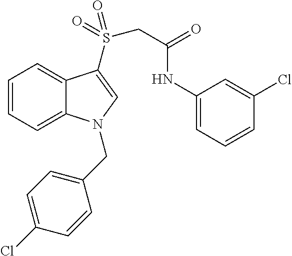 [SMILES: O=C(CS(=O)(=O)c1cn(Cc2ccc(Cl)cc2)c2ccccc12)Nc1cccc(Cl)c1] | 253 | 31.25 | 473.37 | 473.3 | [M + H]⁺ | 3 |
| 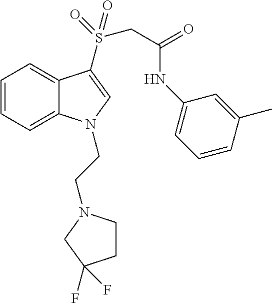 [SMILES: Cc1cccc(NC(=O)CS(=O)(=O)c2cn(CCN3CCC(F)(F)C3)c3ccccc23)c1] | 254 | 10.49 | 461.53 | 462.5 | [M + H]⁺ | 3 |
| 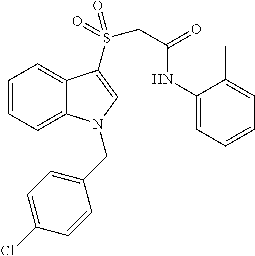 [SMILES: Cc1ccccc1NC(=O)CS(=O)(=O)c1cn(Cc2ccc(Cl)cc2)c2ccccc12] | 255 | 12.84 | 452.95 | 453.3 | [M + H]⁺ | 3 |
| 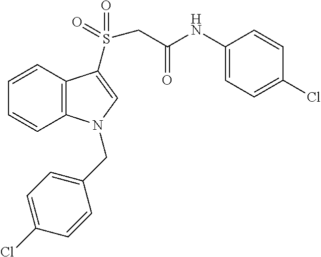 [SMILES: O=C(CS(=O)(=O)c1cn(Cc2ccc(Cl)cc2)c2ccccc12)Nc1ccc(Cl)cc1] | 256 | 13.33 | 473.37 | 473.2 | [M + H]⁺ | 3 |

### Example 14

### Synthesis of Representative Compound 312

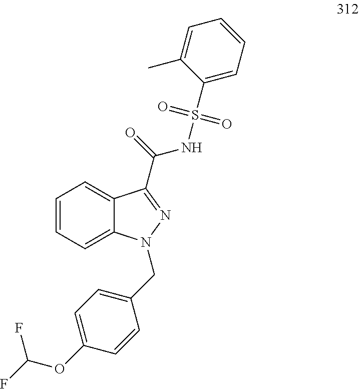 [SMILES: Cc1ccccc1S(=O)(=O)NC(=O)c1nn(Cc2ccc(OC(F)F)cc2)c2ccccc12]

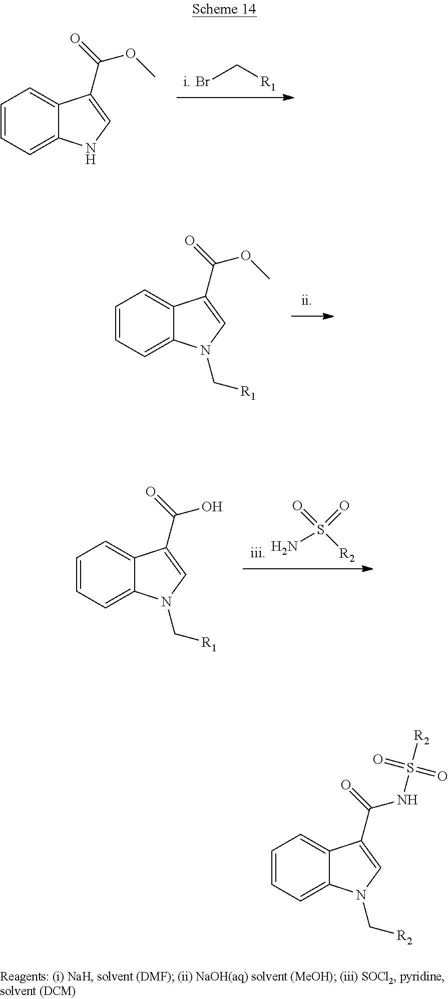

### Step 14-1. Methyl 1-(4-(difluoromethoxy) benzyl)-1H-indole-3-carboxylate (INT-14A)

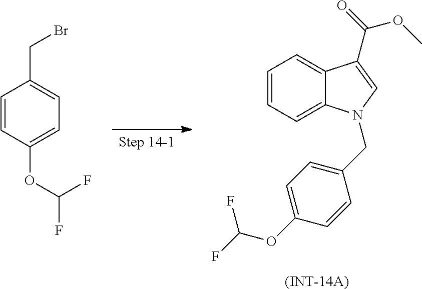

A 50-mL round bottom flask methyl 1H-indole-3-carboxylate (309 mg, 1.0 equiv., 1.75 mmol) in DMF (5 mL) was added 60% NaH in oil (95 mg, 1.5 equiv., 2.38 mmol) and then stirred at rt for 5 min. To this mixture was added 1-(bromomethyl)-4-(difluoromethoxy) benzene (300 mg, 1.1 equiv., 1.59 mmol). The reaction was stirred at rt for 2 h (additional 20 mg 1-(bromomethyl)-4-(difluoromethoxy)benzene added a 30 min). The reaction was quenched with saturated aqueous NH₄Cl (50 mL). Product was extracted with EtOAc (60 mL) and organic phase was washed with brine (30 mL), dried over Na₂SO₄, filtered, and concentrated. The resulting crude residue was purified by silica gel chromatography using ISCO eluting with 0-10% MeOH/DCM to yield 400 mg (76% yield) of methyl 1-(4-(difluoromethoxy)benzyl)-1H-indole-3-carboxylate (INT-14A). LCMS (m/z) calculated for C₁₈H₁₅F₂NO₃: 331.10; found 332.4 [M+H]⁺, t_{R}=4.94 min (method 3).

### Step 14-2. 1-(4-(difluoromethoxy)benzyl)-1H-indole-3-carboxylic acid (INT-14B)

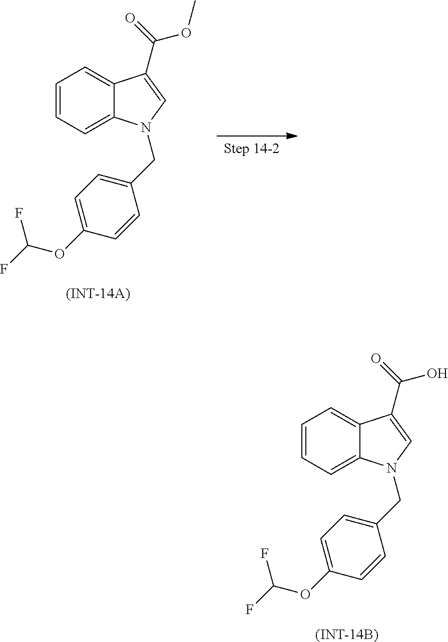

To a 100-mL round bottom flask containing a solution of methyl 1-(4-(difluoromethoxy)benzyl)-1H-indole-3-carboxylate, (INT-14A), (400 mg, 1.0 equiv., 1.21 mmol) in MeOH (40 mL) was added an aqueous solution of 1 M NaOH (6.0 mL, 5 equiv., 6.0 mmol) and H₂O (5 mL). The mixture was stirred at 100° C. for 1.5 h. The reaction was concentrated to remove MeOH, then diluted with water (50 mL). The solution was acidified to pH 1 using 1 M HCl(aq). Product was extracted with EtOAc (60 mL), and the organic solution was washed with brine (50 mL), dried over Na₂SO₄, filtered, and concentrated to yield 330 mg (86% yield) of 1-(4-(difluoromethoxy)benzyl)-1H-indole-3-carboxylic acid (INT-14B). LCMS (m/z) calculated for C₁₇H₁₃F₂NO₃: 317.09; found 318.3 [M+H]⁺, t_{R}=4.38 min (method 3).

### Step 14-3. 1-(4-(difluoromethoxy)benzyl)-N-(o-tolylsulfonyl)-1H-indole-3-carboxamide (Compound 312)

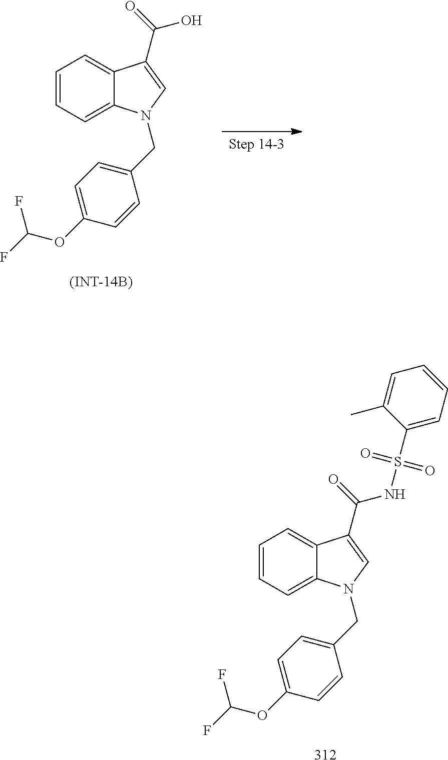

To a 50-mL round bottom flask containing 1-(4-(difluoromethoxy)benzyl)-1H-indole-3-carboxylic acid, (INT-14B), (50 mg, 1.0 equiv., 0.16 mmol) was added SOCl₂ (5 mL) and stirred at rt for 5 min. Volatile was removed under vacuo, and the residue was added 2-methylbenzenesulfonamide (30 mg, 1.1 equiv., 0.17 mmol), DMAP (21 mg, 1.1 equiv., 0.17 mmol), and anhydrous DCM (5 mL). At 1 h, 2-methylbenzenesulfonamide (30 mg, 1 equiv., 0.17 mmol) was added followed by Et₃N (50 μmol). After an additional 1 h, solvents were removed under vacuo. The residue was diluted with EtOAc (50 mL) and washed consecutive with 1 M HCl (aq., 30 mL), 1 M NaOH (aq., 30 mL), saturated NaHCO₃ (aq., 30 mL), and brine (aq., 30 mL), dried over Na₂SO₄, filtered, and concentrated. The resulting crude residue was purified by silica gel chromatography using ISCO eluting with 0-100% EtOAc/hexanes. Product fractions were combined, concentrated and the residue was dissolved in CH₃CN/H₂O and lyophilized to yield 26 mg (36%) of 1-(4-(difluoromethoxy)benzyl)-N-(o-tolylsulfonyl)-1H-indole-3-carboxamide (Compound 312) as a solid. LCMS (m/z) calculated for C₂₄H₂₀F₂N₂O₄S: 470.11; found 471.4 [M+H]⁺, t_{R}=11.81 min (method 3). ¹H NMR (400 MHz, DMSO-d6) δ 12.12 (s, 1H), 8.56 (s, 1H), 8.06 (d, J=8 Hz, 1H), 7.96 (d, J=8 Hz, 1H), 7.61-7.55 (m, 2H), 7.46 (t, J=8 Hz, 1H), 7.41-7.03 (m, 8H), 5.50 (s, 2H), 2.65 (s, 3H).

The compounds listed in Table 14 were made using the procedures of Scheme 14.

TABLE 14

| | Cpd. | Purity RT | | Observed | | Purity |
|---|---|---|---|---|---|---|
| Structure | No. | (min) | MW | m/z | Ion | Method |
| 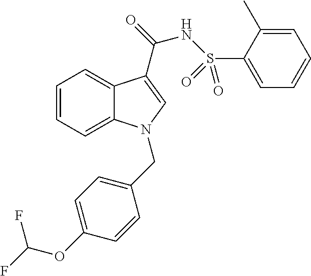 [SMILES: Cc1ccccc1S(=O)(=O)NC(=O)c1cn(Cc2ccc(OC(F)F)cc2)c2ccccc12] | 312 | 13.2 | 470.49 | 471.4 | [M + H]⁺ | 3 |
| 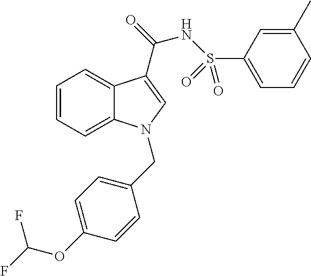 [SMILES: Cc1cccc(S(=O)(=O)NC(=O)c2cn(Cc3ccc(OC(F)F)cc3)c3ccccc23)c1] | 313 | 13.06 | 470.49 | 471.5 | [M + H]⁺ | 3 |

### Example 15

### Synthesis of Compound 315

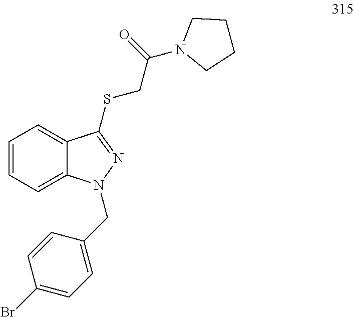 [SMILES: O=C(CSc1nn(Cc2ccc(Br)cc2)c2ccccc12)N1CCCC1]

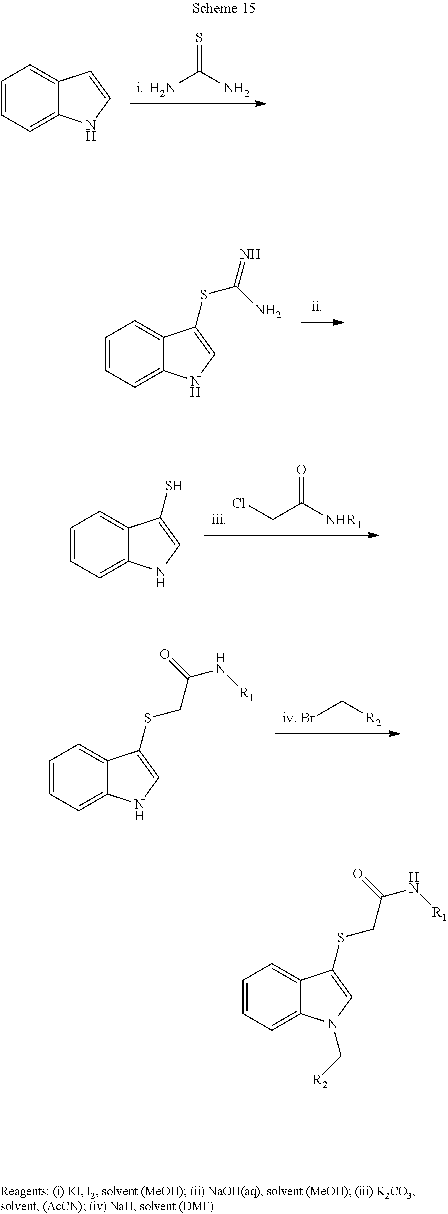

### Step 15-1. 1H-indol-3-yl carbamimidothioate (INT-15A)

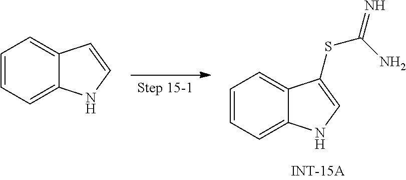 [SMILES: c1ccc2[nH]ccc2c1 ; "Step 15-1" ; N=C(N)Sc1c[nH]c2ccccc12]

To a stirring solution of 1H-indole (2.0 g, 1.0 equiv., 17.0 mmol) and thiourea (1.3 g, 1.0 equiv., 17.0 mmol) in MeOH (60 mL) was added dropwise a solution of potassium iodide (2.8 g, 1.0 equiv., 17.0 mmol) and diiodine (4.3 g, 1.0 equiv., 17.0 mmol) in water (60 mL). Required additional MeOH (20 mL) and H₂O (20 mL) to dissolve the iodine. The reaction mixture was stirred at rt for 2 h. TLC of starting indole Rf=0.80 (3:2 EtOAc/hexanes). LCMS showed reaction completion. The mixture was filtered, and the filtrate was concentrated in vacuo to afford 1.6 g (63% yield) of 1H-indol-3-yl carbamimidothioate (INT-15-A) as a white solid that was carried on without further purification. LCMS (m/z) calculated for C₉H₉N₃S: 191.2; found 192.1 [M+H]⁺, t_{R}=1.63 min (method 2).

### Step 15-2. 1H-indole-3-thiol (INT-15B)

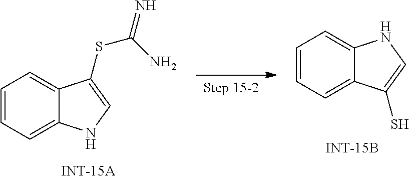

To 1H-indol-3-yl carbamimidothioate, (INT-15A), (3.3 g, 1.0 equiv., 17.0 mmol) was added 2N NaOH (60 mL). The reaction mixture was heated at 85° C. for 30 min. LCMS showed reaction completion. The mixture was cooled to rt with an ice bath and acidified with 6N HCl (25 mL). The resulting precipitate was filtered, washed with water (2×60 mL), filtered, and dried in vacuo to afford crude product. The crude product was purified by silica gel chromatography (0->50% EtOAc in hexanes) to afford 1.6 g (63% yield) 1H-indole-3-thiol (INT-15B) as a white solid. LCMS (m/z) calculated for C₈H₇NS: 149.2; found 150.1 [M+H]⁺, t_{R}=3.97 min (Method 2).

### Step 15-3. 2-((1H-indol-3-yl)thio)-1-(pyrrolidin-1-yl)ethan-1-one (INT-15C)

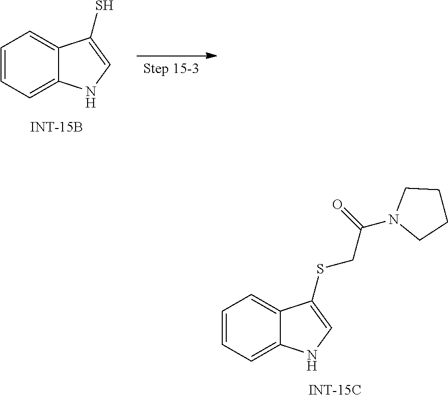

To a stirring solution of 1H-indole-3-thiol, (INT-15B), (50.0 mg, 1.0 equiv., 0.34 mmol) in acetonitrile (2 mL) was added potassium carbonate (460.0 mg, 10.0 equiv., 3.4 mmol) followed by 2-chloro-1-(pyrrolidin-1-yl)ethan-1-one (49.0 mg, 10.0 equiv., 0.34 mmol). The vial was capped and heated at 85° C. overnight. LCMS showed reaction completion. Filtered through Celite rinsing with EtOAc. Purified by silica gel chromatography (0-100% 10% MeOH in EtOAc and hexanes) to afford 2-((1H-indol-3-yl)thio)-1-(pyrrolidin-1-yl)ethan-1-one (INT-15C) as a solid. LCMS (m/z) calculated for C₁₄H₁₆N₂O₂S: 260.3; found 261.1 [M+H]⁺, t_{R}=6.61 min (Method 2).

### Step 15-4. 2-(4(1H-indol-3-yl)thio)-1-(pyrrolidin-1-yl)ethan-1-one (Compound 315)

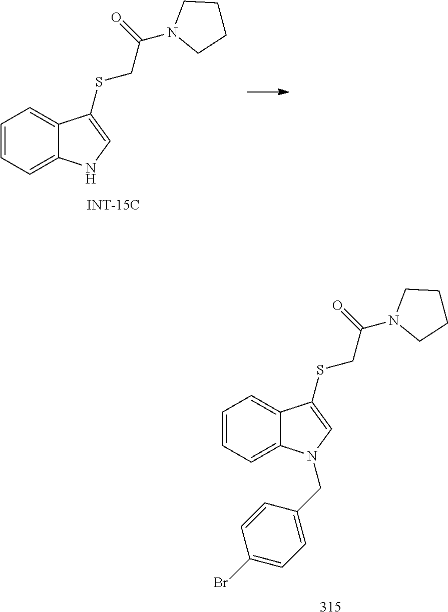

To a stirring solution of 2-((1H-indol-3-yl)thio)-1-(pyrrolidin-1-yl)ethan-1-one, (INT 15-C), (78.7 mg, 1.0 equiv., 0.3 mmol) in 15 mL DMF was added sodium hydride (15 mg, 60% wt, 1.2 equiv., 0.36 mmol) followed by 1-bromo-4-(bromomethyl)benzene (90.7 mg, 1.2 equiv., 0.36 mmol). The reaction mixture was stirred at rt for 4 h. LCMS showed reaction completion. Saturated ammonium chloride was added dropwise, the aqueous layer was extracted with EtOAc (3×3 mL), dried over sodium sulfate, filtered, and concentrated in vacuo to afford crude product. 56.6 mg colorless oil. Purified by silica gel chromatography (0-70% 10% MeOH in EtOAc and hexanes). Impurity came off right before it but had a much higher Rf Impurity with a similar retention time on the LCMS. 56.6 mg colorless oil. Added ˜0.25 mL EtOAc followed by ˜0.25 mL of Et₂O. The product crystallized to yield 57 mg (44% yield) of 2-(4(1H-indol-3-yl)thio)-1-(pyrrolidin-1-yl)ethan-1-one (Compound 315) as a solid. LCMS (m/z) calculated for C₂₁H₂₁BrN₂OS: 429.8; found 431.1 [M+H], t_{R}=10.43 min (Method 2).

### Example 16

### Synthesis of Compound 530

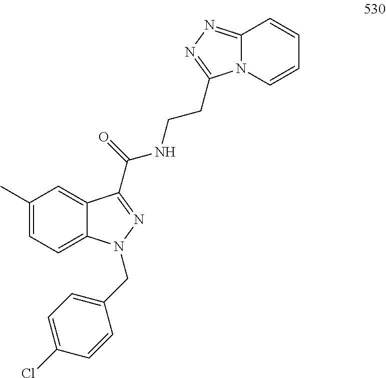 [SMILES: Cc1ccc2c(c1)c(C(=O)NCCc1nnc3ccccn13)nn2Cc1ccc(Cl)cc1]

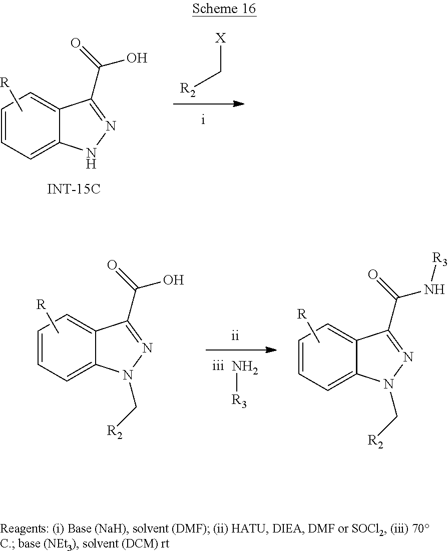

### Step 16-1. Synthesis of 1-(4-chlorobenzyl)-5-methyl-1H-indazole-3-carboxylic acid

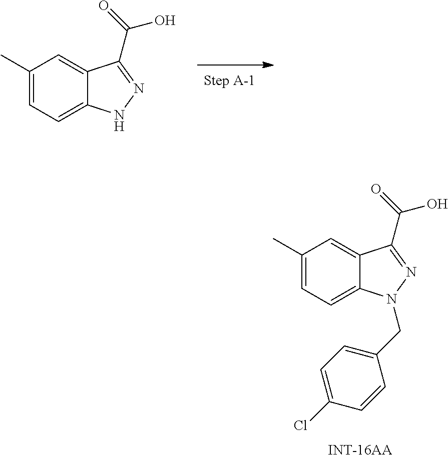

To a stirring solution of 5-methyl-1H-indazole-3-carboxylic acid (200 mg, 1 Eq, 1.14 mmol) at 0° C. in DMF (2 mL) was added NaH (60% in mineral oil, 84 mg, 3.1 Eq, 3.5 mmol). After stirring for 15 min at rt, a solution of 1-chloro-4-(chloromethyl)benzene (192 mg, 1.05 eq, 1.2 mmol) in DMF (2 mL) was added. After 1 h, the reaction mixture was quenched with H₂O (5 mL) and washed with EtOAc (3×10 mL). The aqueous layer was pH adjusted to pH 2 (6 N HCl) and then extracted with EtOAc. The organic layer was dried over sodium sulfate, filtered, and concentrated in vacuo to afford crude material. The crude material was purified by SiO₂ chromatography (0-10% MeOH/DCM) to afford 200 mg (59% yield) of 1-(4-chlorobenzyl)-5-methyl-1H-indazole-3-carboxylic acid (INT-16AA) as a white solid; LCMS (m/z) calculated for C₁₆H₁₃ClN₂O₂: 300.07; found 301.0 [M+H]⁺, t_{R}=6.36 min (Method 3).

### Step 16-2. Synthesis of N-(2-([1,2,4]triazolo[4,3-a]pyridin-3-yl)ethyl)-1-(4-chlorobenzyl)-5-methyl-1H-indazole-3-carboxamide

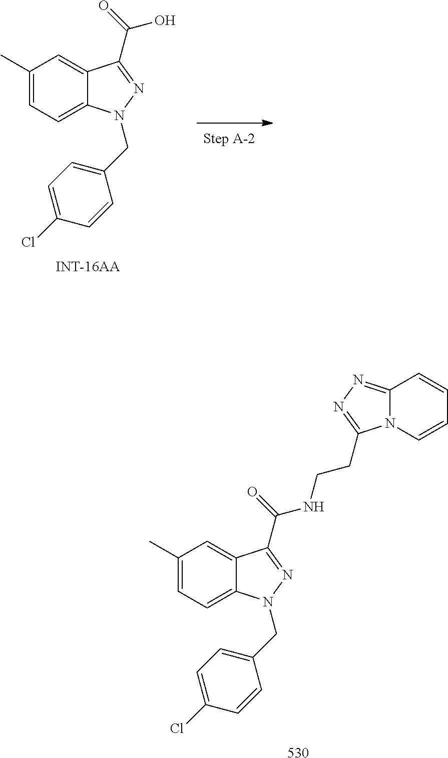

To a stirring solution of 1-(4-chlorobenzyl)-5-methyl-1H-indazole-3-carboxylic acid (100 mg, 1 Eq, 0.33 mmol) in DMF (0.5 mL) was added HATU (139 mg, 1.1 Eq, 0.37 mmol). After 15 min, 2-([1,2,4] triazolo[4,3-a]pyridin-3-yl)ethan-1-amine (59 mg, 1.1 eq, 0.37 mmol) and DIEA (129 mg, 3 eq, 1 mmol) were added. After 3 h, the reaction mixture was quenched with water and extracted with EtOAc. The organic layer was separated, dried over sodium sulfate, filtered, and concentrated in vacuo to afford crude material. The crude product was purified by silica gel column chromatography (0-10% MeOH/DCM) to afford 25 mg (17% yield) of N-(2-([1,2,4]triazolo[4,3-a]pyridin-3-yl)ethyl)-1-(4-chlorobenzyl)-5-methyl-1H-indazole-3-carboxamide (Compound 530) as a white solid; LCMS (m/z) calculated for C₂₄H₂₁ClN₆O: 444.1; found 444.9 [M+H]⁺, t_{R}=12.03 min (Method 3).

The compounds listed in Table 16 were made using the procedures of Scheme 16.

TABLE 16

| | Cpd. | Purity RT | | Observed | | Purity |
|---|---|---|---|---|---|---|
| Structure | No. | (min) | MW | m/z | Ion | Method |
| 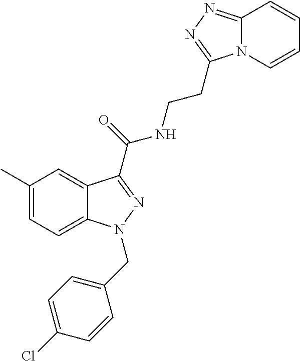 [SMILES: Cc1ccc2c(c1)c(C(=O)NCCc1nnc3ccccn13)nn2Cc1ccc(Cl)cc1] | 530 | 12.03 | 444.92 | 444.9 | [M + H]⁺ | 3 |
| 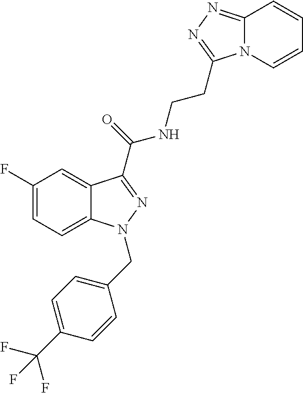 [SMILES: O=C(NCCc1nnc2ccccn12)c1nn(Cc2ccc(C(F)(F)F)cc2)c2ccc(F)cc12] | 531 | 11.79 | 482.443 | 483.1 | [M + H]⁺ | 3 |
| 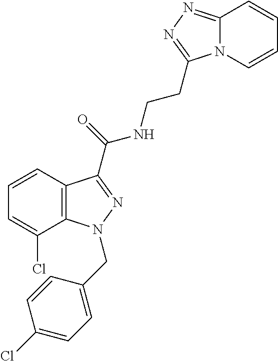 [SMILES: O=C(NCCc1nnc2ccccn12)c1nn(Cc2ccc(Cl)cc2)c2c(Cl)cccc12] | 539 | 12.91 | 465.34 | 464.5 | [M + H]⁺ | 3 |
| 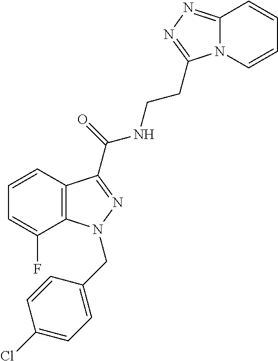 [SMILES: O=C(NCCc1nnc2ccccn12)c1nn(Cc2ccc(Cl)cc2)c2c(F)cccc12] | 540 | 11.6 | 448.89 | 448.8 | [M + H]⁺ | |
| 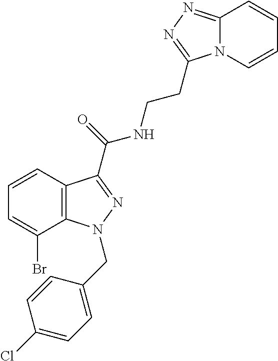 [SMILES: O=C(NCCc1nnc2ccccn12)c1nn(Cc2ccc(Cl)cc2)c2c(Br)cccc12] | 541 | 12.12 | 509.79 | 508.9 | [M + H]⁺ | 3 |
| 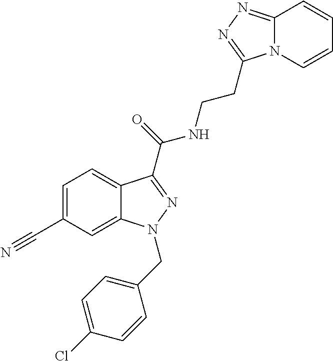 [SMILES: N#Cc1ccc2c(C(=O)NCCc3nnc4ccccn34)nn(Cc3ccc(Cl)cc3)c2c1] | 542 | 10.02 | 455.91 | 456.3 | [M + H]⁺ | 3 |
| 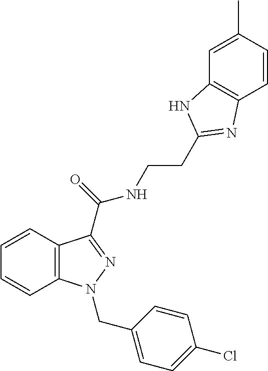 [SMILES: Cc1ccc2nc(CCNC(=O)c3nn(Cc4ccc(Cl)cc4)c4ccccc34)[nH]c2c1] | 560 | 11.92 | 443.94 | 443.9 | [M + H]⁺ | 3 |
| 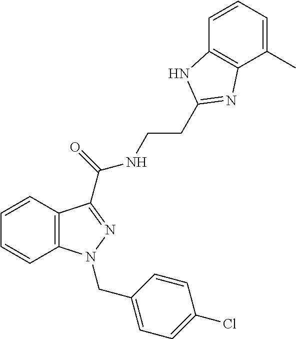 [SMILES: Cc1cccc2[nH]c(CCNC(=O)c3nn(Cc4ccc(Cl)cc4)c4ccccc34)nc12] | 561 | 11.87 | 443.94 | 444 | [M + H]⁺ | 3 |
| 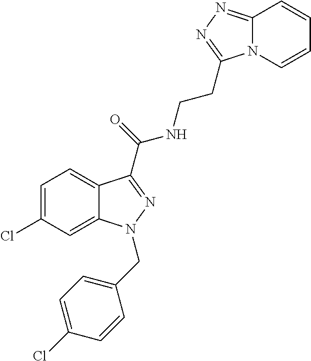 [SMILES: O=C(NCCc1nnc2ccccn12)c1nn(Cc2ccc(Cl)cc2)c2cc(Cl)ccc12] | 562 | 12.62 | 465.34 | 464.8 | [M + H]⁺ | 3 |
| 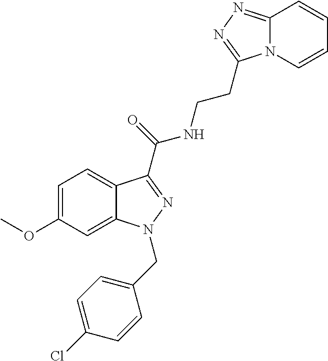 [SMILES: COc1ccc2c(C(=O)NCCc3nnc4ccccn34)nn(Cc3ccc(Cl)cc3)c2c1] | 563 | 11.69 | 460.92 | 461.1 | [M + H]⁺ | 3 |
| 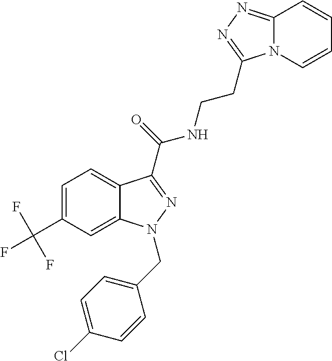 [SMILES: O=C(NCCc1nnc2ccccn12)c1nn(Cc2ccc(Cl)cc2)c2cc(C(F)(F)F)ccc12] | 564 | 12.61 | 498.89 | 498.4 | [M + H]⁺ | 3 |
| 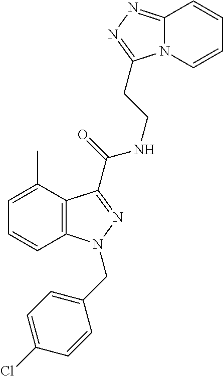 [SMILES: Cc1cccc2c1c(C(=O)NCCc1nnc3ccccn13)nn2Cc1ccc(Cl)cc1] | 565 | 11.99 | 444.92 | 444.8 | [M + H]⁺ | 3 |
| 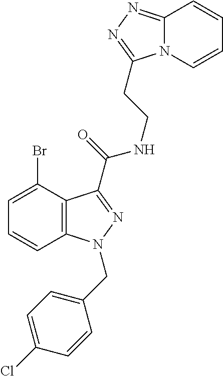 [SMILES: O=C(NCCc1nnc2ccccn12)c1nn(Cc2ccc(Cl)cc2)c2cccc(Br)c12] | 566 | 10.96 | 509.79 | 511 | [M + H]⁺ | 3 |

### Example 17

### Synthesis of Compound 532

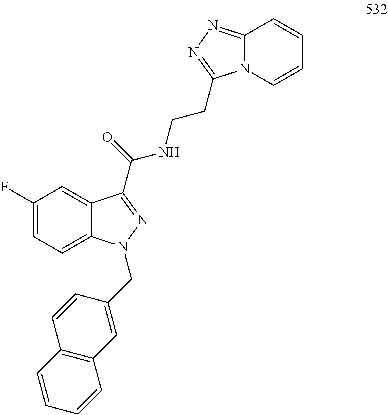 [SMILES: O=C(NCCc1nnc2ccccn12)c1nn(Cc2ccc3ccccc3c2)c2ccc(F)cc12]

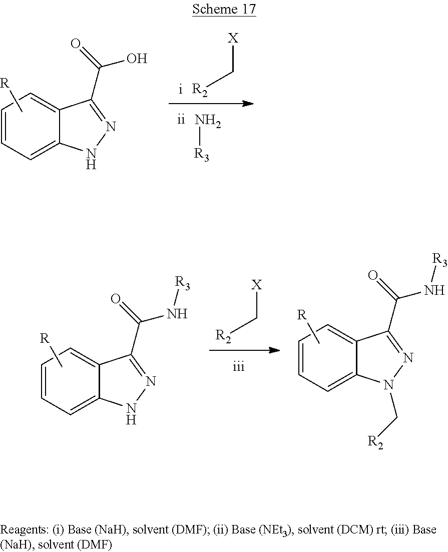

### Step 17-1. Synthesis of N-(2-([1,2,4]triazolo[4,3-a]pyridin-3-yl)ethyl)-5-fluoro-1H-indazole-3-carboxamide

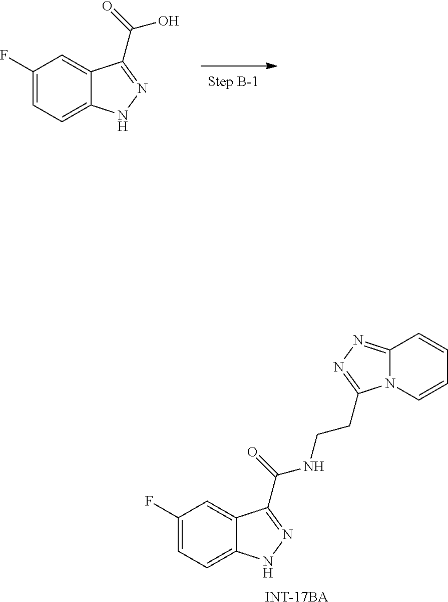

To a vial containing 5-fluoro-1H-indazole-3-carboxylic acid (50 mg, 1 Eq, 0.28 mmol) was added thionyl chloride (84 mg, 20 Eq, 5.5 mmol). After stirring for 1 h at 80° C., the reaction mixture was concentrated in vacuo. The resulting residue was diluted with DCM and re-concentrated multiple times. The resulting precipitate was dissolved in DCM (1 mL) and 2-([1,2,4]triazolo[4,3-a]pyridin-3-yl)ethan-1-amine (45 mg, 1.0 eq, 0.28 mmol) and TEA (84 mg, 3 eq, 0.83 mmol) were added. After 0.5 h, the reaction mixture was filtered to collect the solid precipitate, which was washed with DCM, acetone, water, acetone and DCM, followed by air drying to provide 48 mg (53% yield) of N-(2-([1,2,4]triazolo[4,3-a]pyridin-3-yl)ethyl)-5-fluoro-1H-indazole-3-carboxamide (INT-17BA) as a light gray solid; LCMS (m/z) calculated for C₁₆H₁₃FN₆O: 324.1; found 325.0 [M+H]⁺, t_{R}=4.75 min (Method 3).

### Step 17-2. Synthesis of N-(2-([1,2,4]triazolo[4,3-a]pyridin-3-yl)ethyl)-5-fluoro-1-(naphthalen-2-ylmethyl)-1H-indazole-3-carboxamide

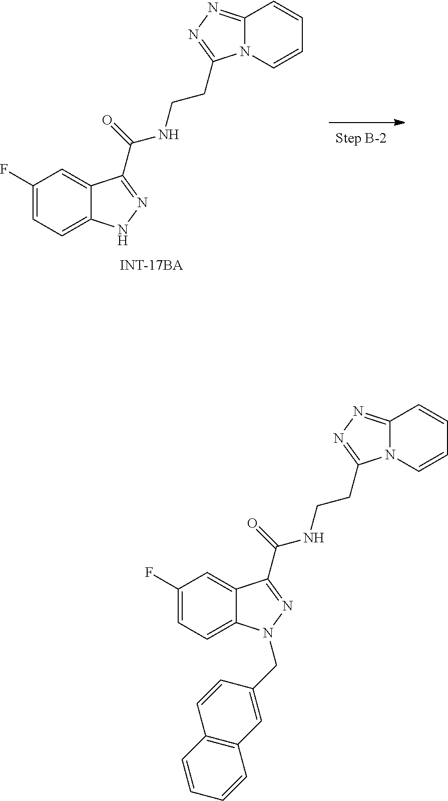

Into a vial containing N-(2-([1,2,4]triazolo[4,3-a]pyridin-3-yl)ethyl)-5-fluoro-1H-indazole-3-carboxamide (48 mg, 1.0 eq, 0.15 mmol) was added DMF (1 mL), followed by NaH (60% solution in mineral oil, 6.5 mg, 1.1 eq, 0.16 mml). After bubbling ceased, 2-(bromomethyl)naphthalene (33 mg, 1.0 eq, 0.15 mmol) was added. After 15 min, the reaction was quenched with water and extracted with DCM (3X). The combined organic extracts were dried over sodium sulfate, filtered, and concentrated in vacuo to afford crude material. The crude product was purified by SiO₂ column chromatography (0-100% 91% MeOH/in EtOAc/DCM) to afford 24 mg (35% yield) of N-(2-([1,2,4]triazolo[4,3-a]pyridin-3-yl)ethyl)-5-fluoro-1-(naphthalen-2-ylmethyl)-1H-indazole-3-carboxamide (Compound 532) as a pale yellow solid; LCMS (m/z) calculated for C₂₇H₂₁FN₆O: 464.2; found 465 [M+H]⁺, t_{R}=12.19 min (Method 3).

The compounds listed in Table 17 were made using the procedures of Scheme 17.

TABLE 17

| | Cpd. | Purity RT | | Observed | | Purity |
|---|---|---|---|---|---|---|
| Structure | No. | (min) | MW | m/z | Ion | Method |
| 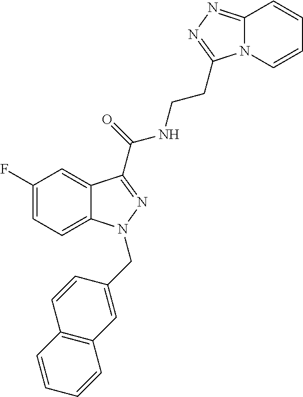 [SMILES: O=C(NCCc1nnc2ccccn12)c1nn(Cc2ccc3ccccc3c2)c2ccc(F)cc12] | 532 | 12.07 | 464.504 | 464.7 | [M + H]⁺ | 3 |

### Example 18

### Synthesis of Representative Compound 402

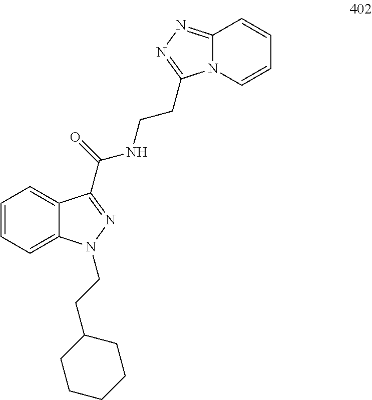 [SMILES: O=C(NCCc1nnc2ccccn12)c1nn(CCC2CCCCC2)c2ccccc12]

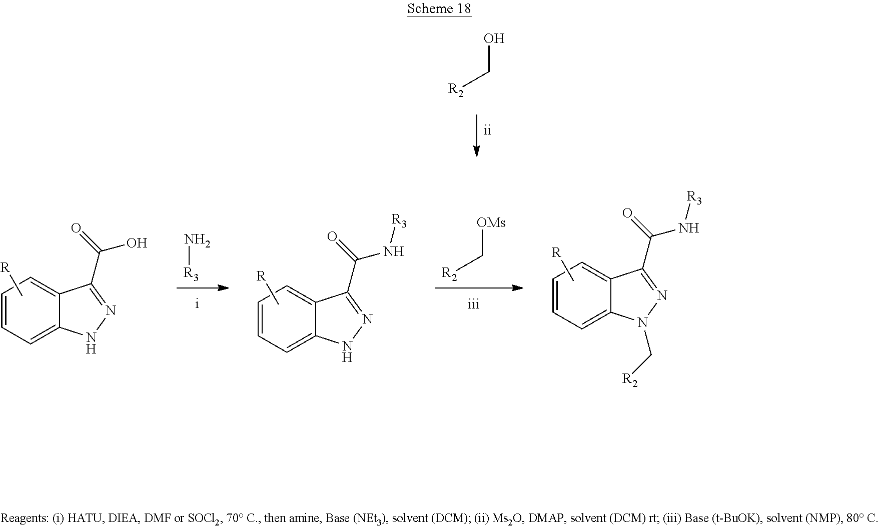

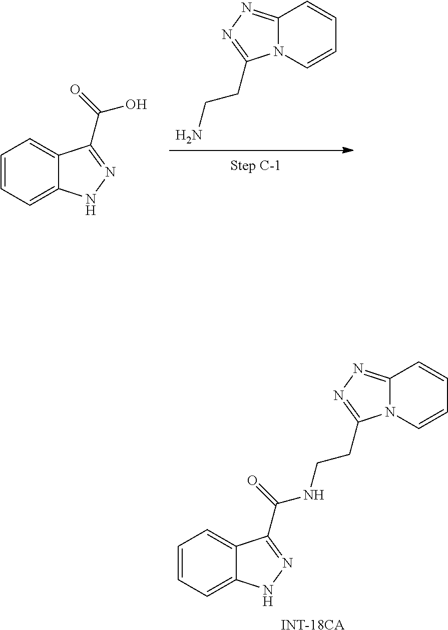

### Step 18-1. Synthesis of N-(2-([1,2,4]triazolo[4,3-a]pyridin-3-yl)ethyl)-1H-indazole-3-carboxamide

Into a solution of 1H-indazole-3-carboxylic acid (5.34 g, 1 eq, 32.91 mmol) in DMF (50 mL) were added EDCI (7.57 g, 1.2 eq, 39.5 mmol), HOBt (5.34 g, 1.2 eq, 39.49 mmol) and TEA (9.99 g, 3 eq, 98.72 mmol, 13.74 mL) at 25° C., followed by 2-([1,2,4]triazolo[4,3-a]pyridin-3-yl)ethanamine (8.0 g, 1 eq, 32.91 mmol, HBr salt). The mixture was stirred at 25° C. for 2 h. The reaction mixture was filtered, and the filter cake was washed with MTBE (3×100 mL) and H₂O (100 mL). The resulting filter cake was collected and dried in vacuo to provide 4 g (40% yield) of N-(2-([1,2,4]triazolo[4,3-a]pyridin-3-yl)ethyl)-1H-indazole-3-carboxamide (INT-18CA) as a white solid; LCMS (m/z) calculated for C₁₆H₁₄N₆O: 306.1; found 307.1 [M+H]⁺, t_{R}=0.28 min (Method 5). ¹H NMR (400 MHz, DMSO-d6) δ 13.58 (s, 1H), 8.62 (t, J=6.0 Hz, 1H), 8.51-8.48 (m, 1H), 8.16 (d, J=8.3 Hz, 1H), 7.73 (td, J=1.0, 9.3 Hz, 1H), 7.61 (d, J=8.4 Hz, 1H), 7.44-7.39 (m, 1H), 7.36-7.32 (m, 1H), 7.26-7.22 (m, 1H), 6.97 (dt, J=0.9, 6.7 Hz, 1H), 3.79 (q, J=6.9 Hz, 2H), 3.40 (t, J=7.0 Hz, 2H).

### Step 18-2. Synthesis of 2-cyclohexylethyl methanesulfonate

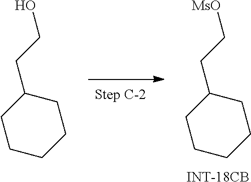

Into a solution of 2-cyclohexylethanol (100 mg, 1 eq, 0.78 mmol, 109 μL) in DCM (5 mL) were added DMAP (9.5 mg, 0.1 eq, 0.078 mmol) and TEA (237 mg, 3 eq, 2.34 mmol, 326 μL). Methylsulfonyl methanesulfonate (150 mg, 1.1 eq, 858 μmol) was added at 0° C. and then the mixture was stirred at 25° C. for 2 h. The reaction mixture was diluted with H₂O (20 mL) and then extracted with DCM (3×10 mL). The combined organic layers were washed with saturated NaCl (2×10 mL), dried over anhydrous sodium sulfate, filtered, and concentrated under vacuum to afford 160 mg (99% yield) of 2-cyclohexylethyl methanesulfonate as a colorless oil that was used without further purification; TLC (EA, ninhydrin stain) R_{f}=0.40.

### Step 18-3. Synthesis of N-(2-([1,2,4]triazolo[4,3-a] pyridin-3-yl)ethyl)-1-(2-0cyclohexylethyl)-1H-indazole-3-carboxamide

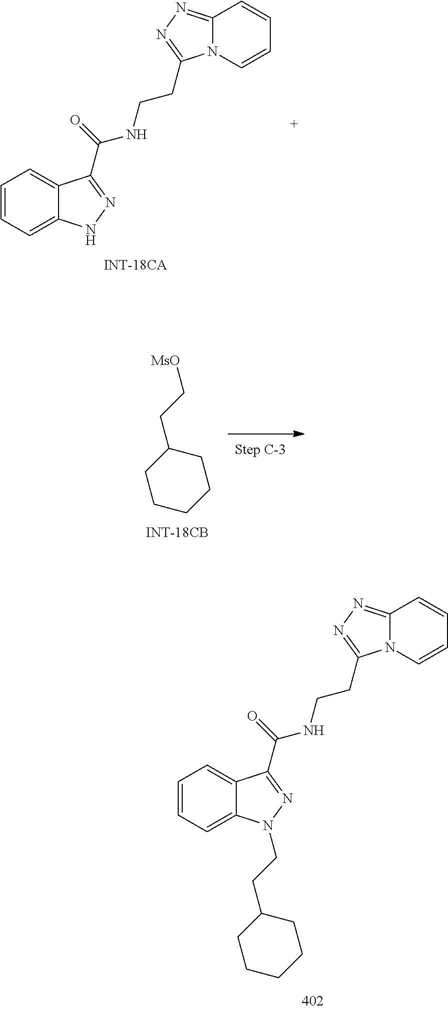

Into a solution of N-[2-([1,2,4]triazolo[4,3-a]pyridin-3-yl)ethyl]-1H-indazole-3-carboxamide (20 mg, 1 eq, 65.29 μmol) and 2-cyclohexylethyl methanesulfonate (13.47 mg, 1 eq, 65.29 μmol) in NMP (1 mL) was added t-BuOK (18.32 mg, 2.5 eq, 163.23 μmol). The reaction was stirred at 80° C. for 2 The reaction mixture was filtered through a filter membrane. The filtrate was purified by reverse phase prep-HPLC (ACN/H₂O with formic acid). The resulting material was lyophilized to afford 11.6 mg (43% yield) of N-(2-([1,2,4]triazolo[4,3-a]pyridin-3-yl)ethyl)-1H-indazole-3-carboxamide (Compound 402) as a pale brown solid; LCMS (m/z) calculated for C₂₄H₂₈N₆O: 416.2; found 417.2 [M+H]⁺, t_{R}=0.46 min (Method 5). ¹H NMR (400 MHz, DMSO-d6) δ=8.49 (d, J=7.1 Hz, 2H), 8.15 (d, J=8.3 Hz, 1H), 7.73 (t, J=8.3 Hz, 2H), 7.47-7.42 (m, 1H), 7.33 (ddd, J=0.9, 6.5, 9.3 Hz, 1H), 7.26 (t, J=7.4 Hz, 1H), 6.98-6.93 (m, 1H), 4.51-4.45 (m, 2H), 3.77 (q, J=6.9 Hz, 2H), 3.39 (br t, J=7.1 Hz, 2H), 1.79-1.71 (m, 4H), 1.65 (br d, J=5.9 Hz, 2H), 1.60 (br d, J=4.0 Hz, 1H), 1.22 (td, J=3.6, 10.8 Hz, 1H), 1.18-1.10 (m, 3H), 1.00-0.89 (m, 2H).

The compounds listed in Table 18 were made using the procedures of Scheme 18.

TABLE 18

| | Cpd. | Purity RT | | Observed | | Purity |
|---|---|---|---|---|---|---|
| Structure | No. | (min) | MW | m/z | Ion | Method |
| 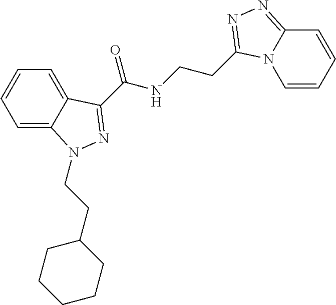 [SMILES: O=C(NCCc1nnc2ccccn12)c1nn(CCC2CCCCC2)c2ccccc12] | 402 | 0.46 | 4A.529 | 417.2 | [M + H]⁺ | 5 |
| 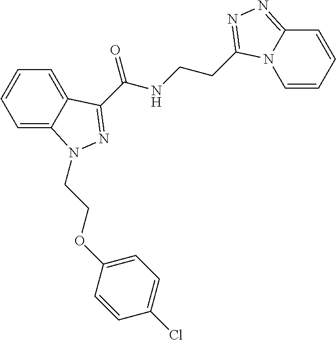 [SMILES: O=C(NCCc1nnc2ccccn12)c1nn(CCOc2ccc(Cl)cc2)c2ccccc12] | 485 | 0.468 | 460.92 | 461.3 | [M + H]⁺ | 5 |
| 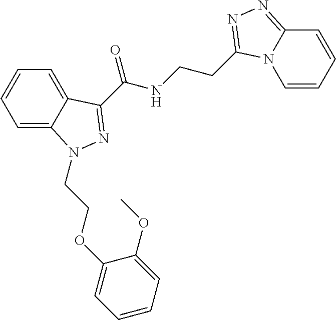 [SMILES: COc1ccccc1OCCn1nc(C(=O)NCCc2nnc3ccccn23)c2ccccc21] | 486 | 0.383 | 456.506 | 457.2 | [M + H]⁺ | 5 |
| 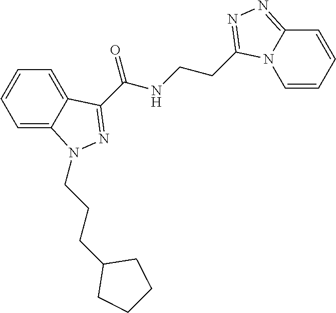 [SMILES: O=C(NCCc1nnc2ccccn12)c1nn(CCCC2CCCC2)c2ccccc12] | 487 | 0.507 | 4A.529 | 417.3 | [M + H]⁺ | 5 |
| 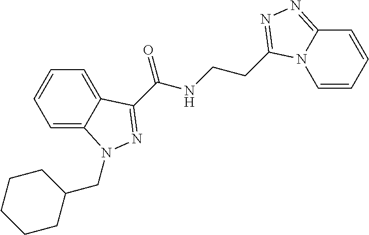 [SMILES: O=C(NCCc1nnc2ccccn12)c1nn(CC2CCCCC2)c2ccccc12] | 488 | 0.471 | 402.502 | 403.3 | [M + H]⁺ | 5 |
| 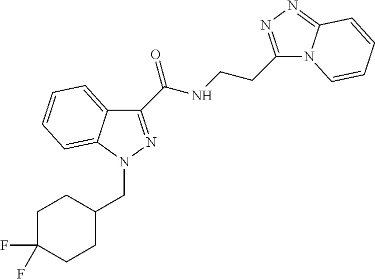 [SMILES: O=C(NCCc1nnc2ccccn12)c1nn(CC2CCC(F)(F)CC2)c2ccccc12] | 489 | 0.5435 | 438.483 | 439.2 | [M + H]⁺ | 5 |
| 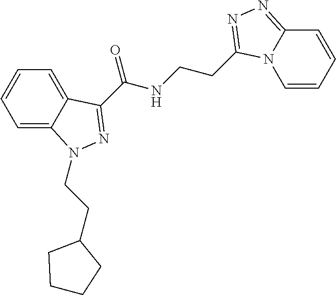 [SMILES: O=C(NCCc1nnc2ccccn12)c1nn(CCC2CCCC2)c2ccccc12] | 504 | 0.481 | 402.502 | 403.3 | [M + H]⁺ | 5 |
| 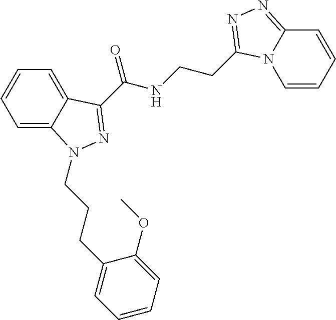 [SMILES: COc1ccccc1CCCn1nc(C(=O)NCCc2nnc3ccccn23)c2ccccc21] | 506 | 0.47 | 454.534 | 455.3 | [M + H]⁺ | 5 |

### Example 19

### Synthesis of Compound 445

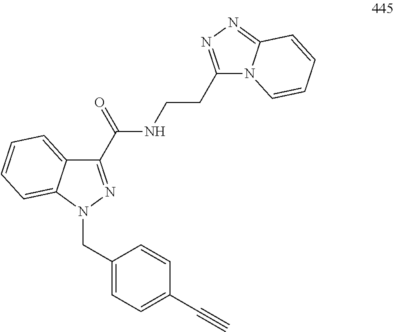 [SMILES: C#Cc1ccc(Cn2nc(C(=O)NCCc3nnc4ccccn34)c3ccccc32)cc1]

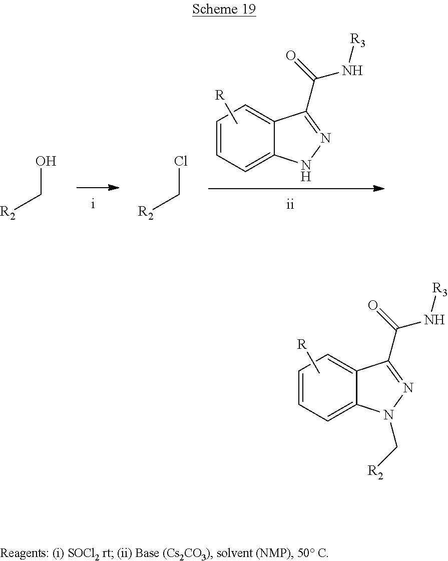

### Step 19-1. Synthesis of 2-cyclohexylethyl methanesulfonate

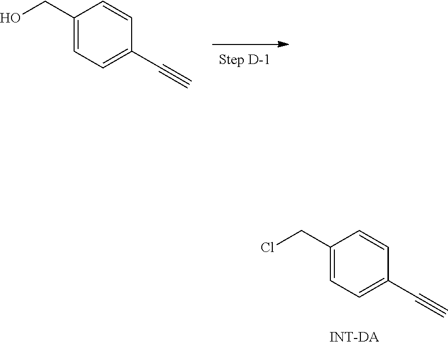

A solution of (4-ethynylphenyl)methanol (50 mg, 1 eq, 0.38 mmol) in SOCl₂ (1 mL) was stirred at 80° C. for 2 h. The reaction mixture was concentrated in vacuo to afford 50 mg (88% yield) of 1-(chloromethyl)-4-ethynyl-benzene as a yellow solid that was used without further purification; TLC (5:1 Pet ether/EA) R_{f}=0.70.

### Step 19-2 Synthesis of N-(2-([1,2,4]triazolo[4,3-a]pyridin-3-yl)ethyl)-1-(4-ethynylbenzyl)-1H-indazole-3-carboxamide

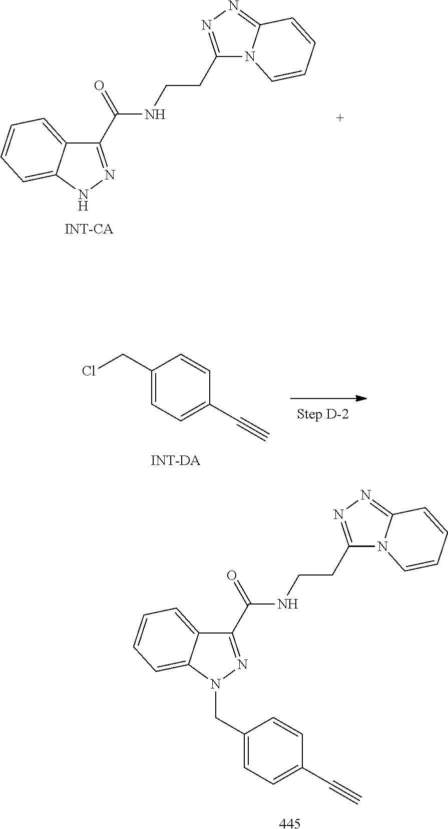

Into a solution of 1-(chloromethyl)-4-ethynyl-benzene (15 mg, 1 eq, 98 μmol) and N-[2-([1,2,4]triazolo[4,3-a]pyridin-3-yl)ethyl]-1H-indazole-3-carboxamide (30 mg, 1 eq, 98 μmol) in NMP (1 mL) was added Cs₂CO₃ (96 mg, 3 eq, 294 μmol). After stirring at 50° C. for 2 h, the reaction mixture was directly purified using reverse-phase prep HPLC (H₂O (formic acid)/ACN) to afford 24 mg (58% yield) of N-(2-([1,2,4]triazolo[4,3-a]pyridin-3-yl)ethyl)-1-(4-ethynylbenzyl)-1H-indazole-3-carboxamide (Compound 445) as an off-white solid; LCMS (m/z) calculated for C₂₅H₂₀N₆O: 420.2; found 421.2 [M+H]⁺, t_{R}=0.41 min (Method 5). ¹H NMR (400 MHz, DMSO-d6) δ=8.61 (t, J=6.0 Hz, 1H), 8.49 (d, J=7.0 Hz, 1H), 8.18 (d, J=8.1 Hz, 1H), 7.75 (dd, J=8.9, 19.8 Hz, 2H), 7.48-7.42 (in, 3H), 7.35-7.26 (m, 2H), 7.22 (d, J=8.3 Hz, 2H), 6.94 (t, J=6.8 Hz, 1H), 5.78 (s, 2H), 4.18 (s, 1H), 3.78 (br d, J=6.4 Hz, 2H), 3.40 (t, J=7.1 Hz, 2H).

The compounds listed in Table 19 were made using the procedures of Scheme 19.

TABLE 19

| | | Purity | | | | |
|---|---|---|---|---|---|---|
| | Cpd. | RT | | Observed | | Purity |
| Structure | No. | (min) | MW | m/z | Ion | Method |
| 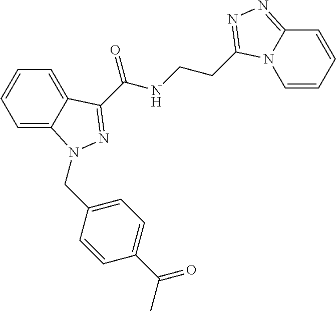 [SMILES: CC(=O)c1ccc(Cn2nc(C(=O)NCCc3nnc4ccccn34)c3ccccc32)cc1] | 413 | 0.444 | 438.491 | 439.3 | [M + H]⁺ | 6 |
| 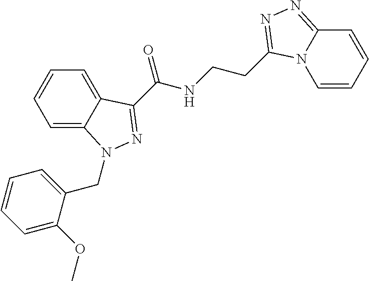 [SMILES: COc1ccccc1Cn1nc(C(=O)NCCc2nnc3ccccn23)c2ccccc21] | 430 | 0.334 | 426.48 | 427.2 | [M + H]⁺ | 5 |
| 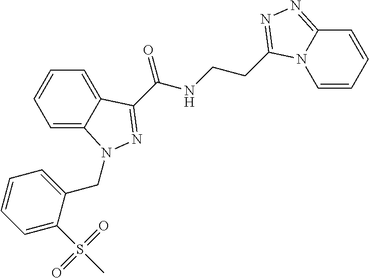 [SMILES: CS(=O)(=O)c1ccccc1Cn1nc(C(=O)NCCc2nnc3ccccn23)c2ccccc21] | 435 | 0.356 | 474.54 | 475.2 | [M + H]⁺ | 5 |
| 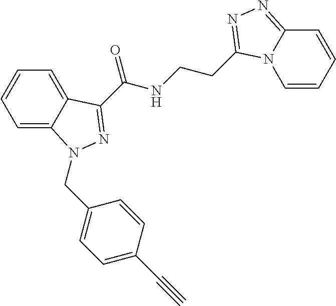 [SMILES: C#Cc1ccc(Cn2nc(C(=O)NCCc3nnc4ccccn34)c3ccccc32)cc1] | 445 | 0.407 | 420.476 | 421.2 | [M + H]⁺ | 5 |
| 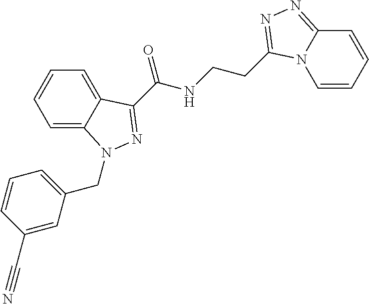 [SMILES: N#Cc1cccc(Cn2nc(C(=O)NCCc3nnc4ccccn34)c3ccccc32)c1] | 446 | 0.369 | 421.464 | 422.2 | [M + H]⁺ | 5 |
| 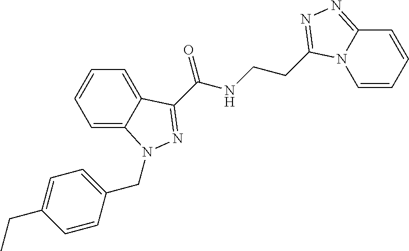 [SMILES: CCc1ccc(Cn2nc(C(=O)NCCc3nnc4ccccn34)c3ccccc32)cc1] | 447 | 0.364 | 424.508 | 425.2 | [M + H]⁺ | 5 |
| 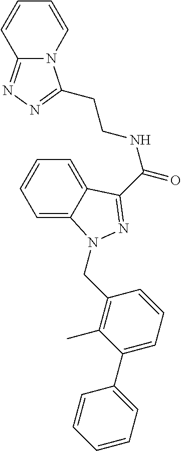 [SMILES: Cc1c(Cn2nc(C(=O)NCCc3nnc4ccccn34)c3ccccc32)cccc1-c1ccccc1] | 449 | 0.526 | 486.579 | 487.1 | [M + H]⁺ | 5 |
| 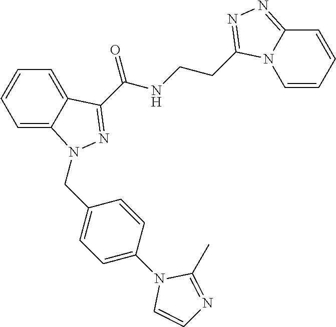 [SMILES: Cc1nccn1-c1ccc(Cn2nc(C(=O)NCCc3nnc4ccccn34)c3ccccc32)cc1] | 450 | 0.755 | 476.544 | 477.2 | [M + H]⁺ | 6 |
| 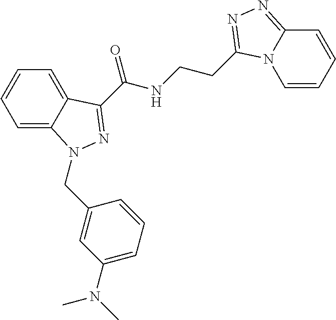 [SMILES: CN(C)c1cccc(Cn2nc(C(=O)NCCc3nnc4ccccn34)c3ccccc32)c1] | 451 | 0.305 | 439.523 | 440.3 | [M + H]⁺ | 6 |
| 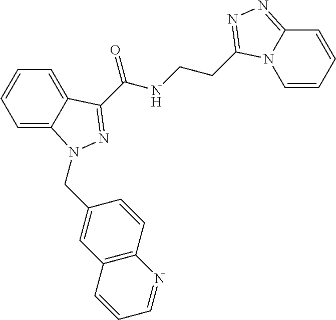 [SMILES: O=C(NCCc1nnc2ccccn12)c1nn(Cc2ccc3ncccc3c2)c2ccccc12] | 458 | 0.36 | 447.502 | 448.1 | [M + H]⁺ | 5 |
| 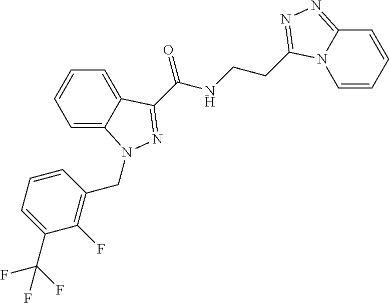 [SMILES: O=C(NCCc1nnc2ccccn12)c1nn(Cc2cccc(C(F)(F)F)c2F)c2ccccc12] | 459 | 0.456 | 482.443 | 483.2 | [M + H]⁺ | 6 |
| 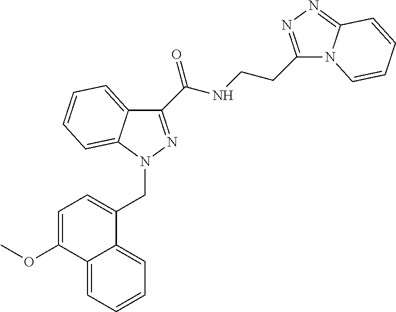 [SMILES: COc1ccc(Cn2nc(C(=O)NCCc3nnc4ccccn34)c3ccccc32)c2ccccc12] | 473 | 0.378 | 476.54 | 477.2 | [M + H]⁺ | 5 |
| 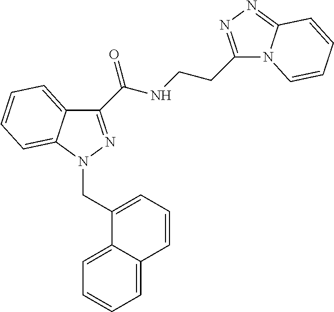 [SMILES: O=C(NCCc1nnc2ccccn12)c1nn(Cc2cccc3ccccc23)c2ccccc12] | 474 | 0.492 | 446.514 | 447.1 | [M + H]⁺ | 5 |
| 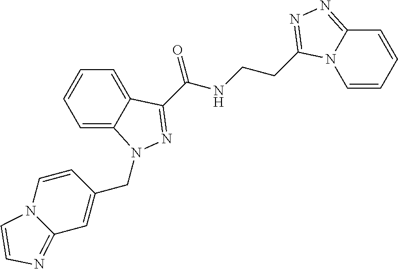 [SMILES: O=C(NCCc1nnc2ccccn12)c1nn(Cc2ccn3ccnc3c2)c2ccccc12] | 476 | 0.291 | 436.479 | 437.3 | [M + H]⁺ | 5 |
| 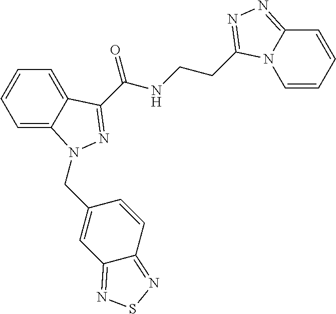 [SMILES: O=C(NCCc1nnc2ccccn12)c1nn(Cc2ccc3nsnc3c2)c2ccccc12] | 477 | 0.418 | 454.51 | 455.1 | [M + H]⁺ | 5 |
| 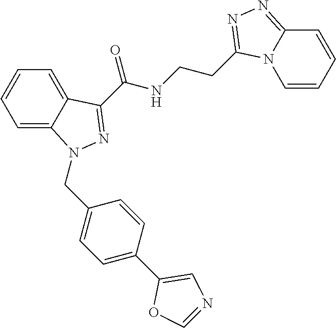 [SMILES: O=C(NCCc1nnc2ccccn12)c1nn(Cc2ccc(-c3cnco3)cc2)c2ccccc12] | 478 | 0.423 | 463.501 | 464.1 | [M + H]⁺ | 5 |
| 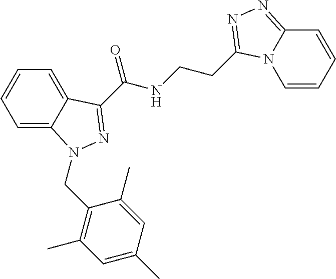 [SMILES: Cc1cc(C)c(Cn2nc(C(=O)NCCc3nnc4ccccn34)c3ccccc32)c(C)c1] | 479 | 0.479 | 438.535 | 439.3 | [M + H]⁺ | 5 |
| 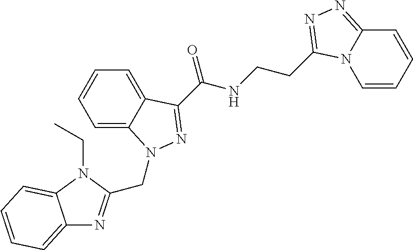 [SMILES: CCn1c(Cn2nc(C(=O)NCCc3nnc4ccccn34)c3ccccc32)nc2ccccc21] | 480 | 0.356 | 464.533 | 465.3 | [M + H]⁺ | 5 |
| 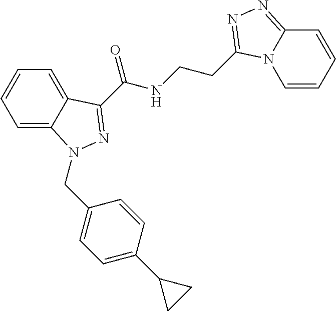 [SMILES: O=C(NCCc1nnc2ccccn12)c1nn(Cc2ccc(C3CC3)cc2)c2ccccc12] | 490 | 0.494 | 436.519 | 437 | [M + H]⁺ | 5 |
| 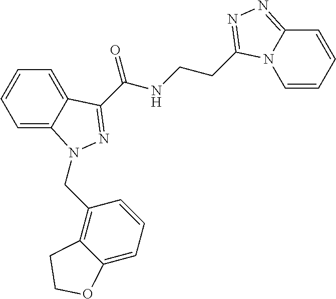 [SMILES: O=C(NCCc1nnc2ccccn12)c1nn(Cc2cccc3c2CCO3)c2ccccc12] | 491 | 0.452 | 438.491 | 439.2 | [M + H]⁺ | 5 |
| 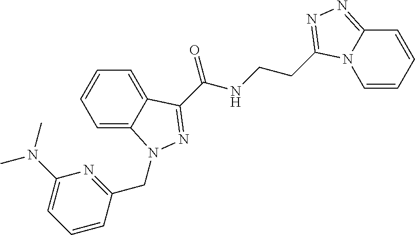 [SMILES: CN(C)c1cccc(Cn2nc(C(=O)NCCc3nnc4ccccn34)c3ccccc32)n1] | 492 | 0.325 | 440.511 | 441.3 | [M + H]⁺ | 5 |
| 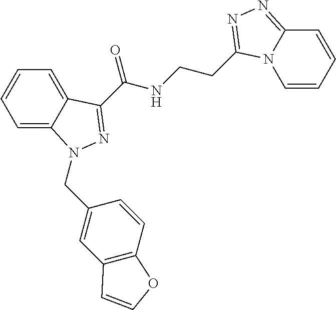 [SMILES: O=C(NCCc1nnc2ccccn12)c1nn(Cc2ccc3occc3c2)c2ccccc12] | 493 | 0.445 | 436.475 | 437.2 | [M + H]⁺ | 5 |
| 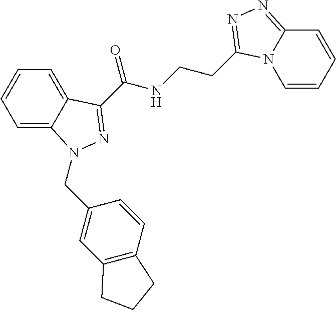 [SMILES: O=C(NCCc1nnc2ccccn12)c1nn(Cc2ccc3c(c2)CCC3)c2ccccc12] | 494 | 0.478 | 436.519 | 437.3 | [M + H]⁺ | 5 |
| 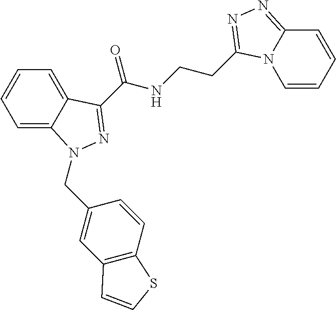 [SMILES: O=C(NCCc1nnc2ccccn12)c1nn(Cc2ccc3sccc3c2)c2ccccc12] | 495 | 0.461 | 452.54 | 453.3 | [M + H]⁺ | 5 |
| 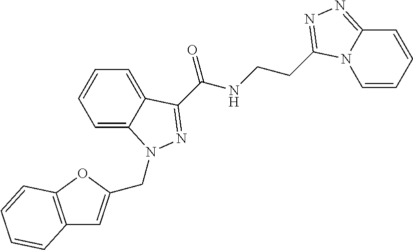 [SMILES: O=C(NCCc1nnc2ccccn12)c1nn(Cc2cc3ccccc3o2)c2ccccc12] | 496 | 0.48 | 436.475 | 437.2 | [M + H]⁺ | 5 |
| 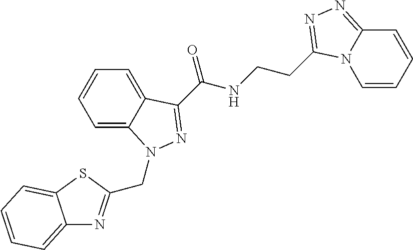 [SMILES: O=C(NCCc1nnc2ccccn12)c1nn(Cc2nc3ccccc3s2)c2ccccc12] | 497 | 0.456 | 453.52 | 454.1 | [M + H]⁺ | 5 |
| 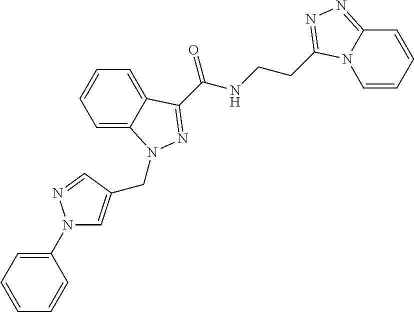 [SMILES: O=C(NCCc1nnc2ccccn12)c1nn(Cc2cnn(-c3ccccc3)c2)c2ccccc12] | 167 | 0.43 | 462.517 | 463.3 | [M + H]⁺ | 5 |
| 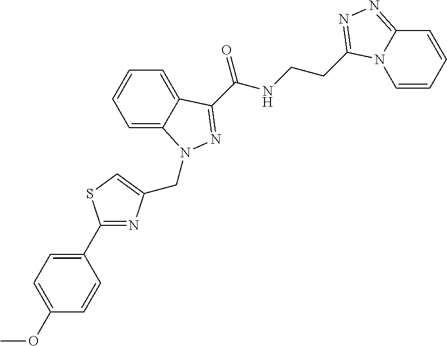 [SMILES: COc1ccc(-c2nc(Cn3nc(C(=O)NCCc4nnc5ccccn45)c4ccccc43)cs2)cc1] | 499 | 0.499 | 509.59 | 510.2 | [M + H]⁺ | 5 |
| 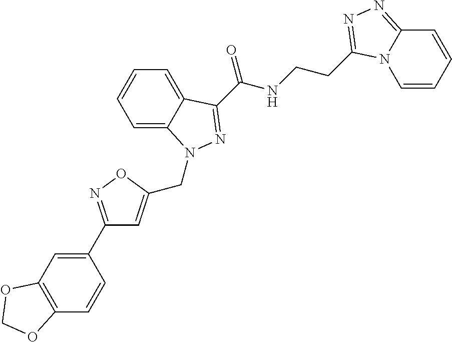 [SMILES: O=C(NCCc1nnc2ccccn12)c1nn(Cc2cc(-c3ccc4c(c3)OCO4)no2)c2ccccc12] | 500 | 0.459 | 507.51 | 508.3 | [M + H]⁺ | 5 |
| 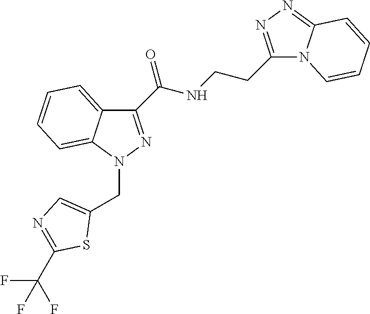 [SMILES: O=C(NCCc1nnc2ccccn12)c1nn(Cc2cnc(C(F)(F)F)s2)c2ccccc12] | 501 | 0.455 | 471.46 | 472.1 | [M + H]⁺ | 5 |
| 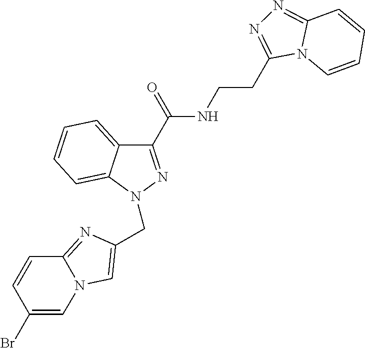 [SMILES: O=C(NCCc1nnc2ccccn12)c1nn(Cc2cn3cc(Br)ccc3n2)c2ccccc12] | 502 | 0.361 | 515.375 | 5A.9 | [M + H]⁺ | 5 |
| 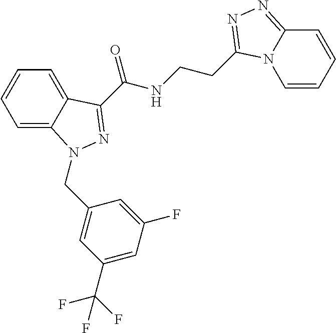 [SMILES: O=C(NCCc1nnc2ccccn12)c1nn(Cc2cc(F)cc(C(F)(F)F)c2)c2ccccc12] | 509 | 0.485 | 482.443 | 483.2 | [M + H]⁺ | 5 |
| 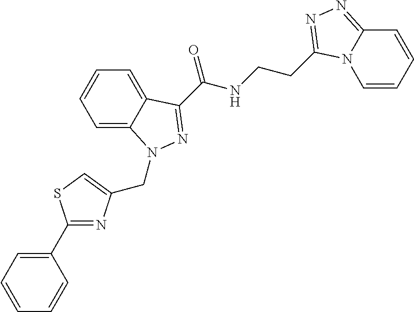 [SMILES: O=C(NCCc1nnc2ccccn12)c1nn(Cc2csc(-c3ccccc3)n2)c2ccccc12] | 512 | 0.473 | 479.56 | 480.3 | [M + H]⁺ | 5 |

### Example 20

### MRGPR X4 Activity

HEK cells stably transfected to express human MIRGPR X4 were maintained in an incubator at 37° C. with 5% CO₂ and grown in DMEM media with 10% fetal bovine serum (FBS) and 100 each of sodium pyruvate, Glutamax, penicillin/streptomycin, and Geneticin. HEK cells stably transfected to express mouse MRGPR A1 were maintained in the same incubator and grown in DMEM media with 10% FBS, 1% each of sodium pyruvate, Glutamax, penicillin/streptomycin, Geneticin, and 2.2 mg/mL Hygromycin.

Cells were plated in a 384-well assay plate at 20,000 cells per well in 12 μL of Opti-MEM and kept in an incubator overnight. On the day of the assay, compounds solubilized at 10 mM in DMSO were added as a 10-point curve (10 uM final top concentration with 1:3 serial dilutions) using a Tecan D300E digital dispenser. Agonists were diluted in assay buffer (final concentrations of 5.7 mM Tris-HCl, 43 mM NaCl, 50 mM LiCl, pH=8) and 2 μL of the appropriate agonist are added to each well. Final concentrations of agonists were 10 μM bilirubin, 20 μM deoxycholic acid, or 100 μM conjugated bilirubin (obtained from Lee Biosolutions, catalog #910-12). Final concentrations of DMSO were kept consistent across the plate. Plates were incubated in the dark for 1 h at 37° C. and then for 30 minutes at room temperature. IP-1 standards and HTRF detection reagents were added according to the IP-One—Gq Kit purchased from Cisbio (part number 62IPAPEJ) and incubated in the dark for 1 h at room temperature. The plate was read on a Molecular Devices SpectraMax iD5 plate reader. The HTRF ratio was calculated from the raw data and graphed using GraphPad Prism to calculate an IC₅₀ value for each compound.

Activity data for selected MRGPR X4 antagonists (versus 20 μM deoxycholic acid agonist) are displayed in Table B. The activity ranges are denoted as follows: “+++++” denotes antagonist activity <100 nM; “++++” denotes antagonist activity between 100 and 500 nM; “+++” denotes activity between 501 and 1000 nM; “++” denotes activity between 1001 and 2500 nM; and “+” denotes activity >2500 nM

TABLE B

| | MRGPRX4 |
|---|---|
| | Antagonist |
| Compound No. | Activity (nM) |
| 1 | +++++ |
| 2 | +++++ |
| 3 | +++++ |
| 4 | +++++ |
| 5 | +++++ |
| 6 | +++++ |
| 7 | +++++ |
| 8 | +++++ |
| 9 | +++++ |
| 10 | +++++ |
| 11 | +++++ |
| 12 | +++++ |
| 13 | +++++ |
| 14 | +++++ |
| 15 | +++++ |
| 16 | +++++ |
| 17 | +++++ |
| 18 | +++++ |
| 19 | +++++ |
| 20 | +++++ |
| 21 | +++++ |
| 22 | +++++ |
| 23 | +++++ |
| 24 | +++++ |
| 25 | +++++ |
| 26 | +++++ |
| 27 | +++++ |
| 28 | ++++ |
| 29 | ++++ |
| 30 | ++++ |
| 31 | ++++ |
| 32 | ++++ |
| 33 | ++++ |
| 34 | ++++ |
| 35 | ++++ |
| 36 | ++++ |
| 37 | ++++ |
| 38 | ++++ |
| 39 | ++++ |
| 40 | ++++ |
| 41 | ++++ |
| 42 | ++++ |
| 43 | ++++ |
| 44 | ++++ |
| 45 | ++++ |
| 46 | ++++ |
| 47 | ++++ |
| 48 | ++++ |
| 49 | ++++ |
| 50 | ++++ |
| 51 | ++++ |
| 52 | ++++ |
| 53 | ++++ |
| 54 | ++++ |
| 55 | ++++ |
| 56 | ++++ |
| 57 | ++++ |
| 58 | ++++ |
| 59 | ++++ |
| 60 | ++++ |
| 61 | ++++ |
| 62 | ++++ |
| 63 | ++++ |
| 64 | ++++ |
| 65 | ++++ |
| 66 | ++++ |
| 67 | ++++ |
| 68 | ++++ |
| 69 | ++++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | ++ |
| 94 | ++ |
| 95 | ++ |
| 96 | ++ |
| 97 | ++ |
| 98 | ++ |
| 99 | ++ |
| 100 | ++ |
| 101 | ++ |
| 102 | ++ |
| 103 | ++ |
| 104 | ++ |
| 105 | ++ |
| 106 | ++ |
| 107 | ++ |
| 108 | ++ |
| 109 | ++ |
| 110 | ++ |
| 111 | ++ |
| 112 | ++ |
| 113 | ++ |
| 114 | ++ |
| 115 | ++ |
| 116 | ++ |
| 117 | ++ |
| 118 | ++ |
| 119 | ++ |
| 120 | ++ |
| 121 | ++ |
| 122 | ++ |
| 123 | + |
| 124 | + |
| 125 | + |
| 126 | + |
| 127 | + |
| 128 | + |
| 129 | + |
| 130 | + |
| 131 | ++++ |
| 132 | ++++ |
| 133 | ++ |
| 134 | ++ |
| 135 | ++ |
| 136 | ++++ |
| 137 | +++++ |
| 138 | +++++ |
| 139 | ++++ |
| 140 | +++ |
| 141 | +++ |
| 142 | + |
| 143 | ++++ |
| 144 | ++++ |
| 145 | +++ |
| 146 | + |
| 147 | + |
| 148 | ++ |
| 149 | +++++ |
| 150 | ++++ |
| 151 | +++++ |
| 152 | +++++ |
| 153 | +++++ |
| 154 | +++++ |
| 155 | ++++ |
| 156 | ++++ |
| 157 | ++++ |
| 158 | ++++ |
| 159 | ++++ |
| 160 | ++++ |
| 161 | ++++ |
| 162 | ++++ |
| 163 | ++++ |
| 164 | ++++ |
| 165 | ++++ |
| 166 | ++++ |
| 167 | ++++ |
| 168 | ++++ |
| 169 | ++++ |
| 170 | ++++ |
| 171 | ++++ |
| 172 | ++++ |
| 173 | ++++ |
| 174 | ++++ |
| 175 | ++++ |
| 176 | ++++ |
| 177 | ++++ |
| 178 | ++++ |
| 179 | ++++ |
| 180 | ++++ |
| 181 | ++++ |
| 182 | ++++ |
| 183 | +++ |
| 184 | +++ |
| 185 | +++ |
| 186 | +++ |
| 187 | +++ |
| 188 | +++ |
| 189 | +++ |
| 190 | +++ |
| 191 | +++ |
| 192 | +++ |
| 193 | +++ |
| 194 | +++ |
| 195 | +++ |
| 196 | +++ |
| 197 | +++ |
| 198 | +++ |
| 199 | +++ |
| 200 | ++ |
| 201 | ++ |
| 202 | ++ |
| 203 | ++ |
| 204 | ++ |
| 205 | ++ |
| 206 | ++ |
| 207 | ++ |
| 208 | ++ |
| 209 | ++ |
| 210 | ++ |
| 211 | ++ |
| 212 | ++ |
| 213 | ++ |
| 214 | ++ |
| 215 | ++ |
| 216 | ++ |
| 217 | ++ |
| 218 | ++ |
| 219 | ++ |
| 220 | ++ |
| 221 | ++ |
| 222 | ++ |
| 223 | ++ |
| 224 | + |
| 225 | + |
| 226 | + |
| 227 | + |
| 228 | + |
| 229 | + |
| 230 | + |
| 231 | + |
| 232 | + |
| 233 | + |
| 234 | + |
| 235 | + |
| 236 | + |
| 237 | ++++ |
| 238 | ++++ |
| 239 | ++++ |
| 240 | ++++ |
| 241 | ++++ |
| 242 | +++ |
| 243 | +++ |
| 244 | +++ |
| 245 | +++ |
| 246 | ++ |
| 247 | ++ |
| 248 | ++ |
| 249 | ++ |
| 250 | ++ |
| 251 | ++ |
| 252 | ++ |
| 253 | ++ |
| 254 | ++ |
| 255 | + |
| 256 | + |
| 257 | + |
| 258 | ++++ |
| 259 | ++++ |
| 260 | +++ |
| 261 | +++ |
| 262 | +++ |
| 263 | +++ |
| 264 | +++ |
| 265 | +++ |
| 266 | +++ |
| 267 | +++ |
| 268 | +++ |
| 269 | ++ |
| 270 | ++ |
| 271 | ++ |
| 272 | ++ |
| 273 | ++ |
| 274 | ++ |
| 275 | ++ |
| 276 | ++ |
| 277 | ++ |
| 278 | ++ |
| 279 | ++ |
| 280 | ++ |
| 281 | ++ |
| 282 | ++ |
| 283 | ++ |
| 284 | ++ |
| 285 | ++ |
| 286 | ++ |
| 287 | ++ |
| 288 | ++ |
| 289 | ++ |
| 290 | ++ |
| 291 | ++ |
| 292 | ++ |
| 293 | ++ |
| 294 | ++ |
| 295 | ++ |
| 296 | + |
| 297 | + |
| 298 | + |
| 299 | + |
| 300 | + |
| 301 | + |
| 302 | + |
| 303 | + |
| 304 | + |
| 305 | + |
| 306 | + |
| 307 | + |
| 308 | + |
| 309 | ++ |
| 310 | + |
| 311 | ++ |
| 312 | ++ |
| 313 | + |
| 314 | ++ |
| 315 | ++ |
| 316 | ++ |
| 317 | +++ |
| 318 | ++ |
| 319 | ++ |
| 320 | ++ |
| 321 | ++ |
| 322 | ++ |
| 323 | ++ |
| 324 | + |
| 325 | + |
| 326 | + |
| 327 | + |
| 328 | + |
| 329 | + |
| 330 | + |
| 331 | + |
| 332 | + |
| 333 | ++ |
| 334 | +++ |
| 335 | + |
| 336 | ++++ |
| 337 | + |
| 338 | ++++ |
| 339 | +++ |
| 340 | + |
| 341 | + |
| 342 | +++ |
| 343 | +++ |
| 344 | + |
| 345 | + |
| 346 | ++ |
| 347 | + |
| 348 | ++ |
| 349 | +++ |
| 350 | ++ |
| 351 | + |
| 352 | ++++ |
| 353 | + |
| 354 | + |
| 355 | + |
| 356 | +++ |
| 357 | +++++ |
| 358 | +++++ |
| 359 | + |
| 360 | + |
| 361 | +++++ |
| 362 | ++ |
| 363 | + |
| 364 | ++++ |
| 365 | + |
| 366 | +++++ |
| 367 | ++++ |
| 368 | ++++ |
| 369 | + |
| 370 | + |
| 371 | + |
| 372 | + |
| 373 | +++ |
| 374 | + |
| 375 | + |
| 376 | +++ |
| 377 | + |
| 378 | + |
| 379 | + |
| 380 | + |
| 381 | ++ |
| 382 | +++ |
| 383 | ++ |
| 384 | + |
| 385 | + |
| 386 | + |
| 387 | + |
| 388 | + |
| 389 | + |
| 390 | + |
| 391 | ++ |
| 392 | ++ |
| 393 | + |
| 395 | + |
| 396 | + |
| 397 | ++++ |
| 398 | + |
| 399 | + |
| 400 | ++++ |
| 401 | +++ |
| 402 | ++++ |
| 403 | ++++ |
| 404 | ++ |
| 405 | + |
| 406 | ++ |
| 407 | ++ |
| 408 | ++++ |
| 409 | ++ |
| 410 | + |
| 411 | +++++ |
| 412 | ++++ |
| 413 | ++++ |
| 414 | ++++ |
| 415 | +++ |
| 416 | +++++ |
| 418 | + |
| 419 | ++++ |
| 420 | ++ |
| 421 | +++ |
| 422 | + |
| 423 | + |
| 424 | +++ |
| 425 | ++ |
| 426 | ++ |
| 427 | + |
| 428 | + |
| 429 | +++ |
| 430 | + |
| 431 | ++ |
| 432 | +++++ |
| 433 | +++ |
| 434 | ++ |
| 435 | + |
| 436 | ++++ |
| 437 | ++++ |
| 438 | ++++ |
| 439 | ++++ |
| 440 | + |
| 441 | ++ |
| 442 | + |
| 443 | + |
| 444 | + |
| 445 | ++++ |
| 446 | ++ |
| 447 | ++++ |
| 448 | + |
| 449 | ++++ |
| 450 | + |
| 451 | ++++ |
| 452 | +++ |
| 453 | ++ |
| 454 | ++ |
| 455 | ++++ |
| 456 | ++++ |
| 457 | + |
| 458 | +++ |
| 459 | ++++ |
| 460 | +++ |
| 46 | ++ |
| 462 | ++ |
| 463 | +++++ |
| 464 | ++ |
| 465 | ++++ |
| 466 | ++++ |
| 467 | ++++ |
| 468 | ++ |
| 469 | ++ |
| 470 | + |
| 471 | + |
| 472 | + |
| 473 | ++++ |
| 474 | ++++ |
| 475 | + |
| 476 | + |
| 477 | +++++ |
| 478 | +++ |
| 479 | ++++ |
| 480 | +++ |
| 481 | +++ |
| 482 | +++++ |
| 483 | +++ |
| 484 | ++ |
| 485 | +++ |
| 486 | +++ |
| 487 | ++++ |
| 488 | +++ |
| 489 | +++ |
| 490 | +++++ |
| 491 | ++++ |
| 492 | + |
| 493 | +++++ |
| 494 | +++++ |
| 495 | +++++ |
| 496 | +++++ |
| 497 | +++++ |
| 498 | ++++ |
| 499 | +++ |
| 500 | ++ |
| 501 | ++++ |
| 502 | + |
| 503 | +++ |
| 504 | +++ |
| 505 | ++ |
| 506 | + |
| 507 | ++++ |
| 508 | ++++ |
| 509 | ++++ |
| 510 | + |
| 511 | ++++ |
| 512 | ++++ |
| 513 | + |
| 514 | ++++ |
| 515 | + |
| 516 | + |
| 517 | ++ |
| 518 | ++ |
| 519 | +++ |
| 520 | + |
| 521 | ++ |
| 522 | +++ |
| 523 | ++ |
| 524 | +++++ |
| 525 | +++++ |
| 526 | +++++ |
| 527 | +++++ |
| 528 | +++++ |
| 529 | +++++ |
| 530 | +++++ |
| 531 | +++++ |
| 532 | +++++ |
| 533 | +++++ |
| 534 | +++++ |
| 535 | +++++ |
| 536 | +++++ |
| 537 | +++++ |
| 538 | +++++ |
| 539 | +++++ |
| 540 | +++++ |
| 541 | +++++ |
| 542 | +++++ |
| 543 | ++++ |
| 544 | ++++ |
| 545 | ++++ |
| 546 | ++++ |
| 547 | +++ |
| 548 | +++ |
| 549 | ++++ |
| 550 | ++++ |
| 551 | +++++ |
| 552 | +++++ |
| 553 | +++++ |
| 554 | +++++ |
| 555 | +++++ |
| 556 | +++++ |
| 557 | ++++ |
| 558 | +++++ |
| 559 | +++++ |
| 560 | ++++ |
| 561 | +++++ |
| 562 | +++++ |
| 563 | +++++ |
| 564 | +++++ |
| 565 | +++++ |
| 566 | ++++ |
| 567 | ++ |
| 568 | +++ |
| 569 | ++++ |
| 570 | + |
| 571 | +++++ |
| 572 | +++++ |
| 573 | +++++ |
| 574 | ++++ |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 63/079,870, filed Sep. 17, 2020, which application is hereby incorporated by reference in its entirety.

1. A method of modulating a Mas-Related G-Protein Receptor (MRGPR) X4 by contacting the MRGPR X4 with an effective amount of a compound having structure (I): 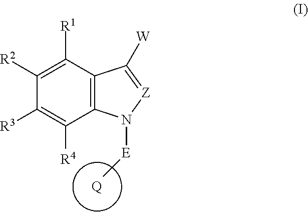
or a pharmaceutically acceptable salt, isomer, hydrate, solvate or isotope thereof, wherein:
W is 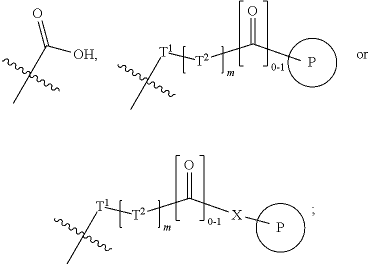
P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ—;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)— or —C(O)NH—;
R^{p} and R^{q} are at each occurrence, independently H, —OH, —NH₂, —N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH, or C₁-C₄ alkyl;
R^{t} is at each occurrence independently H, C₁-C₄ alkyl, or cycloalkyl;
R^{t′} is at each occurrence independently H, OH, C₁-C₄ alkyl, cycloalkyl or R and R together with the atom to which they are bonded form a ring;
R¹, R², R³, and R⁴ are at each occurrence independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

2. A method for treating an MRGPR X4-dependent condition by administering to a subject in need thereof an effective amount of a compound having structure (I): 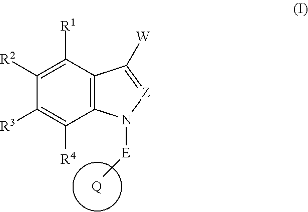
or a pharmaceutically acceptable salt, isomer, hydrate, solvate or isotope thereof, wherein:
W is 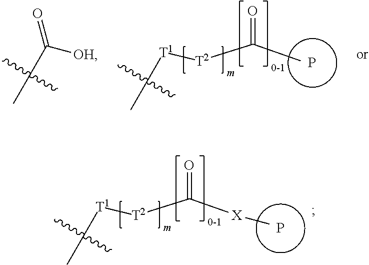
P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ—;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)— or —C(O)NH—;
R^{p} and R^{q} are at each occurrence, independently H, —OH, —NH₂, —N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH, or C₁-C₄ alkyl;
R^{t} is at each occurrence independently H, C₁-C₄ alkyl, or cycloalkyl;
R^{t′} is at each occurrence independently H, OH, C₁-C₄ alkyl, cycloalkyl or R and R^{t′} together with the atom to which they are bonded form a ring;
R¹, R², R³, and R⁴ are at each occurrence, independently H, —OH, —NH₂, halo, —CO₂H, —C(O)Me, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

3. The method of claim 2, wherein the MRGPR X4-dependent condition is an itch associated condition, a pain associated condition, or an autoimmune disorder.

4-8. (canceled)

9. The method of claim 2, wherein the compound of structure (I) has formula (IA): 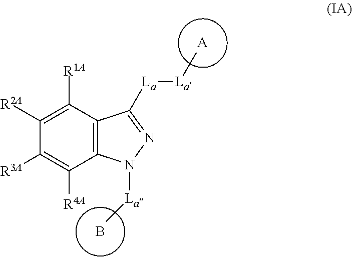
or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{a};
B is C₆-C₁₀ aryl, C₃-C₁₀ cycloalkyl, 3-10 membered monocyclic or bicyclic heterocyclyl, or 5-10 membered monocyclic or bicyclic heteroaryl, each of which is optionally substituted with one or more R^{a′};
Lₐ is —C(O)NH—, or —N(H)C(O)—;
L_{a′} is a bond, —CH₂C(O)—, or —(C(R^{2a})(R^{2a′}))₂—;
L_{a″} is —(C(R^{3a})(R^{3a′}))ₙ;
n is 1 or 2;
R^{a} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{3a} is at each occurrence independently H, OH or C₁-C₄ alkyl;
R^{3a′} is at each occurrence independently H, OH or C₁-C₄ alkyl;
R^{2a} is at each occurrence independently H or C₁-C₄ alkyl;
R^{2a′} is at each occurrence H;
R^{a′} is at each occurrence independently H, —OH, —NH₂, halo, —CO₂H, —C(O)Me-, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl; and
R^{1A}, R^{2A}, R^{3A} and R^{4A} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or heteroaryl.

10. The method of claim 2, wherein the compound of structure (I) has formula (IB): 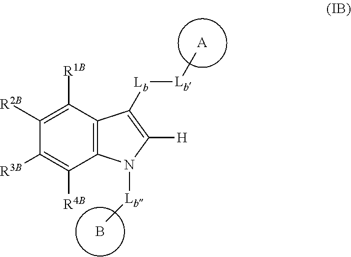
or a pharmaceutically acceptable salt, isomer, hydrate, solvate or isotope thereof, wherein:
A is C₆-C₁₀ aryl, C₃-C₁₀ cycloalkyl, 7-10 membered monocyclic or bicyclic heterocyclyl, 5-10 membered monocyclic or bicyclic heteroaryl, each of which is optionally substituted with one or more R^{b};
B is phenyl or heterocyclyl, optionally substituted with one or more R^{b′};
L_{b} is —C(O)NH—;
L_{b′} is —(C(R^{2b})(R^{2b′}))—, —(C(R^{2b})(R^{2b′}))₂— or —(C(R^{2b})(R^{2b′}))₃—
L_{b″} is —(CH₂)ₙ—;
n is 1 or 2;
R^{b} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2b} is at each occurrence independently H, C₁-C₄ alkyl or cycloalkyl;
R^{2b′} is at each occurrence independently H, OH, C₁-C₄ alkyl, cycloalkyl or R^{2b} and R^{2b′} together with the atom to which they are bonded form a ring;
R^{b′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
R^{1B}, R^{2B}, R^{3B}, and R^{4B} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

11. The method of claim 2, wherein the compound of structure (I) has formula (IC): 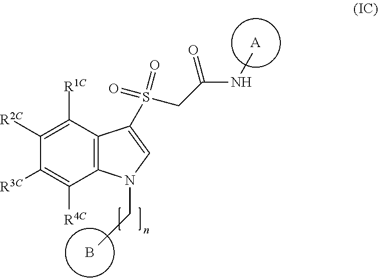
or a pharmaceutically acceptable salt, isomer, hydrate, solvate or isotope thereof, wherein:
A is phenyl, pyridyl or C₁-C₄ alkyl, each of which is optionally substituted with one or more R^{c};
B is phenyl or pyrrolidine optionally substituted with one or more R^{c′};
R^{c} is at each occurrence independently H, —OH, —NH₂, halo, —CO₂H, alkoxy, cyano, or C₁-C₆ alkyl;
R^{c′} is at each occurrence independently H, or chloro;
R^{1C}, R^{2C}, R^{3C}, and R^{4C} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₂-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 1 or 2.

12. The method of claim 2, wherein the compound of structure (I) has formula (ID): 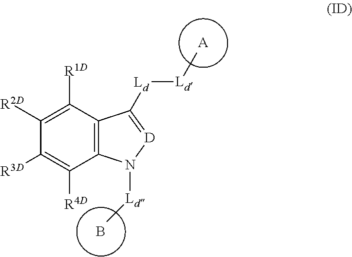
or a pharmaceutically acceptable salt, oisomer, hydrate, solvate or isotope thereof, wherein:
A is phenyl, or 5-6 membered heteroaryl, each of which is optionally substituted with one or more R^{d};
B is phenyl optionally substituted with one or more R^{d′};
D is N or C;
L_{d} is —C(O)NHS(O)₂—;
L_{d′} is —(C(R^{2d})(R^{2d′}))ₙ—
L_{d″} is —(CH₂)—;
R^{d} is at each occurrence independently H, —OH, —NH₂, halo, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 5-10 membered heteroaryl;
R^{2d} is at each occurrence H;
R^{2d′} is at each occurrence independently H or C₁-C₄ alkyl;
R^{d′} is at each occurrence independently H, —OH, —NH₂, halo, —CO₂H, —C(O)Me-alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl;
R^{1D}, R^{2D}, R^{3D}, and R^{4D} are at each occurrence, independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 0 or 1.

13. The method of claim 2, wherein the compound of structure (I) has formula (IE): 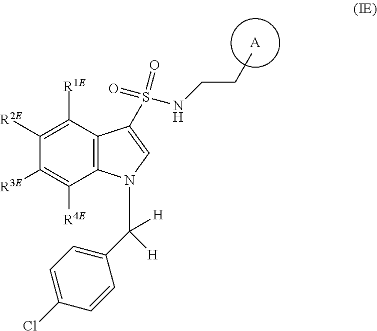
or a pharmaceutically acceptable salt, isomer, hydrate, solvate or isotope thereof, wherein:
A is phenyl optionally substituted with one or more R^{e};
R^{e} is at each occurrence independently H, —OH, —NH₂, halo, —CO₂H, alkoxy, cyano, or C₁-C₆ alkyl; and
R^{1E}, R^{2E}, R^{3E}, and R^{4E} are at each occurrence independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl.

14. The method of claim 2, wherein
the compound of structure (I) has formula (IF): 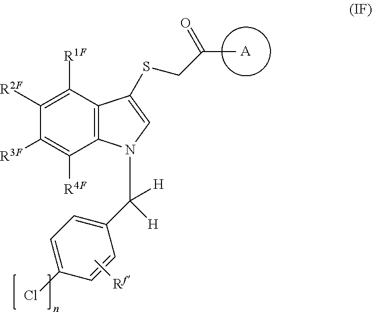
or a pharmaceutically acceptable salt, isomer, hydrate, solvate or isotope thereof, wherein:
A is an isolated pyrrolidine bonded through its nitrogen atom to the carbon chain, and which is optionally substituted with one or more R^{f};
R^{f} is H, —OH, halo, alkoxy or C₁-C₆ alkyl;
R^{f′} is chloro or bromo;
R^{1F}, R^{2F}, R^{3F}, and R^{4F} are at each occurrence, independently H, —OH, —NH₂, chloro, fluoro, —CO₂H, alkoxy, cyano, C₁-C₆ alkyl, C₁-C₆ haloalkyl, C₁-C₆ haloalkoxy, C₁-C₆ alkynyl, hydroxylalkly, alkoxyalkyl, aminylalkyl, alkylaminylalkyl, cyanoalkyl, carboxyalkyl, aminylcarbonylalkyl, aryl, cycloalkyl, heterocyclyl or 6-10 membered heteroaryl; and
n is 0 or 1.

15. The method of claim 2, wherein the compound of structure (I) has formula (IG): 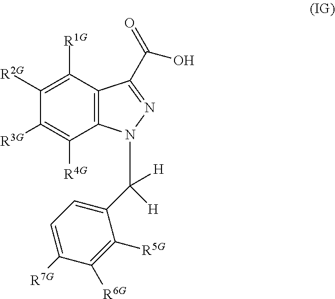
or a pharmaceutically acceptable salt, isomer, hydrate, solvate or isotope thereof, wherein:
R^{1G} is H or fluoro;
R^{2G} is H, chloro or fluoro;
R^{3G} is H or fluoro;
R^{4G} is H;
R^{5G} is H or chloro;
R^{6G} is H, methoxy or trifluoromethoxy; and
R^{7G} is H, chloro, methyl, isopropyl, trifluoromethoxy, trifluoromethyl or difluoromethoxy.

16. The method of claim 2, wherein the compound of structure (I) has formula (IH): 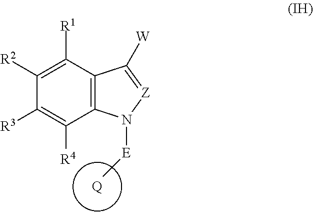
or a pharmaceutically acceptable salt, isomer, hydrate, solvate or isotope thereof, wherein:
W is 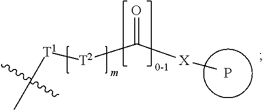
P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence, independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl;
R^{t′} is at each occurrence independently H, OH, C₁-C₄ alkyl, cycloalkyl or R and R together with the atom to which they are bonded form a ring;
R¹, R², R³, and R⁴ are at each occurrence, independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

17. A pharmaceutical composition comprising a pharmaceutically acceptable carrier and a compound having structure (I): 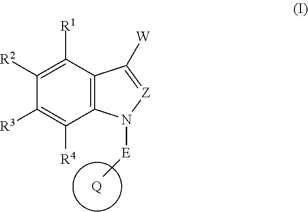
or a pharmaceutically acceptable salt, isomer, hydrate, solvate or isotope thereof, wherein:
W is 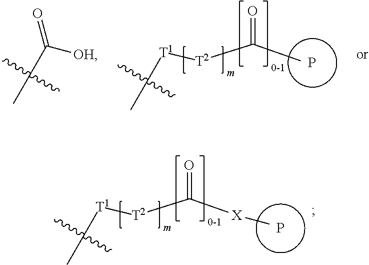
P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)—, —C(O)NH—;
R^{p} and R^{q} are at each occurrence, independently H, —OH, —NH₂, N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH or C₁-C₄ alkyl;
R^{t} is at each occurrence independently H, C₁-C₄ alkyl or cycloalkyl;
R^{t′} is at each occurrence independently H, OH, C₁-C₄ alkyl, cycloalkyl or R and R together with the atom to which they are bonded form a ring;
R¹, R², R³, and R⁴ are at each occurrence, independently H, —OH, —NH₂, halo, —CO₂H, —C(O)Me, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

18-25. (canceled)

26. A pharmaceutical composition comprising a pharmaceutically acceptable carrier and a compound of Table A, or a pharmaceutically acceptable salt, isomer, hydrate, solvate or isotope thereof.

27-35. (canceled)

36. A compound having structure (I): 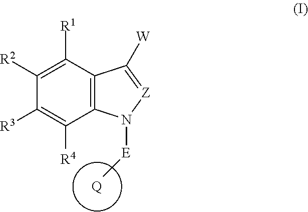
or a pharmaceutically acceptable salt, isomer, hydrate, solvate or isotope thereof, wherein:
W is 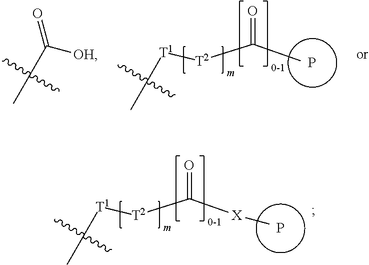
P is C₁-C₄ alkyl, cycloalkyl, aryl, heteroaryl or heterocyclyl, each of which is optionally substituted with one or more R^{p};
Q is cycloalkyl, aryl, heteroaryl or heterocyclyl, each of which is optionally substituted with one or more R^{q};
T¹ is —C(O)NH—, —N(H)C(O)—, —S(O)₂CH₂C(O)N(H)—, —C(O)N(H)S(O)₂—, —S(O)₂N(H)—, or —SCH₂C(O)—;
T² is —(C(R^{t})(R^{t′}))—;
E is —(C(H)R^{e})ₙ— or —(C(H)R^{e})ₙ—X²—(C(R^{t})(R^{t′})ₚ—;
Z is C or N;
X is —X¹—(C(R^{t})(R^{t′})ₚ;
X¹ is —O—, —NH—, —N(H)C(O)—, —C(O)NH— or —S(═O)₂—;
X² is —O—, —C(═O)—, —N(H)C(O)— or —C(O)NH—;
R^{p} and R^{q} are at each occurrence, independently H, —OH, —NH₂, —N(Me)₂, —NHC(═NH)NH₂, —S(═O)₂Me, —NHS(═O)₂Me, halo, oxo, —CO₂H, —C(O)Me, —C(O)NHMe, alkoxy, haloalkoxy, cyano, C₁-C₆ alkyl, C₁-C₆ haloalkyl, C₁-C₆ haloalkoxy, C₂-C₆ alkenyl, C₂-C₆ alkynyl, hydroxylalkyl, alkoxyalkyl, aminylalkyl, alkylaminylalkyl, cyanoalkyl, carboxyalkyl, aminylcarbonylalkyl, cycloalkyl, aryl, heteroaryl or heterocyclyl;
R^{e} is at each occurrence independently H, OH, or C₁-C₄ alkyl;
R^{t} is at each occurrence independently H, C₁-C₄ alkyl, or cycloalkyl;
R^{t′} is at each occurrence independently H, OH, C₁-C₄ alkyl, cycloalkyl or R and R together with the atom to which they are bonded form a ring;
R¹, R², R³, and R⁴ are at each occurrence independently H, —OH, —NH₂, halo, —C(O)Me, —CO₂H, alkoxy, cyano, C₁-C₆ alkyl, C₁-C₆ haloalkyl, C₁-C₆ haloalkoxy, C₂-C₆ alkenyl, C₂-C₆ alkynyl, hydroxylalkly, alkoxyalkyl, aminylalkyl, alkylaminylalkyl, cyanoalkyl, carboxyalkyl, aminylcarbonylalkyl, cycloalkyl, aryl, heteroaryl or heterocyclyl;
m is 0, 1, 2 or 3;
n is 0, 1, 2 or 3; and
p is 0 or 1.

37. The compound of claim 36, wherein the compound is selected from any one of the compounds listed in Table A, or a pharmaceutically acceptable salt, isomer, hydrate, solvate or isotope thereof.
